(19) United States
(12) Patent Application Publication
(10) Pub. No.: US 2026/0256940 A1
(43) Pub. Date: Sep. 3, 2026

(54) MITOCHONDRIA AS A TARGETED DELIVERY PLATFORM

(71) Applicant: cellvie AG, Zürich (CH)

(72) Inventors: Dedy SEPTIADI, Zürich (CH); Oleksandr LYTOVCHENKO, MöIndal (SE); Nina DUMAUTHIOZ, Bruxelles (BE)

(21) Appl. No.: 18/873,306

(22) PCT Filed: Jun. 12, 2023
(86) PCT No.: PCT/EP2023/065696

§ 371 Date: Dec. 9, 2024

(30) Foreign Application Priority Data
Jun. 10, 2022 (EP) 22178524.9
Dec. 6, 2022 (EP) 22211826.7
May 10, 2023 (EP) 23172686.0

(51) Int. Cl. A61K 47/69; A61K 9/00; A61K 9/51; A61K 31/7105; A61K 31/711; A61K 38/00; A61K 47/68; A61P 9/10; C07K 16/18

(52) U.S. Cl. CPC A61K 47/6901; A61K 9/0019; A61K 9/0078; A61K 9/5192; A61K 31/7105; A61K 31/711; A61K 38/00; A61K 47/6807; A61K 47/6835; A61P 9/10; C07K 16/18; C07K 2317/82

(57) ABSTRACT

The current invention focuses on ways to deliver various payloads including nucleic acids molecules (such as oligonucleotides), polypeptides (such as proteins), drugs or a combination thereof. As such, the invention relates to, inter alia, a mitochondrion comprising one or more payload(s) attached to the outer membrane of the mitochondrion, wherein the payload(s) is indirectly or directly electrostatically attached to the outer membrane of the mitochondrion. The invention further involves combining mitochondria comprising one or more payload(s) attached to the outer membrane of the mitochondrion with a protective layer that envelopes/encapsules and/or coats the mitochondrion and payload to provide a further delivery platform. This methodology is particularly effective for increasing the uptake and efficiency of the one or more payload(s) for therapeutic purposes.

The current invention focuses on ways to deliver various payloads including nucleic acids molecules (such as oligonucleotides), polypeptides (such as proteins), drugs or a combination thereof. As such, the invention relates to, inter alia, a mitochondrion comprising one or more payload(s) attached to the outer membrane of the mitochondrion, wherein the payload(s) is indirectly or directly electrostatically attached to the outer membrane of the mitochondrion. The invention further involves combining mitochondria comprising one or more payload(s) attached to the outer membrane of the mitochondrion with a protective layer that envelopes/encapsules and/or coats the mitochondrion and payload to provide a further delivery platform. This methodology is particularly effective for increasing the uptake and efficiency of the one or more payload(s) for therapeutic purposes.

The delivery of nucleic acid molecules, such as DNA and RNA, polypeptides, such as proteins, drugs, or a combination thereof, into cells and tissue remains a significant challenge in the field of biotechnology. Direct injection of naked DNA and RNA has been shown to have low transfection efficiency in vitro, ex-vivo and in vivo (NPL1). DNA and RNA molecules are large in size and have poor stability in biological media, making them vulnerable to degradation by nucleases. Viral vectors in combination with synthetic lipids or nanoparticles have been used as a delivery platform, but the majority of these combination-products often evoke unwanted immune responses, have low transfection efficiency, and may be toxic in the long-term (NPL2, NPL3). Additionally, when interacting with blood, the formation of a protein corona can lead to aberrant biodistribution, mistargeting, unexpected toxicity, and low therapeutic efficacy (NPL4).

Isolated mitochondria have been found to be biocompatible and non-toxic materials, which may be effectively taken up by cells through endocytosis as reported in a study by Pacak et al. (NPL5). These organelles also have a specific distribution, targeting specific organs, such as, but not limited to, the heart, lung, or kidney (NPL6). Mitochondria are also immuno-silent (NPL7), thus may be an attractive delivery platform. However, mitochondria have not been successfully employed as a vehicle for the delivery of various payloads.

Accordingly, there is an urgent need for the development of biocompatible vectors or delivery platforms that can overcome the above limitations.

The technical problem is solved by the embodiments provided herein and as presented in the claims.

Accordingly, the invention, inter alia, relates to the following items.
1. A mitochondrion comprising one or more payload(s) attached to the outer membrane of the mitochondrion, wherein the payload(s) is indirectly or directly electrostatically attached to the outer membrane of the mitochondrion.
2. The mitochondrion of item 1, wherein the payload is one or more of:
   i) a nucleic acid molecule;
   ii) a polypeptide;
   iii) a drug; or
   iv) a combination of one or more of (i) to (iii).
3. The mitochondrion of item 1 or 2, wherein the payload is charged.
4. The mitochondrion of any one of items 1 to 3, wherein the payload has the same net charge as the net charge of the mitochondrion.
5. The mitochondrion of item 4, wherein the payload and mitochondrion both have a net negative charge and wherein the payload is attached to the mitochondrion via a positively-charged species.
6. The mitochondrion of item 5, wherein the positively-charged species is a polycationic species.
7. The mitochondrion of item 6, wherein the polycationic species is a linear or branched polycationic polymer.
8. The mitochondrion according to item 7, wherein the linear or branched polycationic polymer is polylysine, histidylated polylysine, polyornithine, polyarginine, high-mobility group protein (HMG) 1 and 17, modified chitosan, cationized human serum albumin, polyethyleneimine (PEI), polypropyleneimine (PPI), a cationic dendrimer, poly(2-(dimethylamino)ethyl methacrylate) (PDMAEMA), a polyallylamine derivative, diethylaminoethyl (DEAE)-dextran, poly(N-alkyl-4-vinylpyridinium), a poly(amidoamine), cationic gelatin, cationic cellulose or a combination thereof.
9. The mitochondrion of item 5, wherein the positively-charged species is a positively-charged nanoparticle.
10. The mitochondrion of item 5, wherein the positively-charged species is a positively-charged particle.
11. The mitochondrion of item 9, wherein the one or more nucleic acid molecule(s) is attached to the surface of the positively-charged nanoparticle or encapsulated in the positively-charged nanoparticle.
12. The mitochondrion of item 10, wherein the one or more nucleic acid molecule(s) is attached to the surface of the positively-charged particle or encapsulated in the positively-charged particle.
13. The mitochondrion of any one of items 9 to 12, wherein the positively-charged nanoparticle and/or particle is a lipid nanoparticle/particle, a dendrimer nanoparticle/particle, a micelle nanoparticle/particle, a protein nanoparticle/particle, a liposome, a non-porous silica nanoparticle/particle, a mesoporous silica nanoparticle/particle, a silicon nanoparticle/particle, a gold nanoparticle/particle, a gold nanowire, a silver nanoparticle/particle, a platinum nanoparticle/particle, a palladium nanoparticle/particle, a titanium dioxide nanoparticle/particle, a carbon nanotube, a carbon dot nanoparticle/particle, a polymer nanoparticle/particle, a zeolite nanoparticle/particle, an aluminium oxide nanoparticle/particle, a hydroxyapatite nanoparticle/particle, a quantum dot nanoparticle/particle, a zinc oxide nanoparticle/particle, a zirconium oxide nanoparticle/particle, graphene or a graphene oxide nanoparticle/particle.
14. The mitochondrion of any one of items 1 to 3, wherein the payload has a different net charge as the net charge of the mitochondrion.
15. The mitochondrion of item 14, wherein the payload and the mitochondrion are attached via a zwitterionic species.
16. The mitochondrion of item 14, wherein the payload is uncharged and wherein the payload is attached to a positively-charged species.
17. The mitochondrion of item 16, wherein the positively-charged species is as defined in any one of items 6 to 13.
18. The mitochondrion of item 2, wherein the one or more nucleic acid molecule(s) is electrostatically linked to an antibody, optionally wherein the antibody is a modified antibody, optionally wherein the modified antibody possesses one or more positive charges.
19. The mitochondrion of item 2, wherein the one or more nucleic acid molecule(s) is encapsulated in a nanoparticle, wherein the nanoparticle is electrostatically linked to an antibody, optionally wherein the antibody is a modified antibody, optionally wherein the modified antibody possesses one or more positive charges.
20. The mitochondrion according to item 18 or 19, wherein the antibody specifically binds to an antigen comprised in the outer membrane of the mitochondrion, wherein the antigen is OPA1, TOM70, TOMM20, Mitofusin 1, Mitofusin 2 or VDAC1.
21. The mitochondrion of any one of items 1 to 20, wherein the mitochondrion is linked to and/or enveloped in a protective layer.
22. The mitochondrion of item 21, wherein the protective layer is a protective polymer.
23. The mitochondrion of item 22, wherein the protective polymer is a linear or branched cationic polymer, optionally wherein the linear or branched cationic polymer is electrostatically linked to the one or more payload(s).
24. The mitochondrion of item 22, wherein the protective polymer is a linear or branched cationic block copolymer, optionally wherein the linear or branched cationic block copolymer is electrostatically linked to the one or more payload(s).
25. The mitochondrion of item 22, wherein the protective polymer is a cationic graft (g) copolymer, optionally wherein the cationic graft (g) copolymer is electrostatically linked to the one or more payload(s).
26. The mitochondrion of item 22, wherein the protective polymer is a linear or branched pegylated (PEG) cationic polymer, optionally wherein the linear or branched pegylated (PEG) cationic polymer is electrostatically linked to the one or more payload(s).
27. The mitochondrion of item 21, wherein the protective layer is a lipid formulation, optionally wherein the lipid formulation is a cationic lipid formulation, further optionally wherein the cationic lipid formulation is electrostatically linked to the one or more payload(s).
28. The mitochondrion of any one of items 21 to 27, wherein the protective layer is linked to a targeting moiety.
29. The mitochondrion of any one of items 21 to 28, wherein the protective layer is linked to an antibody, optionally wherein the protective layer linked to an antibody is electrostatically linked to the one or more payload(s) or wherein the protective layer linked to an antibody is covalently linked to the one or more payload(s).
30. The mitochondrion of any one of items 21 to 28, wherein the protective layer is linked to a carbohydrate, optionally wherein the protective layer linked to a carbohydrate is electrostatically linked to the one or more payload(s) or wherein the protective layer linked to a carbohydrate is covalently linked to the one or more payload(s).
31. The mitochondrion of item 23, wherein the linear or branched cationic polymer is polyethyleneimine, RGD-modified polyethyleneimine, polylysine, RGD-modified polylysine, polyornithine, RGD-modified polyornithine, polyarginine, RGD modified polyarginine, polypropyleneimine, RGD-modified polypropyleneimine, polyallylamine, RGD-modified polyallylamine, chitosan, RGD-modified chitosan, poly(2-(dimethylamino)ethyl methacrylate), RGD-modified poly(2-(dimethylamino)ethyl methacrylate), poly(amidoamine)s, RGD-modified poly(amidoamine)s or a combination thereof.
32. The mitochondrion of item 24, wherein the cationic block copolymer is poly(ethylene glycol)-block-polyethyleneimine, RGD-modified poly(ethylene glycol)-block-polyethyleneimine, poly(ethylene glycol)-block-polylysine, RGD-modified poly(ethylene glycol)-block-polylysine, poly(ethylene glycol)-block-polyornithine, RGD-modified poly(ethylene glycol)-block-polyornithine, poly(ethylene glycol)-block-polyarginine, RGD-modified poly(ethylene glycol)-block-polyarginine, poly(ethylene glycol)-block-polypropyleneimine, RGD-modified poly(ethylene glycol)-block-polypropyleneimine, poly(ethylene glycol)-block-polyallylamine, RGD-modified poly(ethylene glycol)-block-polyallylamine, poly(ethylene glycol)-block-poly(2-(dimethylamino)ethyl methacrylate), RGD-modified poly(ethylene glycol)-block-poly(2-(dimethylamino)ethyl methacrylate), poly(ethylene glycol)-block-poly(amidoamine)s, RGD-modified poly(ethylene glycol)-block-poly(amidoamine)s or a combination thereof.
33. The mitochondrion of item 25, wherein the cationic graft (g) copolymer is poly(ethylene glycol)-g-polyethyleneimine, RGD-modified poly(ethylene glycol)-g-polyethyleneimine, poly(ethylene glycol)-g-polylysine, RGD-modified poly(ethylene glycol)-g-polylysine, poly(ethylene glycol)-g-polyornithine, RGD-modified poly(ethylene glycol)-g-polyornithine, poly(ethylene glycol)-g-polyarginine, RGD-modified poly(ethylene glycol)-g-polyarginine, poly(ethylene glycol)-g-polypropyleneimine, RGD-modified poly(ethylene glycol)-g-polypropyleneimine, poly(ethylene glycol)-g-polyallylamine, RGD-modified poly(ethylene glycol)-g-polyallylamine, poly(ethylene glycol)-g-poly(2-(dimethylamino)ethyl methacrylate), RGD-modified poly(ethylene glycol)-g-poly(2-(dimethylamino)ethyl methacrylate), poly(ethylene glycol)-g-poly(amidoamine)s, RGD-modified poly(ethylene glycol)-g-poly(amidoamine)s or a combination thereof.
34. The mitochondrion of item 26, wherein the pegylated (PEG) cationic polymer is pegylated-polyethyleneimine, RGD-modified pegylated polyethyleneimine, pegylated polylysine, RGD-modified pegylated polylysine, histidylated polylysine, pegylated polyornithine, RGD-modified pegylated polyornithine, pegylated polyarginine, RGD-modified pegylated polyarginine, pegylated polypropyleneimine, RGD-modified pegylated polypropyleneimine, pegylated polyallylamine, RGD-modified pegylated polyallylamine, pegylated chitosan, RGD-modified pegylated chitosan, pegylated poly(2-(dimethylamino)ethyl methacrylate), RGD-modified pegylated poly(2-(dimethylamino)ethyl methacrylate), pegylated poly(amidoamine)s RGD-modified pegylated poly(amidoamine)s or a combination thereof.
35. The mitochondrion of item 27, wherein the lipid formulation comprises DC-cholesterol (3β-[N—(N′,N′-Dimethylaminoethane)-carbamoyl]cholesterol hydrochloride), DLinDMA (1,2-dilinoleyloxy-3-dimethylaminopropane), DLinMC3DMA (dilinoleylmethyl-4-dimethylaminobutyrate), DODMA (1,2-dioleyloxy-3-dimethylaminopropane), DOGS (dioctadecylamidoglycylspermine), DOSPA (2,3-dioleyloxy-N-[2 (sperminecarboxamido)ethyl]-N,N-dimethyl-1-propanaminium), DOTAP (1,2-dioleoyl-3-trimethylammonium-propane chloride), DOTMA (1,2-di-O-octadecenyl-3-trimethylammonium propane chloride), UGG (unsaturated guanidinium glycoside), DOPE (1,2-Dioleoyl-sn-glycerophosphoethanolamine), lipofectamine or a combination thereof.
36. The mitochondrion of item 35, wherein the lipid formulation further comprises another lipid, preferably wherein said lipid is cholesterol, a substituted or unsubstituted cholesterol, a cholesterol derivative, such as a hydroxylated cholesterol derivative (e.g., a hydroxycholesterol), a PEG-lipid, DMPC (1,2-Dimyristoyl-sn-glycero-3-phosphocholine), DSPC (1,2-Distearoyl-sn-glycero-3-phosphocholine), DODAP (1,2-dioleoyl-3-dimethylammonium propane), DDA (dimethyldioctadecylammonium), 1,2-dioleoyl-sn-glycero-3-phosphate, 1,2-dimyristoyl-sn-glycero-3-phosphate, bis(monooleoylglycerol)phosphate or a combination thereof.
37. The mitochondrion of item 22, wherein the mitochondrion is linked to and/or enveloped in a zwitterionic protective polymer, optionally wherein the zwitterionic protective polymer is electrostatically linked to the one or more payload(s).
38. The mitochondrion of item 37, wherein the zwitterionic protective polymer is selected from: poly(2-methacryloyloxyethyl phosphorylcholine) (PMPC), polyethyleneimine-g-poly(2-methacryloyloxyethyl phosphorylcholine) (PEI-g-PMPC), co-assembly of cationic (carboxyl-functionalized) and anionic (amino-functionalized) copolyesters based on poly(ε-caprolactone)-block-poly(butylene fumarate)-block-poly(ε-caprolactone) (PCL-b-PBF-b-PCL), poly(lactic-co-glycolic acid) (PLGA)-PCB block copolymers (PLGA-b-PCB).
39. A composition comprising a plurality of mitochondria according to any one of items 1 to 38.
40. A pharmaceutical composition comprising a plurality of mitochondria according to any one of items 1 to 38 and a pharmaceutically acceptable carrier.
41. The pharmaceutical composition of item 40, wherein the pharmaceutical composition is formulated as a solution.
42. The pharmaceutical composition of item 40, wherein the pharmaceutical composition is formulated as an aerosol.
43. The mitochondrion of any one of items 1 to 38, the composition according to item 39 or the pharmaceutical composition according to any one of items 40 to 42 for use as a medicament.
44. The mitochondrion of any one of items 1 to 38, the composition according to item 39 or the pharmaceutical composition according to any one of items 40 to 42 for use in gene therapy.
45. The mitochondrion according to any one of items 1 to 38, the composition according to item 39 or the pharmaceutical composition according to any one of items 40 to 42 for use in the treatment of cardiovascular diseases, in particular for use in the treatment of ischemic heart disease, ischemia-reperfusion injury, or atherosclerosis.
46. The mitochondrion according to any one of items 1 to 38, the composition according to item 39 or the pharmaceutical composition according to any one of items 40 to 42 for use in the treatment of aging related diseases, in particular for use in the treatment of, sarcopenia, Parkinson's disease or Hutchinson-Gilford progeria syndrome.
47. The mitochondrion according to any one of items 1 to 38, the composition according to item 39 or the pharmaceutical composition according to any one of items 40 to 42 for use in the treatment of kidney diseases, in particular for use in the treatment of autosomal dominant polycystic kidney disease, Alport syndrome, Nephronophthisis, or Fabry disease.
48. The mitochondrion according to any one of items 1 to 38, the composition according to item 39 or the pharmaceutical composition according to any one of items 40 to 42 for use in the treatment of cancer.
49. The mitochondrion according to any one of items 1 to 38, the composition according to item 39 or the pharmaceutical composition according to any one of items 40 to 42 for use in in vitro, ex vivo, or in vivo genome editing.
50. The mitochondrion according to any one of items 1 to 38, the composition according to item 39 or the pharmaceutical composition according to any one of items 40 to 43 for use in radiation therapy.
51. A method for delivering a payload to a target organ, the method comprising a step of administering the pharmaceutical composition according to any one of items 40 to 42 into the bloodstream of a subject in need, wherein the pharmaceutical composition is administered into the bloodstream upstream of the target organ.
52. A method for delivering a payload to the lung, the method comprising a step of administering the pharmaceutical composition according to item 42 to a subject in need, wherein the pharmaceutical composition is administered by inhalation.
53. A method for attaching a payload to the outer membrane of a mitochondrion, the method comprising the steps of:
   a) providing a preparation of mitochondria;
   b) contacting the mitochondria provided in step (a) with at least one payload in the presence of a positively-charged species; and
   c) attaching the at least one payload to the mitochondria via the positively-charged species.
54. The method of item 53, wherein
   a) the at least one payload is simultaneously contacted with the positively-charged species and the mitochondria;
   b) the at least one payload is contacted with the positively-charged species to form a positively-charged complex before the positively-charged complex is contacted with the mitochondria; or
   c) the mitochondrion is contacted with the positively-charged species and subsequently contacted with the at least one payload.
55. The method of item 52 or 54, wherein the mitochondria are contacted with the at least one payload and the positively-charged species in a suitable buffer.
56. The method of item 55, wherein the buffer comprises or consists of HEPES, EGTA, Trehalose, CHES and sodium phosphate dibasic dihydrate, preferably wherein buffer comprises a mixture of a Solution X comprising or consisting of HEPES, EGTA and Trehalose and of a Solution Y comprising or consisting of CHES and sodium phosphate dibasic dihydrate, more preferably, wherein the buffer comprises a 4:1 mixture of Solution X comprising or consisting of 20 mM HEPES, 1 mM EGTA and 300 mM Trehalose (pH 7.2) and Solution Y comprising or consisting of 0.1 M CHES (pH 10) and 0.2 M sodium phosphate dibasic dihydrate.
57. The method of any one of items 53 to 56, wherein the mitochondria are contacted with the at least one payload, and the positively-charged species at room temperature for at least 5 minutes, such as at least 10 minutes, 20, 30, 40, 50, 60 or 120 minutes.
58. The method of any one of items 53 to 57, wherein the mitochondria are contacted with the at least one payload and the positively-charged species in the dark.
59. The method of any one of items 53 to 58, wherein the payload is a nucleic acid molecule which is DNA or RNA.
60. The method of any one of items 53 to 59, wherein the positively-charged species is a polycationic species, wherein the polycationic species is a linear or branched polycationic polymer, optionally wherein the linear or branched polycationic polymer is electrostatically linked to the at least one payload(s).
61. The method of item 60, wherein the linear or branched polycationic polymer is polylysine, histidylated polylysine, polyornithine, polyarginine, high-mobility group protein (HMG) 1 and 17, a modified chitosan, cationized human serum albumin, polyethyleneimine (PEI), polypropyleneimine (PPI), a cationic dendrimer, poly(2-(dimethylamino)ethyl methacrylate) (PDMAEMA), a polyallylamine derivative, diethylaminoethyl (DEAE)-dextran poly(N-alkyl-4-vinylpyridinium), a poly(amidoamine), cationic gelatin, cationic cellulose or a combination thereof.
62. The method of any one of items 53 to 59, wherein the positively-charged species is a positively-charged nanoparticle.
63. The method of item 62, wherein the method comprises a further step of
   a) attaching the at least one payload to the surface of the positively-charged nanoparticle; or
   b) encapsulating the at least one payload within the positively-charged nanoparticle.
64. The method of item 62 or 63, wherein the positively-charged nanoparticle is a lipid nanoparticle, a dendrimer nanoparticle, a micelle nanoparticle, a protein nanoparticle, a liposome, a non-porous silica nanoparticle, a mesoporous silica nanoparticle, a silicon nanoparticle, a gold nanoparticle, a gold nanowire, a silver nanoparticle, a platinum nanoparticle, a palladium nanoparticle, a titanium dioxide nanoparticle, a carbon nanotube, a carbon dot nanoparticle, a polymer nanoparticle, a zeolite nanoparticle, an aluminium oxide nanoparticle, a hydroxyapatite nanoparticle, a quantum dot nanoparticle, a zinc oxide nanoparticle, a zirconium oxide nanoparticle, graphene or a graphene oxide nanoparticle.
65. A method for preparing a mitochondrion comprising a payload, wherein the method comprises the steps of:
   a) providing a preparation of mitochondria;
   b) contacting the mitochondria with
      i) a positively-charged species if the payload and mitochondrion both have a net negative charge;
      ii) the payload if the payload has a different net charge as the net charge of the mitochondrion, optionally further a zwitterionic species; or
      iii) a payload attached to a positively-charged species if the payload is uncharged;
   c) obtaining mitochondria according to any one of items 1 to 20.
66. The method of item 65 further comprising subsequent to step c) a step of contacting the mitochondria with components to form a protective layer, and a step of obtaining mitochondria according to any one of items 22 to 38.
67. The method of any one of items 53 to 66, wherein an amount of 50 μg to 200 μg of mitochondria are contacted with 0.1 to 50 μmol of the payload and 0.02 to 10 μg, preferably 0.02 to 5 μg, of the positively-charged species.
68. The method according to any one of items 53 to 66, wherein the mitochondrion comprises a positively-charged species, wherein the positively-charged species is a polycationic polymer according to any of the previous items, and wherein the ratio of the polycationic polymer to the protective layer is about 1:2.
69. The method according to any of the previous method items, wherein 50 μg to 200 μg of mitochondria are contacted with 0.1 to 50 μmol of payload and 0.2 to 10 μg of the protective layer.
70. A method for delivering a payload to the kidney, the method comprising a step of administering the pharmaceutical composition according to items 40 to 50 into the renal artery of a subject in need.
71. A method for delivering a payload to the heart, the method comprising a step of administering the pharmaceutical composition according to items 40 to 50 into the intracoronary of a subject in need.
72. A method for delivering a payload to the liver, the method comprising a step of administering the pharmaceutical composition according to items 40 to 50 into the hepatic artery or portal vein of a subject in need.
73. A method for delivering a payload to the pancreas, the method comprising a step of administering the pharmaceutical composition according to items 40 to 50 into the hepatic artery of a subject in need.
74. A method for delivering a payload to the duodenum, the method comprising a step of administering the pharmaceutical composition according to items 40 to 50 into the hepatic artery of a subject in need.
75. A method for delivering a payload to the spleen, the method comprising a step of administering the pharmaceutical composition according to items 40 to 50 into the splenic artery of a subject in need.
76. A method for delivering a payload to the lung, the method comprising a step of administering the pharmaceutical composition according to items 40 to 50 into the pulmonary artery of a subject in need.
77. A method for delivering a payload to the intestines, the method comprising a step of administering the pharmaceutical composition according to items 40 to 50 into the superior mesenteric artery of a subject in need.
78. A method for delivering a payload to the bladder, the method comprising a step of administering the pharmaceutical composition according to items 40 to 50 into the superior and inferior vesical arteries of a subject in need.
79. A method for delivering a payload to a target organ, the method comprising a step of administering the pharmaceutical composition according to items 40 to 50 into a subject in need, wherein the pharmaceutical composition is administered into the kidney or bladder or intestines or pancreas or duodenum or liver or lung or spleen through direct injection.

Accordingly, in its broadest aspect, the invention relates to a mitochondrion to which one or more payloads, such as one or more nucleic acid molecule(s), one or more polypeptide(s), and/or one or more drug(s) are attached by innovative means.

More specifically, the current invention relates to and/or utilizes mitochondria complexed with oligonucleotides, nucleic acids, such as DNA or RNA (e.g., mRNA and/or siRNA), polypeptides, proteins, drugs, or a combination thereof, as a platform for targeted and safe delivery into cells and tissues (FIG. 1-2). The process for producing this mitochondrial complex involves, for example, functionalizing the mitochondria with cationic species, such as cationic polymers, then with oligonucleotides, nucleic acid molecules, polypeptides, proteins and/or drugs. The mitochondrial complex of the present invention may comprise one or more additional protective layer, such as a protective polymer layer comprised of cationic copolymers which are linked to the mitochondrion or envelope the mitochondrion in order to protect the attached payload (e.g., nucleic acid molecules, such as oligonucleotides, polypeptides, such as proteins and/or drugs) from degradation and enables efficient internalization of, for example, the mitochondria-oligonucleotide complex or of the mitochondria-protein complex. The mitochondria-payload complex, such as the mitochondria-oligonucleotide complex, of the invention can escape the digestive organelles (i.e., lysosomes) upon internalization. The isolated mitochondria of the mitochondria-based system of the present invention are ideal to transport different nucleic acids such as DNA/RNA molecules or proteins and allow a high biological activity of the DNA/RNA (e.g., translation, transcription, protein expression, knockdown) upon release inside the cells, while maintaining low cytotoxicity. Hereby, it is demonstrated that the mRNA translation efficiency exceeds 70% compared to the commonly used lipofectamine (100%) as control in various cell types, including human epithelial lung cells (A549; 79%) and human cardiac fibroblasts (HCF, 70%). Furthermore, it is illustrated that mitochondrial delivery of siRNA comprising a protective layer results in a greater protein knockdown compared to lipofectamine-siRNA and compared to the previous generation products as described in the European patent applications No. 22178524.9 and No. 22211826.7. Additionally, the use of mitochondria for simultaneous delivery of one or more, e.g., two or more, different oligonucleotides (e.g., mRNA and siRNA) or an oligonucleotide and drug (e.g., anionic drug) or one or more, e.g., two of more, oligonucleotides and one or more drugs, e.g., two or more, anionic drugs (e.g., siRNA and PX-12) for oncology application is provided. Accordingly, the delivery platform for mitochondria of the present invention offers several advantages:
1. It is a native and safe method for delivering payloads, such as nucleic acid molecules such as DNA, RNA, polypeptides, or drugs.
2. It has been shown to be successful for in vivo, ex vivo, or in vitro delivery, as evidenced by high levels of transcription, translation, and protein expression/knockdown.
3. The payload-mitochondria complex has a stabilizing effect on the payload, in particular DNA or RNA, in contrast to naked nucleic acids, which is further improved by the addition of a protective layer to the mitochondria-oligonucleotide complex.
4. The platform does not cause an immune response or cytotoxicity when internalized into cells, in contrast to commonly used delivery systems such as viral vectors.
5. It may be administered to a cell, a tissue, or systemically through different routes, such as injection or aerosol. Moreover, the mitochondrion may be administered as a single dose or as at least 2 or more doses.
6. It delivers payloads, such as nucleic acid molecules, polypeptides or drugs with high colloidal stability via mitochondria.
7. The new generation of the mitochondrial delivery platform comprising a protective layer is effective in delivering payloads, such as nucleic acid molecules, polypeptides, drugs achieving higher transcription of mRNA, and/or higher protein knockdown by siRNA compared to previously available means.
8. It allows for combination therapy where various payloads, such as at least two different nucleic acid molecules, polypeptides, drugs, oligonucleotides may be delivered simultaneously by a single mitochondrion.
9. It allows for combination therapy where at least two or more different payloads, such as nucleic acid molecules, polypeptides, drugs may be delivered simultaneously by a single mitochondrion.
10. It allows for combination therapy where at least one or more different payloads, such as nucleic acid molecules, polypeptides, drugs, and in particular combinations thereof may be delivered simultaneously by a single mitochondrion.
11. It allows for combination therapy where at least one or more different nucleic acid molecules and one or more drugs may be delivered simultaneously by a single mitochondrion.
12. It allows for combination therapy where at least two or more different polypeptides may be delivered simultaneously by a single mitochondrion.
13. It allows for combination therapy where at least two or more different proteins may be delivered simultaneously by a single mitochondrion.
14. It allows for combination therapy where at least one or more different polypeptides and one or more drugs may be delivered simultaneously by a single mitochondrion.
15. It allows for combination therapy where at least one or more different proteins and one or more drugs may be delivered simultaneously by a single mitochondrion.
16. It allows for combination therapy where at least one or more different nucleic acid molecules (e.g., oligonucleotides) with at least one or more polypeptides (e.g., proteins) may be delivered simultaneously by a single mitochondrion.
17. It allows for a combination therapy where at least one or more different nucleic acid molecules (e.g., oligonucleotides) with at least one or more polypeptides (e.g., proteins) together with at least one or more drugs may be delivered simultaneously by a single mitochondrion.
18. It allows for combination therapy where at least one or more siRNA and at least one or more anionic drugs may be delivered simultaneously by a single mitochondrion.
19. It allows for combination therapy where at least two or more drugs, e.g., anionic drug, may be delivered simultaneously by a single mitochondrion.

The disclosures in the context of the present invention described herein are applicable to the corresponding uses and vice versa.

In one aspect, the present invention provides a mitochondrion comprising one or more nucleic acid molecule(s) attached to the outer membrane of the mitochondrion, wherein the one or more nucleic acid molecule(s) a) is electrostatically attached to the outer membrane of the mitochondrion via a positively-charged species; or b) is covalently linked to the outer membrane of the mitochondrion; or c) is linked to an antibody that specifically binds to an antigen comprised in the outer membrane of the mitochondrion; or d) is linked to a mitochondria-targeting small molecule.

Mitochondria possess a negatively-charged surface, which, according to one aspect of the present invention, may be functionalized with cationic molecules, turning the surface charge of mitochondria's outer membrane to entirely positive values (i.e., net positive values) or partially positive values. That is, while mitochondria generally have a negative surface charge, parts of the surface or all of the surface may be masked by/attached to positively-charged molecules as provided herein. The surface charge of the mitochondrion that is recognized by another molecule may accordingly be positive. Positively-charged mitochondria, that is mitochondria with a net surface charge that is positive or mitochondria that have positively-charged surface areas, may be conjugated with negatively-charged payload molecules such as nucleic acid molecules, polypeptides, drugs or combinations thereof.

A mitochondrion is a double-membrane-bound organelle found in most eukaryotic organisms. Accordingly, a mitochondrion of the present invention may be a mitochondrion of any eukaryotic organism. A mitochondrion may be a mitochondrion of an animal, plant, yeast or fungi. A mitochondrion may be a mitochondrion of a human. A mitochondrion of the invention may be obtained by any means, such cell culture. Accordingly, a mitochondrion of the invention may be obtained by in vitro cell culture. Preferably, a mitochondrion may be obtained from in vitro 2D or 3D cell culture. A mitochondrion of the present invention can also be obtained from a tissue. A mitochondrion obtained from a tissue may be obtained from any tissue of a eukaryotic organism. Accordingly, a mitochondrion may be obtained from cells or tissue of a eukaryotic organism maintained in a culture. A mitochondrion may be obtained from an animal, plant, yeast or fungi cell maintained in in vitro cell culture. Preferably, a mitochondrion is obtained from a human tissue or cell culture. More preferably a mitochondrion is obtained from a human in vitro cell culture. In preferred embodiments, a mitochondrion is obtained from an animal tissue or cell culture, in particular from murine tissue or cell culture. Preferably a mitochondrion is obtained from a mouse in vitro cell culture. A mitochondrion may be obtained from mouse embryonic fibroblasts (MEF). A mitochondrion may be obtained from MEFs maintained in in vitro cell culture. A mitochondrion may be obtained from MEFs maintained in in vitro cell culture comprising Dulbecco's modified Eagle medium (DMEM) medium. In some embodiments, a mitochondrion is obtained from, e.g., human cardiac fibroblasts (HCF). A mitochondrion may be obtained from HCFs maintained in in vitro cell culture. A mitochondrion may be obtained from HCFs maintained in in vitro cell culture comprising fibroblast medium-2. In further embodiments, a mitochondrion may be obtained from HepG2 cells. A mitochondrion may be obtained from HepG2 maintained in in vitro cell culture. A mitochondrion may be obtained from HepG2 maintained in in vitro cell culture comprising Roswell Park Memorial Institute (RPMI) medium.

A mitochondrion of the present invention can also be freshly obtained by isolating the mitochondrion from a cell culture or a tissue, e.g., of eukaryotes. Mitochondrion obtained from tissue may be derived from placental, liver, muscle or pig tissue. Accordingly, a mitochondrion may be obtained by fresh isolation from an animal, plant, yeast or fungi cell culture or tissue. Preferably, a mitochondrion may be obtained by fresh isolation from a human cell culture or tissue. A mitochondrion may be obtained by fresh isolation from HCFs or HepG2s. A mitochondrion may be obtained by fresh isolation from HCFs or HepG2s maintained in in vitro cell culture. A mitochondrion may be obtained by fresh isolation from HCFs maintained in in vitro cell culture comprising fibroblast medium-2. A mitochondrion may be obtained by fresh isolation from HepG2 maintained in in vitro cell culture comprising RPMI medium. Moreover, a mitochondrion may be obtained by fresh isolation from murine cell culture or tissue. A mitochondrion may be obtained by fresh isolation from a mouse in vitro cell culture. A mitochondrion may be obtained by fresh isolation from MEFs. A mitochondrion may be obtained by fresh isolation from MEFs maintained in in vitro cell culture. A mitochondrion may be obtained by fresh isolation from MEFs maintained in in vitro cell culture comprising DMEM medium.

The mitochondria may be autologous (i.e., autogeneic or autogenous). In some embodiments the mitochondria are autogenous or autologous mitochondria with genetic modification. In some other embodiments, the mitochondria are autologous and linked to an imaging, diagnostic or a pharmaceutical agent (such as nucleic acid molecules, polypeptides and/or drugs). In some other embodiments, the agent is embedded or incorporated into the autologous mitochondria. In some other embodiments, the mitochondria are allogeneic. In some embodiments the mitochondria are allogeneic mitochondria with genetic modification. In some other embodiments, the mitochondria are allogeneic mitochondria, which are linked to an imaging, diagnostic or a pharmaceutical agent. In some other embodiments, the agent is embedded or incorporated into the allogeneic mitochondria. In some other embodiments, the mitochondria are xenogeneic. In some embodiments the mitochondria are xenogeneic mitochondria with genetic modification. In some other embodiments, the mitochondria are xenogeneic mitochondria, which are linked to an imaging, diagnostic or a pharmaceutical agent. In some other embodiments, the agent is embedded or incorporated into the xenogeneic mitochondria. In certain aspects, herein contemplated are (specifically in a therapeutic context, e.g., as ex vivo methods) methods wherein mitochondria are obtained/isolated from a subject (patient), the mitochondria are modified by attaching one or more payloads, such as nucleic acid molecule(s) (such as oligonucleotides), and/or one or more polypeptide(s) (such as proteins) and/or one or more drug(s) to the outer membrane of the mitochondria as described by the herein provided methods and subsequently are administered to the same subject (patient).

A mitochondrion of the present invention can also be obtained from a frozen stock of mitochondria. Accordingly, a mitochondrion obtained from a frozen stock of mitochondria is thawed before being used in the means and methods of the present invention. A mitochondrion may be obtained from a frozen stock comprising mitochondria of any eukaryotes, such as an animal, plant, yeast or fungi. A mitochondrion may be obtained from a frozen stock comprising human mitochondria. A mitochondrion may be obtained from a frozen stock comprising mitochondria obtained by fresh isolation or cell culture or tissue culture. A mitochondrion may be obtained from a frozen stock comprising human mitochondria obtained by fresh isolation or cell culture or tissue culture. A mitochondrion may be obtained from a frozen stock comprising human mitochondria obtained from HCFs by fresh isolation or cell culture or tissue culture. Preferably, a mitochondrion may be obtained from a frozen stock comprising human mitochondria obtained from HCFs in vitro cell culture. More preferably, a mitochondrion may be obtained from a frozen stock comprising human mitochondria obtained from HCFs in vitro cell culture comprising fibroblast medium-2.

A mitochondrion of the present invention may be labeled or un-labeled. A labeled mitochondrion allows for later detection, an un-labeled mitochondrion reflects its native nature. A mitochondrion may be labeled by any means known to the skilled person. Accordingly, a mitochondrion may be labeled by a dye. A mitochondrion may be labeled by a dye comprising rosamine, tetramethylrosamine, X-rosamine, dihydrotetramethylrosamine, dihydro-X-rosamine, carbocyanine or derivatives thereof. A mitochondrion can also be labeled with small molecules or small particles. Accordingly, a mitochondrion of the present invention may be labeled with ¹⁸F-rhodamine 6G or iron oxide nanoparticles or gold nanoparticles or gold nanostars or silver nanoparticles.

The “mitochondria” to be used herein refer to viable mitochondria that are (essentially) free of eukaryotic cell material, such as extraneous eukaryotic cell material, e.g., which have been isolated/purified from cells or a cell culture. Thus, only minimal amounts of cellular components (other than mitochondria) are present in (a composition of) mitochondria to be used herein. Preferably, no other cellular components than mitochondria are present in (a composition of) mitochondria to be used herein. In this sense, the “mitochondria” to be used herein are “isolated mitochondria” and the terms “mitochondria” and “isolated mitochondria” may be used interchangeably. Any current art-known technique may be used for isolation of mitochondria, such as for example, subcellular fractioning by repeated differential centrifugation (DC) or density gradient centrifugation (DGC), or differential filtration (McCully, WO2015192020A1). A mitochondrion of the present invention is useful for delivering nucleic acids (such as oligonucleotides), polypeptides (such as proteins) and/or drugs to cells. Accordingly, a mitochondrion of the present invention is preferably alive or viable and possesses a negative membrane potential. In the sense of the present invention “being alive” means having or maintaining a metabolism or another biological function or structure.

As used herein, the term “viable mitochondria” is used herein to describe viable mitochondria, which are intact, active, functioning and respiration-competent mitochondria. According to some embodiments, “viable mitochondria” refers to mitochondria that exhibit biological functions, such as, for example, respiration as well as ATP and/or protein synthesis.

As used herein, the term “intact mitochondria” is used throughout the specification to describe mitochondria, which comprise an integer outer and inner membrane, an integer inter-membrane space, integer cristae (formed by the inner membrane) and an integer matrix. Alternatively, intact mitochondria are mitochondria which preserve their structure and ultrastructure. In another aspect, intact mitochondria contain active respiratory chain complexes I-V embedded in the inner membrane, maintain membrane potential and capability to synthesize ATP.

A mitochondrion of the present invention may be functionalized with targeting molecules (such as small targeting molecules, targeting aptamers, targeting peptide, carbohydrate, sugar, and targeting antibody), drugs, reporter molecules/nanoparticles (e.g., fluorescence molecules, metallic nanoparticles, magnetic nanoparticles to say some) or contract agents, imaging agents, diagnostic agents or pharmaceutical agents.

In the sense of the present invention the terms “nanoparticle”, “nanoformulation” and “nanobody” may be used interchangeably. In some embodiments a nanoparticle is a lipid nanoparticle. Exemplary nanoparticles of the present invention are a lipid nanoparticle, a dendrimer nanoparticle, a micelle nanoparticle, a protein nanoparticle, a liposome, a non-porous silica nanoparticle, a mesoporous silica nanoparticle, a silicon nanoparticle, a gold nanoparticle, a gold nanowire, a silver nanoparticle, a platinum nanoparticle, a palladium nanoparticle, a titanium dioxide nanoparticle, a carbon nanotube, a carbon dot nanoparticle, a polymer nanoparticle, a zeolite nanoparticle, an aluminium oxide nanoparticle, a hydroxyapatite nanoparticle, a quantum dot nanoparticle, a zinc oxide nanoparticle, a zirconium oxide nanoparticle, graphene or a graphene oxide nanoparticle. The skilled person is aware that nanoparticles can comprise different charges or may be functionalised to have a certain charge. In the sense of the present invention a nanoparticle may be functionalized with a positively-charged species, such as a positively-charged functional group (e.g., a quaternary ammonium group) or a polycationic species resulting in a positively-charged nanoparticle. Moreover, in some embodiments, the nanoparticle may be chemically modified to be positively-charged, the chemical modification may be e.g., the protonation of a chemical group comprised in the nanoparticle. In the sense of the present invention “functionalized” can mean “attached to some moiety or compound that has a function”, such as a biological function, e.g., a targeting function, protecting function or modulating function. Accordingly, a mitochondrion may be functionalized by attaching different agents that convey desired functions, such as changing the charge of the nanoparticle.

In the context of the present invention, the term “particle” or “positively-charged particle” preferably refers to a lipid particle, a dendrimer particle, a micelle particle, a protein particle, a liposome, a non-porous silica particle, a mesoporous silica particle, a silicon particle, a gold particle, a gold wire, a silver particle, a platinum particle, a palladium particle, a titanium dioxide particle, a carbon tube, a carbon dot particle, a polymer particle, a zeolite particle, an aluminium oxide particle, a hydroxyapatite particle, a quantum dot particle, a zinc oxide particle, a zirconium oxide particle, graphene or a graphene oxide particle.

A mitochondrion of the present invention is especially useful since it may be stored without deteriorating and/or disintegrating, i.e., being stable, for a long period of time. Accordingly, the present invention provides a mitochondrion comprising one or more payloads, such as nucleic acid molecule(s), polypeptide(s), drug(s) or combinations thereof attached to the outer membrane of the mitochondrion, wherein the one or more payloads, such as nucleic acid molecule(s), polypeptide(s), drug(s) or combinations thereof:
a) is electrostatically attached to the outer membrane of the mitochondrion via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule, preferably, wherein the mitochondrion is stored at low temperature, such as −80° C. or −20° C., in a conjugation buffer. A mitochondrion of the present invention may be stored at low temperature, e.g., at −20° C., preferably at −80° C., in a conjugation buffer for at least 5 days, 1 week, 2 weeks, 3 weeks, 1 month, 2 months, 3 months or 4 months, for example for 6 months or longer, without disintegrating or decomposing. A mitochondrion comprising one or more payloads, such as nucleic acid molecule(s), polypeptide(s), drug(s) or combinations thereof attached to the outer membrane may be stored in conjugation buffer to maintain high colloidal stability (e.g., no agglomeration/aggregation or disintegration). A mitochondrion comprising one or more payloads, such as nucleic acid molecule(s), polypeptide(s), drug(s) or combinations thereof attached to the outer membrane is preferably, stored in conjugation buffer at low temperatures (e.g., at −20° C., preferably at −80° C.) in the dark for preservation, e.g., for at least two months after the complex formation.

A mitochondrion of the present invention may be encapsulated inside alginate/hydrogel capsules. Encapsulation in alginate/hydrogel capsules can increase the storage time of a mitochondrion of the present invention, i.e., avoid disintegration and increase stability.

A mitochondrion of the present invention may be contacted by payloads, such as nucleic acid molecule(s), polypeptide(s), drug(s) or combinations thereof in a solution, such as a buffer. A buffer of the present invention is preferably an aqueous solution of any compounds feasible for conjugation of a payload to a mitochondrion. The buffer used for the contacting step is preferably a conjugation buffer. A conjugation buffer of the present invention may be an aqueous solution. Solvents used in aqueous solutions of the present invention may be aqueous solvents, such as buffers, including water, deionized water, double distilled water, DNAse and RNAse free water, DNAse and RNAse free deionized water, DNAse and RNAse free double distilled water. A conjugation buffer of the present invention can comprise a mixture of Solution X and Solution Y. Solution X can comprise or consist of N-2-hydroxyethylpiperazine-N′-2-ethanesulfonic acid (i.e., HEPES), ethylene glycol-bis(β-aminoethyl ether)-N,N,N′,N′-tetraacetic acid (i.e., EGTA) and Trehalose. Solution Y can comprise or consist of N-cyclohexyl-2-aminoethanesulfonic acid (i.e., CHES) and sodium phosphate dibasic dihydrate. Solution X and Solution Y may be aqueous solutions.

In the sense of the present invention the compositions of Solution X and Y may be used in any amount and at any pH that is feasible to achieve successful conjugation of a mitochondrion and its payload, such as a nucleic acid or a polypeptide. In some embodiments, Solution X comprises or consists of 5 to 150 mM HEPES, 0.1 to 10 mM EGTA and 150 to 500 mM Trehalose (pH 6 to 9) and optionally an aqueous solvent. In some embodiments Solution X comprises or consists of 5, 8, 11, 14, 17, 20, 23, 26, 29, 32, 35, 38, 41, 44, 47, 50, 53, 56, 59, 62, 65, 68, 71, 74, 77, 80, 83, 86, 89, 92, 95, 98, 101, 104, 107, 110, 113, 116, 119, 122, 125, 128, 131, 134, 137, 140, 143, 146, 149 or 150 mM HEPES, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9 or 10 mM EGTA and 150, 157, 164, 171, 178, 185, 192, 199, 206, 213, 220, 227, 234, 241, 248, 255, 262, 269, 276, 283, 290, 297, 304, 311, 318, 325, 332, 339, 346, 353, 360, 367, 374, 381, 388, 395, 402, 409, 416, 423, 430, 437, 444, 451, 458, 465, 472, 479, 486, 493 or 500 mM Trehalose (pH 6, 6.5, 7, 7.5, 8, 8.5 or 9) and optionally an aqueous solvent. Preferably, Solution X comprises or consists of 20 mM HEPES, 1 mM EGTA and 300 mM Trehalose (pH 7.2) and optionally an aqueous solvent.

In some embodiments Solution Y comprises or consists of 0.01 to 0.2 M CHES (pH 8 to 12) and 0.02 to 0.6 M sodium phosphate dibasic dihydrate and optionally an aqueous solvent. In some embodiments Solution Y comprises or consists of 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19 or 0.2 M CHES (pH 8, 8.5, 9, 9.5, 10, 10.5, 11, 11.5 or 12), and optionally an aqueous solvent. Preferably Solution Y comprises or consists of 0.1 M CHES (pH 10) and 0.2 M sodium phosphate dibasic dihydrate and optionally an aqueous solvent.

In the sense of the present invention Solution X and Y may be mixed at any rate that is feasible to achieve successful conjugation of a mitochondrion and its payload, such as a nucleic acid or a polypeptide. A mixture of Solution X and Y can result in a conjugation buffer of the present invention. Accordingly, in some embodiments a conjugation buffer comprises a 2:1 to 10:1 mixture of Solution X and Solution Y. In some embodiments a conjugation buffer comprises a 2:1, 3:1, 4:1, 5:1, 6:1, 7:1, 8:1, 9:1 or 10:1 mixture of Solution X and Solution Y. Preferably a conjugation buffer comprises a 4:1 mixture of Solution X and Solution Y.

In some embodiments a conjugation buffer of the present invention has a pH of 7.5 to 11. In some embodiments a conjugation buffer of the present invention has a pH of 7.5, 8, 8.5, 9, 9.5, 10, 10.5 or 11.

In a preferred embodiment a conjugation buffer of the present invention comprises a mixture of Solution X comprising or consisting of 20 mM HEPES+1 mM EGTA+300 mM Trehalose (pH 7.2) and Solution Y comprising or consisting of 0.1 M CHES (pH 10)+0.2 M sodium phosphate dibasic dihydrate and optionally an aqueous solvent. In a further preferred embodiment, a conjugation buffer of the present invention comprises a 4:1 mixture of Solution X comprising or consisting of 20 mM HEPES, 1 mM EGTA and 300 mM Trehalose (pH 7.2) and Solution Y comprising or consisting of 0.1 M CHES (pH 10) and 0.2 M sodium phosphate dibasic dihydrate and optionally an aqueous solvent.

The conjugation buffer of the present invention may be used to store a mitochondrion of the present invention, and its compositions and pharmaceutical compositions therefor. A conjugation buffer used for storage is referred to as a storage buffer and comprises the ingredients as defined herein above. Accordingly, in a preferred embodiment, a storage buffer of the present invention comprises a 4:1 mixture of Solution X comprising or consisting of 20 mM HEPES, 1 mM EGTA and 300 mM Trehalose (pH 7.2) and Solution Y comprising or consisting of 0.1 M CHES (pH 10) and 0.2 M sodium phosphate dibasic dihydrate.

Nucleic acid molecules of the present invention may be stored in a buffer comprising or consisting of an aqueous solvent and Solution X. Nucleic acid molecules of the present invention may be stored in a DNA/RNA buffer comprising DNase/RNase-free water, PBS and Solution X comprising or consisting of 20 mM HEPES+1 mM EGTA+300 mM Trehalose (pH 7.2).

A mitochondrion of the present invention may be complexed with one or more payloads, such as nucleic acid molecule(s), polypeptide(s), drug(s) or combinations thereof attached to the outer membrane of the mitochondrion.

In some embodiments, the present invention provides a mitochondrion comprising one or more nucleic acid molecule(s), wherein the nucleic acid molecule is DNA or RNA.

In general, a nucleic acid of the present invention may be any nucleic acid, such as naturally occurring nucleic acids or synthetic nucleic acids. A nucleic acid may be an endogenous or exogenous nucleic acid. A nucleic acid is a polymer composed of nucleotides which are monomers comprising a 5-carbon sugar, a phosphate group, and a nitrogenous base, such as adenine, cytosine, guanine, thymine, and uracil. It is also envisioned herein, that a nucleic acid may be a modified nucleic acid. A nucleic acid of the present invention may be a deoxyribonucleic acid (DNA) or a ribonucleic acid (RNA). Accordingly, a nucleic acid of the present invention may be an oligonucleotide comprising DNA or RNA of any length. Accordingly, the term “nucleic acid molecule”, or any grammatical variations thereof, as used herein, may be used interchangeably with the term “oligonucleotide”. In some embodiments an oligonucleotide of the present invention can comprise 10 to 15000 base pairs. A nucleic acid may be a single-stranded DNA (ssDNA). In some embodiments a single stranded DNA can comprise 10 to 15000 nucleotides. A nucleic acid may be double stranded (dsDNA). A nucleic acid of the present invention may be linear. A nucleic acid may be circular. Accordingly, a nucleic acid may be a circular DNA (cDNA). A nucleic acid may be a plasmid DNA (pDNA). The nucleic acid of the present invention may be in different structural forms. Accordingly, the nucleic acid may be a A-DNA, B-DNA (Watson-Crick), Z-DNA, C-DNA, D-DNA or E-DNA. The nucleic acid of the present invention can comprise different segments. Accordingly, the nucleic acid may be a DNA comprising a sense segment that carries translatable sequence. The nucleic acid may be a DNA comprising an antisense segment that is complementary to a sense segment. A nucleic acid of the present invention may be of natural origin or may be synthetic. Accordingly, a DNA can originate from any natural source, e.g., organisms, such as eukaryotes. A DNA can originate from an animal, plant, bacteria, or yeast. Preferably, the DNA is human or substantially similar to a human DNA.

A nucleic acid of the present invention can also be an RNA. Accordingly, a nucleic acid of the present invention may be an oligonucleotide comprising RNA of any length. In some embodiments an RNA of the present invention can comprise 10 to 10000 nucleotides. A nucleic acid may be a single-stranded RNA (ssRNA). A nucleic acid may be double stranded (dsRNA). An RNA of the present invention may be linear. An RNA may be circular. RNA molecules can comprise protein coding RNA, such as mRNA or non-coding RNA, such as siRNA. Accordingly, a RNA of the present invention may be a messenger RNA (mRNA). A RNA of the present invention may be a non-coding RNA involved in RNA interference (RNAi) such as small interference RNA (siRNA) and micro RNA (miRNA). An RNA can also be other small RNAs selected from the group of small nucleolar RNAs (snoRNAs), small nuclear RNA (snRNA) including U1 spliceosomal RNA, U2 spliceosomal RNA, U4 spliceosomal RNA, U5 spliceosomal RNA, and U6 spliceosomal RNA, exRNAs, scaRNAs and long ncRNAs such as Xist and HOTAIR. An RNA can also be a non-coding RNA (ncRNA) such as a transfer RNA (tRNA) or a ribosomal RNA. An RNA of the present invention may be complementary to a DNA sequence in an animal, plant, bacteria, or yeast. Preferably, an RNA may be complementary to a DNA sequence in a human. Preferably, a RNA may be complementary to a DNA sequence in a gene of a human. An RNA of the present invention can also be complementary to an RNA sequence in an animal, plant, bacteria, or yeast. Preferably, an RNA may be complementary to a RNA sequence in a human. Preferably, an RNA may be complementary to a human mRNA sequence.

An RNA of the present invention may be of natural origin or may be synthetic. Accordingly, An RNA may be artificial, such as a short hairpin RNA (shRNA). An RNA can originate from any natural source. An RNA may be endogenous or exogenous. An RNA can originate from an animal, plant, bacteria, or yeast. An RNA may be a human RNA or substantially similar to a human RNA. An RNA may be a human mRNA. An RNA may be a siRNA complementary to a human mRNA. Preferably an RNA is a siRNA complementary to the glyceraldehyde 3-phosphate dehydrogenase (GAPDH) mRNA, optionally the human GAPDH mRNA. Preferably, an RNA is a siRNA complementary to the MDM2 mRNA, optionally, the human MDM2 proto-oncogene (MDM2) mRNA. In one embodiment an RNA may be a siRNA complementary to the mRNA of the hexokinase 1 mRNA, optionally the human hexokinase 1 mRNA. Preferably, an RNA of the present invention is a mRNA encoding a human peptide, such as a human polypeptide and/or protein.

A nucleic acid molecule of the present invention may be functionalized with targeting molecules (such as small targeting molecules, targeting aptamers, targeting peptide, carbohydrate, sugar, and targeting antibody), drugs, reporter molecules/nanoparticles (e.g., fluorescence molecules, metallic nanoparticles, magnetic nanoparticles to say some) or contrast agents.

In some embodiments, payloads, such as nucleic acid molecule(s), polypeptide(s), drug(s) or combinations thereof of the present invention are formulated into a nanoparticle, particle, cationic lipid formulation (e.g., lipid nanoformulation), block-copolymer, cationic lipid or cationic polymer. The payloads, such as nucleic acid molecule(s), polypeptide(s), drug(s) or combinations thereof may be attached to the surface of the nanoparticle or particle, or encapsulated in the nanoparticle or particle.

The present invention provides a mitochondrion-payload complex, in particular a nucleic acid molecule(s), a polypeptide(s), a drug(s) or a complex of a combinations thereof useful for delivery into cells. The present invention also provides for attachment of payloads, such as nucleic acid molecule(s), polypeptide(s), drug(s) or combinations thereof to a mitochondrion. One or more payloads, such as nucleic acid molecule(s), polypeptide(s), drug(s) or combinations thereof may be electrostatically attached to the outer membrane of a mitochondrion, in particular in cases where the payload(s), such as nucleic acid molecule(s), polypeptide(s), drug(s) or combinations thereof have an overall positive surface charge. In cases where the payloads, such as nucleic acid molecule(s), polypeptide(s), drug(s) or combinations thereof have an overall negative surface charge, the electrostatic attachment may be promoted via a positively-charged species. In one embodiment the positively-charged species is a polycationic species. In another embodiment the positively-charged species is a positively-charged nanoparticle or particle. Electrostatic interactions comprise the attractive or repulsive interactions between charged molecules and/or surfaces of, for example, subcellular organelles, such as membrane surfaces of mitochondria. In the sense of the present invention, a mitochondrion can electrostatically interact with payloads, such as nucleic acid molecule(s), polypeptide(s), drug(s) or combinations thereof forming a complex comprising a mitochondrion and one or more payloads, such as nucleic acid molecule(s), polypeptide(s), drug(s) or combinations thereof. Accordingly, electrostatic interaction may be used to attach a positively-charged entity to a negatively-charged entity. In this regard, it is known that an isolated mitochondrion has a negative net surface charge. In the sense of the present invention, the mitochondrion may be positively or negatively-charged or neutral depending on the complex of the mitochondrion with the various agents provided herein. In the sense of the present invention, the nucleic acid may be positively or negatively-charged. Either of the above constellations can lead to a successful attachment via electrostatic interaction as long as the mitochondrion and the payloads, such as nucleic acid molecule(s), polypeptide(s), drug(s) or combinations thereof carry opposite charges or do not carry the same charges. In this regard, it is understood that the charge may depend on the pH. The skilled person is aware of how to handle pH-dependent charges. Generally, mitochondria surfaces possess a negative surface charge profile. Similarly, DNA and RNA are generally negatively-charged molecules.

According to the present invention, the mitochondria surface can also be functionalized with a positively-charged species to establish electrostatic attachment of a payloads, such as nucleic acid molecule(s), polypeptide(s), drug(s) or combinations thereof. Accordingly, one or more payloads, such as nucleic acid molecule(s), polypeptide(s), drug(s) or combinations thereof may be electrostatically attached to the outer membrane of a mitochondrion via a positively-charged species. One or more payloads, such as nucleic acid molecule(s), polypeptide(s), drug(s) or combinations thereof may be electrostatically attached to the outer membrane of a mitochondrion via a polycationic species, wherein the polycationic species is linear or branched polycationic polymer. As used herein, the term “polycation” refers to a moiety having positive charges at a plurality of sites and whose overall charge is positive. Payloads, such as nucleic acid molecule(s), polypeptide(s), drug(s) or combinations thereof may be electrostatically attached to the outer membrane of a mitochondrion via a linear or branched polycationic polymer, wherein the linear or branched cationic polymer is polylysine, histidylated polylysine, polyornithine, polyarginine, high-mobility group protein (HMG) 1 and 17, modified chitosan, cationized human serum albumin, polyethyleneimine (PEI), polypropyleneimine (PPI), a cationic dendrimer, poly(2-(dimethylamino)ethyl methacrylate) (PDMAEMA), a polyallylamine derivative, diethylaminoethyl (DEAE)-dextran, poly(N-alkyl-4-vinylpyridinium), a poly(amidoamine), cationic gelatin, cationic cellulose or a combination thereof.

In the sense of the present invention, the negative surface charge profile of mitochondria can also be useful for attaching one or more payloads, such as nucleic acid molecule(s), polypeptide(s), drug(s) or combinations thereof electrostatically to the outer membrane of a mitochondrion via a positively-charged nanoparticle. Accordingly, the positively-charged nanoparticle comprising one or more nucleic acid molecule(s) may be electrostatically attached to the negatively-charged surface of the mitochondrion. Accordingly, one or more nucleic acid molecule(s) may be electrostatically attached to the outer membrane of a mitochondrion via a positively-charged nanoparticle. One or more nucleic acid molecule(s) may be electrostatically attached to the outer membrane of a mitochondrion via a positively-charged particle. As the skilled person is aware, the difference between a nanoparticle and a particle relates generally to a difference in size, where nanoparticles typically have a size between 1 and 100 nm, whereas particles typically have a size between 100 nm and 2.5 μm. However, as the skilled person is also aware, the distinction between nanoparticles and particles based on their size is not consistently held in the art and different size distinctions may be made depending on the class of the particle. In some embodiments, the particles of the present invention may be microparticles or microspheres. Payloads, such as nucleic acid molecule(s), polypeptide(s), drug(s) or combinations thereof may be attached to the surface of a positively-charged nanoparticle or a, for example, positively-charged particle or be encapsulated by a positively-charged nanoparticle or a, for example, positively-charged particle. Accordingly, one or more payloads, such as nucleic acid molecule(s), polypeptide(s), drug(s) or combinations thereof may be electrostatically attached to the outer membrane of a mitochondrion via a positively-charged nanoparticle, wherein the one or more payloads, such as nucleic acid molecule(s), polypeptide(s), drug(s) or combinations thereof is attached to the surface of the positively-charged nanoparticle or encapsulated in the positively-charged nanoparticle. One or more payloads, such as nucleic acid molecule(s), polypeptide(s), drug(s) or combinations thereof may be electrostatically attached to the outer membrane of a mitochondrion via a positively-charged particle, wherein the one or more payloads, such as nucleic acid molecule(s), polypeptide(s), drug(s) or combinations thereof is attached to the surface of the positively-charged particle or encapsulated in the positively-charged particle.

In general, the invention is not limited to any specific nanoparticles or particles for attachment to mitochondria and attachment of payloads, such as nucleic acid molecule(s), polypeptide(s), drug(s) or combinations thereof or encapsulation of the same. Accordingly, one or more payloads, such as nucleic acid molecule(s), polypeptide(s), drug(s) or combinations thereof may be attached to the surface of or encapsulated in: a lipid nanoparticle, a dendrimer nanoparticle, a micelle nanoparticle, a protein nanoparticle, a liposome, a non-porous silica nanoparticle, a mesoporous silica nanoparticle, a silicon nanoparticle, a gold nanoparticle, a gold nanowire, a silver nanoparticle, a platinum nanoparticle, a palladium nanoparticle, a titanium dioxide nanoparticle, a carbon nanotube, a carbon dot nanoparticle, a polymer nanoparticle, a zeolite nanoparticle, an aluminum oxide nanoparticle, a hydroxyapatite nanoparticle, a quantum dot nanoparticle, a zinc oxide nanoparticle, a zirconium oxide nanoparticle, graphene or a graphene oxide nanoparticle. Furthermore, one or more nucleic acid molecule(s) may be attached to the surface of or encapsulated in: a lipid particle, a dendrimer particle, a micelle particle, a protein particle, a liposome, a non-porous silica particle, a mesoporous silica particle, a silicon particle, a gold particle, a gold wire, a silver particle, a platinum particle, a palladium particle, a titanium dioxide particle, a carbon tube (such as a carbon microtube), a carbon dot particle, a polymer particle, a zeolite particle, an aluminum oxide particle, a hydroxyapatite particle, a quantum dot particle, a zinc oxide particle, a zirconium oxide particle, graphene or a graphene oxide particle.

The skilled person is aware that the above means of electrostatic attachment to or encapsulation in a nanoparticle or particle may be applied to all products, methods, apparatus or uses described herein.

In the sense of the present invention, payloads, such as nucleic acid molecule(s), polypeptide(s), drug(s) or combinations thereof can also be covalently linked to the outer membrane of a mitochondrion. A covalent bond or covalent link or covalent interaction is formed by a chemical bond that involves sharing of electron pairs between atoms. Payloads, such as nucleic acid molecule(s), polypeptide(s), drug(s) or combinations thereof, in particular polypeptide(s) such as proteins may be attached to a mitochondrion via a peptide bond, such as an amide bond (e.g., a carboxamide bond or carbamide bond). A mitochondrion of the present invention possessing amino groups of mitochondria membrane-associated proteins/peptides may be covalently linked with N-hydroxysuccinimide ester (NHS)-functionalized nanoparticles/particles, NHS-modified nucleic acid molecules or NHS-modified molecules forming covalently bound ligand and more stable conjugate. Alternatively, a mitochondrion of the present invention possessing a carboxyl group as part of a mitochondria associated protein may be covalently linked with an amine group comprised on the nanoparticles/particles or nucleic acid molecules. In general, a mitochondrion may be covalently linked via any chemical group that can form chemical bonds, preferably with primary amines, e.g., by acylation or alkylation. The present invention is not particularly limited to any such groups. Exemplary chemical groups useful in the sense of the present invention are isothiocyanates, isocyanates, acyl azides, sulfonyl chlorides, aldehydes, glyoxals, epoxides, oxiranes, carbonates, aryl halides, imidoesters, carbodiimides, anhydrides, and fluorophenyl esters. Accordingly, in some embodiments a mitochondrion of the present invention may be covalently linked to isothiocyanates, isocyanates, acyl azides, sulfonyl chlorides, aldehydes, glyoxals, epoxides, oxiranes, carbonates, aryl halides, imidoesters, carbodiimides, anhydrides, or fluorophenyl esters. Accordingly, in some embodiments in a mitochondrion comprising payloads, such as nucleic acid molecule(s), polypeptide(s), drug(s) or combinations thereof, the payload may be covalently linked to isothiocyanates, isocyanates, acyl azides, sulfonyl chlorides, aldehydes, glyoxals, epoxides, oxiranes, carbonates, aryl halides, imidoesters, carbodiimides, anhydrides, or fluorophenyl esters. Accordingly, in some embodiments a polypeptide of the present invention may be covalently linked to isothiocyanates, isocyanates, acyl azides, sulfonyl chlorides, aldehydes, glyoxals, epoxides, oxiranes, carbonates, aryl halides, imidoesters, carbodiimides, anhydrides, or fluorophenyl esters. The skilled person is aware that the selection of a chemical group (i.e., a functional group) comprised in the payload, such as nucleic acid molecule(s), polypeptide(s), drug(s) or combinations thereof, nanoparticle or particle which links said payloads, such as nucleic acid molecule(s), polypeptide(s), drug(s) or combinations thereof, particle or nanoparticle may be dictated by the available chemical group on the surface of the mitochondrion (e.g., on a polypeptide or protein comprised in the outer membrane of the mitochondrion) and vice versa.

Accordingly, one or more payloads, such as nucleic acid molecule(s), polypeptide(s), drug(s) or combinations thereof may be covalently linked to the outer membrane of a mitochondrion. Preferably, one or more payloads, such as nucleic acid molecule(s), polypeptide(s), drug(s) or combinations thereof may be linked to a polypeptide in the outer membrane of a mitochondrion via an amide bond. One or more payloads, such as nucleic acid molecule(s), polypeptide(s), drug(s) or combinations thereof may be linked to a polypeptide in the outer membrane of a mitochondrion via an amide bond, wherein the one or more payloads, such as nucleic acid molecule(s), polypeptide(s), drug(s) or combinations thereof has been modified to undergo formation of the amide bond with an amine function comprised in the polypeptide in the outer membrane of the mitochondrion. One or more payloads, such as nucleic acid molecule(s), polypeptide(s), drug(s) or combinations thereof may be linked to a polypeptide in the outer membrane of a mitochondrion via an ester bond, wherein the one or more payloads, such as nucleic acid molecule(s), polypeptide(s), drug(s) or combinations thereof has been modified to undergo formation of the ester bond with an carboxylic function (e.g., comprised in the polypeptide in the outer membrane of the mitochondrion. A payload, such as nucleic acid molecule(s), polypeptide(s), drug(s) or combinations thereof can also be attached to a mitochondrion by covalently linking a nanoparticle comprising a payload, such as nucleic acid molecule(s), polypeptide(s), drug(s) or combinations thereof to a mitochondrion. The nanoparticle may be any nanoparticle known to the skilled person and may be charged (i.e., positively-charged or negatively-charged) or uncharged (i.e., having a neutral charge). In preferred embodiments, said nanoparticle is a positively-charged nanoparticle as described hereinabove. Accordingly, one or more payloads, such as nucleic acid molecule(s), polypeptide(s), drug(s) or combinations thereof may be linked to a polypeptide in the outer membrane of a mitochondrion via an amide bond wherein the one or more payloads, such as nucleic acid molecule(s), polypeptide(s), drug(s) or combinations thereof is encapsulated in a nanoparticle, and wherein the nanoparticle comprises a functional group that allows covalent linkage of the nanoparticle to a polypeptide in the outer membrane of the mitochondrion. Accordingly, one or more payloads, such as nucleic acid molecule(s), polypeptide(s), drug(s) or combinations thereof may be linked to a polypeptide in the outer membrane of a mitochondrion via an ester bond, wherein the one or more payloads, such as nucleic acid molecule(s), polypeptide(s), drug(s) or combinations thereof is encapsulated in a nanoparticle, and wherein the nanoparticle comprises a functional group that allows covalent linkage of the nanoparticle to a polypeptide in the outer membrane of the mitochondrion A payload, such as nucleic acid molecule(s), polypeptide(s), drug(s) or combinations thereof may also be attached to or encapsulated in a positively-charged nanoparticle, such as a polycationic nanoparticle. The nanoparticle, such as the positively-charged nanoparticle, comprising the payload, such as nucleic acid molecule(s), polypeptide(s), drug(s) or combinations thereof may be covalently linked to an antibody that specifically binds to an antigen comprised in the outer membrane of a mitochondrion. The nanoparticle, such as the positively-charged nanoparticle comprising the payload, such as nucleic acid molecule(s), polypeptide(s), drug(s) or combinations thereof may comprise phospholipids with reactive groups which enable covalent linkage to an antibody that specifically binds to an antigen comprised in the outer membrane of a mitochondrion. Accordingly, one or more payloads, such as nucleic acid molecule(s), polypeptide(s), drug(s) or combinations thereof may be encapsulated in a nanoparticle, wherein the nanoparticle is covalently linked to an antibody.

In the sense of the present invention, payloads, such as nucleic acid molecule(s), polypeptide(s), drug(s) or combinations thereof can also be linked to an antibody that specifically binds to an antigen comprised in the outer membrane of the mitochondrion. Such an antibody comprising the payload, such as nucleic acid molecule(s), polypeptide(s), drug(s) or combinations thereof preferably binds to an antigen comprised in the outer membrane of the mitochondrion, thereby facilitating the formation of the delivery platform. The present invention is not limited to any specific antigens, in general, the invention may be performed with an antibody specifically binding any antigen comprised in the outer membrane of a mitochondrion, thereby facilitating formation of a mitochondrion-nucleic acid complex (i.e., a mitochondrion comprising one or more payloads, such as nucleic acid molecule(s), polypeptide(s), drug(s) or combinations thereof). An antibody (interchangeably used in plural form) as used herein is an immunoglobulin molecule capable of specific binding to a target, such as a carbohydrate, polynucleotide, lipid, polypeptide, etc., through at least one antigen recognition site, located in the variable region of the immunoglobulin molecule. The preferred target herein is an antigen comprised in the outer membrane of a mitochondrion, particularly in a human mitochondrion. As used herein, the term “antibody” encompasses not only intact (i.e., full-length) monoclonal antibodies, but also antigen-binding fragments (such as Fab, Fab′, F(ab′)2, Fv, single chain variable fragment (scFv)), mutants thereof, fusion proteins comprising an antibody portion, humanized antibodies, chimeric antibodies, diabodies, linear antibodies, single chain antibodies, single domain antibodies (e.g., camel or llama VHH antibodies), multi-specific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins may be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

An antibody that “specifically binds” (used interchangeably herein) to a target or an epitope is a term well understood in the art, and methods to determine such specific binding are also well known in the art. A molecule is said to exhibit “specific binding” if it reacts or associates more frequently, more rapidly, with greater duration and/or with greater affinity with a particular target antigen than it does with alternative targets. An antibody “specifically binds” to a target antigen if it binds with greater affinity, avidity, more readily, and/or with greater duration than it binds to other substances. For example, an antibody that specifically (or preferentially) binds to an epitope is an antibody that binds this epitope with greater affinity, avidity, more readily, and/or with greater duration than it binds to other epitopes. It is also understood by reading this definition that, for example, an antibody that specifically binds to a first target antigen may or may not specifically or preferentially bind to a second target antigen. As such, “specific binding” or “preferential binding” does not necessarily require (although it can include) exclusive binding. Generally, but not necessarily, reference to binding means preferential binding.

In general, one or more payloads, such as nucleic acid molecule(s), polypeptide(s), drug(s) or combinations thereof may be linked to any antibody that specifically binds to an antigen comprised in a mitochondrion. Exemplary antigens include, but are not limited to AIF, GCSH, MRPL40, TIMM23, ATP5A, HSP60, OPA1, TOM70, ATP5F1, OXA1L, TOMM20, BCS1L, Mitofilin, Prohibitin, TUFM, COX4, Mitofusin 1, SDHB, UQCRC1, COX5b, Mitofusin 2, SSBP1, VDAC1.

Preferably, one or more payloads, such as nucleic acid molecule(s), polypeptide(s), drug(s) or combinations thereof may be linked to any antibody that specifically binds to an antigen comprised in the outer membrane of a mitochondrion. Accordingly, one or more payloads, such as nucleic acid molecule(s), polypeptide(s), drug(s) or combinations thereof may be linked to an antibody specifically binding to an antigen comprised in the outer membrane of a mitochondrion, wherein the preferred antigen is any one of OPA1, TOM70, TOMM20, Mitofusin 1, Mitofusin 2, VDAC1.

A payload, such as nucleic acid molecule(s), polypeptide(s), drug(s) or combinations thereof may be covalently linked to an antibody forming a payload-antibody complex which can bind to an antigen of a mitochondrion. Accordingly, a payload, such as nucleic acid molecule(s), polypeptide(s), drug(s) or combinations thereof may be covalently linked to an antibody forming a payload-antibody complex which can bind to an antigen comprised in the outer membrane of a mitochondrion. In some embodiments a DNA or RNA molecule may be covalently linked to an antibody forming a payload-antibody complex which can bind to an antigen of a mitochondrion. In some embodiments a DNA or RNA molecule may be covalently linked to an antibody forming a payload-antibody complex which can bind to an antigen comprised in the outer membrane of a mitochondrion.

A payload, such as nucleic acid molecule(s), polypeptide(s), drug(s) or combinations thereof can also be electrostatically linked to an antibody specifically binding to an antigen comprised in the outer membrane of a mitochondrion. Accordingly, one or more payloads, such as nucleic acid molecule(s), polypeptide(s), drug(s) or combinations thereof may be linked to an antibody specifically binding to an antigen comprised in the outer membrane of a mitochondrion, wherein the one or more payloads, such as nucleic acid molecule(s), polypeptide(s), drug(s) or combinations thereof is electrostatically linked to a modified antibody, wherein the modified antibody possesses one or more positive charges.

A payload, such as nucleic acid molecule(s), polypeptide(s), drug(s) or combinations thereof may be electrostatically linked to a modified antibody, such as an antibody comprising a positive charge, forming a payload-antibody complex which can bind to an antigen of a mitochondrion. Accordingly, a payload, such as nucleic acid molecule(s), polypeptide(s), drug(s) or combinations thereof may be electrostatically linked to a modified antibody, such as an antibody comprising a positive charge, forming a payload-antibody complex which can bind to an antigen comprised in the outer membrane of a mitochondrion. In some embodiments a DNA or RNA molecule may be electrostatically linked to a modified antibody, such as an antibody comprising a positive charge, forming a nucleic acid-antibody complex which can bind to an antigen of a mitochondrion. In some embodiments a DNA or RNA molecule may be electrostatically linked to a modified antibody, such as an antibody comprising a positive charge, forming a nucleic acid-antibody complex which can bind to an antigen comprised in the outer membrane of a mitochondrion.

An antibody specifically binding to an antigen comprised in the outer membrane of a mitochondrion may be used to attach a nanoparticle, such as a lipid nanoparticle, comprising payloads, such as nucleic acid molecule(s), polypeptide(s), drug(s) or combinations thereof thereby facilitating the attachment of the payload, such as nucleic acid molecule(s), polypeptide(s), drug(s) or combinations thereof to a mitochondrion. Accordingly, one or more payloads, such as nucleic acid molecule(s), polypeptide(s), drug(s) or combinations thereof may be linked to an antibody that specifically binds to an antigen comprised in the outer membrane of the mitochondrion, wherein the one or more payloads, such as nucleic acid molecule(s), polypeptide(s), drug(s) or combinations thereof is encapsulated in a nanoparticle, and wherein the nanoparticle is covalently linked to the antibody. Said nanoparticle may be any nanoparticle known to the skilled person and may be charged (i.e., positively- or negatively-charged) or uncharged (i.e., having an overall neutral charge). One or more payloads, such as nucleic acid molecule(s), polypeptide(s), drug(s) or combinations thereof may be linked to an antibody that specifically binds to an antigen comprised in the outer membrane of the mitochondrion, wherein the nanoparticle is electrostatically linked to a modified antibody, wherein the modified antibody possesses one or more positive charges. Where the nanoparticle is electrostatically linked to a modified antibody having one or more positive charges, said nanoparticle preferably possesses a negative charge. In some embodiments, one or more payloads, such as nucleic acid molecule(s), polypeptide(s), drug(s) or combinations thereof may be linked to an antibody that specifically binds to an antigen comprised in the outer membrane of the mitochondrion, wherein the nanoparticle is electrostatically linked to a modified antibody, wherein the modified antibody possesses one or more negative charges. Where the nanoparticle is electrostatically linked to a modified antibody having one or more negative charges, said nanoparticle preferably possesses a positive charge. The nanoparticle possessing a positive charge is preferably the positively-charged nanoparticle as described hereinabove.

A payload, such as nucleic acid molecule(s), polypeptide(s), drug(s) or combinations thereof can also be linked to an entity which is then linked to an antibody. Such entity may be biotin, which is linked to an avidin conjugated antibody. Accordingly, one or more payloads, such as nucleic acid molecule(s), polypeptide(s), drug(s) or combinations thereof may be linked to an antibody specifically binding to an antigen comprised in the outer membrane of a mitochondrion, wherein the one or more payloads, such as nucleic acid molecule(s), polypeptide(s), drug(s) or combinations thereof is covalently linked to biotin, wherein biotin is linked to an avidin conjugated antibody. Further, a payload, such as nucleic acid molecule(s), polypeptide(s), drug(s) or combinations thereof can also be linked to an entity which is then linked to an antibody when being encapsulated into a nanoparticle. Accordingly, one or more payloads, such as nucleic acid molecule(s), polypeptide(s), drug(s) or combinations thereof may be linked to an antibody specifically binding to an antigen comprised in the outer membrane of a mitochondrion, wherein one or more payloads, such as nucleic acid molecule(s), polypeptide(s), drug(s) or combinations thereof is encapsulated in a nanoparticle, wherein the nanoparticle is covalently linked to biotin, wherein biotin is linked to avidin conjugated antibody. The invention is not limited to an avidin conjugated antibody, as the skilled person is aware, avidin may be substituted with a structural analogue such as streptavidin or neutravidin. Streptavidin typically has about 30% sequence identity to avidin, but an almost identical secondary, tertiary and quaternary structure. Neutravidin is a deglycosylated analogue of avidin.

A payload, such as nucleic acid molecule(s), polypeptide(s), drug(s) or combinations thereof can also be linked to an entity which is then linked to an antibody. Such entity may be an activated ester, which is linked to an antibody. Accordingly, one or more payloads, such as nucleic acid molecule(s), polypeptide(s), drug(s) or combinations thereof may be linked to an antibody specifically binding to an antigen comprised in the outer membrane of a mitochondrion, wherein the one or more payloads, such as nucleic acid molecule(s), polypeptide(s), drug(s) or combinations thereof is covalently linked to an activated ester, wherein the activated ester is linked to the antibody via an amide bond. Further, a payload, such as nucleic acid molecule(s), polypeptide(s), drug(s) or combinations thereof can also be linked to an entity which is then linked to an antibody when being encapsulated into a nanoparticle. Accordingly, one or more payloads, such as nucleic acid molecule(s), polypeptide(s), drug(s) or combinations thereof may be linked to an antibody specifically binding to an antigen comprised in the outer membrane of a mitochondrion, wherein the nanoparticle is covalently linked to activated ester, wherein activated ester is linked to the antibody via amide bond. Single stranded nucleic acid molecules such as ssDNA or ssRNA may be hybridized with one or more complementary single-stranded nucleic acid molecule attached on or to an antibody which specifically binds to a mitochondrion thereby facilitating the attachment of the nucleic acid and the formation of the delivery platform. Accordingly, one or more nucleic acid molecule(s) may be linked to an antibody that specifically binds to an antigen comprised in the outer membrane of the mitochondrion, wherein the nucleic acid molecule is a single-stranded nucleic acid molecule (ssDNA or ssRNA), wherein the single-stranded nucleic acid molecule is hybridized with one or more complementary single-stranded nucleic acid molecule attached on or to an antibody modified with one or more complementary single-stranded nucleic acid molecules.

In the sense of the present invention, the antibody may be “modified with one or more complementary single-stranded nucleic acid molecule”, which means that an antibody is modified with a single-stranded nucleic acid molecule that can hybridize with another single-stranded nucleic acid molecule, i.e., that the nucleic acid may be attached to said antibody via the hybridization.

In the sense of the present invention “modified antibody” also means that an antibody is modified to possess one or more positive charges, e.g., to attach a negatively-charged nucleic acid.

In another aspect of the present invention, payloads, such as nucleic acid molecule(s), polypeptide(s), drug(s) or combinations thereof may be linked to a mitochondria-targeting small molecule to facilitate attachment of the payloads, such as nucleic acid molecule(s), polypeptide(s), drug(s) or combinations thereof and formation of the delivery platform. Accordingly, one or more payloads, such as nucleic acid molecule(s), polypeptide(s), drug(s) or combinations thereof may be attached to the outer membrane of the mitochondrion, wherein the one or more payloads, such as nucleic acid molecule(s), polypeptide(s), drug(s) or combinations thereof is linked to a mitochondria-targeting small molecule. In the sense of the present invention, any mitochondria-targeting small molecule may be used to facilitate attachment. Exemplary mitochondria-targeting small molecules are selected from: triphenylphosphonium (TPP), dequalinium (DQA), E-4-(1H-Indol-3-ylvinyl)-N-Methylpyridineiodide (F16), Rhodamine 19, biguanidine and guanidine. Accordingly, one or more payloads, such as nucleic acid molecule(s), polypeptide(s), drug(s) or combinations thereof may be linked to a mitochondria-targeting small molecule, wherein the mitochondria targeting small molecules is selected from triphenylphosphonium (TPP), dequalinium (DQA), E-4-(1H-Indol-3-ylvinyl)-N-Methylpyridineiodide (F16), Rhodamine 19, biguanidine and/or guanidine. In a preferred embodiment, one or more payloads, such as nucleic acid molecule(s), polypeptide(s), drug(s) or combinations thereof may be linked to triphenylphosphonium (TPP). As used herein, a “targeting moiety” refers to a moiety that is capable of specifically binding to (i) a molecule on the surface of a target cell or (ii) a molecule that is capable of specifically binding to a molecule on the surface of a target cell, such as a cell within a target tissue of a subject. A molecule (e.g., cell surface molecule) that specifically binds to a targeting moiety is also referred to herein as a “binding partner.” In some embodiments of copolymers and related compositions and methods as described herein, a targeting moiety specifically binds to a molecule on the surface of the target cell.

The present invention is, inter alia, based on electrostatic interaction. The charge of a payload, such as nucleic acid molecule(s), polypeptide(s), drug(s) or combinations thereof or a mitochondrion may be modified with e.g., cationic molecules or polymers. Thus, the charge of a payload, such as nucleic acid molecule(s), polypeptide(s), drug(s) or combinations thereof or a mitochondrion may be e.g., inverted. Considering the above, the skilled person understands that the products, methods, apparatus and uses provided herein can also be performed when charges of a payload, such as nucleic acid molecule(s), polypeptide(s), drug(s) or combinations thereof and mitochondrion are modulated, such as inverted. Accordingly, the present invention also provides a mitochondrion comprising one or more payloads, such as nucleic acid molecule(s), polypeptide(s), drug(s) or combinations thereof attached to the outer membrane of the mitochondrion, wherein the one or more payloads, such as nucleic acid molecule(s), polypeptide(s), drug(s) or combinations thereof is electrostatically attached to the outer membrane of the mitochondrion, wherein:
(a) polycations or positively-charged species are attached to the outer surface of a mitochondrion resulting in a positively-charged mitochondrion surface; and
(b) one or more payloads, such as nucleic acid molecule(s), polypeptide(s), drug(s) or combinations thereof is electrostatically attached to the positively-charged mitochondrion surface via the positively-charged species. A mitochondrion comprising one or more payloads, such as nucleic acid molecule(s), polypeptide(s), drug(s) or combinations thereof attached to the outer membrane of the mitochondrion, wherein the one or more payloads, such as nucleic acid molecule(s), polypeptide(s), drug(s) or combinations thereof is electrostatically attached to the outer membrane of the mitochondrion, wherein the surface of the mitochondrion is positively-charged, wherein:

(a) the one or more payloads, such as nucleic acid molecule(s), polypeptide(s), drug(s) or combinations thereof is attached to or encapsulated in a positively-charged nanoparticle; and

(b) the positively-charged nanoparticle comprising the one or more payloads, such as nucleic acid molecule(s), polypeptide(s), drug(s) or combinations thereof is electrostatically attached to a mitochondrion.

The present invention is not particularly limited to any nanoparticles and may be any nanoparticle as described hereinabove.

In some embodiments, the mitochondrion according to the invention comprising one or more payloads, such as nucleic acid molecule(s), polypeptide(s), drug(s) or combinations thereof as described hereinabove, may be linked to and/or enveloped in a protective layer.

The term “protective layer”, as used herein, refers to a layer which partially or wholly covers, coats, and/or encapsulates (i.e., envelops) the mitochondrion according to the present invention. The protective layer of the present invention is used to modify the mitochondrion to improve the pharmacokinetic and pharmacodynamic properties of the mitochondrion. In particular, the protective layer may, inter alia, increase plasma half-life of the mitochondrion, protect the mitochondrial payload, e.g., the one or more nucleic acid molecule(s), from degradation, i.e., the decomposition of the payloads, such as nucleic acid molecule(s), polypeptide(s), drug(s) or combinations thereof into its component parts upon in vivo administration. Furthermore, the protective layer may enhance the stability of the payload, for example, the protective layer can have a stabilising effect on the one or more payloads, such as nucleic acid molecule(s), polypeptide(s), drug(s) or combinations thereof. The protective layer also prevents or reduces an immune response or cytotoxicity when the mitochondrion is internalized into cells. As can be seen from the appended examples (see e.g., Example 24 to 29), the mitochondrial delivery platform comprising a protective layer is more effective in delivering, e.g., payloads, such as nucleic acid molecule(s), polypeptide(s), drug(s) or combinations thereof, achieving higher transcription of mRNA and higher protein knockdown by siRNA compared to previous approaches.

The protective layer of the present invention preferably comprises polymer or lipid constituents or molecules. In some embodiments, the protective layer envelops the mitochondrion according to the present invention, forming a mitochondrion enveloped particle. In further embodiments, the protective layer partially covers or coats the mitochondrion according to the present invention, forming a mitochondrion having a protective layer surface coating. The protective layer may be linked to the mitochondrion either electrostatically or covalently, directly (e.g., directly linked to the outer membrane of the mitochondrion) or indirectly (e.g., linked via another entity to the outer membrane to the mitochondrion). In preferred embodiments, the protective layer envelops the mitochondrion comprising the one or more payloads, such as nucleic acid molecule(s), polypeptide(s), drug(s) or combinations thereof attached to the outer membrane of the mitochondrion, preferably wherein the one or more payloads, such as nucleic acid molecule(s), polypeptide(s), drug(s) or combinations thereof is electrostatically attached to the outer membrane of the mitochondrion via a polycationic species, in particular a linear or branched polycationic polymer according to the present invention. The protective layer enveloping the mitochondrion may also be linked to the mitochondrion, directly e.g., by electrostatic interaction to the outer membrane of the mitochondrion or by covalent linkage to the outer membrane of the mitochondrion, wherein the covalent linkage may be to a polypeptide in the outer membrane of the mitochondrion via, e.g., an amide bond. The protective layer may also be covalently linked to an antibody that specifically binds to an antigen comprised in the outer membrane of the mitochondrion. In further embodiments, the protective layer is linked to the outer membrane of the mitochondrion without enveloping the mitochondrion. The protective layer is linked to the outer membrane of the mitochondrion in particular when the mitochondrion comprises one or more payloads, such as nucleic acid molecule(s), polypeptide(s), drug(s) or combinations thereof attached to the surface of or encapsulated in a nanoparticle, particle, positively-charged particle or positively-charged nanoparticle. In embodiments where the mitochondrion comprises one or more positively-charged particle(s), positively-charged nanoparticle(s), particles or nanoparticle(s), the skilled person will recognize that the surface of the outer membrane of the mitochondrion may not be fully accessible to the molecules comprising the protective layer, thus preventing the full encapsulation (i.e., envelopment) of the mitochondrion.

In some embodiments, the mitochondrion according to the invention comprising one or more payloads, such as nucleic acid molecule(s), polypeptide(s), drug(s) or combinations thereof as described hereinabove, may be linked to and/or enveloped in a protective layer, wherein the protective layer is a protective polymer.

The term “polymer,” as used herein and as defined by F W Billmeyer, J R. in Textbook of Polymer Science, second edition, 1971, refers to a relatively large molecule made up of smaller chemical repeat units, which have undergone a polymerization reaction to provide a polymer product. Chemicals that react with each other to form the repeat units of a polymer are known herein as “monomers,” and a polymer is said herein to be made of “polymerized units” of the monomers that reacted to form the repeat units. The chemical reaction or reactions in which monomers react to become polymerized units of a polymer are known herein as “polymerizing” or “polymerization”. Typically, polymers comprise 11 or more monomers. Polymers may have structures that are linear, branched, star shaped, looped, hyperbranched, crosslinked, or a combination thereof; polymers may have a single type of repeat monomer units (“homopolymers”), or they may have more than one type of repeat monomer units (“copolymers”). Copolymers may have various types of repeat monomer units arranged randomly, in sequence, in blocks, in other arrangements, or in any mixture or combination thereof. Generally, polymers have weight-average molecular weight (Mw) of 1,000 or more. Polymer molecular weights may be measured by standard methods such as, for example, size exclusion chromatography or intrinsic viscosity. The broadest range value for MW is between 1′000 (one thousand) Daltons and 2′000′000 (2 million) Daltons, preferably between 1′000 and 500′000 Daltons. A preferred range for the polycationic species is an MW between 10′000 and 70′000 Daltons. For the protective polymer a preferred MW is about 15′000 Daltons.

In some embodiments, the protective polymer is a linear or branched cationic polymer, optionally the linear or branched cationic polymer is electrostatically linked to the one or more payloads, such as nucleic acid molecule(s), polypeptide(s), drug(s) or combinations thereof. In some embodiments, the protective polymer is a linear or branched cationic polymer, optionally the linear or branched cationic polymer is covalently linked to the one or more payloads, such as nucleic acid molecule(s), polypeptide(s), drug(s) or combinations thereof.

The term “linear or branched cationic polymer”, as used herein, refers to a linear or branched cationic homopolymer. The term “linear polymer”, as used herein, refers to a polymer comprising repeat monomer units that are attached to each other to form a straight linear structure, while “branched polymers” comprise a linear polymer chain substituted with one or more polymer chains (either short or long polymer chains). As defined herein, a cationic homopolymer is a polymer that contains one or more cationic monomer(s) as polymerized units.

In some embodiments, one or more cationic monomer(s) are used that contain a cation that exists in cationic form when in solution at some range of pH values useful for the application of the present invention, while that cation may be in neutral form at some other pH values. In some embodiments, at least one cationic monomer is used that is in neutral form during polymerization; in such embodiments, after polymerization, conditions surrounding the polymer (such as, for example, pH) are altered so that the polymerized unit resulting from that cationic monomer acquires a positive charge. Independently, in some embodiments, one or more cationic monomers are used that contain a cationic group that is permanently in cationic form (i.e., a cation that remains in cationic form at all pH values below 9). Cations that are permanently in cationic form include, for example, quaternary ammonium salts. In some embodiments, one or more cationic polymer is used in which every cationic group is permanently in cationic form. In some embodiments, every cationic group in every cationic polymer that is used is permanently in cationic form. The anion or anions corresponding to the cation(s) may be in solution, in a complex with the cation (such as a nucleic acid-cationic polymer complex or a mitochondrion-polymer complex), located elsewhere on the polymer, or a combination thereof. The anion corresponding to the cation of a suitable cationic monomer may be any type of anion. Suitable anions include, but are not limited to, halides (including, for example, chloride, bromide, or iodide), hydroxide, phosphate, sulfate, hydrosulfate, ethyl sulfate, methyl sulfate, formate, acetate, or any mixture thereof. Moreover, the anions may be substituted in the process of forming the mitochondrion and protective layer, i.e., the polymer comprising the protective layer may have one type of anion prior to contact with the mitochondrion, which is then substituted for another type of anion, e.g., payloads, such as nucleic acid molecule(s), polypeptide(s), drug(s) or combinations thereof or the mitochondrion of the present invention.

In some embodiments, the linear or branched cationic polymer may be electrostatically linked to the one more payloads, such as nucleic acid molecule(s), polypeptide(s), drug(s) or combinations thereof, thus the one or more payloads, such as nucleic acid molecule(s), polypeptide(s), drug(s) or combinations thereof may be bound to the interior surface of the protective cationic polymer layer. In some embodiments, the linear or branched cationic polymer may be covalently linked to the one more payloads, such as nucleic acid molecule(s), polypeptide(s), drug(s) or combinations thereof, thus the one or more payloads, such as nucleic acid molecule(s), polypeptide(s), drug(s) or combinations thereof may be bound to the interior surface of the protective cationic polymer layer.

The linear or branched cationic polymer is not particularly limited and may be any suitable linear or branched cationic polymer. In preferred embodiments, the linear or branched cationic polymer is polyethyleneimine, RGD-modified polyethyleneimine, polylysine, RGD-modified polylysine, polyornithine, RGD-modified polyornithine, polyarginine, RGD modified polyarginine, polypropyleneimine, RGD-modified polypropyleneimine, polyallylamine, RGD-modified polyallylamine, chitosan, RGD-modified chitosan, poly(2-(dimethylamino)ethyl methacrylate), RGD-modified poly(2-(dimethylamino)ethyl methacrylate), poly(amidoamine)s, RGD-modified poly(amidoamine)s or a combination thereof.

In some embodiments, the protective polymer is a linear or branched cationic copolymer, optionally the linear or branched cationic copolymer is electrostatically linked to the one or more payloads, such as nucleic acid molecule(s), polypeptide(s), drug(s) or combinations thereof.

The term “copolymer” refers to a copolymer as described hereinabove. The copolymer of the present invention may be linear (e.g., block copolymer, alternating copolymer, periodic copolymer, statistical copolymer, stereoblock copolymer or gradient copolymer) or branched (e.g., graft or star copolymer).

In some embodiments, the protective polymer is a linear or branched cationic block copolymer, optionally wherein the linear or branched cationic block copolymer is electrostatically linked to the one or more payloads, such as nucleic acid molecule(s), polypeptide(s), drug(s) or combinations thereof. In some embodiments, the protective polymer is a linear or branched cationic block copolymer, optionally wherein the linear or branched cationic block copolymer is covalently linked to the one or more payloads, such as nucleic acid molecule(s), polypeptide(s), drug(s) or combinations thereof.

As used herein, the term “block copolymer” is a copolymer that comprises more than one species of monomer, wherein the monomers are present in blocks. Each block of the monomer comprises repeating sequences of the monomer. Moreover, a block is a portion of the polymer, comprising repeat monomer units, that has at least one feature which is not present in the adjacent blocks. A formula representative of a block copolymer is: -(A)ₐ-(B)_{b}-(C)_{c}-(D)_{d} . . . (Z)_{z}—, wherein A, B, C, D, through Z represent monomer units and the subscripts “a”, “b”, “c”, “d” through “z”, represent the number of repeating units of A, B, C, D through Z, respectively. The representative formula is not meant to limit the structure of the block copolymer used in the present invention. The block copolymer of the present invention may be a diblock, triblock, tetrablock etc. copolymer. Moreover, the block copolymer can also be linear or branched block copolymers.

In some embodiments, the cationic block copolymer is poly(ethylene glycol)-block-polyethyleneimine, RGD-modified poly(ethylene glycol)-block-polyethyleneimine, poly(ethylene glycol)-block-polylysine, RGD-modified poly(ethylene glycol)-block-polylysine, poly(ethylene glycol)-block-polyornithine, RGD-modified poly(ethylene glycol)-block-polyornithine, poly(ethylene glycol)-block-polyarginine, RGD-modified poly(ethylene glycol)-block-polyarginine, poly(ethylene glycol)-block-polypropyleneimine, RGD-modified poly(ethylene glycol)-block-polypropyleneimine, poly(ethylene glycol)-block-polyallylamine, RGD-modified poly(ethylene glycol)-block-polyallylamine, poly(ethylene glycol)-block-poly(2-(dimethylamino)ethyl methacrylate), RGD-modified poly(ethylene glycol)-block-poly(2-(dimethylamino)ethyl methacrylate), poly(ethylene glycol)-block-poly(amidoamine)s, RGD-modified poly(ethylene glycol)-block-poly(amidoamine)s or a combination thereof.

In some embodiments, the protective polymer is a cationic graft (g) copolymer, optionally the cationic graft (g) copolymer is electrostatically linked to the one or more nucleic acid molecule(s). In some embodiments, the protective polymer is a cationic graft (g) copolymer, optionally the cationic graft (g) copolymer is covalently linked to the one or more nucleic acid molecule(s).

The term “graft copolymer”, as used herein, refers to branched polymers formed when polymer or copolymer chains are chemically attached as side chains to a polymeric backbone. Typically, the side chains are of a different polymeric composition than the backbone chain. Graft copolymers have unique properties including, for example, mechanical film properties resulting from thermodynamically driven microphase separation of the polymer.

In some embodiments, the cationic graft (g) copolymer is poly(ethylene glycol)-g-polyethyleneimine, RGD-modified poly(ethylene glycol)-g-polyethyleneimine, poly(ethylene glycol)-g-polylysine, RGD-modified poly(ethylene glycol)-g-polylysine, poly(ethylene glycol)-g-polyornithine, RGD-modified poly(ethylene glycol)-g-polyornithine, poly(ethylene glycol)-g-polyarginine, RGD-modified poly(ethylene glycol)-g-polyarginine, poly(ethylene glycol)-g-polypropyleneimine, RGD-modified poly(ethylene glycol)-g-polypropyleneimine, poly(ethylene glycol)-g-polyallylamine, RGD-modified poly(ethylene glycol)-g-polyallylamine, poly(ethylene glycol)-g-poly(2-(dimethylamino)ethyl methacrylate), RGD-modified poly(ethylene glycol)-g-poly(2-(dimethylamino)ethyl methacrylate), poly(ethylene glycol)-g-poly(amidoamine)s, RGD-modified poly(ethylene glycol)-g-poly(amidoamine)s or a combination thereof.

In further embodiments, the protective polymer is a linear or branched pegylated (PEG) cationic polymer, optionally the linear or branched pegylated (PEG) cationic polymer is electrostatically linked to the one or more payloads, such as nucleic acid molecule(s), polypeptide(s), drug(s) or combinations thereof. In further embodiments, the protective polymer is a linear or branched pegylated (PEG) cationic polymer, optionally the linear or branched pegylated (PEG) cationic polymer is covalently linked to the one or more payloads, such as nucleic acid molecule(s), polypeptide(s), drug(s) or combinations thereof.

As used herein, the term PEGylated cationic polymer refers to a cationic polymer modified with poly(ethylene glycol) (PEG) or a derivative thereof via a covalent bond or non-covalent force (such as ionic interaction or hydrogen bonding). The modification of materials with groups derived from PEG (also referred to as polyethylene oxide) is known as PEGylation. PEGylation of bioactive entities may prevent degradation of the entities, in particular by proteolytic enzymes. Other advantages of PEGylation include, but are not limited to, increased water solubility, increased bioavailability, increased blood circulation, decreased aggregation, decreased immunogenicity, reduced toxicity, and decreased frequency of administration. In some embodiments, the pegylated (PEG) cationic polymer is pegylated-polyethyleneimine, RGD-modified pegylated polyethyleneimine, pegylated polylysine, RGD-modified pegylated polylysine, histidylated polylysine, pegylated polyornithine, RGD-modified pegylated polyornithine, pegylated polyarginine, RGD-modified pegylated polyarginine, pegylated polypropyleneimine, RGD-modified pegylated polypropyleneimine, pegylated polyallylamine, RGD-modified pegylated polyallylamine, pegylated chitosan, RGD-modified pegylated chitosan, pegylated poly(2-(dimethylamino)ethyl methacrylate), RGD-modified pegylated poly(2-(dimethylamino)ethyl methacrylate), pegylated poly(amidoamine)s RGD-modified pegylated poly(amidoamine)s or a combination thereof.

In some embodiments, the protective layer is a lipid formulation, optionally wherein the lipid formulation is a cationic lipid formulation, further optionally wherein the cationic lipid formulation is electrostatically linked to the one or more payloads, such as nucleic acid molecule(s), polypeptide(s), drug(s) or combinations thereof.

Lipid formulations comprise lipid molecules which form a lipid particle (such as a liposome) or a lipid layer. The lipid formulation of the present invention may be linked to and/or envelope the mitochondrion of the present invention. The lipid formulation may partially cover or coat the mitochondrion of the present invention or envelop the mitochondrion of the invention. In some embodiments, the lipid formulation which envelops the mitochondrion of the invention is a liposome.

The term “liposome” as used herein, is a structure having a one or more lipid membrane(s) enclosing, inter alia, an aqueous interior comprising the mitochondrion of the invention. The present invention may comprise both single-layered liposomes, which are referred to as unilamellar, and multi-layered liposomes, which are referred to as multilamellar. The choice of lipid formulations and the lipids comprised therein is dependent on a variety of considerations, including, inter alia, stability, physicochemical properties, payload loading efficiency, payload release efficiency and toxicity. The lipids comprised in the lipid formulation of the present invention may be any lipid which are capable of linking to and/or enveloping the mitochondrion of the present invention, these include, but are not limited to fatty acids, glycerolipids, glycerophospholipids, sphingolipids and sterols. The lipid comprised in the lipid formulation may be an amphipathic lipid which comprises both hydrophilic (polar) and hydrophobic (non-polar) groups. Amphipathic lipids include, but are not limited to, phospholipids, aminolipids, and sphingolipids. The lipid comprised in the lipid formulation of the present invention may comprise one or more saturated or unsaturated acyl groups of various carbon chain lengths. In preferred embodiments, the one or more lipid(s) comprised in the lipid formulation comprises one or more saturated, monounsaturated or diunsaturated fatty acids having a carbon chain length of between C14 and C22. The lipids of the present invention may also comprise a mixture of saturated and unsaturated fatty acids chains.

As used herein, the term “cationic lipid” comprised in the cationic lipid formulation, refers to a lipid having one or more fatty acid or fatty alkyl chain(s) and a cationic or a cationic ionizable group (i.e., a functional group), such as an amino group (including alkylamino, dialkylamino, trialkylamino and quaternary alkylamino groups). A cationic group refers to a group, which is positively-charged at physiological pH (e.g., at about a pH of 7.4). A cationic ionizable group refers to a group which may be protonated to form a cationic lipid at or below physiological pH, for example, at a pH below about 6.5, which is the typical pH within an endosome. One advantage of the protonation of the cationic ionizable group in the endosome is that it facilitates membrane fusion and subsequent cytosolic release. In certain embodiments, the cationic ionizable lipid has a pKa of the protonatable group in the range of about 6 to about 7. The overall pKa of a lipid formulation is dependent not only on the pKa of each lipid but also on the molar ratio of the lipids. Each lipid has a distinct pKa which may be changed by modifying its ionizable group. Therefore, one strategy to adjust the overall pKa of a lipid formulation is to chemically modify the lipid. Another strategy is to use a mixture of two or more lipids with different pKa and adjust their ratio to achieve the desirable apparent pKa. Cationic lipid formulations may also be electrostatically linked to the one or more nucleic acid molecule(s). Cationic lipids include, but are not limited to, DOSPA (2,3-dioleyloxy-N-[2 (sperminecarboxamido)ethyl]-N,N-dimethyl-1-propanaminium), DC-cholesterol (3β-[N—(N′,N′-Dimethylaminoethane)-carbamoyl]cholesterol hydrochloride), DOTAP (1,2-dioleoyl-3-trimethylammonium-propane chloride), DOTMA (1,2-di-O-octadecenyl-3-trimethylammonium propane chloride), UGG (unsaturated guanidinium glycoside), DOPE (1,2-Dioleoyl-sn-glycerophosphoethanolamine) and lipofectamine. Lipofectamine (also referred to as lipofectamine 2000) typically comprises a 3:1 mixture of DOSPA and DOPE. The lipid formulation may also comprise one or more neutral lipid(s), wherein the neutral lipid molecules are either in an uncharged or neutral zwitterionic form at physiological pH. Neutral lipids include, but are not limited to, DLinDMA (1,2-dilinoleyloxy-3-dimethylaminopropane), DLinMC3DMA (dilinoleylmethyl-4-dimethylaminobutyrate), DODMA (1,2-dioleyloxy-3-dimethylaminopropane) and DOGS (dioctadecylamidoglycylspermine). As is understood by the skilled person, the neutral lipids may also be ionizable cationic lipids under conditions in which the neutral lipids are protonated.

The lipid formulation of the present invention may also comprise one or more anionic lipids. Anionic lipids suitable for the lipid formulations of the invention include, but are not limited to, phosphatidylglycerol, cardiolipin, diacylphosphatidylserine, diacylphosphatidic acid, N-acyl phosphatidylethanolamine, N-succinyl phosphatidylethanolamine, N-glutaryl phosphatidylethanolamine and lysylphosphatidylglycerol,

In some embodiments, the lipid formulation comprises DC-cholesterol (3β-[N—(N′,N′-Dimethylaminoethane)-carbamoyl]cholesterol hydrochloride), DLinDMA (1,2-dilinoleyloxy-3-dimethylaminopropane), DLinMC3DMA (dilinoleylmethyl-4-dimethylaminobutyrate), DODMA (1,2-dioleyloxy-3-dimethylaminopropane), DOGS (dioctadecylamidoglycylspermine), DOSPA (2,3-dioleyloxy-N-[2 (sperminecarboxamido)ethyl]-N,N-dimethyl-1-propanaminium), DOTAP (1,2-dioleoyl-3-trimethylammonium-propane chloride), DOTMA (1,2-di-O-octadecenyl-3-trimethylammonium propane chloride)), UGG (unsaturated guanidinium glycoside), DOPE (1,2-Dioleoyl-sn-glycerophosphoethanolamine), lipofectamine or a combination thereof.

Moreover, the lipid formulation of the invention may further comprise one or more of another lipid, preferably wherein said lipid is cholesterol, a substituted or unsubstituted cholesterol, a cholesterol derivative, such as a hydroxylated cholesterol derivative (e.g., a hydroxycholesterol), a PEG-lipid, DMPC (1,2-Dimyristoyl-sn-glycero-3-phosphocholine), DSPC (1,2-Distearoyl-sn-glycero-3-phosphocholine), DODAP (1,2-dioleoyl-3-dimethylammonium propane), DDA (dimethyldioctadecylammonium), 1,2-dioleoyl-sn-glycero-3-phosphate, 1,2-dimyristoyl-sn-glycero-3-phosphate, bis(monooleoylglycerol)phosphate or a combination thereof.

The lipid formulations of the present invention may further include one or more additional lipid(s). Additional lipids may be included in the lipid formulation for a variety of purposes, such as to prevent lipid oxidation, attach ligands onto the lipid formulation surface, stabilize the lipid formulation or improve payload delivery. The additional lipid comprised in the lipid formulation may be any lipid, including but not limited to, amphipathic, neutral, cationic, and anionic lipids. Stabilizing lipids, in the context of the present invention, may refer to lipids which render the lipid formulation resistant to chemical change. Stabilizing lipids include, but are not limited to, sterols such as cholesterol, a substituted or unsubstituted cholesterol, a cholesterol derivative, such as a hydroxylated cholesterol derivative (e.g., a hydroxycholesterol), a PEG-lipid, such as PEG coupled to phosphatidylethanolamine, PEG conjugated to ceramide and a lipid selected to reduce aggregation of lipid molecules during formation, which may result from steric stabilization of particles which prevents charge-induced aggregation during formation. Examples of molecules which may be conjugated to a lipid to reduce aggregation of particles during formation include PEG, monosialoganglioside (Gm1), polyamide oligomers (PAO), such as ATTA. It should be noted that aggregation preventing compounds do not necessarily require lipid conjugation to function properly. Free PEG or free ATTA in solution may be sufficient to prevent aggregation. If the lipid formulation is stable after formation, the PEG or ATTA may be dialyzed away before administration to a subject.

In some embodiments, the lipid formulation of the present invention comprises a mixture of any one of the lipids mentioned hereinabove, and exemplary lipid formulation may comprise a cationic lipid, neutral lipid (other than a cationic lipid), a sterol (e.g., cholesterol) and a PEG-modified lipid.

In some embodiments, the mitochondrion of the present invention is linked to and/or enveloped in a zwitterionic protective polymer, optionally wherein the zwitterionic protective polymer is electrostatically linked to the one or more payloads, such as nucleic acid molecule(s), polypeptide(s), drug(s) or combinations thereof.

The zwitterionic protective polymer may be a homopolymer or copolymer as described hereinabove. A zwitterionic polymer comprises one or more positive charge(s) and one or more negative charge(s) wherein the overall (i.e., net) charge of the polymer is substantially electronically neutral. In one embodiment, the zwitterionic polymer is a zwitterionic copolymer, wherein the ratio of the number of positively-charged repeating units to the number of the negatively-charged repeating units is from about 1:1.1 to about 1:0.5. In one embodiment, the ratio of the number of positively-charged repeating units to the number of the negatively-charged repeating units is from about 1:1.1 to about 1:0.7. In one embodiment, the ratio of the number of positively-charged repeating units to the number of the negatively-charged repeating units is from about 1:1.1 to about 1:0.9.

In preferred embodiments, the zwitterionic protective polymer is selected from: poly(2-methacryloyloxyethyl phosphorylcholine) (PMPC), polyethyleneimine-g-poly(2-methacryloyloxyethyl phosphorylcholine) (PEI-g-PMPC), co-assembly of cationic (carboxyl-functionalized) and anionic (amino-functionalized) copolyesters based on poly(ε-caprolactone)-block-poly(butylene e fumarate)-block-poly(ε-caprolactone) (PCL-b-PBF-b-PCL), poly(lactic-co-glycolic acid) (PLGA)-PCB block copolymers (PLGA-b-PCB).

In some embodiments, the protective layer is linked to a targeting moiety, optionally wherein the protective layer linked to targeting moiety is electrostatically linked to the one or more payloads, such as nucleic acid molecule(s), polypeptide(s), drug(s) or combinations thereof. In preferred embodiments, the targeting moiety is an antibody or carbohydrate molecule. In further preferred embodiments, the targeting moiety is comprised on the outer surface of the protective layer. The outer surface of the protective layer is the surface in contact with the environment, wherein the inner surface is in proximity to or in contact with the mitochondrion and payloads comprised therein. In some embodiments, the protective layer is linked to a targeting moiety, optionally wherein the protective layer linked to targeting moiety is covalently linked to the one or more payloads, such as nucleic acid molecule(s), polypeptide(s), drug(s) or combinations thereof. In preferred embodiments, the targeting moiety is an antibody or carbohydrate molecule. In further preferred embodiments, the targeting moiety is comprised on the outer surface of the protective layer.

In further embodiments, the protective layer is linked to an antibody, optionally wherein the protective layer linked to an antibody is electrostatically linked to the one or more payloads, such as nucleic acid molecule(s), polypeptide(s), drug(s) or combinations thereof.

Moreover, in some embodiments the protective layer is linked to a carbohydrate, optionally wherein the protective layer linked to a carbohydrate is electrostatically linked to the one or more payloads, such as nucleic acid molecule(s), polypeptide(s), drug(s) or combinations thereof.

The carbohydrate or antibody linked to the protective layer of the present invention is preferably a targeting moiety, i.e., a moiety that targets a cell or tissue or a molecule comprised therein via an affinity type interaction. Targeting mechanisms generally require that the targeting moiety be positioned on the surface of the protective layer in such a manner that the targeting moiety is available for interaction with the target, for example, a cell surface receptor. Targeting moieties enhance the association of the entities to which they are linked with the target cells, tissues, specific cell types or molecules comprised therein, such as cell-surface molecules. The targeting moiety may be a carbohydrate, such as, lactose, galactose, N-acetyl galactoseamine (NAG), mannose, mannose-6-phosphate (M6P) or a derivative thereof but is not limited to these examples. As used herein, the term “antibody” encompasses not only intact (i.e., full-length) monoclonal antibodies, but also antigen-binding fragments (such as Fab, Fab′, F(ab′)2, Fv, single chain variable fragment (scFv)), mutants thereof, fusion proteins comprising an antibody portion, humanized antibodies, chimeric antibodies, diabodies, linear antibodies, single chain antibodies, single domain antibodies (e.g., camel or llama VHH antibodies), multi-specific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins may be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known. Exemplary targeting antibodies include, but are not limited to, monoclonal antibodies, whole antibodies or antibody fragments. Antibodies as targeting antibodies can be any antibody as defined herein above. Standard methods for linking the targeting moiety, e.g., the carbohydrate or antibody, may be used. For example, the targeting moiety may be covalently attached to the protective layer via an amide bond, thioester bond, a disulfide bond or a hydrazone bond. Covalent attachment of the targeting moiety may be performed prior to the formation of the protective layer, by covalent attachment of the targeting moiety to a polymer or lipid comprised in the protective layer, alternatively the targeting moiety may be covalently attached to the protective layer after it has been formed. Methods for attaching targeting moieties are well understood by the skilled person and described in numerous review articles (e.g., Z. Zhao et al., Cell, 2020, 181, p 151-167; M. J. Mitchell et al., Nature Reviews Drug Discovery, 2021, 20, p 101-124). The targeting moieties as part of the present invention are not particularly limited, and may include molecules other than carbohydrates or antibodies, such as peptides, proteins, vitamins and small molecules. The targeting moieties may be electrostatically linked, e.g., to the payload or the protective polymer.

In another aspect, any of the mitochondria described herein may be incorporated into a composition. Accordingly, the present invention provides a composition comprising the mitochondrion of the present invention, wherein the mitochondrion comprises one or more payloads, such as nucleic acid molecule(s), polypeptide(s), drug(s) or combinations thereof attached to the outer membrane of the mitochondrion, wherein the one or more payloads, such as nucleic acid molecule(s), polypeptide(s), drug(s) or combinations thereof:
a) is electrostatically attached to the outer membrane of the mitochondrion via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule.

A composition can include any of the mitochondria described herein and any additional compound useful for facilitating delivery.

The mitochondria and compositions of the present invention may be formulated into a pharmaceutical composition comprising an acceptable carrier, such as a pharmaceutically acceptable carrier. As used herein, the term “pharmaceutically acceptable” refers to those compounds, materials, compositions, and/or dosage forms which are, within the scope of sound medical judgment, suitable for use in contact with the tissues, organs, and/or bodily fluids of the subject without excessive toxicity, irritation, allergic response, or other problems or complications commensurate with a reasonable benefit/risk ratio. As used herein, the term “pharmaceutically acceptable carrier” refers to solvents, dispersion media, coatings, antibacterial agents, antifungal agents, isotonic and absorption delaying agents, or the like that are physiologically compatible. The compositions may include a pharmaceutically acceptable salt, e.g., an acid addition salt or a base addition salt.

Accordingly, the present invention relates to a pharmaceutical composition comprising the mitochondrion of the present invention as described hereinabove and a pharmaceutically acceptable carrier. A pharmaceutical composition can comprise a mitochondrion as described herein and a pharmaceutically acceptable carrier, wherein the pharmaceutical composition is formulated as a solution. A pharmaceutical composition can comprise a mitochondrion as described herein and a pharmaceutically acceptable carrier, wherein the pharmaceutical composition is formulated as an aerosol.

The products, compositions and mitochondria described herein may be used in therapy. A mitochondrion used in the present application for therapy may be used in an allogeneic or autologous manner. The present invention provides a mitochondrion, compositions, and pharmaceutical compositions for use in the treatment of a disease that may benefit from the use of healthy mitochondria and the combination of healthy mitochondria and nucleic acid molecules. It is envisioned to increase expression of certain target proteins, for example through a delivery of messenger RNA (mRNA) or decrease of certain target proteins through a delivery of small interference RNA (siRNA). Accordingly, the present invention provides treatments of cardiovascular diseases (CVD) in human such as ischemic heart disease, ischemia-reperfusion injury, and atherosclerosis, treatments of aging related diseases such as sarcopenia, Parkinson's disease and Hutchinson-Gilford progeria syndrome (HGPS), treatments of kidney diseases, such as autosomal dominant polycystic kidney disease, Alport syndrome, Nephronophthisis, and Fabry disease, methods and treatments using in vitrolin vivo gene transfection and editing using CRISPR-Cas9, gene therapy treatments for diseases such as cystic fibrosis and cancer treatments.

The terms “medicament” and “pharmaceutical composition” are used interchangeably herein. Accordingly, definitions and explanations provided herein in relation to “pharmaceutical compositions”, apply, mutatis mutandis, to the term “medicament”. Accordingly, the present invention provides a mitochondrion for use as a medicament, comprising one or more payloads, such as nucleic acid molecule(s), polypeptide(s), drug(s) or combinations thereof attached to the outer membrane of the mitochondrion, wherein the one or more payloads, such as nucleic acid molecule(s), polypeptide(s), drug(s) or combinations thereof:
a) is electrostatically attached to the outer membrane of the mitochondrion via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule.

The present invention provides a composition for use as a medicament comprising a mitochondrion, comprising one or more payloads, such as nucleic acid molecule(s), polypeptide(s), drug(s) or combinations thereof attached to the outer membrane of the mitochondrion, wherein the one or more payloads, such as nucleic acid molecule(s), polypeptide(s), drug(s) or combinations thereof:
a) is electrostatically attached to the outer membrane of the mitochondrion via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule.

The present invention provides a pharmaceutical composition for use as a medicament comprising a mitochondrion, comprising one or more payloads, such as nucleic acid molecule(s), polypeptide(s), drug(s) or combinations thereof attached to the outer membrane of the mitochondrion, wherein the one or more payloads, such as nucleic acid molecule(s), polypeptide(s), drug(s) or combinations thereof)
a) is electrostatically attached to the outer membrane of the mitochondrion via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule.

The mitochondrion, compositions and pharmaceutical compositions of the present invention may be used for gene therapy. The present invention provides a delivery platform for payloads, such as nucleic acid molecule(s), polypeptide(s), drug(s) or combinations thereof which is especially useful for in vivo, ex vivo or in vitro gene therapy. The skilled person is aware that an in vivo gene therapy or gene editing method can relate to a therapy or gene editing method in a subject, an ex vivo gene therapy or gene editing method can relate to a therapy or gene editing method in e.g., an organ artificially maintained outside of a subject and an in vitro gene therapy or gene editing method can relate to a therapy or gene editing method e.g., in a cell or tissue in a culture. “Gene therapy” as used herein relates to the modification of a subject's gene to treat or cure a disease. The terms “gene editing” and “genome editing” may be used herein interchangeably. Accordingly, the present invention provides a mitochondrion for use in gene therapy, comprising one or more payloads, such as nucleic acid molecule(s), polypeptide(s), drug(s) or combinations thereof attached to the outer membrane of the mitochondrion, wherein the one or more payloads, such as nucleic acid molecule(s), polypeptide(s), drug(s) or combinations thereof:
a) is electrostatically attached to the outer membrane of the mitochondrion via a positively-charged species; or
b) is covalently linked to the outer membrane of the mitochondrion; or
c) is linked to an antibody that specifically binds to an antigen comprised in the outer membrane of the mitochondrion;
d) is linked to a mitochondria-targeting small molecule.

The present invention provides a composition for use in gene therapy comprising a mitochondrion, comprising one or more payloads, such as nucleic acid molecule(s), polypeptide(s), drug(s) or combinations thereof attached to the outer membrane of the mitochondrion, wherein the one or more payloads, such as nucleic acid molecule(s), polypeptide(s), drug(s) or combinations thereof:
a) is electrostatically attached to the outer membrane of the mitochondrion via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule.

The present invention provides a pharmaceutical composition for use in gene therapy comprising a plurality of a mitochondrion, comprising one or more payloads, such as nucleic acid molecule(s), polypeptide(s), drug(s) or combinations thereof attached to the outer membrane of the mitochondrion, wherein the one or more payloads, such as nucleic acid molecule(s), polypeptide(s), drug(s) or combinations thereof
a) is electrostatically attached to the outer membrane of the mitochondrion via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule.

Accordingly, the present invention provides a mitochondrion for use in in vitro, ex vivo or in vivo genome editing, comprising one or more payloads, such as nucleic acid molecule(s), polypeptide(s), drug(s) or combinations thereof attached to the outer membrane of the mitochondrion, wherein the one or more payloads, such as nucleic acid molecule(s), polypeptide(s), drug(s) or combinations thereof:
a) is electrostatically attached to the outer membrane of the mitochondrion via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule.

The present invention provides a composition for use in in vitro, ex vivo or in vivo genome editing comprising a plurality of a mitochondrion, comprising one or more payloads, such as nucleic acid molecule(s), polypeptide(s), drug(s) or combinations thereof attached to the outer membrane of the mitochondrion, wherein the one or more payloads, such as nucleic acid molecule(s), polypeptide(s), drug(s) or combinations thereof
a) is electrostatically attached to the outer membrane of the mitochondrion via a positively-charged species; or
b) is covalently linked to the outer membrane of the mitochondrion; or
c) is linked to an antibody that specifically binds to an antigen comprised in the outer membrane of the mitochondrion; or)
d) is linked to a mitochondria-targeting small molecule.

The present invention provides a pharmaceutical composition for use in in vitro, ex vivo or in vivo genome editing comprising a plurality of a mitochondrion, comprising one or more payloads, such as nucleic acid molecule(s), polypeptide(s), drug(s) or combinations thereof attached to the outer membrane of the mitochondrion, wherein the one or more payloads, such as nucleic acid molecule(s), polypeptide(s), drug(s) or combinations thereof:
a) is electrostatically attached to the outer membrane of the mitochondrion via a positively-charged species; or)
b) is covalently linked to the outer membrane of the mitochondrion; or
c) is linked to an antibody that specifically binds to an antigen comprised in the outer membrane of the mitochondrion; or
d) is linked to a mitochondria-targeting small molecule.

The mitochondrion, compositions and pharmaceutical compositions of the present invention may be used in the treatment of a condition or disease in a subject. The term “subject” in general relates to any individual, such as an animal. In the sense of the present invention an individual is preferably a mammal, most preferably a human. The terms “individual”, “subject” and/or “patient” may be used interchangeably.

A disease may be any condition or status of an individual where health is absent. A disease may also be a status of discomfort or malaise. According to the present invention a disease can preferably be a cardiovascular disease, aging related disease, kidney disease, or cancer. In preferred embodiments the disease is ischemic heart disease, atherosclerosis, muscular dystrophy, Parkinson's disease, or Hutchinson-Gilford progeria syndrome.

The terms “treatment”, “treating” and the like are used herein to generally mean obtaining a desired pharmacological and/or physiological effect. The effect may be prophylactic in terms of completely or partially preventing a disease or symptom thereof and/or preventing the progression of a disease or symptom thereof. The term “treatment” as used herein may be understood to relate to any form of therapy.

Accordingly, the present invention provides a mitochondrion for use in the treatment of cardiovascular diseases, comprising one or more payloads, such as nucleic acid molecule(s), polypeptide(s), drug(s) or combinations thereof attached to the outer membrane of the mitochondrion, wherein the one or more payloads, such as nucleic acid molecule(s), polypeptide(s), drug(s) or combinations thereof:
a) is electrostatically attached to the outer membrane of the mitochondrion via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule.

Preferably, the present invention provides a mitochondrion for use in the treatment of ischemic heart disease, ischemia-reperfusion injury, or atherosclerosis, comprising one or more payloads, such as nucleic acid molecule(s), polypeptide(s), drug(s) or combinations thereof attached to the outer membrane of the mitochondrion, wherein the one or more payloads, such as nucleic acid molecule(s), polypeptide(s), drug(s) or combinations thereof:
a) is electrostatically attached to the outer membrane of the mitochondrion via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule.

The present invention provides a composition for use in the treatment of cardiovascular diseases, comprising a plurality of a mitochondrion, comprising one or more payloads, such as nucleic acid molecule(s), polypeptide(s), drug(s) or combinations thereof attached to the outer membrane of the mitochondrion, wherein the one or more payloads, such as nucleic acid molecule(s), polypeptide(s), drug(s) or combinations thereof
a) is electrostatically attached to the outer membrane of the mitochondrion via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule.

Preferably, the present invention provides a composition for use in the treatment of ischemic heart disease, ischemia-reperfusion injury, or atherosclerosis, comprising a plurality of a mitochondrion, comprising one or more payloads, such as nucleic acid molecule(s), polypeptide(s), drug(s) or combinations thereof attached to the outer membrane of the mitochondrion, wherein the one or more payloads, such as nucleic acid molecule(s), polypeptide(s), drug(s) or combinations thereof:
a) is electrostatically attached to the outer membrane of the mitochondrion via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule.

The present invention provides a pharmaceutical composition for use in the treatment of cardiovascular diseases comprising a plurality of a mitochondrion, comprising one or more payloads, such as nucleic acid molecule(s), polypeptide(s), drug(s) or combinations thereof attached to the outer membrane of the mitochondrion, wherein the one or more payloads, such as nucleic acid molecule(s), polypeptide(s), drug(s) or combinations thereof:
a) is electrostatically attached to the outer membrane of the mitochondrion via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule.

Preferably, the present invention provides a pharmaceutical composition for use in the treatment of ischemic heart disease, ischemia-reperfusion injury, or atherosclerosis comprising a plurality of a mitochondrion, comprising one or more payloads, such as nucleic acid molecule(s), polypeptide(s), drug(s) or combinations thereof attached to the outer membrane of the mitochondrion, wherein the one or more payloads, such as nucleic acid molecule(s), polypeptide(s), drug(s) or combinations thereof:
a) is electrostatically attached to the outer membrane of the mitochondrion via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule.

Accordingly, the present invention provides a mitochondrion for use in the treatment of aging related diseases, comprising one or more payloads, such as nucleic acid molecule(s), polypeptide(s), drug(s) or combinations thereof attached to the outer membrane of the mitochondrion, wherein the one or more payloads, such as nucleic acid molecule(s), polypeptide(s), drug(s) or combinations thereof:
a) is electrostatically attached to the outer membrane of the mitochondrion via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule.

Preferably, the present invention provides a mitochondrion for use in the treatment of sarcopenia, Parkinson's disease or Hutchinson-Gilford progeria syndrome, comprising one or more payloads, such as nucleic acid molecule(s), polypeptide(s), drug(s) or combinations thereof attached to the outer membrane of the mitochondrion, wherein the one or more payloads, such as nucleic acid molecule(s), polypeptide(s), drug(s) or combinations thereof:
a) is electrostatically attached to the outer membrane of the mitochondrion via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule.

The present invention provides a composition for use in the treatment of aging related diseases, comprising a plurality of a mitochondrion, comprising one or more payloads, such as nucleic acid molecule(s), polypeptide(s), drug(s) or combinations thereof attached to the outer membrane of the mitochondrion, wherein the one or more payloads, such as nucleic acid molecule(s), polypeptide(s), drug(s) or combinations thereof:
a) is electrostatically attached to the outer membrane of the mitochondrion via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule.

Preferably, the present invention provides a composition for use in the treatment sarcopenia, Parkinson's disease or Hutchinson-Gilford progeria syndrome, comprising a plurality of a mitochondrion, comprising one or more payloads, such as nucleic acid molecule(s), polypeptide(s), drug(s) or combinations thereof attached to the outer membrane of the mitochondrion, wherein the one or more payloads, such as nucleic acid molecule(s), polypeptide(s), drug(s) or combinations thereof:
a) is electrostatically attached to the outer membrane of the mitochondrion via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule.

The present invention provides a pharmaceutical composition for use in the treatment of aging related diseases comprising a plurality of a mitochondrion, comprising one or more payloads, such as nucleic acid molecule(s), polypeptide(s), drug(s) or combinations thereof attached to the outer membrane of the mitochondrion, wherein the one or more payloads, such as nucleic acid molecule(s), polypeptide(s), drug(s) or combinations thereof:
a) is electrostatically attached to the outer membrane of the mitochondrion via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule.

Preferably, the present invention provides a pharmaceutical composition for use in the treatment of sarcopenia, Parkinson's disease or Hutchinson-Gilford progeria syndrome comprising a plurality of a mitochondrion, comprising one or more payloads, such as nucleic acid molecule(s), polypeptide(s), drug(s) or combinations thereof attached to the outer membrane of the mitochondrion, wherein the one or more payloads, such as nucleic acid molecule(s), polypeptide(s), drug(s) or combinations thereof:
a) is electrostatically attached to the outer membrane of the mitochondrion via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule.

Accordingly, the present invention provides a mitochondrion for use in the treatment of kidney diseases, comprising one or more payloads, such as nucleic acid molecule(s), polypeptide(s), drug(s) or combinations thereof attached to the outer membrane of the mitochondrion, wherein the one or more payloads, such as nucleic acid molecule(s), polypeptide(s), drug(s) or combinations thereof:
a) is electrostatically attached to the outer membrane of the mitochondrion via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule.

Preferably, the present invention provides a mitochondrion for use in the treatment of autosomal dominant polycystic kidney disease, Alport syndrome, Nephronophthisis, or Fabry disease, comprising one or more payloads, such as nucleic acid molecule(s), polypeptide(s), drug(s) or combinations thereof attached to the outer membrane of the mitochondrion, wherein the one or more payloads, such as nucleic acid molecule(s), polypeptide(s), drug(s) or combinations thereof:
a) is electrostatically attached to the outer membrane of the mitochondrion via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule.

The present invention provides a composition for use in the treatment of kidney diseases, comprising a plurality of a mitochondrion, comprising one or more payloads, such as nucleic acid molecule(s), polypeptide(s), drug(s) or combinations thereof attached to the outer membrane of the mitochondrion, wherein the one or more payloads, such as nucleic acid molecule(s), polypeptide(s), drug(s) or combinations thereof
a) is electrostatically attached to the outer membrane of the mitochondrion via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule.

Preferably, the present invention provides a composition for use in the treatment of autosomal dominant polycystic kidney disease, Alport syndrome, Nephronophthisis, or Fabry disease, comprising a plurality of a mitochondrion, comprising one or more payloads, such as nucleic acid molecule(s), polypeptide(s), drug(s) or combinations thereof attached to the outer membrane of the mitochondrion, wherein the one or more payloads, such as nucleic acid molecule(s), polypeptide(s), drug(s) or combinations thereof:
a) is electrostatically attached to the outer membrane of the mitochondrion via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule.

The present invention provides a pharmaceutical composition for use in the treatment of kidney diseases comprising a plurality of a mitochondrion, comprising one or more payloads, such as nucleic acid molecule(s), polypeptide(s), drug(s) or combinations thereof attached to the outer membrane of the mitochondrion, wherein the one or more payloads, such as nucleic acid molecule(s), polypeptide(s), drug(s) or combinations thereof:
a) is electrostatically attached to the outer membrane of the mitochondrion via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule.

Preferably, the present invention provides a pharmaceutical composition for use in the treatment of autosomal dominant polycystic kidney disease, Alport syndrome, Nephronophthisis, or Fabry disease comprising a plurality of a mitochondrion, comprising one or more payloads, such as nucleic acid molecule(s), polypeptide(s), drug(s) or combinations thereof attached to the outer membrane of the mitochondrion, wherein the one or more payloads, such as nucleic acid molecule(s), polypeptide(s), drug(s) or combinations thereof:
a) is electrostatically attached to the outer membrane of the mitochondrion via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule.

Accordingly, the present invention provides a mitochondrion for use in the treatment of cancer, comprising one or more payloads, such as nucleic acid molecule(s), polypeptide(s), drug(s) or combinations thereof attached to the outer membrane of the mitochondrion, wherein the one or more payloads, such as nucleic acid molecule(s), polypeptide(s), drug(s) or combinations thereof:
a) is electrostatically attached to the outer membrane of the mitochondrion via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule.

The present invention provides a composition for use in the treatment of cancer comprising a plurality of a mitochondrion, comprising one or more payloads, such as nucleic acid molecule(s), polypeptide(s), drug(s) or combinations thereof attached to the outer membrane of the mitochondrion, wherein the one or more payloads, such as nucleic acid molecule(s), polypeptide(s), drug(s) or combinations thereof
a) is electrostatically attached to the outer membrane of the mitochondrion via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule.

The present invention provides a pharmaceutical composition for use in the treatment of cancer comprising a plurality of a mitochondrion, comprising one or more payloads, such as nucleic acid molecule(s), polypeptide(s), drug(s) or combinations thereof attached to the outer membrane of the mitochondrion, wherein the one or more payloads, such as nucleic acid molecule(s), polypeptide(s), drug(s) or combinations thereof:
a) is electrostatically attached to the outer membrane of the mitochondrion via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule.

As described hereinabove, the mitochondrion, the composition or pharmaceutical composition of the invention is for use in the treatment of various diseases including cardiovascular diseases, ischemia-reperfusion injury, kidney diseases, cancer, mitochondrial dysfunction disorders, metabolic disorders, autoimmune disorders, infectious diseases, inflammatory diseases, muscular diseases and aging related diseases.

The cardiovascular disease is preferably selected from ischemic heart disease, myocardial ischemia, atherosclerosis, myocardial infarction, acute coronary syndrome heart failure, and hypertensive heart disease.

The ischemia-reperfusion injury may be any disease that involves ischemia, preferably the ischemia-reperfusion injury is selected from a liver ischemia-reperfusion injury, an ischemic injury-compartmental syndrome, a chronic ischemia, hypertension and any injury involving ischemia, e.g., myocardial infarction, stroke, organ transplant, and the like.

The kidney disease is preferably selected from autosomal dominant polycystic kidney disease, Alport syndrome, Nephronophthisis, and Fabry disease.

The cancer is preferably selected from acute lymphoblastic leukemia (ALL), acute myeloid leukemia (AML), alveolar rhabdomyosarcoma, bladder cancer (e.g., bladder carcinoma), bone cancer, brain cancer (e.g., glioblastoma), breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, fibrosarcoma, gastrointestinal carcinoid tumor, head and neck cancer (e.g., head and neck squamous cell carcinoma), Hodgkin's lymphoma, hypopharynx cancer, kidney cancer, larynx cancer, leukemia, liquid tumors, liver cancer, lung cancer (e.g., non-small cell lung carcinoma and lung adenocarcinoma), lymphoma, mesothelioma, mastocytoma, melanoma, multiple myeloma, nasopharynx cancer, non-Hodgkin lymphoma, B-chronic lymphocytic leukemia, hairy cell leukemia, Burkitt's lymphoma, ovarian cancer, pancreatic cancer, peritoneum, omentum, and mesentery cancer, pharynx cancer, prostate cancer, rectal cancer, renal cancer, skin cancer, small intestine cancer, soft tissue cancer, solid tumors, synovial sarcoma, gastric cancer, testicular cancer, thyroid cancer, and ureter cancer.

The autoimmune disorder is preferably selected from multiple sclerosis, diabetes, irritable bowel syndrome (IBS), Celiac disease, Crohn's disease, rheumatoid arthritis, systemic lupus erythematosus, autoimmune vasculitis, myasthenia gravis, pernicious anemia, Hashimoto's thyroiditis, type 1 diabetes, autoimmune Addison's disease, Grave's disease, Sjogren's syndrome, psoriasis, and celiac diseases.

The inflammatory disease is preferably selected from rheumatoid arthritis, inflammatory skin diseases such as psoriasis, inflammatory bowel diseases such as colitis, and inflammatory lung diseases such as asthma and bronchitis.

The mitochondrial dysfunction disorder is preferably selected from a disease caused by mutation in the mtDNA such as Keams-Sayre syndrome, mitochondrial encephalomyopathy, lactic acidosis and stroke-like episodes (MELAS) syndrome, Leber's hereditary optic neuropathy, Pearson syndrome, progressive external ophthalmoplegia, mitochondrial myopathy, diabetes mellitus and deafness (DAD), Leigh syndrome, “Neuropathy, ataxia, retinitis pigmentosa, and ptosis” (NARP), myoneurogenic gastrointestinal encephalopathy (MNGIE), myoclonic epilepsy with ragged red fibers syndrome), (MERRF encephalomyopathy, lactic acidosis, Parkinson's disease, and stroke-like symptoms (MELAS syndrome), etc. MERRF syndrome, MELAS syndrome, Leber's disease, Barth syndrome and diabetes.

The metabolic disorder is preferably selected from obesity and its associated metabolic diseases (e.g., type 2 diabetes). Metabolic disorders may be treated or prevented by administering the mitochondrion, the composition or the pharmaceutical composition of the present invention to white adipose tissue in a subject. White adipose tissue or white fat is one of the two types of adipose tissue found in mammals. It is often used by the body as a store of energy and includes many white adipocytes. The other kind of adipose tissue is brown adipose tissue. The function of brown adipose tissue is to transfer energy from food into heat. White adipocytes often contain a single lipid droplet. In contrast, brown adipocytes contain numerous smaller droplets and a much higher number of mitochondria. With the recognition that adult humans have in brown adipose tissue an organ with substantial capacity to dissipate energy, targeting brown adipose tissue thermogenesis is now viewed as a way to treat or prevent metabolic disorders, such as obesity and its associated metabolic diseases (e.g., type 2 diabetes). The use of brown adipose tissue to treat obesity and diabetes is described, e.g., in Cypess, Aaron M., and C. Ronald Kahn. “Brown fat as a therapy for obesity and diabetes.” Current opinion in endocrinology, diabetes, and obesity 17.2 (2010): 143, which is incorporated by reference in its entirety. As one major difference between brown adipocytes and white adipocytes is the number of mitochondria in the cell, the present disclosure provides methods of treating and preventing metabolic disorders by administering the mitochondrion, composition or pharmaceutical composition comprising the mitochondrion to the white adipose tissue in the subject. The administration of the mitochondrion of the present invention to the white adipocytes can convert the white adipocytes to brown adipocytes, thus converting white adipose tissue to brown adipose tissue. The infectious disease is preferably selected from viral infection (e.g., HIV, HCV, RSV), a bacterial infection, a fungal infection and sepsis.

The muscular disorders are preferably selected from Duchenne muscular dystrophy (DMD), Becker muscular dystrophy (BMD), Outlier muscular dystrophy (OMD), Emery-Dreifuss muscular dystrophy (EDMD), Limb-Girdle muscular dystrophy (LGMD), facioscapulohumeral muscular dystrophy (FSH or FSHD; also known as Landouzy-Dejerine), myotonic dystrophy (MMD; also known as Steinert's disease), oculopharyngeal muscular dystrophy (OPMD), distal muscular dystrophy (DD) and congenital muscular dystrophy (CMD). Muscular disorders may also encompass diseases or disorders that involve or can involve voluntary muscle cell death or inflammation, including the myositis disorders polymyositis, dermamyositis and inclusion body myositis, as well as myopathies.

The aging related disease is preferably selected from neurodegenerative diseases (e.g., Parkinson's disease, Alzheimer's disease, Huntington's disease, dementia, etc.), sarcopenia, Hutchinson-Gilford progeria syndrome, osteopenia, osteoporosis, arthritis, atherosclerosis, cardiovascular disease, hypertension, cataracts, presbyopia, glaucoma, type 2 diabetes, metabolic syndrome, alopecia, chronic inflammation, immunosenescence, and age-related visual decline.

The mitochondrion, compositions and pharmaceutical compositions of the present invention may be used in radiation therapy. In particular, the mitochondria of the present invention may be used to deliver a radioactive agent which may be used for radiation therapy. Such a radioactive agent for radiation therapy may be delivered by the delivery system of the present invention into solid tumors. The present invention is not particularly limited to any agent for radiation therapy. Iodine 131 is an exemplary agent for radiation therapy of thyroid cancer. Accordingly, the present invention provides a mitochondrion for use in radiation therapy, comprising one or more payloads, such as nucleic acid molecule(s), polypeptide(s), drug(s) or combinations thereof attached to the outer membrane of the mitochondrion, wherein the one or more payloads, such as nucleic acid molecule(s), polypeptide(s), drug(s) or combinations thereof:
a) is electrostatically attached to the outer membrane of the mitochondrion via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule.

The present invention provides a composition for use in radiation therapy comprising a plurality of a mitochondrion, comprising one or more payloads, such as nucleic acid molecule(s), polypeptide(s), drug(s) or combinations thereof attached to the outer membrane of the mitochondrion, wherein the one or more payloads, such as nucleic acid molecule(s), polypeptide(s), drug(s) or combinations thereof:
a) is electrostatically attached to the outer membrane of the mitochondrion via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule.

The present invention provides a pharmaceutical composition for use in radiation therapy comprising a plurality of a mitochondrion, comprising one or more payloads, such as nucleic acid molecule(s), polypeptide(s), drug(s) or combinations thereof attached to the outer membrane of the mitochondrion, wherein the one or more payloads, such as nucleic acid molecule(s), polypeptide(s), drug(s) or combinations thereof:
a) is electrostatically attached to the outer membrane of the mitochondrion via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule.

The present invention provides a mitochondrion for use in radiation therapy, comprising one or more radioactive agent attached to the outer membrane of the mitochondrion, wherein the one or more radioactive:
a) is electrostatically attached to the outer membrane of the mitochondrion via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule.

In yet another aspect, the present invention provides methods for delivering payloads, such as nucleic acid molecule(s), polypeptide(s), drug(s) or combinations thereof to an organ in a subject by administering the delivery platform of the present invention to a subject. The terms “administering”, “introducing” and “delivering” are used interchangeably in the context of the present invention, e.g., the delivery platform of the present invention, i.e. mitochondrion payload complex may be introduced into a subject by a method or route that results in at least partial localization of the introduced complex at a desired site, such as a site where it is appreciated to produce a desired effect, such as a treatment or therapy. The mitochondrion, compositions or pharmaceutical compositions of the present invention may be administered via a route such as, but not limited to, enteral (into the intestine), gastroenteral, epidural (into the dura matter), oral (by way of the mouth), transdermal, peridural, intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intravenous bolus, intravenous drip, intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracorneal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intramyocardial (within the myocardium), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aureus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration, which is then covered by a dressing that occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), intramyocardial (entering the myocardium), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis and spinal.

Modes of administration include injection, infusion, instillation, and/or ingestion. “Injection” includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In some examples, the route is intravenous. A mitochondrion, composition or pharmaceutical composition of the present invention is administered as a single dose or as at least 2 or more consecutive doses. Preferably, a mitochondrion, composition or pharmaceutical composition of the present invention is administered intravenously or by inhalation. Preferably, a mitochondrion, composition or pharmaceutical composition of the present invention is administered into the bloodstream upstream of the target organ. Preferably, a mitochondrion, composition or pharmaceutical composition of the present invention is administered into an organ. Preferably, a mitochondrion, composition or pharmaceutical composition of the present invention is administered directly into a target organ, such as an organ where therapy is desired. Preferably, a mitochondrion, composition or pharmaceutical composition of the present invention is administered directly into a target organ by injecting the mitochondrion, composition, or pharmaceutical composition to the organ of interest. Preferably, a mitochondrion, composition or pharmaceutical composition of the present invention is administered by inhalation.

In certain embodiments, the target organ is the kidney. In certain embodiments, a mitochondrion, composition or pharmaceutical composition of the present invention is delivered to the kidney of a subject. For that, it is preferred that the mitochondrion, composition or pharmaceutical composition of the present invention is administered upstream of the kidney, i.e., into the renal artery of the subject. Alternatively, the mitochondrion, composition or pharmaceutical composition of the present invention is injected directly into the kidney.

In certain embodiments, the target organ is the heart. In certain embodiments, a mitochondrion, composition or pharmaceutical composition of the present invention is delivered to the heart of a subject. For that, it is preferred that the mitochondrion, composition or pharmaceutical composition of the present invention is administered upstream of the heart, i.e., into the intracoronary of the subject. Alternatively, the mitochondrion, composition or pharmaceutical composition of the present invention is injected directly into the heart.

In certain embodiments, the target organ is the liver. In certain embodiments, a mitochondrion, composition or pharmaceutical composition of the present invention is delivered to the liver of a subject. For that, it is preferred that the mitochondrion, composition or pharmaceutical composition of the present invention is administered upstream of the liver, i.e., into the hepatic artery or portal vein of the subject. Alternatively, the mitochondrion, composition or pharmaceutical composition of the present invention is injected directly into the liver.

In certain embodiments, the target organ is the pancreas. In certain embodiments, a mitochondrion, composition or pharmaceutical composition of the present invention is delivered to the pancreas of a subject. For that, it is preferred that the mitochondrion, composition or pharmaceutical composition of the present invention is administered upstream of the pancreas, i.e., into the hepatic artery of the subject. Alternatively, the mitochondrion, composition or pharmaceutical composition of the present invention is injected directly into the pancreas.

In certain embodiments, the target organ is the duodenum. In certain embodiments, a mitochondrion, composition or pharmaceutical composition of the present invention is delivered to the duodenum of a subject. For that, it is preferred that the mitochondrion, composition or pharmaceutical composition of the present invention is administered upstream of the duodenum, i.e., into the hepatic artery of the subject. Alternatively, the mitochondrion, composition or pharmaceutical composition of the present invention is injected directly into the duodenum.

In certain embodiments, the target organ is the spleen. In certain embodiments, a mitochondrion, composition or pharmaceutical composition of the present invention is delivered to the spleen of a subject. For that, it is preferred that the mitochondrion, composition or pharmaceutical composition of the present invention is administered upstream of the spleen, i.e., into the splenic artery of the subject. Alternatively, the mitochondrion, composition or pharmaceutical composition of the present invention is injected directly into the spleen.

In certain embodiments, the target organ is the lung. In certain embodiments, a mitochondrion, composition or pharmaceutical composition of the present invention is delivered to the lung of a subject. For that, it is preferred that the mitochondrion, composition or pharmaceutical composition of the present invention is administered upstream of the lung, i.e., into the pulmonary artery of the subject. Alternatively, the mitochondrion, composition or pharmaceutical composition of the present invention is injected directly into the lung.

In certain embodiments, the target organ is the intestines. In certain embodiments, a mitochondrion, composition or pharmaceutical composition of the present invention is delivered to the intestines of a subject. For that, it is preferred that the mitochondrion, composition or pharmaceutical composition of the present invention is administered upstream of the intestines, i.e., into the superior mesenteric artery of the subject. Alternatively, the mitochondrion, composition or pharmaceutical composition of the present invention is injected directly into the intestines.

In certain embodiments, the target organ is the bladder. In certain embodiments, a mitochondrion, composition or pharmaceutical composition of the present invention is delivered to the bladder of a subject. For that, it is preferred that the mitochondrion, composition or pharmaceutical composition of the present invention is administered upstream of the bladder, i.e., into the superior and inferior vesical arteries of the subject. Alternatively, the mitochondrion, composition or pharmaceutical composition of the present invention is injected directly into the bladder.

For the delivery of mitochondria, compositions or pharmaceutical compositions, administration by injection or infusion may be made. A mitochondrion, compositions or pharmaceutical compositions may be administered systemically. The phrases “systemic administration,” “administered systemically”, “peripheral administration” and “administered peripherally” refer to the administration of a mitochondrion, compositions or pharmaceutical compositions other than directly into a target site, cell, tissue, or organ, such that it enters, instead, the subject's circulatory system and, thus, is subject to metabolism and other like processes. It is preferred, that a mitochondrion, composition or pharmaceutical composition of the present invention is delivered to a cell via a direct incubation with the cell in a cell culture medium. In a further preferred embodiment, a mitochondrion, composition or pharmaceutical composition of the present invention is delivered directly to a site where treatment is desired by injection. In a further preferred embodiment, a mitochondrion, composition or pharmaceutical composition of the present invention is delivered systemically by intravenous injection. In a further preferred embodiment, a mitochondrion, composition or pharmaceutical composition of the present invention is delivered by injection into the bloodstream upstream of a target organ where therapy is desired. In a further preferred embodiment, a nebulized mitochondrion, a nebulized composition or nebulized pharmaceutical composition of the present invention is delivered by inhalation.

A mitochondrion, composition or pharmaceutical composition of the present invention may be administered into the bloodstream upstream of the target organ. Accordingly, the present invention provides a method for delivering a nucleic acid molecule to a target organ, the method comprising a step of administering a pharmaceutical composition comprising a mitochondrion comprising one or more payloads, such as nucleic acid molecule(s), polypeptide(s), drug(s) or combinations thereof attached to the outer membrane of the mitochondrion, wherein the one or more payloads, such as nucleic acid molecule(s), polypeptide(s), drug(s) or combinations thereof:
a) is electrostatically attached to the outer membrane of the mitochondrion via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule; and a pharmaceutically acceptable carrier, into the bloodstream of a subject in need, wherein the pharmaceutical composition is administered into the bloodstream upstream of the target organ. The present invention provides a method for delivering a payload, such as nucleic acid molecule(s), polypeptide(s), drug(s) or combinations thereof to a target organ, the method comprising a step of administering a pharmaceutical composition comprising a mitochondrion comprising one or more payloads, such as nucleic acid molecule(s), polypeptide(s), drug(s) or combinations thereof attached to the outer membrane of the mitochondrion, wherein the one or more payloads, such as nucleic acid molecule(s), polypeptide(s), drug(s) or combinations thereof:
a) is electrostatically attached to the outer membrane of the mitochondrion via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule; and a pharmaceutically acceptable carrier, into the bloodstream of a subject having a cardiovascular disease, an aging related disease, a kidney disease, or cancer, wherein the pharmaceutical composition is administered into the bloodstream upstream of the target organ. The present invention provides a method for delivering a payload, such as nucleic acid molecule(s), polypeptide(s), drug(s) or combinations thereof to a target organ, the method comprising a step of administering a pharmaceutical composition comprising a mitochondrion comprising one or more payloads, such as nucleic acid molecule(s), polypeptide(s), drug(s) or combinations thereof attached to the outer membrane of the mitochondrion, wherein the one or more payloads, such as nucleic acid molecule(s), polypeptide(s), drug(s) or combinations thereof:
a) is electrostatically attached to the outer membrane of the mitochondrion via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule; and a pharmaceutically acceptable carrier, into the bloodstream of a subject having ischemic heart disease, atherosclerosis, sarcopenia, Parkinson's disease, Hutchinson-Gilford progeria syndrome or cancer, wherein the pharmaceutical composition is administered into the bloodstream upstream of the target organ.

A mitochondrion, composition or pharmaceutical composition of the present invention may be administered by inhalation. Accordingly, the present invention provides a method for delivering a payload, such as nucleic acid molecule(s), polypeptide(s), drug(s) or combinations thereof to the lung, the method comprising a step of administering a pharmaceutical composition comprising a mitochondrion comprising one or more payloads, such as nucleic acid molecule(s), polypeptide(s), drug(s) or combinations thereof attached to the outer membrane of the mitochondrion, wherein the one or more payloads, such as nucleic acid molecule(s), polypeptide(s), drug(s) or combinations thereof:
a) is electrostatically attached to the outer membrane of the mitochondrion via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule; and a pharmaceutically acceptable carrier to a subject in need, wherein the pharmaceutical composition is administered by inhalation.

The present invention provides a method for delivering a payload, such as nucleic acid molecule(s), polypeptide(s), drug(s) or combinations thereof to the lung, the method comprising a step of administering a pharmaceutical composition comprising a mitochondrion comprising one or more payloads, such as nucleic acid molecule(s), polypeptide(s), drug(s) or combinations thereof attached to the outer membrane of the mitochondrion, wherein the one or more payloads, such as nucleic acid molecule(s), polypeptide(s), drug(s) or combinations thereof:
a) is electrostatically attached to the outer membrane of the mitochondrion via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule; and a pharmaceutically acceptable carrier to a subject having a cardiovascular disease, an aging related disease, kidney disease, or cancer, wherein the pharmaceutical composition is administered by inhalation.

The present invention provides a method for delivering a payload, such as nucleic acid molecule(s), polypeptide(s), drug(s) or combinations thereof to the lung, the method comprising a step of administering a pharmaceutical composition comprising a mitochondrion comprising one or more payloads, such as nucleic acid molecule(s), polypeptide(s), drug(s) or combinations thereof attached to the outer membrane of the mitochondrion, wherein the one or more payloads, such as nucleic acid molecule(s), polypeptide(s), drug(s) or combinations thereof:
a) is electrostatically attached to the outer membrane of the mitochondrion via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule; and a pharmaceutically acceptable carrier to a subject having ischemic heart disease, atherosclerosis, muscular dystrophy, Parkinson's disease, Hutchinson-Gilford progeria syndrome or cancer, wherein the pharmaceutical composition is administered by inhalation.

In certain embodiments, a mitochondrion comprising one or more payloads, such as nucleic acid molecule(s), polypeptide(s), drug(s) or combinations thereof, or a composition or pharmaceutical composition comprising a mitochondrion comprising one or more payloads, such as nucleic acid molecule(s), polypeptide(s), drug(s) or combinations thereof, is delivered to the kidney of a subject. In certain embodiments, delivery into the kidney is achieved through injection into the renal artery or through direct injection into the kidney. In certain embodiments, delivery into the kidney is achieved through injection into the renal artery or through direct injection into the kidney and the one or more payloads, such as nucleic acid molecule(s), polypeptide(s), drug(s) or combinations thereof is electrostatically attached to the outer membrane of the mitochondrion via a positively-charged species. In certain embodiments, delivery into the kidney is achieved through injection into the renal artery or through direct injection into the kidney and the one or more payloads, such as nucleic acid molecule(s), polypeptide(s), drug(s) or combinations thereof is covalently linked to the outer membrane of the mitochondrion. In certain embodiments, delivery into the kidney is achieved through injection into the renal artery or through direct injection into the kidney and the one or more payloads, such as nucleic acid molecule(s), polypeptide(s), drug(s) or combinations thereof is linked to an antibody that specifically binds to an antigen comprised in the outer membrane of the mitochondrion. In certain embodiments, delivery into the kidney is achieved through injection into the renal artery or through direct injection into the kidney and the one or more payloads, such as nucleic acid molecule(s), polypeptide(s), drug(s) or combinations thereof is linked to a mitochondria-targeting small molecule.

In certain embodiments, a mitochondrion comprising one or more payloads, such as nucleic acid molecule(s), polypeptide(s), drug(s) or combinations thereof, or a composition or pharmaceutical composition comprising a mitochondrion comprising one or more payloads, such as nucleic acid molecule(s), polypeptide(s), drug(s) or combinations thereof, is delivered to the heart of a subject. In certain embodiments, delivery into the heart is achieved through injection into the intracoronary or through direct injection into the heart. In certain embodiments, delivery into the heart is achieved through injection into the intracoronary or through direct injection into the heart and the one or more payloads, such as nucleic acid molecule(s), polypeptide(s), drug(s) or combinations thereof is electrostatically attached to the outer membrane of the mitochondrion via a positively-charged species. In certain embodiments, delivery into the heart is achieved through injection into the intracoronary or through direct injection into the heart and the one or more payloads, such as nucleic acid molecule(s), polypeptide(s), drug(s) or combinations thereof is covalently linked to the outer membrane of the mitochondrion. In certain embodiments, delivery into the heart is achieved through injection into the intracoronary or through direct injection into the heart and the one or more payloads, such as nucleic acid molecule(s), polypeptide(s), drug(s) or combinations thereof is linked to an antibody that specifically binds to an antigen comprised in the outer membrane of the mitochondrion. In certain embodiments, delivery into the heart is achieved through injection into the intracoronary or through direct injection into the heart and the one or more payloads, such as nucleic acid molecule(s), polypeptide(s), drug(s) or combinations thereof is linked to a mitochondria-targeting small molecule.

In certain embodiments, a mitochondrion comprising one or more payloads, such as nucleic acid molecule(s), polypeptide(s), drug(s) or combinations thereof, or a composition or pharmaceutical composition comprising a mitochondrion comprising one or more payloads, such as nucleic acid molecule(s), polypeptide(s), drug(s) or combinations thereof, is delivered to the liver of a subject. In certain embodiments, delivery into the liver is achieved through injection into the hepatic artery or portal vein or through direct injection into the liver. In certain embodiments, delivery into the liver is achieved through injection into the hepatic artery or portal vein or through direct injection into the liver and the one or more payloads, such as nucleic acid molecule(s), polypeptide(s), drug(s) or combinations thereof is electrostatically attached to the outer membrane of the mitochondrion via a positively-charged species. In certain embodiments, delivery into the liver is achieved through injection into the hepatic artery or portal vein or through direct injection into the liver and the one or more payloads, such as nucleic acid molecule(s), polypeptide(s), drug(s) or combinations thereof is covalently linked to the outer membrane of the mitochondrion. In certain embodiments, delivery into the liver is achieved through injection into the hepatic artery or portal vein or through direct injection into the liver and the one or more payloads, such as nucleic acid molecule(s), polypeptide(s), drug(s) or combinations thereof is linked to an antibody that specifically binds to an antigen comprised in the outer membrane of the mitochondrion. In certain embodiments, delivery into the liver is achieved through injection into the hepatic artery or portal vein or through direct injection into the liver and the one or more payloads, such as nucleic acid molecule(s), polypeptide(s), drug(s) or combinations thereof is linked to a mitochondria-targeting small molecule.

In certain embodiments, a mitochondrion comprising one or more payloads, such as nucleic acid molecule(s), polypeptide(s), drug(s) or combinations thereof, or a composition or pharmaceutical composition comprising a mitochondrion comprising one or more payloads, such as nucleic acid molecule(s), polypeptide(s), drug(s) or combinations thereof, is delivered to the pancreas of a subject. In certain embodiments, delivery into the pancreas is achieved through injection into the hepatic artery or through direct injection into the pancreas. In certain embodiments, delivery into the pancreas is achieved through injection into the hepatic artery or through direct injection into the pancreas and the one or more payloads, such as nucleic acid molecule(s), polypeptide(s), drug(s) or combinations thereof is electrostatically attached to the outer membrane of the mitochondrion via a positively-charged species. In certain embodiments, delivery into the pancreas is achieved through injection into the hepatic artery or through direct injection into the pancreas and the one or more payloads, such as nucleic acid molecule(s), polypeptide(s), drug(s) or combinations thereof is covalently linked to the outer membrane of the mitochondrion. In certain embodiments, delivery into the pancreas is achieved through injection into the hepatic artery or through direct injection into the pancreas and the one or more payloads, such as nucleic acid molecule(s), polypeptide(s), drug(s) or combinations thereof is linked to an antibody that specifically binds to an antigen comprised in the outer membrane of the mitochondrion. In certain embodiments, delivery into the pancreas is achieved through injection into the hepatic artery or through direct injection into the pancreas and the one or more payloads, such as nucleic acid molecule(s), polypeptide(s), drug(s) or combinations thereof is linked to a mitochondria-targeting small molecule.

In certain embodiments, a mitochondrion comprising one or more payloads, such as nucleic acid molecule(s), polypeptide(s), drug(s) or combinations thereof, or a composition or pharmaceutical composition comprising a mitochondrion comprising one or more payloads, such as nucleic acid molecule(s), polypeptide(s), drug(s) or combinations thereof, is delivered to the duodenum of a subject. In certain embodiments, delivery into the duodenum is achieved through injection into the hepatic artery or through direct injection into the duodenum. In certain embodiments, delivery into the duodenum is achieved through injection into the hepatic artery or through direct injection into the duodenum and the one or more payloads, such as nucleic acid molecule(s), polypeptide(s), drug(s) or combinations thereof is electrostatically attached to the outer membrane of the mitochondrion via a positively-charged species. In certain embodiments, delivery into the duodenum is achieved through injection into the hepatic artery or through direct injection into the duodenum and the one or more payloads, such as nucleic acid molecule(s), polypeptide(s), drug(s) or combinations thereof is covalently linked to the outer membrane of the mitochondrion. In certain embodiments, delivery into the duodenum is achieved through injection into the hepatic artery or through direct injection into the duodenum and the one or more payloads, such as nucleic acid molecule(s), polypeptide(s), drug(s) or combinations thereof is linked to an antibody that specifically binds to an antigen comprised in the outer membrane of the mitochondrion. In certain embodiments, delivery into the duodenum is achieved through injection into the hepatic artery or through direct injection into the duodenum and the one or more payloads, such as nucleic acid molecule(s), polypeptide(s), drug(s) or combinations thereof is linked to a mitochondria-targeting small molecule.

In certain embodiments, a mitochondrion comprising one or more payloads, such as nucleic acid molecule(s), polypeptide(s), drug(s) or combinations thereof, or a composition or pharmaceutical composition comprising a mitochondrion comprising one or more payloads, such as nucleic acid molecule(s), polypeptide(s), drug(s) or combinations thereof, is delivered to the spleen of a subject. In certain embodiments, delivery into the spleen is achieved through injection into the splenic artery or through direct injection into the spleen. In certain embodiments, delivery into the spleen is achieved through injection into the splenic artery or through direct injection into the spleen and the one or more payloads, such as nucleic acid molecule(s), polypeptide(s), drug(s) or combinations thereof is electrostatically attached to the outer membrane of the mitochondrion via a positively-charged species. In certain embodiments, delivery into the spleen is achieved through injection into the splenic artery or through direct injection into the spleen and the one or more payloads, such as nucleic acid molecule(s), polypeptide(s), drug(s) or combinations thereof is covalently linked to the outer membrane of the mitochondrion. In certain embodiments, delivery into the spleen is achieved through injection into the splenic artery or through direct injection into the spleen and the one or more payloads, such as nucleic acid molecule(s), polypeptide(s), drug(s) or combinations thereof is linked to an antibody that specifically binds to an antigen comprised in the outer membrane of the mitochondrion. In certain embodiments, delivery into the spleen is achieved through injection into the splenic artery or through direct injection into the spleen and the one or more payloads, such as nucleic acid molecule(s), polypeptide(s), drug(s) or combinations thereof is linked to a mitochondria-targeting small molecule.

In certain embodiments, a mitochondrion comprising one or more payloads, such as nucleic acid molecule(s), polypeptide(s), drug(s) or combinations thereof, or a composition or pharmaceutical composition comprising a mitochondrion comprising one or more payloads, such as nucleic acid molecule(s), polypeptide(s), drug(s) or combinations thereof, is delivered to the lung of a subject. In certain embodiments, delivery into the lung is achieved through injection into the pulmonary artery or through direct injection into the lung. In certain embodiments, delivery into the lung is achieved through injection into the pulmonary artery or through direct injection into the lung and the one or more payloads, such as nucleic acid molecule(s), polypeptide(s), drug(s) or combinations thereof is electrostatically attached to the outer membrane of the mitochondrion via a positively-charged species. In certain embodiments, delivery into the lung is achieved through injection into the pulmonary artery or through direct injection into the lung and the one or more payloads, such as nucleic acid molecule(s), polypeptide(s), drug(s) or combinations thereof is covalently linked to the outer membrane of the mitochondrion. In certain embodiments, delivery into the lung is achieved through injection into the pulmonary artery or through direct injection into the lung and the one or more payloads, such as nucleic acid molecule(s), polypeptide(s), drug(s) or combinations thereof is linked to an antibody that specifically binds to an antigen comprised in the outer membrane of the mitochondrion. In certain embodiments, delivery into the lung is achieved through injection into the pulmonary artery or through direct injection into the lung and the one or more payloads, such as nucleic acid molecule(s), polypeptide(s), drug(s) or combinations thereof is linked to a mitochondria-targeting small molecule.

In certain embodiments, a mitochondrion comprising one or more payloads, such as nucleic acid molecule(s), polypeptide(s), drug(s) or combinations thereof, or a composition or pharmaceutical composition comprising a mitochondrion comprising one or more payloads, such as nucleic acid molecule(s), polypeptide(s), drug(s) or combinations thereof, is delivered to the intestines of a subject. In certain embodiments, delivery into the intestines is achieved through injection into the superior mesenteric artery or through direct injection into the intestines. In certain embodiments, delivery into the intestines is achieved through injection into the superior mesenteric artery or through direct injection into the intestines and the one or more payloads, such as nucleic acid molecule(s), polypeptide(s), drug(s) or combinations thereof is electrostatically attached to the outer membrane of the mitochondrion via a positively-charged species. In certain embodiments, delivery into the intestines is achieved through injection into the superior mesenteric artery or through direct injection into the intestines and the one or more payloads, such as nucleic acid molecule(s), polypeptide(s), drug(s) or combinations thereof is covalently linked to the outer membrane of the mitochondrion. In certain embodiments, delivery into the intestines is achieved through injection into the superior mesenteric artery or through direct injection into the intestines and the one or more payloads, such as nucleic acid molecule(s), polypeptide(s), drug(s) or combinations thereof is linked to an antibody that specifically binds to an antigen comprised in the outer membrane of the mitochondrion. In certain embodiments, delivery into the intestines is achieved through injection into the superior mesenteric artery or through direct injection into the intestines and the one or more payloads, such as nucleic acid molecule(s), polypeptide(s), drug(s) or combinations thereof is linked to a mitochondria-targeting small molecule.

In certain embodiments, a mitochondrion comprising one or more payloads, such as nucleic acid molecule(s), polypeptide(s), drug(s) or combinations thereof, or a composition or pharmaceutical composition comprising a mitochondrion comprising one or more payloads, such as nucleic acid molecule(s), polypeptide(s), drug(s) or combinations thereof, is delivered to the bladder of a subject. In certain embodiments, delivery into the bladder is achieved through injection into the superior and inferior vesical arteries or through direct injection into the bladder. In certain embodiments, delivery into the bladder is achieved through injection into the superior and inferior vesical arteries or through direct injection into the bladder and the one or more payloads, such as nucleic acid molecule(s), polypeptide(s), drug(s) or combinations thereof is electrostatically attached to the outer membrane of the mitochondrion via a positively-charged species. In certain embodiments, delivery into the bladder is achieved through injection into the superior and inferior vesical arteries or through direct injection into the bladder and the one or more payloads, such as nucleic acid molecule(s), polypeptide(s), drug(s) or combinations thereof is covalently linked to the outer membrane of the mitochondrion. In certain embodiments, delivery into the bladder is achieved through injection into the superior and inferior vesical arteries or through direct injection into the bladder and the one or more payloads, such as nucleic acid molecule(s), polypeptide(s), drug(s) or combinations thereof is linked to an antibody that specifically binds to an antigen comprised in the outer membrane of the mitochondrion. In certain embodiments, delivery into the bladder is achieved through injection into the superior and inferior vesical arteries or through direct injection into the bladder and the one or more payloads, such as nucleic acid molecule(s), polypeptide(s), drug(s) or combinations thereof is linked to a mitochondria-targeting small molecule.

In one aspect the invention provides a method for attaching a payloads, such as nucleic acid molecule(s), polypeptide(s), drug(s) or combinations thereof to a mitochondrion thereby producing the mitochondrion of the present invention. In the sense of the present invention “contacting” means bringing a first substance into close physical proximity with a second substance so that both can perform a reaction. For example, the mitochondrion may be contacted with payloads, such as nucleic acid molecule(s), polypeptide(s), drug(s) or combinations thereof in the presence of the positively-charged species in a solution, such as a buffer.

In the sense of the invention any payload, such as a nucleic acid, a polypeptide or a drug may be attached to a mitochondrion. The payload may be attached to the mitochondrion by contacting the mitochondrion with the payload, e.g. a nucleic acid, drug or a polypeptide. In the sense of the present invention the mitochondrion and the payload may be contacted in the presence of a positively-charged species, such as a polycationic species. The step of contacting a mitochondrion with a nucleic acid, drug or a polypeptide and/or a positively-charged species may be performed at any reaction conditions feasible for successful complex formation, i.e. successful attachment of the nucleic acid, drug or polypeptide.

A mitochondrion may be contacted with a payloads, such as nucleic acid molecule(s), polypeptide(s), drug(s) or combinations thereof in a buffer, such as a conjugation buffer. A mitochondrion may be contacted with a payloads, such as nucleic acid molecule(s), polypeptide(s), drug(s) or combinations thereof and a positively-charged species, such as a polycationic species, in a buffer, such as a conjugation buffer. A mitochondrion may be contacted with a polypeptide in a buffer, such as a conjugation buffer. A mitochondrion may be contacted with a polypeptide and a positively-charged species, such as a polycationic species, in a buffer, such as a conjugation buffer.

In some embodiments the concentration of mitochondria is 0.1 to 5 mg/mL. In some embodiments the concentration of mitochondria in a conjugation buffer is 0.1 to 5 mg/mL. In some embodiments the concentration of mitochondria is 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9 or 5 mg/mL. In some embodiments the concentration of mitochondria in a conjugation buffer is 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9 or 5 mg/mL. In a preferred embodiment the concentration of mitochondria in a conjugation buffer is 2 mg/mL. In a preferred embodiment the concentration of mitochondria in a conjugation buffer is 4 mg/mL. In some embodiments the concentration of mitochondria is 0.5 to 30 billion/mL. In some embodiments the concentration of mitochondria in a conjugation buffer is 0.5 to 30 billion/mL. In some embodiments the concentration of mitochondria is 0.5, 0.6, 0.7, 0.8, 0.9, 1.0, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5.0, 5.2, 5.5, 5.8, 6.0, 6.2, 6.4, 6.8, 7.0, 7.2, 7.5, 7.8, 8.0, 8.2, 8.4, 8.6, 8.8, 9.0, 9.2, 9.4, 9.6, 9.8, 10.0, 10.2, 10.4, 10.6, 10.8, 11.0, 11.2, 11.4, 11.6, 11.8, 12.0, 12.2, 12.5, 12.9, 13.2, 13.4, 13.6, 13.8, 14.0, 15.0, 16.0, 17.0, 18.0, 20.0, 22.0, 24.0, 26.0, 28.0, or 30 billion/mL. In some embodiments the concentration of mitochondria in a conjugation buffer is 0.5, 0.6, 0.7, 0.8, 0.9, 1.0, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5.0, 5.2, 5.5, 5.8, 6.0, 6.2, 6.4, 6.8, 7.0, 7.2, 7.5, 7.8, 8.0, 8.2, 8.4, 8.6, 8.8, 9.0, 9.2, 9.4, 9.6, 9.8, 10.0, 10.2, 10.4, 10.6, 10.8, 11.0, 11.2, 11.4, 11.6, 11.8, 12.0, 12.2, 12.5, 12.9, 13.2, 13.4, 13.6, 13.8, 14.0, 15.0, 16.0, 17.0, 18.0, 20.0, 22.0, 24.0, 26.0, 28.0, or 30 billion/mL. In a preferred embodiment the concentration of mitochondria in a conjugation buffer is 6 billion/mL. In a preferred embodiment the concentration of mitochondria in a conjugation buffer is 12 billion/mL. In a preferred embodiment the concentration of mitochondria in a conjugation buffer is 15 billion/mL.

In some embodiments a mitochondrion is contacted with 0.002 to 5000 μmol of payloads, such as nucleic acid molecule(s), polypeptide(s), drug(s) or combinations thereof. In some embodiments a mitochondrion is contacted with 0.002 to 5000 μmol of payloads, such as nucleic acid molecule(s), polypeptide(s), drug(s) or combinations thereof in a conjugation buffer. In some embodiments a mitochondrion is contacted with 0.002, 50, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950, 3000, 3050, 3100, 3150, 3200, 3250, 3300, 3350, 3400, 3450, 3500, 3550, 3600, 3650, 3700, 3750, 3800, 3850, 3900, 3950, 4000, 4050, 4100, 4150, 4200, 4250, 4300, 4350, 4400, 4450, 4500, 4550, 4600, 4650, 4700, 4750, 4800, 4850, 4900, 4950, 5000 μmol of payloads, such as nucleic acid molecule(s), polypeptide(s), drug(s) or combinations thereof, optionally in a conjugation buffer. In some embodiments a mitochondrion is contacted with 0.1, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 μmol of payloads, such as nucleic acid molecule(s), polypeptide(s), drug(s) or combinations thereof, optionally in a conjugation buffer. In a preferred embodiment a mitochondrion is contacted with 0.1 to 50 μmol of payloads, such as nucleic acid molecule(s), polypeptide(s), drug(s) or combinations thereof. In a preferred embodiment a mitochondrion is contacted with 0.1 to 50 μmol of payloads, such as nucleic acid molecule(s), polypeptide(s), drug(s) or combinations thereof in a conjugation buffer.

In some embodiments a mitochondrion is contacted with 0.002 to 5 μg/μL of payloads, such as nucleic acid molecule(s), polypeptide(s), drug(s) or combinations thereof. In some embodiments a mitochondrion is contacted with 0.002 to 5 μg/μL of payloads, such as nucleic acid molecule(s), polypeptide(s), drug(s) or combinations thereof in a conjugation buffer. In some embodiments a mitochondrion is contacted with 0.002, 0.004, 0.008, 0.05, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9 or 5 μg/μL of nucleic acid molecules, optionally in a conjugation buffer. In some embodiments a mitochondrion is contacted with 0.002, 0.004, 0.008, 0.05, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9 or 5 μg/μL of payloads, such as nucleic acid molecule(s), polypeptide(s), drug(s) or combinations thereof, optionally in a conjugation buffer. In a preferred embodiment a mitochondrion is contacted with 0.1 to 2 μg/μL of payloads, such as nucleic acid molecule(s), polypeptide(s), drug(s) or combinations thereof. In a preferred embodiment a mitochondrion is contacted with 0.1 to 2 μg/μL of payloads, such as nucleic acid molecule(s), polypeptide(s), drug(s) or combinations thereof in a conjugation buffer.

In some embodiments a mitochondrion is contacted with 0.004 to 40 mg/mL of positively-charged species. In some embodiments a mitochondrion is contacted with 0.004 to 40 mg/mL of positively-charged species in a conjugation buffer. In some embodiments a mitochondrion is contacted with 0.004, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 mg/mL of positively-charged species. In some embodiments a mitochondrion is contacted with 0.004, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 mg/mL of positively-charged species in a conjugation buffer. In a preferred embodiment, a mitochondrion is contacted with 0.02 to 1.0 mg/mL of positively-charged species, optionally in a conjugation buffer. In a preferred embodiment, a mitochondrion is contacted with 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.5, 0.6, 0.7, 0.8, 0.9 or 1.0 mg/mL of positively-charged species, optionally in a conjugation buffer.

In some embodiments a mitochondrion is contacted with 0.004 to 40 mg/mL of protective polymer. In some embodiments a mitochondrion is contacted with 0.004 to 40 mg/ml of protective polymer in a conjugation buffer. In some embodiments a mitochondrion is contacted with 0.004, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 mg/mL of protective polymer. In some embodiments a mitochondrion is contacted with 0.004, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 mg/mL of protective polymer in a conjugation buffer. In a preferred embodiment, a mitochondrion is contacted with 0.02 to 2 mg/mL of protective polymer, optionally in a conjugation buffer. In a preferred embodiment, a mitochondrion is contacted with 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.5, 0.6, 0.7, 0.8, 0.9, 1.0, 1.2, 1.5, 1.8, 2.0 mg/mL of protective polymer, optionally in a conjugation buffer.

In a further preferred embodiment, a mitochondrion is contacted with 0.002 to 5000 μmol of payloads, such as nucleic acid molecule(s), polypeptide(s), drug(s) or combinations thereof and 0.004 to 40 mg/mL of positively-charged species, wherein the concentration of mitochondrion is 0.1 to 5 mg/mL, optionally in a conjugation buffer.

In a further preferred embodiment, a mitochondrion is contacted with 0.1 to 50 μmol of payloads, such as nucleic acid molecule(s), polypeptide(s), drug(s) or combinations thereof and 0.02 to 1.0 mg/mL of positively-charged species, wherein the concentration of mitochondrion is 1 mg/mL, optionally in a conjugation buffer.

In a further preferred embodiment, a mitochondrion is contacted with 0.1 to 50 μmol of payloads, such as nucleic acid molecule(s), polypeptide(s), drug(s) or combinations thereof and 0.02 to 1.0 mg/mL of positively-charged species, wherein the concentration of mitochondrion is 1 mg/mL in a conjugation buffer. In a further preferred embodiment, 50 μg of mitochondria is contacted with 0.1 to 50 μmol of payloads, such as nucleic acid molecule(s), polypeptide(s), drug(s) or combinations thereof and 0.02 to 1.0 mg/mL of positively-charged species, wherein the concentration of mitochondrion is 1 mg/mL in a conjugation buffer.

In some embodiments 50 μg of mitochondria are contacted with 0.1 to 50 μmol of a payloads, such as nucleic acid molecule(s), polypeptide(s), drug(s) or combinations thereof or peptide and 0.02 to 1.0 mg/mL of the positively-charged species. In some embodiments 50 μg of mitochondria are contacted with 0.1 to 50 μmol of the plurality of payloads, such as nucleic acid molecule(s), polypeptide(s), drug(s) or combinations thereof linked to a mitochondria-targeting small molecule. In a preferred embodiment 50 μg of mitochondria are contacted with 0.1 to 50 μmol of the plurality of payloads, such as nucleic acid molecule(s), polypeptide(s), drug(s) or combinations thereof and 0.02 to 1.0 mg/mL of the positively-charged species.

In some embodiments 50 μg of mitochondria are contacted with 0.1 to 50 μmol of siRNA or mRNA. In some embodiments 50 μg of mitochondria are contacted with 0.1 to 50 μmol of fluorescently-labeled ssDNA or ssRNA or plasmid DNA. In some embodiments 50 μg of mitochondria are contacted with 0.1 to 2 μL of 10 mg/mL poly-L-lysine. The concentration of mitochondria is preferably 1 mg of mitochondria per 1 mL conjugation buffer, i.e. 1 mg/mL. The skilled person is aware that the above embodiments may be combined to facilitate successful conjugation of a payload, such as a nucleic acid or polypeptide, to a mitochondrion. In some embodiments, an amount of 50 μg to 200 μg of mitochondria are contacted with 0.1 to 50 μmol of the nucleic acid molecules and 0.02 to 10 μg, preferably 0.02 to 5 μg, of the positively-charged species.

In some embodiments, an amount of 50 μg to 200 μg of mitochondria are contacted with 0.1 to 50 μmol of the nucleic acid molecules linked to a mitochondria-targeting small molecule.
50 microgram of mitochondria corresponds to ca. 150 million of mitochondria. 1 mg/mL of mitochondria (based on Qubit protein assay) corresponds to ca. 3B mitochondria/mL (based on particle counter)
The concentration of the preparation of protective polymer used for the preparation of the mitochondrion of the invention is 1 mg/mL. The amount of protective polymer is between 0.1 mg and 10 mg.
The concentration of the preparation of nanoparticle used for the preparation of the mitochondrion of the invention is 1 mg/mL. The amount of protective polymer is between 0.1 mg and 10 mg.

Accordingly, the present invention provides a method for attaching a payload, such as nucleic acid molecule(s), polypeptide(s), drug(s) or combinations thereof to the outer membrane of a mitochondrion, the method comprising the steps of:
a) providing a preparation of mitochondria;
b) contacting the mitochondria provided in step (a) with at least one payload, such as nucleic acid molecule(s), polypeptide(s), drug(s) or combinations thereof in the presence of a positively-charged species;
c) attaching the at least one payload, such as nucleic acid molecule(s), polypeptide(s), drug(s) or combinations thereof to the mitochondria via the positively-charged species.

The at least one payloads, such as nucleic acid molecule(s), polypeptide(s), drug(s) or combinations thereof may be contacted with the positively-charged species and the mitochondria simultaneously, the at least one payloads, such as nucleic acid molecule(s), polypeptide(s), drug(s) or combinations thereof may be first contacted with a positively-charged species to form a positively-charged complex before the positively-charged complex is contacted with the mitochondria or the mitochondrion can be contacted first with the positively-charged species and subsequently contacted with the at least one payloads, such as nucleic acid molecule(s), polypeptide(s), drug(s) or combinations thereof. Accordingly, the present invention provides a method for attaching a payloads, such as nucleic acid molecule(s), polypeptide(s), drug(s) or combinations thereof to the outer membrane of a mitochondrion, the method comprising the steps of:
a) providing a preparation of mitochondria;
b) contacting the mitochondria provided in step (a) with at least one payloads, such as nucleic acid molecule(s), polypeptide(s), drug(s) or combinations thereof in the presence of a positively-charged species, wherein
the at least one payloads, such as nucleic acid molecule(s), polypeptide(s), drug(s) or combinations thereof is simultaneously contacted with the positively-charged species and the mitochondria; or
the at least one payloads, such as nucleic acid molecule(s), polypeptide(s), drug(s) or combinations thereof is contacted with the positively-charged species to form a positively-charged complex before the positively-charged complex is contacted with the mitochondria; or
the mitochondrion is contacted with the positively-charged species and subsequently contacted with the at least one payloads, such as nucleic acid molecule(s), polypeptide(s), drug(s) or combinations thereof; and
c) attaching the at least one payloads, such as nucleic acid molecule(s), polypeptide(s), drug(s) or combinations thereof to the mitochondria via the positively-charged species. Within the method of the present invention, the method can involve contacting the mitochondrion with the at least one payloads, such as nucleic acid molecule(s), polypeptide(s), drug(s) or combinations thereof in the presence of the positively-charged species, wherein
   a) the at least one payloads, such as nucleic acid molecule(s), polypeptide(s), drug(s) or combinations thereof, is simultaneously contacted with the positively-charged species and the mitochondria;
   b) wherein the at least one payloads, such as nucleic acid molecule(s), polypeptide(s), drug(s) or combinations thereof, is contacted with the positively-charged species to form a positively-charged complex before the positively-charged complex is contacted with the mitochondria; or
   c) the mitochondrion is contacted with the positively-charged species and subsequently contacted with the at least one payloads, such as nucleic acid molecule(s), polypeptide(s), drug(s) or combinations thereof.

In preferred embodiments, the method of the invention involves contacting the mitochondrion with the at least one payloads, such as nucleic acid molecule(s), polypeptide(s), drug(s) or combinations thereof in the presence of the positively-charged species, wherein the mitochondrion is contacted with the positively-charged species and subsequently contacted with the at least one payloads, such as nucleic acid molecule(s), polypeptide(s), drug(s) or combinations thereof.

The step of contacting mitochondria with the at least one payloads, such as nucleic acid molecule(s), polypeptide(s), drug(s) or combinations thereof and a positively-charged species may be performed in a suitable buffer. Accordingly, the present invention provides a method for attaching a payloads, such as nucleic acid molecule(s), polypeptide(s), drug(s) or combinations thereof to the outer membrane of a mitochondrion, the method comprising the steps of:
a) providing a preparation of mitochondria;
b) contacting the mitochondria provided in step (a) with at least one payloads, such as nucleic acid molecule(s), polypeptide(s), drug(s) or combinations thereof in the presence of a positively-charged species;
c) attaching the at least one payloads, such as nucleic acid molecule(s), polypeptide(s), drug(s) or combinations thereof to the mitochondria via the positively-charged species, wherein the mitochondria are contacted with the at least one payloads, such as nucleic acid molecule(s), polypeptide(s), drug(s) or combinations thereof and the positively-charged species in a suitable buffer.

Preferably, the present invention provides a method for attaching a payloads, such as nucleic acid molecule(s), polypeptide(s), drug(s) or combinations thereof to the outer membrane of a mitochondrion, the method comprising the steps of:
a) providing a preparation of mitochondria;
b) contacting the mitochondria provided in step (a) with one or more payloads, such as nucleic acid molecule(s), polypeptide(s), drug(s) or combinations thereof in the presence of a positively-charged species;
c) attaching the at least one payloads, such as nucleic acid molecule(s), polypeptide(s), drug(s) or combinations thereof to the mitochondria via the positively-charged species, wherein the mitochondria are contacted with the one or more payloads, such as nucleic acid molecule(s), polypeptide(s), drug(s) or combinations thereof and the polycationic species in a buffer comprising a 4:1 mixture of Solution X comprising or consisting of 20 mM HEPES, 1 mM EGTA and 300 mM Trehalose (pH 7.2) and Solution Y comprising or consisting of 0.1 M CHES (pH 10) and 0.2 M sodium phosphate dibasic dihydrate.

The contacting step of the present invention is not particularly limited to any reaction conditions, times, or reaction times. In general, any reaction conditions facilitating the attachment of a payloads, such as nucleic acid molecule(s), polypeptide(s), drug(s) or combinations thereof to a mitochondrion via a positively-charged species may be used thereby facilitating the formation of the delivery complex may be. However, it is preferred, that the mitochondria are contacted with the at least one payloads, such as nucleic acid molecule(s), polypeptide(s), drug(s) or combinations thereof and the positively-charged species at room temperature for more than 5 minutes, preferably in the dark. Accordingly, the present invention provides a method for attaching a payloads, such as nucleic acid molecule(s), polypeptide(s), drug(s) or combinations thereof to the outer membrane of a mitochondrion, the method comprising the steps of:
a) providing a preparation of mitochondria;
b) contacting the mitochondria provided in step (a) with at least one payloads, such as nucleic acid molecule(s), polypeptide(s), drug(s) or combinations thereof in the presence of a positively-charged species, wherein the mitochondria are contacted with the plurality of nucleic acid molecules and the positively-charged species at room temperature for at least 5 minutes, such as at least 10 minutes, 20 or 30 minutes;
c) attaching the at least one payloads, such as nucleic acid molecule(s), polypeptide(s), drug(s) or combinations thereof to the mitochondria via the positively-charged species.

The present invention provides a method for attaching a payloads, such as nucleic acid molecule(s), polypeptide(s), drug(s) or combinations thereof to the outer membrane of a mitochondrion, the method comprising the steps of:
a) providing a preparation of mitochondria;
b) contacting the mitochondria provided in step (a) with the at least one payloads, such as nucleic acid molecule(s), polypeptide(s), drug(s) or combinations thereof in the presence of a positively-charged species, wherein the mitochondria are contacted with the at least one payloads, such as nucleic acid molecule(s), polypeptide(s), drug(s) or combinations thereof and the positively-charged species in the dark;
c) attaching the at least one payloads, such as nucleic acid molecule(s), polypeptide(s), drug(s) or combinations thereof to the mitochondria via the positively-charged species.

The present invention provides a method for attaching a payloads, such as nucleic acid molecule(s), polypeptide(s), drug(s) or combinations thereof to the outer membrane of a mitochondrion, the method comprising the steps of:
a) providing a preparation of mitochondria;
b) contacting the mitochondria provided in step (a) with at least one payloads, such as nucleic acid molecule(s), polypeptide(s), drug(s) or combinations thereof in the presence of a positively-charged species, wherein the mitochondria are contacted with the plurality of payloads, such as nucleic acid molecule(s), polypeptide(s), drug(s) or combinations thereof and the positively-charged species at room temperature for at least 5 minutes, such as at least 10 minutes, 20 or 30 minutes in the dark;
c) attaching the at least one payloads, such as nucleic acid molecule(s), polypeptide(s), drug(s) or combinations thereof to the mitochondria via the positively-charged species.

Preferably, the present invention provides a method for attaching a payloads, such as nucleic acid molecule(s), polypeptide(s), drug(s) or combinations thereof to the outer membrane of a mitochondrion, the method comprising the steps of:
a) providing a preparation of mitochondria;
b) contacting the mitochondria provided in step (a) with at least one payloads, such as nucleic acid molecule(s), polypeptide(s), drug(s) or combinations thereof in the presence of a positively-charged species, wherein the mitochondria are contacted with the at least one payloads, such as nucleic acid molecule(s), polypeptide(s), drug(s) or combinations thereof and the positively-charged species at room temperature for 30 minutes in the dark;
c) attaching the at least one payloads, such as nucleic acid molecule(s), polypeptide(s), drug(s) or combinations thereof to the mitochondria via the positively-charged species.

As described herein above, the present invention provides a mitochondrion comprising one or more payloads, such as nucleic acid molecule(s), polypeptide(s), drug(s) or combinations thereof attached to the outer membrane of the mitochondrion. The nucleic acid molecules are preferably DNA or RNA. Accordingly, the present invention also provides a method for attaching a DNA molecule to the outer membrane of a mitochondrion, the method comprising the steps of:
a) providing a preparation of mitochondria;
b) contacting the mitochondria provided in step (a) with at least one DNA molecule in the presence of a positively-charged species;
c) attaching at least one DNA molecule to the mitochondria via the positively-charged species.

The present invention also provides a method for attaching a RNA molecule to the outer membrane of a mitochondrion, the method comprising the steps of:
a) providing a preparation of mitochondria;
b) contacting the mitochondria provided in step (a) with at least one RNA molecule in the presence of a positively-charged species;
c) attaching at least one RNA molecule to the mitochondria via the positively-charged species.

As described herein above, the present invention provides for attachment of nucleic acid molecules to a mitochondrion via a positively-charged species, preferably a polycationic species. Accordingly, the present invention provides a method for attaching a nucleic acid molecule to the outer membrane of a mitochondrion, the method comprising the steps of:
a) providing a preparation of mitochondria;
b) contacting the mitochondria provided in step (a) with at least one nucleic acid molecules in the presence of a polycationic species;
c) attaching the at least one nucleic acid molecule to the mitochondria via the polycationic species.

In the sense of the present invention a polycationic species may be a linear or branched polycationic polymer. A linear or branched polycationic polymer may be electrostatically linked to a payloads, such as nucleic acid molecule(s), polypeptide(s), drug(s) or combinations thereof, such as a DNA or RNA molecule. The present invention is not particularly limited to any polycationic polymers. In general, any polycationic polymers facilitating the attachment of payloads, such as nucleic acid molecule(s), polypeptide(s), drug(s) or combinations thereof to a mitochondrion thereby facilitating the formation of the delivery complex may be used. However, it is preferred that a linear or branched polycationic polymer is polylysine, histidylated polylysine, polyornithine, polyarginine, high-mobility group protein (HMG) 1 and 17, modified chitosan, cationized human serum albumin, polyethyleneimine (PEI), polypropyleneimine (PPI), a cationic dendrimer, poly(2-(dimethylamino)ethyl methacrylate) (PDMAEMA), a polyallylamine derivative, diethylaminoethyl (DEAE)-dextran, poly(N-alkyl-4-vinylpyridinium), a poly(amidoamine), cationic gelatin, cationic cellulose or a combination thereof. Accordingly, the present invention provides a method for attaching a payloads, such as nucleic acid molecule(s), polypeptide(s), drug(s) or combinations thereof to the outer membrane of a mitochondrion, the method comprising the steps of:
a) providing a preparation of mitochondria;
b) contacting the mitochondria provided in step (a) with at least one payloads, such as nucleic acid molecule(s), polypeptide(s), drug(s) or combinations thereof in the presence of a linear or branched polycationic polymer;
c) attaching the at least one payloads, such as nucleic acid molecule(s), polypeptide(s), drug(s) or combinations thereof to the mitochondria via the polycationic species.

The present invention provides a method for attaching a payloads, such as nucleic acid molecule(s), polypeptide(s), drug(s) or combinations thereof to the outer membrane of a mitochondrion, the method comprising the steps of:
a) providing a preparation of mitochondria;
b) contacting the mitochondria provided in step (a) with at least one payloads, such as nucleic acid molecule(s), polypeptide(s), drug(s) or combinations thereof in the presence of a linear or branched polycationic polymer which is electrostatically linked to a payloads, such as nucleic acid molecule(s), polypeptide(s), drug(s) or combinations thereof comprised in the plurality of nucleic acids;
c) attaching the at least one payloads, such as nucleic acid molecule(s), polypeptide(s), drug(s) or combinations thereof to the mitochondria via the positively-charged species.

The present invention provides a method for attaching a payloads, such as nucleic acid molecule(s), polypeptide(s), drug(s) or combinations thereof to the outer membrane of a mitochondrion, the method comprising the steps of:

a) providing a preparation of mitochondria;

b) contacting the mitochondria provided in step (a) with at least one payloads, such as nucleic acid molecule(s), polypeptide(s), drug(s) or combinations thereof in the presence of polycationic polymer, wherein the polycationic polymer is polylysine, histidylated polylysine, polyornithine, polyarginine, high-mobility group protein (HMG) 1 and 17, modified chitosan, cationized human serum albumin, polyethyleneimine (PEI), polypropyleneimine (PPI), a cationic dendrimer, poly(2-(dimethylamino)ethyl methacrylate) (PDMAEMA), a polyallylamine derivative, diethylaminoethyl (DEAE)-dextran, poly(N-alkyl-4-vinylpyridinium), a poly(amidoamine), cationic gelatin, cationic cellulose or a combination thereof, optionally, wherein the polycationic polymer which is electrostatically linked to the at least one payloads, such as nucleic acid molecule(s), polypeptide(s), drug(s) or combinations thereof;
c) attaching the at least one payloads, such as nucleic acid molecule(s), polypeptide(s), drug(s) or combinations thereof to the mitochondria via the positively-charged species.

As described herein above, the negative surface charge profile of mitochondria can also be useful for attaching one or more payloads, such as nucleic acid molecule(s), polypeptide(s), drug(s) or combinations thereof electrostatically to the outer membrane of a mitochondrion via a positively-charged nanoparticle or positively-charged particle. Payloads, such as nucleic acid molecule(s), polypeptide(s), drug(s) or combinations thereof may be attached to the surface of a positively-charged nanoparticle/particle or may be encapsulated in the same. The present invention is not particularly limited to any nanoparticles or particles. In general, any positively-charged nanoparticles/particles facilitating the attachment of payloads, such as nucleic acid molecule(s), polypeptide(s), drug(s) or combinations thereof to a mitochondrion thereby facilitating the formation of the delivery complex may be used. However, it is preferred that a positively-charged nanoparticle/particle is a lipid nanoparticle/particle, a dendrimer nanoparticle/particle, a micelle nanoparticle/particle, a protein nanoparticle/particle, a liposome, a non-porous silica nanoparticle/particle, a mesoporous silica nanoparticle/particle, a silicon nanoparticle/particle, a gold nanoparticle/particle, a gold nanowire/wire, a silver nanoparticle/particle, a platinum nanoparticle/particle, a palladium nanoparticle/particle, a titanium dioxide nanoparticle/particle, a carbon nanotube/tube, a carbon dot nanoparticle/particle, a polymer nanoparticle/particle, a zeolite nanoparticle/particle, an aluminium oxide nanoparticle/particle, a hydroxyapatite nanoparticle/particle, a quantum dot nanoparticle/particle, a zinc oxide nanoparticle/particle, a zirconium oxide nanoparticle/particle, graphene or a graphene oxide nanoparticle/particle. Accordingly, the present invention provides a method for attaching a payloads, such as nucleic acid molecule(s), polypeptide(s), drug(s) or combinations thereof to the outer membrane of a mitochondrion, the method comprising the steps of:
a) providing a preparation of mitochondria;
b) contacting the mitochondria provided in step (a) with at least one payloads, such as nucleic acid molecule(s), polypeptide(s), drug(s) or combinations thereof in the presence of a positively-charged nanoparticle;
c) attaching the at least one payloads, such as nucleic acid molecule(s), polypeptide(s), drug(s) or combinations thereof to the mitochondria via the positively-charged species.

The present invention provides a method for attaching a payloads, such as nucleic acid molecule(s), polypeptide(s), drug(s) or combinations thereof to the outer membrane of a mitochondrion, the method comprising the steps of:
a) providing a preparation of mitochondria;
b) contacting the mitochondria provided in step (a) with at least one payloads, such as nucleic acid molecule(s), polypeptide(s), drug(s) or combinations thereof in the presence of a positively-charged nanoparticle;
c) attaching the at least one payloads, such as nucleic acid molecule(s), polypeptide(s), drug(s) or combinations thereof to the mitochondria via the positively-charged nanoparticle. Within the method of the present invention, the method can involve contacting the mitochondrion with the at least one payloads, such as nucleic acid molecule(s), polypeptide(s), drug(s) or combinations thereof in the presence of the positively-charged nanoparticle, wherein the method further comprises
a) attaching the at least one payloads, such as nucleic acid molecule(s), polypeptide(s), drug(s) or combinations thereof to the surface of the positively-charged nanoparticle; or
b) encapsulating the at least one payloads, such as nucleic acid molecule(s), polypeptide(s), drug(s) or combinations thereof within the positively-charged nanoparticle.

Accordingly, the present invention provides a method for attaching a payloads, such as nucleic acid molecule(s), polypeptide(s), drug(s) or combinations thereof to the outer membrane of a mitochondrion, the method comprising the steps of:
a) providing a preparation of mitochondria;
b) contacting the mitochondria provided in step (a) with at least one payloads, such as nucleic acid molecule(s), polypeptide(s), drug(s) or combinations thereof in the presence of a positively-charged nanoparticle, wherein prior to step (b), a further step of:
   a′) attaching the at least one payloads, such as nucleic acid molecule(s), polypeptide(s), drug(s) or combinations thereof to the surface of the positively-charged nanoparticle; or
   b′) encapsulating the at least one payloads, such as nucleic acid molecule(s), polypeptide(s), drug(s) or combinations thereof within the positively-charged nanoparticle, is performed;
c) attaching the at least one payloads, such as nucleic acid molecule(s), polypeptide(s), drug(s) or combinations thereof to the mitochondria via the positively-charged nanoparticle.

The present invention provides a method for attaching a payloads, such as nucleic acid molecule(s), polypeptide(s), drug(s) or combinations thereof to the outer membrane of a mitochondrion, the method comprising the steps of:
a) providing a preparation of mitochondria;
b) contacting the mitochondria provided in step (a) with at least one payloads, such as nucleic acid molecule(s), polypeptide(s), drug(s) or combinations thereof in the presence of a positively-charged nanoparticle, wherein prior to step (b), a further step of:
   a′) attaching the at least one payloads, such as nucleic acid molecule(s), polypeptide(s), drug(s) or combinations thereof to the surface of the positively-charged nanoparticle; or
   b′) encapsulating the at least one payloads, such as nucleic acid molecule(s), polypeptide(s), drug(s) or combinations thereof within the positively-charged nanoparticle, is performed,
c) attaching the at least one payloads, such as nucleic acid molecule(s), polypeptide(s), drug(s) or combinations thereof to the mitochondria via the positively-charged nanoparticle; wherein the positively-charged nanoparticle is a lipid nanoparticle, a dendrimer nanoparticle, a micelle nanoparticle, a protein nanoparticle, a liposome, a non-porous silica nanoparticle, a mesoporous silica nanoparticle, a silicon nanoparticle, a gold nanoparticle, a gold nanowire, a silver nanoparticle, a platinum nanoparticle, a palladium nanoparticle, a titanium dioxide nanoparticle, a carbon nanotube, a carbon dot nanoparticle, a polymer nanoparticle, a zeolite nanoparticle, an aluminium oxide nanoparticle, a hydroxyapatite nanoparticle, a quantum dot nanoparticle, a zinc oxide nanoparticle, a zirconium oxide nanoparticle, graphene or a graphene oxide nanoparticle.

In another aspect, the present invention provides methods for covalently attaching a payloads, such as nucleic acid molecule(s), polypeptide(s), drug(s) or combinations thereof to the outer membrane of a mitochondrion. As described herein above, the present invention provides payloads, such as nucleic acid molecule(s), polypeptide(s), drug(s) or combinations thereof that may be covalently linked to the outer membrane of a mitochondrion be it directly or indirectly, such as via an intermediate entity. An exemplary intermediate entity comprises an activated ester such as a N-hydroxysuccinimide (NHS) ester. Accordingly, the present invention provides a method for covalently attaching a payloads, such as nucleic acid molecule(s), polypeptide(s), drug(s) or combinations thereof to the outer membrane of a mitochondrion, the method comprising the steps of:
a) providing a preparation of mitochondria;
b) providing a payloads, such as nucleic acid molecule(s), polypeptide(s), drug(s) or combinations thereof that has been modified to comprise an activated ester; and
c) attaching the payloads, such as nucleic acid molecule(s), polypeptide(s), drug(s) or combinations thereof provided in step (b) to an amine comprised in a polypeptide in the outer membrane of the mitochondria.

Preferably, the present invention provides a method for covalently attaching a payloads, such as nucleic acid molecule(s), polypeptide(s), drug(s) or combinations thereof to the outer membrane of a mitochondrion, the method comprising the steps of:
a) providing a preparation of mitochondria;
providing a payloads, such as nucleic acid molecule(s), polypeptide(s), drug(s) or combinations thereof that has been modified to comprise a N-hydroxysuccinimide (NHS) ester; and
c) attaching the payloads, such as nucleic acid molecule(s), polypeptide(s), drug(s) or combinations thereof provided in step (b) to an amine comprised in a polypeptide in the outer membrane of the mitochondria.

In another aspect the present invention provides a method for covalently attaching a payloads, such as nucleic acid molecule(s), polypeptide(s), drug(s) or combinations thereof to the outer membrane of a mitochondrion, the method comprising the steps of:
a) providing a preparation of mitochondria;
b) providing a payloads, such as nucleic acid molecule(s), polypeptide(s), drug(s) or combinations thereof that has been modified to comprise a chemical group; and
c) attaching the payloads, such as nucleic acid molecule(s), polypeptide(s), drug(s) or combinations thereof provided in step (b) to an amine comprised in a polypeptide in the outer membrane of the mitochondria via the chemical group.

The present invention provides a method for covalently attaching a payloads, such as nucleic acid molecule(s), polypeptide(s), drug(s) or combinations thereof to the outer membrane of a mitochondrion, the method comprising the steps of:
a) providing a preparation of mitochondria;
b) providing a payloads, such as nucleic acid molecule(s), polypeptide(s), drug(s) or combinations thereof that has been modified to comprise chemical group selected from isothiocyanate, isocyanate, acyl azide, sulfonyl chloride, aldehyde, glyoxal, epoxides, oxirane, carbonate, aryl halide, imidoester, carbodiimide, anhydride and fluorophenyl ester; and
c) attaching the nucleic acid molecule provided in step (b) to an amine comprised in a polypeptide in the outer membrane of the mitochondria via the chemical group.

As described hereinabove, payloads, such as nucleic acid molecule(s), polypeptide(s), drug(s) or combinations thereof may be attached to or encapsulated in a nanoparticle which then may be covalently attached to a mitochondrion via a covalent bond e.g., an amide bond.

The invention provides a method for covalently attaching a payloads, such as nucleic acid molecule(s), polypeptide(s), drug(s) or combinations thereof to the outer membrane of a mitochondrion, the method comprising the steps of:
a) providing a preparation of mitochondria;
b) encapsulating a payloads, such as nucleic acid molecule(s), polypeptide(s), drug(s) or combinations thereof in a nanoparticle, wherein the surface of the nanoparticle comprises an activated ester; and
c) attaching the nanoparticle provided in step (b) to an amine comprised in a polypeptide in the outer membrane of the mitochondria.

Preferably, the invention provides a method for covalently attaching a payloads, such as nucleic acid molecule(s), polypeptide(s), drug(s) or combinations thereof to the outer membrane of a mitochondrion, the method comprising the steps of:
a) providing a preparation of mitochondria;
b) encapsulating a payloads, such as nucleic acid molecule(s), polypeptide(s), drug(s) or combinations thereof in a nanoparticle, wherein the surface of the nanoparticle comprises a N-hydroxysuccinimide (NHS) ester; and
c) attaching the nanoparticle provided in step (b) to an amine comprised in a polypeptide in the outer membrane of the mitochondria.

The nanoparticle is not particularly limited and may be any nanoparticle known to the skilled person. In some embodiments, the nanoparticle is a positively-charged nanoparticle as described hereinabove. In another aspect, the invention provides a method for covalently attaching a payloads, such as nucleic acid molecule(s), polypeptide(s), drug(s) or combinations thereof to the outer membrane of a mitochondrion, the method comprising the steps of:
a) providing a preparation of mitochondria;
b) encapsulating a payloads, such as nucleic acid molecule(s), polypeptide(s), drug(s) or combinations thereof in a nanoparticle, wherein the surface of the nanoparticle comprises a chemical group; and
c) attaching the nanoparticle provided in step (b) to an amine comprised in a polypeptide in the outer membrane of the mitochondria via the chemical group.

The invention provides a method for covalently attaching a payloads, such as nucleic acid molecule(s), polypeptide(s), drug(s) or combinations thereof to the outer membrane of a mitochondrion, the method comprising the steps of:
a) providing a preparation of mitochondria;
b) encapsulating a payloads, such as nucleic acid molecule(s), polypeptide(s), drug(s) or combinations thereof in a nanoparticle, wherein the surface of the nanoparticle comprises a chemical group selected from an isothiocyanate, isocyanate, acyl azide, sulfonyl chloride, aldehyde, glyoxal, epoxides, oxirane, carbonate, aryl halide, imidoester, carbodiimide, anhydride and fluorophenyl ester; and
c) attaching the nanoparticle provided in step (b) to an amine comprised in a polypeptide in the outer membrane of the mitochondria via the chemical group.

In further embodiments, the present invention provides methods for attaching a payloads, such as nucleic acid molecule(s), polypeptide(s), drug(s) or combinations thereof to the outer membrane of a mitochondrion, wherein the nucleic acid is linked to an antibody that specifically binds to an antigen comprised in the outer membrane of the mitochondrion. Accordingly, the present invention provides a method for attaching at least one payloads, such as nucleic acid molecule(s), polypeptide(s), drug(s) or combinations thereof to the outer membrane of a mitochondrion, the method comprising the steps of:
a) providing a preparation of mitochondria;
b) contacting the mitochondria provided in step (a) comprising an antigen in their outer membrane with at least one payloads, such as nucleic acid molecule(s), polypeptide(s), drug(s) or combinations thereof linked to an antibody; and
c) attaching the at least one payloads, such as nucleic acid molecule(s), polypeptide(s), drug(s) or combinations thereof to the mitochondria via the antibody, wherein the antibody specifically binds to the antigen comprised in the outer membrane of the mitochondrion.

In some embodiments, the present invention provides a method for attaching one or more payloads, such as nucleic acid molecule(s), polypeptide(s), drug(s) or combinations thereof to the outer membrane of a mitochondrion, the method comprising the steps of:
a) providing a preparation of mitochondria;
b) contacting the mitochondria provided in step (a) comprising an antigen in their outer membrane with at least one payloads, such as nucleic acid molecule(s), polypeptide(s), drug(s) or combinations thereof encapsulated in a nanoparticle, wherein the nanoparticle is covalently linked to an antibody; and
c) attaching the at least one nucleic acid molecule to the mitochondria via the antibody, wherein the antibody specifically binds to the antigen comprised in the outer membrane of the mitochondrion.

In some embodiments, the present invention provides a method for attaching one or more payloads, such as nucleic acid molecule(s), polypeptide(s), drug(s) or combinations thereof to the outer membrane of a mitochondrion, the method comprising the steps of:
a) providing a preparation of mitochondria;
b) contacting the mitochondria provided in step (a) comprising an antigen in their outer membrane with at least one payloads, such as nucleic acid molecule(s), polypeptide(s), drug(s) or combinations thereof, wherein the nucleic acid molecule is electrostatically linked to a modified antibody, wherein the modified antibody possesses one or more positive charge(s); and
c) attaching the at least one nucleic acid molecule to the mitochondria via the antibody, wherein the antibody specifically binds to the antigen comprised in the outer membrane of the mitochondrion.

In some embodiments, the present invention provides a method for attaching one or more payloads, such as nucleic acid molecule(s), polypeptide(s), drug(s) or combinations thereof to the outer membrane of a mitochondrion, the method comprising the steps of:
a) providing a preparation of mitochondria;
b) contacting the mitochondria provided in step (a) comprising an antigen in their outer membrane with at least one payloads, such as nucleic acid molecule(s), polypeptide(s), drug(s) or combinations thereof, wherein the nucleic acid molecule is covalently linked to biotin, wherein biotin is linked to an avidin conjugated antibody; and
c) attaching the at least one payloads, such as nucleic acid molecule(s), polypeptide(s), drug(s) or combinations thereof to the mitochondria via the antibody, wherein the antibody specifically binds to the antigen comprised in the outer membrane of the mitochondrion.

In some embodiments, the present invention provides a method for attaching one or more payloads, such as nucleic acid molecule(s), polypeptide(s), drug(s) or combinations thereof to the outer membrane of a mitochondrion, the method comprising the steps of:
a) providing a preparation of mitochondria;
b) contacting the mitochondria provided in step (a) comprising an antigen in their outer membrane with at least one payloads, such as nucleic acid molecule(s), polypeptide(s), drug(s) or combinations thereof, wherein the nucleic acid molecule is covalently linked to an activated ester, wherein the activated ester is linked to the antibody via an amide bond; and
c) attaching the at least one payloads, such as nucleic acid molecule(s), polypeptide(s), drug(s) or combinations thereof to the mitochondria via the antibody, wherein the antibody specifically binds to the antigen comprised in the outer membrane of the mitochondrion.

In some embodiments, the present invention provides a method for attaching one or more nucleic acid molecule(s), wherein the one or more nucleic acid molecule(s) is a single-stranded nucleic acid molecule (ssDNA or ssRNA), to the outer membrane of a mitochondrion, the method comprising the steps of:
a) providing a preparation of mitochondria;
b) contacting the mitochondria provided in step (a) comprising an antigen in their outer membrane with at least one single-stranded nucleic acid molecule, wherein the single stranded nucleic acid molecule is hybridizable with one or more complementary single-stranded nucleic acid molecule(s) attached on or to an antibody; and
c) attaching the at least one single stranded nucleic acid molecule to the mitochondria via the antibody, wherein the antibody specifically binds to the antigen comprised in the outer membrane of the mitochondrion.

In some embodiments, the present invention provides a method for attaching one or more payloads, such as nucleic acid molecule(s), polypeptide(s), drug(s) or combinations thereof to the outer membrane of a mitochondrion, the method comprising the steps of:
a) providing a preparation of mitochondria;
b) contacting the mitochondria provided in step (a) comprising an antigen in their outer membrane with at least one payloads, such as nucleic acid molecule(s), polypeptide(s), drug(s) or combinations thereof, wherein the one or more nucleic acid molecule(s) is encapsulated in a nanoparticle, wherein the nanoparticle is electrostatically linked to a modified antibody, wherein the modified antibody possesses one or more positive charge(s); and
c) attaching the at least one nucleic acid molecule to the mitochondria via the antibody, wherein the antibody specifically binds to the antigen comprised in the outer membrane of the mitochondrion.

In some embodiments, the present invention provides a method for attaching one or more payloads, such as nucleic acid molecule(s), polypeptide(s), drug(s) or combinations thereof to the outer membrane of a mitochondrion, the method comprising the steps of:
a) providing a preparation of mitochondria;
b) contacting the mitochondria provided in step (a) comprising an antigen in their outer membrane with at least one payloads, such as nucleic acid molecule(s), polypeptide(s), drug(s) or combinations thereof, wherein the one or more payloads, such as nucleic acid molecule(s), polypeptide(s), drug(s) or combinations thereof is encapsulated in a nanoparticle, wherein the nanoparticle is electrostatically linked to a modified antibody, wherein the modified antibody possesses one or more negative charge(s); and
c) attaching the at least one payloads, such as nucleic acid molecule(s), polypeptide(s), drug(s) or combinations thereof to the mitochondria via the antibody, wherein the antibody specifically binds to the antigen comprised in the outer membrane of the mitochondrion.

In some embodiments, the present invention provides a method for attaching one or more payloads, such as nucleic acid molecule(s), polypeptide(s), drug(s) or combinations thereof to the outer membrane of a mitochondrion, the method comprising the steps of:
a) providing a preparation of mitochondria;
b) contacting the mitochondria provided in step (a) comprising an antigen in their outer membrane with at least one payloads, such as nucleic acid molecule(s), polypeptide(s), drug(s) or combinations thereof, wherein the one or more nucleic acid molecule(s) is encapsulated in a nanoparticle, wherein the nanoparticle is covalently linked to biotin, wherein biotin is linked to an avidin conjugated antibody; and
c) attaching the at least one payloads, such as nucleic acid molecule(s), polypeptide(s), drug(s) or combinations thereof to the mitochondria via the antibody, wherein the antibody specifically binds to the antigen comprised in the outer membrane of the mitochondrion. In some embodiments, the present invention provides a method for attaching one or more payloads, such as nucleic acid molecule(s), polypeptide(s), drug(s) or combinations thereof to the outer membrane of a mitochondrion, the method comprising the steps of:
a) providing a preparation of mitochondria;
b) contacting the mitochondria provided in step (a) comprising an antigen in their outer membrane with at least one payloads, such as nucleic acid molecule(s), polypeptide(s), drug(s) or combinations thereof, wherein the one or more nucleic acid molecule(s) is encapsulated in a nanoparticle, wherein the nanoparticle is covalently linked to an activated ester, wherein the activated ester is linked to the antibody via amide bond; and
c) attaching the at least one nucleic acid molecule to the mitochondria via the antibody, wherein the antibody specifically binds to the antigen comprised in the outer membrane of the mitochondrion.

The antigen comprised in the outer membrane of the mitochondrion may be any antigen capable of binding the antibody linked to the payloads, such as nucleic acid molecule(s), polypeptide(s), drug(s) or combinations thereof. In preferred embodiments, the antigen is OPA1, TOM70, TOMM20, Mitofusin 1, Mitofusin 2 or VDAC1.

The invention also provides a method for attaching a payload, such as nucleic acid molecule(s), polypeptide(s), drug(s) or combinations thereof to the outer membrane of a mitochondrion via a mitochondria-targeting small molecule, the method comprising the steps of:
a) providing a preparation of mitochondria;
b) contacting the mitochondria provided in step (a) with at least one payloads, such as nucleic acid molecule(s), polypeptide(s), drug(s) or combinations thereof linked to a mitochondria-targeting small molecule; and
c) attaching the at least one payloads, such as nucleic acid molecule(s), polypeptide(s), drug(s) or combinations thereof to the mitochondria via a mitochondria-targeting small molecule.

The mitochondrion-targeting small molecule may be any mitochondrion-targeting small molecule. Preferably, the mitochondria-targeting small molecule is selected from: triphenylphosphonium (TPP), dequalinium (DQA), E-4-(1H-Indol-3-ylvinyl)-N-Methylpyridineiodide (F16), Rhodamine 19, biguanidine and guanidine.

In the sense of the present invention, the nucleic acid molecules do not necessarily relate to identical nucleic acid molecules, i.e. molecules of identical sequence. Although it is appreciated to deliver sequence identical nucleic acid molecules in some aspects, in other aspects of the invention at least two or more different nucleic acid molecules may be attached to the outer membrane of a mitochondrion.

In some embodiments, the method of the invention further comprises linking to and/or enveloping the mitochondrion comprising the one or more payloads, such as nucleic acid molecule(s), polypeptide(s), drug(s) or combinations thereof with a protective layer. The mitochondrion comprising the one or more nucleic acid molecule(s) may be any mitochondrion as described hereinabove and any protective layer as described hereinabove. The method of linking and/or enveloping the mitochondrion in a protective preferably involves contacting the mitochondrion with the components which form the protective layer, e.g., the protective polymer or protective lipid layer as described hereinabove. In preferred embodiments, the invention is a method for attaching a nucleic acid molecule to the outer membrane of a mitochondrion, wherein the method comprises the steps of:
a) providing a preparation of mitochondria;
b) contacting the mitochondria provided in step (a) with at least one nucleic acid molecule in the presence of a positively-charged species;
c) attaching the at least one nucleic acid molecule(s) to the mitochondria via the positively-charged species; and
d) linking and/or enveloping the mitochondrion provided in steps (a) to (c) with a protective layer.

In some embodiments, the protective layer is a protective polymer. The protective polymer is as described herein above.

In some embodiments of the method of the present invention, the protective polymer is a linear or branched cationic polymer, optionally wherein the linear or branched cationic polymer is electrostatically linked to the one or more nucleic acid molecule(s). Preferably, the linear or branched cationic polymer is polyethyleneimine, RGD-modified polyethyleneimine, polylysine, RGD-modified polylysine, polyornithine, RGD-modified polyornithine, polyarginine, RGD modified polyarginine, polypropyleneimine, RGD-modified polypropyleneimine, polyallylamine, RGD-modified polyallylamine, chitosan, RGD-modified chitosan, poly(2-(dimethylamino)ethyl methacrylate), RGD-modified poly(2-(dimethylamino)ethyl methacrylate), poly(amidoamine)s, RGD-modified poly(amidoamine) s or a combination thereof.

In some embodiments of the method of the present invention, the protective polymer is a linear or branched cationic block copolymer, optionally wherein the linear or branched cationic block copolymer is electrostatically linked to the one or more nucleic acid molecule(s). Preferably, the cationic block copolymer is poly(ethylene glycol)-block-polyethyleneimine, RGD-modified poly(ethylene glycol)-block-polyethyleneimine, poly(ethylene glycol)-block-polylysine, RGD-modified poly(ethylene glycol)-block-polylysine, poly(ethylene glycol)-block-polyornithine, RGD-modified poly(ethylene glycol)-block-polyornithine, poly(ethylene glycol)-block-polyarginine, RGD-modified poly(ethylene glycol)-block-polyarginine, poly(ethylene glycol)-block-polypropyleneimine, RGD-modified poly(ethylene glycol)-block-polypropyleneimine, poly(ethylene glycol)-block-polyallylamine, RGD-modified poly(ethylene glycol)-block-polyallylamine, poly(ethylene glycol)-block-poly(2-(dimethylamino)ethyl methacrylate), RGD-modified poly(ethylene glycol)-block-poly(2-(dimethylamino)ethyl methacrylate), poly(ethylene glycol)-block-poly(amidoamine)s, RGD-modified poly(ethylene glycol)-block-poly(amidoamine)s or a combination thereof.

In some embodiments of the method of the present invention, the protective polymer is a linear or branched cationic graft (g) copolymer, optionally wherein the linear or branched cationic graft (g) copolymer is electrostatically linked to the one or more nucleic acid molecule(s). Preferably, the cationic graft (g) copolymer is poly(ethylene glycol)-g-polyethyleneimine, RGD-modified poly(ethylene glycol)-g-polyethyleneimine, poly(ethylene glycol)-g-polylysine, RGD-modified poly(ethylene glycol)-g-polylysine, poly(ethylene glycol)-g-polyornithine, RGD-modified poly(ethylene glycol)-g-polyornithine, poly(ethylene glycol)-g-polyarginine, RGD-modified poly(ethylene glycol)-g-polyarginine, poly(ethylene glycol)-g-polypropyleneimine, RGD-modified poly(ethylene glycol)-g-polypropyleneimine, poly(ethylene glycol)-g-polyallylamine, RGD-modified poly(ethylene glycol)-g-polyallylamine, poly(ethylene glycol)-g-poly(2-(dimethylamino)ethyl methacrylate), RGD-modified poly(ethylene glycol)-g-poly(2-(dimethylamino)ethyl methacrylate), poly(ethylene glycol)-g-poly(amidoamine)s, RGD-modified poly(ethylene glycol)-g-poly(amidoamine)s or a combination thereof.

In some embodiments of the method of the present invention, the protective polymer is a linear or branched pegylated (PEG) cationic polymer, optionally wherein the linear or branched pegylated (PEG) cationic polymer is electrostatically linked to the one or more nucleic acid molecule(s). Preferably, the pegylated (PEG) cationic polymer is pegylated-polyethyleneimine, RGD-modified pegylated polyethyleneimine, pegylated polylysine, RGD-modified pegylated polylysine, histidylated polylysine, pegylated polyornithine, RGD-modified pegylated polyornithine, pegylated polyarginine, RGD-modified pegylated polyarginine, pegylated polypropyleneimine, RGD-modified pegylated polypropyleneimine, pegylated polyallylamine, RGD-modified pegylated polyallylamine, pegylated chitosan, RGD-modified pegylated chitosan, pegylated poly(2-(dimethylamino)ethyl methacrylate), RGD-modified pegylated poly(2-(dimethylamino)ethyl methacrylate), pegylated poly(amidoamine)s RGD-modified pegylated poly(amidoamine)s or a combination thereof.

In some embodiments of the method of the present invention, the protective layer is a lipid formulation, optionally wherein the lipid formulation is a cationic lipid formulation, further optionally wherein the cationic lipid formulation is electrostatically linked to the one or more nucleic acid molecule(s). Preferably, wherein the lipid formulation comprises DC-cholesterol (3β-[N—(N′,N′-Dimethylaminoethane)-carbamoyl]cholesterol hydrochloride), DLinDMA (1,2-dilinoleyloxy-3-dimethylaminopropane), DLinMC3DMA (dilinoleylmethyl-4-dimethylaminobutyrate), DODMA (1,2-dioleyloxy-3-dimethylaminopropane), DOGS (dioctadecylamidoglycylspermine), DOSPA (2,3-dioleyloxy-N-[2 (sperminecarboxamido)ethyl]-N,N-dimethyl-1-propanaminium), DOTAP (1,2-dioleoyl-3-trimethylammonium-propane chloride), DOTMA (1,2-di-O-octadecenyl-3-trimethylammonium propane chloride), UGG (unsaturated guanidinium glycoside), DOPE (1,2-Dioleoyl-sn-glycerophosphoethanolamine), lipofectamine or a combination thereof. In further embodiments, the lipid formulation further comprises another lipid, preferably wherein said lipid is cholesterol, a substituted or unsubstituted cholesterol, a cholesterol derivative, such as a hydroxylated cholesterol derivative (e.g., a hydroxycholesterol), a PEG-lipid, DMPC (1,2-Dimyristoyl-sn-glycero-3-phosphocholine), DSPC (1,2-Distearoyl-sn-glycero-3-phosphocholine), DODAP (1,2-dioleoyl-3-dimethylammonium propane), DDA (dimethyldioctadecylammonium), 1,2-dioleoyl-sn-glycero-3-phosphate, 1,2-dimyristoyl-sn-glycero-3-phosphate, bis(monooleoylglycerol)phosphate or a combination thereof.

In some embodiments of the method of the present invention the protective polymer is a zwitterionic protective polymer, optionally wherein the zwitterionic protective polymer is electrostatically linked to the one or more nucleic acid molecule(s). Preferably, the zwitterionic protective polymer is selected from: poly(2-methacryloyloxyethyl phosphorylcholine) (PMPC), polyethyleneimine-g-poly(2-methacryloyloxyethyl phosphorylcholine) (PEI-g-PMPC), co-assembly of cationic (carboxyl-functionalized) and anionic (amino-functionalized) copolyesters based on poly(ε-caprolactone)-block-poly(butylene fumarate)-block-poly(ε-caprolactone) (PCL-b-PBF-b-PCL), poly(lactic-co-glycolic acid) (PLGA)-PCB block copolymers (PLGA-b-PCB).

In some embodiments of the method of the present invention, the protective layer is linked to a targeting moiety, optionally wherein the protective layer linked to a targeting moiety is electrostatically linked to the one or more nucleic acid molecule(s). The linkage and targeting and targeting moiety is as described hereinabove. Preferably the targeting moiety is an antibody or carbohydrate molecule.

In some embodiments of the method of the present invention, the protective layer is linked to an antibody, optionally wherein the protective layer is linked to an antibody, wherein the antibody is electrostatically linked to the one or more nucleic acid molecule(s).

In some embodiments of the method of the invention, the protective layer is linked to a carbohydrate, optionally wherein the protective layer linked to a carbohydrate is electrostatically linked to the one or more nucleic acid molecule(s).

In preferred embodiments, the invention is a method wherein the mitochondrion comprises a positively-charged species, wherein the positively-charged species is a polycationic polymer, and wherein the wight ratio of the polycationic polymer to the protective layer is between about 1:2.

The method according to claim 100, wherein 50 μg to 200 μg of mitochondria are contacted with 0.1 to 50 μmol of nucleic acid molecules and 0.2 to 10 μg of the protective layer.
50 microgram of mitochondria corresponds to ca. 150 million of mitochondria. 1 mg/mL of mitochondria (based on Qubit protein assay) corresponds to ca. 3B mitochondria/mL (based on particle counter).
The concentration of the preparation of protective polymer used for the preparation of the mitochondrion of the invention is 1 mg/mL. The amount of protective polymer is between 0.1 mg and 10 mg.
The concentration of the preparation of nanoparticle used for the preparation of the mitochondrion of the invention is 1 mg/mL. The amount of protective polymer is between 0.1 mg and 10 mg.

In further embodiments, the method of the present invention may involve a centrifugation step. The centrifugation step, within the context of the present invention enables the removal of the components comprising the mitochondrion delivery vehicle, e.g., unattached payload, such as the nucleic acid molecule, the positively-charged species or the protective layer to facilitate the formation of the delivery vehicle. As the skilled person is aware, the centrifugation step may be performed after any step which requires removal of excess components of the delivery vehicle, e.g. excess payload, excess positive-charged species, excess protective layer.

### Accordingly, the Method of the Invention May Comprise the Steps of:

a) providing a preparation of mitochondria;
b) contacting the mitochondria provided in step (a) with at least one nucleic acid molecule in the presence of a positively-charged species;
c) attaching the at least one nucleic acid molecule(s) to the mitochondria via the positively-charged species;
d) centrifuging the mitochondrion provided in step (c); and
e) optionally linking and/or enveloping the mitochondrion provided in step (d) in a protective layer.

### In Further Embodiments, the Method of the Invention May Comprise the Steps of:

a) providing a preparation of mitochondria;
b) contacting the mitochondria provided in step (a) with a positively-charged species;
c) optionally centrifuging the mitochondrion provided in step (b);
d) contacting the mitochondrion provided in steps (a) to (c) with at least one nucleic acid molecule;
e) attaching the at least one nucleic acid molecule(s) to the mitochondria via the positively-charged species;
f) optionally centrifuging the mitochondrion provided in step (d); and
g) optionally linking and/or enveloping the mitochondrion provided in step (d) in a protective layer.

### In Further Embodiments, the Method of the Invention May Comprise the Steps of:

a) providing a preparation of mitochondria;
b) contacting at least one nucleic acid molecule with a positively-charged species to form a positively-charged complex;
c) contacting the mitochondrion of (a) with the positively-charged complex of (b);
d) attaching the at least one nucleic acid molecule to the mitochondria via the positively-charged species;
f) optionally centrifuging the mitochondrion provided in step (d); and
g) optionally linking and/or enveloping the mitochondrion provided in step (d) in a protective layer.

### In Some Embodiments, the Method of the Invention May Comprise the Steps of:

a) providing a preparation of mitochondria;
b) providing a nucleic acid molecule that has been modified to comprise an activated ester; and
c) attaching the nucleic acid molecule provided in step (b) to an amine comprised in a polypeptide in the outer membrane of the mitochondria;
d) centrifuging the mitochondrion provided in step (c); and
e) optionally linking and/or enveloping the mitochondrion provided in step (d) in a protective layer.

### In Some Embodiments, the Method of the Invention May Comprise the Steps of:

a) providing a preparation of mitochondria;
b) encapsulating a nucleic acid molecule in a nanoparticle, wherein the surface of the nanoparticle comprises a chemical group capable of covalently attaching to a polypeptide in the outer membrane of the mitochondrion;
c) attaching the nucleic acid molecule provided in step (b) to a polypeptide in the outer membrane of the mitochondria;
d) centrifuging the mitochondrion provided in step (c); and
e) optionally linking and/or enveloping the mitochondrion provided in step (d) in a protective layer.

### In Some Embodiments, the Method of the Invention May Comprise the Steps of:

a) providing a preparation of mitochondria;
b) contacting the mitochondria provided in step (a) comprising an antigen in their outer membrane with at least one nucleic acid molecule linked to an antibody;
c) attaching the at least one nucleic acid molecule to the mitochondria via the antibody, wherein the antibody specifically binds to the antigen comprised in the outer membrane of the mitochondrion;
d) centrifuging the mitochondrion provided in step (c); and
e) optionally linking and/or enveloping the mitochondrion provided in step (d) in a protective layer.

### In Some Embodiments, the Method of the Invention May Comprise the Steps of:

a) providing a preparation of mitochondria;
b) contacting the mitochondria provided in step (a) with at least one nucleic acid molecule linked to a mitochondria-targeting small molecule;
c) attaching the at least one nucleic acid molecule to the mitochondria via a mitochondria-targeting small molecule;
d) centrifuging the mitochondrion provided in step (c); and
e) optionally linking and/or enveloping the mitochondrion provided in step (d) in a protective layer.

The present invention also provides a mitochondrion comprising one or more polypeptide attached to the outer membrane of the mitochondrion. Accordingly, the products, methods, apparatus and uses of the present invention may be carried out by attaching a polypeptide to a mitochondrion instead of or together with nucleic acid molecules. The terms “peptide”, “polypeptide”, and “protein” are used interchangeably herein, and refer to a polymeric form of amino acids of any length, which can include coded and non-coded amino acids, chemically or biochemically modified or derivatized amino acids, and polypeptides having modified peptide backbones. In some embodiments the polypeptide of the present invention comprises 3 to 38000 amino acids. As used herein, the term “protein” refers to a macromolecule comprising one or more polypeptide chains. A protein may also comprise non-peptidic components, such as carbohydrate groups. Carbohydrates and other non-peptidic substituents may be added to a protein by the cell in which the protein is produced and will vary with the type of cell. Some proteins are defined herein in terms of their amino acid backbone structures. As used herein the term “peptide” refers to a polypeptide having 2-100 amino acid monomers.

The present invention is not particularly limited to any polypeptide. Any polypeptide of interest may be used as a payload attached to the outer membrane of a mitochondrion. Accordingly, the present invention provides polypeptides attached to the outer membrane of a mitochondrion useful for e.g., therapy and/or gene editing. In general, any polypeptide of interest may be attached to the outer membrane of a mitochondrion. In the sense of the present invention, the mitochondrion may be positively or negatively-charged. In the sense of the present invention, the polypeptide may be positively or negatively-charged. A positively-charged polypeptide may be attached to a negatively-charged mitochondrion or entity. A negatively-charged polypeptide may be attached to a positively-charged mitochondrion or entity. Either of the above constellations can lead to a successful attachment via electrostatic interaction as long as the mitochondrion and the polypeptide carry opposite charges or do not carry the same charges in the respective pH of the milieu where the polypeptide is contacted with the mitochondrion or entity, e.g. at physiological pH (approx. 7.2). In preferred embodiments, the positively-charged polypeptide comprises lysine, arginine or histidine. In further embodiments, the negatively-charged polypeptide comprises aspartate or glutamate.

Accordingly, the present invention provides a mitochondrion comprising one or more polypeptide(s) attached to the outer membrane of the mitochondrion, wherein the one or more polypeptide(s):
a) is electrostatically attached to the outer membrane of the mitochondrion; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule.

In some embodiments the polypeptide is negatively-charged. In other embodiments, the polypeptide is positively-charged.

The present invention provides a mitochondrion-polypeptide complex useful for delivery of polypeptides into cells, tissues or organs. The present invention also provides for attachment of polypeptides to a mitochondrion, such polypeptides may be charged. One or more polypeptide(s) may be electrostatically attached to the outer membrane of a mitochondrion. One or more polypeptide(s) may be electrostatically attached to the outer membrane of a mitochondrion via a positively-charged species. One or more positively-charged polypeptide may be electrostatically attached to the outer membrane of a negatively-charged mitochondrion. One or more negatively-charged polypeptide may be electrostatically attached to the outer membrane of a mitochondrion via a positively-charged species.

A mitochondrion can electrostatically interact with a polypeptide thereby forming a complex comprising a mitochondrion and one or more polypeptide. Accordingly, electrostatic interaction may be used to attach a positively-charged entity to a negatively-charged entity. In the sense of the present invention, the mitochondrion may be positively or negatively-charged. In the sense of the present invention, the polypeptide may be positively or negatively-charged. Either of the above constellations can lead to a successful attachment via electrostatic interaction as long as the mitochondrion and the polypeptide carry opposite charges or do not carry the same charges. Mitochondria possess a negatively-charged surface to which positively-charged polypeptides may be electrostatically attached. In one aspect, the present invention provides a mitochondrion comprising one or more polypeptide(s) attached to the outer membrane of the mitochondrion. The polypeptide may be electrostatically attached to the outer membrane. The polypeptide may be a charged polypeptide. The polypeptide may be a positively-charged polypeptide.

Mitochondria possess a negatively-charged surface which may be functionalized with cationic molecules, turning the surface charge of mitochondria's outer membrane to positive values (i.e., either partially or entirely positive values). Subsequently, positively-charged mitochondria may be conjugated with negatively-charged polypeptides. In one aspect, the present invention provides a mitochondrion comprising one or more polypeptide(s) attached to the outer membrane of the mitochondrion, wherein the one or more polypeptide is electrostatically attached to the outer membrane of the mitochondrion via a positively-charged species. The present invention provides a mitochondrion comprising one or more polypeptide(s) attached to the outer membrane of the mitochondrion, wherein the one or more polypeptide(s) is electrostatically attached to the outer membrane of the mitochondrion via a positively-charged species and wherein the polypeptide is negatively-charged.

A polypeptide of the present invention is preferably positively or negatively-charged. As used herein “charge” or “charged” relates to the overall or net charge on a peptide or protein, i.e., the sum of the charges in the peptide or protein. The skilled person is aware how to determine the net charge of a given polypeptide in a given pH (e.g., at physiological pH (approx. 7.2). In the sense of the present invention the net charge of a polypeptide of the present invention is preferably negative or positive when being contacted with a mitochondrion. Accordingly, one or more polypeptide(s) may be electrostatically attached to the outer membrane of a mitochondrion via a positively-charged species. One or more polypeptide(s) may be electrostatically attached to the outer membrane of a mitochondrion via a polycationic species, wherein polycationic species is linear or branched polycationic polymer. One or more polypeptide may be electrostatically attached to the outer membrane of a mitochondrion via a linear or branched polycationic polymer is polylysine, histidylated polylysine, polyornithine, polyarginine, high-mobility group protein (HMG) 1 and 17, modified chitosan, cationized human serum albumin, polyethyleneimine (PEI), polypropyleneimine (PPI), a cationic dendrimer, poly(2-(dimethylamino)ethyl methacrylate) (PDMAEMA), a polyallylamine derivative, diethylaminoethyl (DEAE)-dextran, poly(N-alkyl-4-vinylpyridinium), a poly(amidoamine), cationic gelatin, cationic cellulose or a combination thereof.

In the sense of the present invention, the negative surface charge profile of mitochondria can also be useful for attaching one or more polypeptide(s) electrostatically to the outer membrane of a mitochondrion via a positively-charged nanoparticle or particle. Accordingly, the positively-charged nanoparticle or particle comprising one or more polypeptide(s) may be electrostatically attached to the negative surface of the mitochondrion.

Accordingly, one or more polypeptide(s) may be electrostatically attached to the outer membrane of a mitochondrion via a positively-charged nanoparticle. One or more polypeptide(s) may be electrostatically attached to the outer membrane of a mitochondrion via a positively-charged particle. For complex formation, a polypeptide may be attached to the surface of a positively-charged nanoparticle or a positively-charged particle or be encapsulated by a positively-charged nanoparticle or a positively-charged particle. Accordingly, one or more polypeptide(s) may be electrostatically attached to the outer membrane of a mitochondrion via a positively-charged nanoparticle, wherein the one or more polypeptide(s) is attached to the surface of the positively-charged nanoparticle or encapsulated in the positively-charged nanoparticle. One or more polypeptide(s) may be electrostatically attached to the outer membrane of a mitochondrion via a positively-charged particle, wherein the one or more polypeptide is attached to the surface of the positively-charged particle or encapsulated in the positively-charged particle.

In general, the invention is not limited to any specific nanoparticles or particles for attachment to mitochondria and attachment of polypeptides or encapsulation of the same. Accordingly, one or more polypeptide(s) may be attached to the surface of or encapsulated in a lipid nanoparticle, a dendrimer nanoparticle, a micelle nanoparticle, a protein nanoparticle, a liposome, a non-porous silica nanoparticle, a mesoporous silica nanoparticle, a silicon nanoparticle, a gold nanoparticle, a gold nanowire, a silver nanoparticle, a platinum nanoparticle, a palladium nanoparticle, a titanium dioxide nanoparticle, a carbon nanotube, a carbon dot nanoparticle, a polymer nanoparticle, a zeolite nanoparticle, an aluminium oxide nanoparticle, a hydroxyapatite nanoparticle, a quantum dot nanoparticle, a zinc oxide nanoparticle, a zirconium oxide nanoparticle, graphene or a graphene oxide nanoparticle.

Moreover, one or more polypeptide(s) may be attached to the surface of a lipid particle, a dendrimer particle, a micelle particle, a protein particle, a liposome, a non-porous silica particle, a mesoporous silica particle, a silicon particle, a gold particle, a gold wire, a silver particle, a platinum particle, a palladium particle, a titanium dioxide particle, a carbon tube (such as a carbon microtube), a carbon dot particle, a polymer particle, a zeolite particle, an aluminum oxide particle, a hydroxyapatite particle, a quantum dot particle, a zinc oxide particle, a zirconium oxide particle, graphene or a graphene oxide particle.

The skilled person is aware that the above means of electrostatic attachment may be applied to all products, methods, apparatus or uses described herein.

A mitochondrion of the present invention is especially useful since it may be stored without disintegrating, i.e. being stable, for a long time. Accordingly, the present invention provides a mitochondrion comprising one or more polypeptides attached to the outer membrane of the mitochondrion, wherein the one or more polypeptide(s):
a) is electrostatically attached to the outer membrane of the mitochondrion, optionally via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule,
   wherein the mitochondrion is stored at −80° C. in a conjugation buffer. A mitochondrion of the present invention may be stored at −80° C. in a conjugation buffer for at least 2 months, 1 month, 3 weeks, 2 weeks, 1 week, or at least 5 days without disintegrating. A mitochondrion comprising one or more polypeptides attached to the outer membrane may be stored in conjugation buffer to maintain high colloidal stability (e.g., no agglomeration/aggregation or disintegration). A mitochondrion comprising one or more polypeptides attached to the outer membrane is stored in conjugation buffer at low temperatures (e.g. −80° C.) in the dark for preservation up to four months after the complex formation.

A polypeptide of the present invention may be functionalized with targeting molecules (such as small targeting molecules, targeting aptamers, targeting peptide, carbohydrate, sugar, and targeting antibody), drugs, reporter molecules/nanoparticles (e.g. fluorescence molecules, metallic nanoparticles, magnetic nanoparticles to say some) or contract agents.

A polypeptide of the present invention may be formulated into a nanoparticle, cationic lipid nanoformulation, block-copolymer, cationic lipid or cationic polymer.

In the sense of the present invention, polypeptides can also be covalently linked to the outer membrane of a mitochondrion. A covalent bond or covalent link or covalent interaction is formed by a chemical bond that involves sharing of electron pairs between atoms. A polypeptide may be attached to a mitochondrion via a peptide bond, such as an amide bond. A mitochondrion of the present invention possessing amino groups of mitochondria membrane-associated proteins may be covalently linked with N-hydroxysuccinimide ester (NHS)-functionalized nanoparticles, NHS-modified oligonucleotides or NHS-modified molecules forming covalently bound ligand and more stable conjugate.

Accordingly, one or more polypeptide(s) may be covalently linked to the outer membrane of a mitochondrion. One or more polypeptide(s) may be linked to a polypeptide in the outer membrane of a mitochondrion via an amide bond. One or more polypeptide(s) may be linked to a polypeptide in the outer membrane of a mitochondrion via an amide bond, wherein the one or more polypeptide(s) has been modified to undergo formation of the amide bond with an amine function comprised in the polypeptide in the outer membrane of the mitochondrion. A polypeptide can also be attached to a mitochondrion by covalently linking a nanoparticle comprising a polypeptide to a mitochondrion. Accordingly, one or more polypeptide(s) may be linked to a polypeptide in the outer membrane of a mitochondrion via an amide bond wherein the one or more polypeptide(s) is encapsulated in a nanoparticle (such as a lipid nanoparticle), and wherein the nanoparticle comprises a functional group that allows covalent linkage of the nanoparticle to a second polypeptide in the outer membrane of the mitochondrion. One or more polypeptide(s) may be covalently linked to a N-hydroxysuccinimide ester. One or more polypeptide(s) may be covalently linked to a N-hydroxysuccinimide ester, wherein the N-hydroxysuccinimide ester facilitates attachment of the polypeptide to an amine comprised in a second polypeptide in the outer membrane of the mitochondrion. One or more polypeptide(s) encapsulated in a nanoparticle comprising a N-hydroxysuccinimide ester, wherein the N-hydroxysuccinimide ester facilitates attachment of the polypeptide to an amine comprised in a second polypeptide in the outer membrane of the mitochondrion via the nanoparticle comprising a N-hydroxysuccinimide ester.

In the sense of the present invention, polypeptides can also be linked to an antibody that specifically binds to an antigen comprised in the outer membrane of the mitochondrion. Such an antibody comprising a polypeptide binds to the mitochondrion thereby facilitating the formation of the delivering platform. The present invention is not limited to any specific antigens or antibodies, in general, the invention may be performed with an antibody specifically binding any antigen comprised in the outer membrane of a mitochondrion, thereby facilitating formation of a mitochondrion-polypeptide complex.

In general, one or more polypeptide(s) may be linked to any antibody that specifically binds to an antigen comprised in a mitochondrion. Exemplary antigens are AIF, GCSH, MRPL40, TIMM23, ATP5A, HSP60, OPA1, TOM70, ATP5F1, OXA1L, TOMM20, BCS1L, Mitofilin, Prohibitin, TUFM, COX4, Mitofusin 1, SDHB, UQCRC1, COX5b, Mitofusin 2, SSBP1, VDAC1.

Preferably one or more polypeptide(s) may be linked to any antibody that specifically binds to an antigen comprised in the outer membrane of a mitochondrion. Accordingly, one or more polypeptide(s) may be linked to an antibody specifically binding to an antigen comprised in the outer membrane of a mitochondrion, wherein the preferred antigen is any one of OPA1, TOM70, TOMM20, Mitofusin 1, Mitofusin 2 or VDAC1.

A polypeptide may be covalently linked to an antibody forming a polypeptide-antibody complex which can bind to an antigen of a mitochondrion. Accordingly, a polypeptide may be covalently linked to an antibody forming a polypeptide-antibody complex which can bind to an antigen comprised in the outer membrane of a mitochondrion.

A polypeptide may be electrostatically linked to a modified antibody, such as an antibody comprising a positive or negative charge, forming a polypeptide-antibody complex which can bind to an antigen of a mitochondrion. Accordingly, a polypeptide may be electrostatically linked to a modified antibody, such as an antibody comprising a positive or negative charge, forming a polypeptide-antibody complex which can bind to an antigen comprised in the outer membrane of a mitochondrion.

An antibody specifically binding to an antigen comprised in the outer membrane of a mitochondrion may be used to attach a nanoparticle, such as a lipid nanoparticle, comprising polypeptides thereby facilitating the attachment of the polypeptide to a mitochondrion. Accordingly, one or more polypeptide(s) may be linked to an antibody that specifically binds to an antigen comprised in the outer membrane of the mitochondrion, wherein the one or more polypeptide is encapsulated in a nanoparticle (such as a lipid nanoparticle), and wherein the nanoparticle is covalently linked to the antibody. One or more polypeptide(s) may be linked to an antibody that specifically binds to an antigen comprised in the outer membrane of the mitochondrion, wherein the nanoparticle is electrostatically linked to the antibody, wherein the antibody is a modified antibody, wherein the modified antibody possesses one or more positive charges. Moreover, one or more polypeptide(s) may be linked to an antibody that specifically binds to an antigen comprised in the outer membrane of the mitochondrion, wherein the nanoparticle is electrostatically linked to the antibody, wherein the antibody is a modified antibody, wherein the modified antibody possesses one or more negative charges.

The nanoparticle comprising the nucleic acid molecule is not particularly limited, and may any nanoparticle as described hereinabove. In some embodiments the nanoparticle is a substantially neutral (i.e., net neutral) nanoparticle. In some embodiments, the nanoparticle is a charged nanoparticle, such as a positively or negatively charged nanoparticle. In some embodiments the nanoparticle is a charged nanoparticle, such as a nanoparticle possessing a positive “zeta potential” or positive “surface charge” or a nanoparticle possessing a negative “zeta potential” or negative “surface charge”.

A polypeptide can also be electrostatically linked to an antibody specifically binding to an antigen comprised in the outer membrane of a mitochondrion. Binding is facilitated via opposite charges of the antibody and polypeptide in the milieu where the antibody is contacted with the polypeptide. Accordingly, a positively-charged polypeptide may be electrostatically linked to a negatively-charged antibody and vice versa. Accordingly, one or more polypeptide(s) may be linked to an antibody specifically binding to an antigen comprised in the outer membrane of a mitochondrion, wherein the one or more polypeptide is electrostatically linked to a modified antibody, wherein the modified antibody possesses one or more positive or negative charges. A polypeptide can also be linked to an entity which is then linked to an antibody. Such entity may be biotin, which is linked to an avidin conjugated antibody. Accordingly, one or more polypeptide(s) may be linked to an antibody specifically binding to an antigen comprised in the outer membrane of a mitochondrion, wherein the one or more polypeptide(s) is covalently linked to biotin, wherein biotin is linked to the antibody, wherein the antibody is an avidin conjugated antibody. Further, a polypeptide can also be linked to an entity which is then linked to an antibody when being attached to or encapsulated into a lipid nanoparticle. Accordingly, one or more polypeptide(s) may be linked to an antibody specifically binding to an antigen comprised in the outer membrane of a mitochondrion, wherein one or more polypeptide is attached to or encapsulated in a nanoparticle, wherein the nanoparticle is covalently linked to biotin, wherein biotin is linked to the antibody, wherein the antibody is avidin conjugated antibody.

A polypeptide can also be linked to an entity which is then linked to an antibody. Such entity may be an activated ester, which is linked to an antibody. Accordingly, one or more polypeptide(s) may be linked to an antibody specifically binding to an antigen comprised in the outer membrane of a mitochondrion, wherein the one or more polypeptide(s) is covalently linked to an activated ester, wherein the activated ester is linked to the antibody via amide bond. Further, a polypeptide can also be linked to an entity which is then linked to an antibody when being attached to or encapsulated into a nanoparticle (such as a lipid nanoparticle). Accordingly, one or more polypeptide(s) may be linked to an antibody specifically binding to an antigen comprised in the outer membrane of a mitochondrion, wherein the nanoparticle is covalently linked to activated ester, wherein activated ester is linked to the antibody via amide bond. In the sense of the present invention “modified antibody” also means that an antibody is modified to possess one or more positive or negative charges, e.g. to attach a negatively or positively-charged polypeptide.

A polypeptide can also be attached to or encapsulated in a positively-charged nanoparticle, such as a polycationic lipid nanoparticle. The positively-charged nanoparticle comprising the polypeptide may be covalently linked to an antibody that specifically binds to an antigen comprised in the outer membrane of a mitochondrion. The positively-charged nanoparticle comprising the polypeptide can comprise phospholipids with reactive groups enabling covalent linkage to an antibody that specifically binds to an antigen comprised in the outer membrane of a mitochondrion. A polypeptide can also be attached to or encapsulated in a negatively-charged nanoparticle, such as a polyanionic lipid nanoparticle. The negatively-charged nanoparticle comprising the polypeptide may be covalently linked to an antibody that specifically binds to an antigen comprised in the outer membrane of a mitochondrion. The negatively-charged nanoparticle comprising the polypeptide can comprise phospholipids with reactive groups enabling covalent linkage to an antibody that specifically binds to an antigen comprised in the outer membrane of a mitochondrion.

In another aspect of the present invention, polypeptides may be linked to a mitochondria-targeting small molecule to facilitate attachment of the polypeptides and formation of the delivery platform. In the sense of the present invention, any mitochondria-targeting small molecule may be used to facilitate attachment. Exemplary mitochondria-targeting small molecules are selected from the group consisting of triphenylphosphonium (TPP), dequalinium (DQA), E-4-(1H-Indol-3-ylvinyl)-N-Methylpyridineiodide (F16), Rhodamine 19, biguanidine and guanidine. Accordingly, one or more polypeptides may be linked to a mitochondria-targeting small molecule, wherein the mitochondria targeting small molecules is selected from the group consisting of triphenylphosphonium (TPP), dequalinium (DQA), E-4-(1H-Indol-3-ylvinyl)-N-Methylpyridineiodide (F16), Rhodamine 19, biguanidine and guanidine. In a preferred embodiment, one or more polypeptide may be linked to triphenylphosphonium (TPP). Preferably, the mitochondria-targeting small molecule is selected from the group consisting of triphenylphosphonium (TPP), dequalinium (DQA), E-4-(1H-Indol-3-ylvinyl)-N-Methylpyridineiodide (F16), Rhodamine 19, biguanidine and guanidine.

The present invention is, inter alia, based on electrostatic interaction. The charge of a polypeptide or a mitochondrion may be functionalized with e.g., cationic molecules or polymers. Thus, the charge of a polypeptide or a mitochondrion may be e.g., inverted. Considering the above, the skilled person understands that the products, methods, apparatus and uses provided herein can also be performed when charges of a polypeptide and mitochondrion are modulated, such as inverted. Accordingly, the present invention also provides a mitochondrion comprising one or more polypeptide(s) attached to the outer membrane of the mitochondrion, wherein the one or more polypeptide(s) is electrostatically attached to the outer membrane of the mitochondrion, wherein:
(a) polycations or positively-charged species are attached to the outer surface of a mitochondrion resulting in a positively-charged mitochondrion surface; and
(b) one or more negatively-charged polypeptide is electrostatically attached to the positively-charged mitochondrion surface.

A mitochondrion comprising one or more polypeptide(s) attached to the outer membrane of the mitochondrion, wherein the one or more polypeptide(s) is electrostatically attached to the outer membrane of the mitochondrion, wherein:
(a) the one or more polypeptide is attached to or encapsulated in a positively-charged nanoparticle; and
(b) the positively-charged nanoparticle comprising the one or more polypeptide is electrostatically attached to a mitochondrion.

The present invention is not particularly limited to any nanoformulations. Any nanoformulation may be used that can facilitate attachment or encapsulation of one or more polypeptide and may be attached to a mitochondrion. Accordingly, a nanoformulation to facilitate attachment or encapsulation of one or more polypeptide are positively or negatively-charged organic or inorganic nanoparticles. Nanoformulations that may be used in the sense of the present invention are lipid nanoparticles, dendrimer nanoparticles, micelle nanoparticles, protein nanoparticles, liposomes, non-porous silica nanoparticles, mesoporous silica nanoparticles, silicon nanoparticles, gold nanoparticles and gold nanowires, silver nanoparticles, platinum nanoparticles, palladium nanoparticles, titanium dioxide nanoformulation, carbon nanotubes, carbon dots, polymer nanoparticles, zeolites nanoparticles, aluminum oxide nanoparticles, hydroxyapatite nanoparticles, quantum dots nanoparticles, zink oxide nanoparticles, zirconium oxide nanoparticles, graphene and/or graphene oxide nanoparticles.

In one embodiment, the present application provides a mitochondrion comprising one or more negatively-charged polypeptide(s) attached to the outer membrane of the mitochondrion, wherein the one or more negatively-charged polypeptide(s) is electrostatically attached to the outer membrane of the mitochondrion via a polycationic species.

In one embodiment, the present application provides a mitochondrion comprising one or more negatively-charged polypeptide(s) attached to the outer membrane of the mitochondrion, wherein the one or more negatively-charged polypeptide(s) is electrostatically attached to the outer membrane of the mitochondrion via a polycationic species, wherein the polycationic species is covalently linked to the one or more negatively-charged polypeptide(s).

In one embodiment, the present application provides a mitochondrion comprising one or more positively-charged polypeptide(s) attached to the outer membrane of the mitochondrion, wherein the one or more positively-charged polypeptide is electrostatically attached to the negatively-charged outer membrane of the mitochondrion.

Moreover, the present invention provides a mitochondrion comprising one or more polypeptide(s), as described hereinabove, wherein the mitochondrion is linked to and/or enveloped in a protective layer. The protective layer is as described hereinabove. In some embodiments, the protective layer is a protective polymer.

In some embodiments, the protective polymer is a linear or branched cationic polymer, optionally wherein the linear or branched cationic polymer is electrostatically linked to the one or more polypeptide(s). In some embodiments, the protective polymer is a linear or branched cationic polymer, optionally wherein the linear or branched cationic polymer is electrostatically linked to the one or more negatively-charged polypeptide(s). Preferably, the linear or branched cationic polymer is polyethyleneimine, RGD-modified polyethyleneimine, polylysine, RGD-modified polylysine, polyornithine, RGD-modified polyornithine, polyarginine, RGD modified polyarginine, polypropyleneimine, RGD-modified polypropyleneimine, polyallylamine, RGD-modified polyallylamine, chitosan, RGD-modified chitosan, poly(2-(dimethylamino)ethyl methacrylate), RGD-modified poly(2-(dimethylamino)ethyl methacrylate), poly(amidoamine)s, RGD-modified poly(amidoamine)s or a combination thereof. The linear or branched cationic polymer is defined hereinabove.

In some embodiments, the protective polymer is a linear or branched cationic block copolymer, optionally wherein the linear or branched cationic block copolymer is electrostatically linked to the one or more polypeptides(s). In some embodiments, the protective polymer is a linear or branched cationic block copolymer, optionally wherein the linear or branched cationic block copolymer is electrostatically linked to the one or more negatively-charged polypeptides(s). Preferably, the cationic block copolymer is poly(ethylene glycol)-block-polyethyleneimine, RGD-modified poly(ethylene glycol)-block-polyethyleneimine, poly(ethylene glycol)-block-polylysine, RGD-modified poly(ethylene glycol)-block-polylysine, poly(ethylene glycol)-block-polyornithine, RGD-modified poly(ethylene glycol)-block-polyornithine, poly(ethylene glycol)-block-polyarginine, RGD-modified poly(ethylene glycol)-block-polyarginine, poly(ethylene glycol)-block-polypropyleneimine, RGD-modified poly(ethylene glycol)-block-polypropyleneimine, poly(ethylene glycol)-block-polyallylamine, RGD-modified poly(ethylene glycol)-block-polyallylamine, poly(ethylene glycol)-block-poly(2-(dimethylamino)ethyl methacrylate), RGD-modified poly(ethylene glycol)-block-poly(2-(dimethylamino)ethyl methacrylate), poly(ethylene glycol)-block-poly(amidoamine)s, RGD-modified poly(ethylene glycol)-block-poly(amidoamine)s or a combination thereof. The linear or branched cationic block polymer is as defined hereinabove.

In some embodiments, the protective polymer is a cationic graft (g) copolymer, optionally wherein the cationic graft (g) copolymer is electrostatically linked to the one or more polypeptide(s). In some embodiments, the protective polymer is a cationic graft (g) copolymer, optionally wherein the cationic graft (g) copolymer is electrostatically linked to the one or more negatively-charged polypeptide(s). Preferably, the cationic graft (g) copolymer is poly(ethylene glycol)-g-polyethyleneimine, RGD-modified poly(ethylene glycol)-g-polyethyleneimine, poly(ethylene glycol)-g-polylysine, RGD-modified poly(ethylene glycol)-g-polylysine, poly(ethylene glycol)-g-polyornithine, RGD-modified poly(ethylene glycol)-g-polyornithine, poly(ethylene glycol)-g-polyarginine, RGD-modified poly(ethylene glycol)-g-polyarginine, poly(ethylene glycol)-g-polypropyleneimine, RGD-modified poly(ethylene glycol)-g-polypropyleneimine, poly(ethylene glycol)-g-polyallylamine, RGD-modified poly(ethylene glycol)-g-polyallylamine, poly(ethylene glycol)-g-poly(2-(dimethylamino)ethyl methacrylate), RGD-modified poly(ethylene glycol)-g-poly(2-(dimethylamino)ethyl methacrylate), poly(ethylene glycol)-g-poly(amidoamine)s, RGD-modified poly(ethylene glycol)-g-poly(amidoamine)s or a combination thereof. The cationic graft (g) copolymer is as described hereinabove.

In some embodiments, the protective polymer is a linear or branched pegylated (PEG) cationic polymer, optionally wherein the linear or branched pegylated (PEG) cationic polymer is electrostatically linked to the one or more polypeptides(s). In some embodiments, the protective polymer is a linear or branched pegylated (PEG) cationic polymer, optionally wherein the linear or branched pegylated (PEG) cationic polymer is electrostatically linked to the one or more negatively-charged polypeptides(s). Preferably, the pegylated (PEG) cationic polymer is pegylated-polyethyleneimine, RGD-modified pegylated polyethyleneimine, pegylated polylysine, RGD-modified pegylated polylysine, histidylated polylysine, pegylated polyornithine, RGD-modified pegylated polyornithine, pegylated polyarginine, RGD-modified pegylated polyarginine, pegylated polypropyleneimine, RGD-modified pegylated polypropyleneimine, pegylated polyallylamine, RGD-modified pegylated polyallylamine, pegylated chitosan, RGD-modified pegylated chitosan, pegylated poly(2-(dimethylamino)ethyl methacrylate), RGD-modified d pegylated poly(2-(dimethylamino)ethyl methacrylate), pegylated poly(amidoamine)s RGD-modified pegylated poly(amidoamine)s or a combination thereof. The pegylated (PEG) cationic polymer is as defined hereinabove.

In some embodiments, the protective polymer is a zwitterionic protective polymer, optionally wherein the zwitterionic protective polymer is electrostatically linked to the one or more polypeptide(s). Preferably, the zwitterionic polymer is selected from: poly(2-methacryloyloxyethyl phosphorylcholine) (PMPC), polyethyleneimine-g-poly(2-methacryloyloxyethyl phosphorylcholine) (PEI-g-PMPC), co-assembly of cationic (carboxyl-functionalized) and anionic (amino-functionalized) copolyesters based on poly(ε-caprolactone)-block-poly(butylene fumarate)-block-poly(ε-caprolactone) (PCL-b-PBF-b-PCL), poly(lactic-co-glycolic acid) (PLGA)-PCB block copolymers (PLGA-b-PCB). The zwitterionic polymer is as defined hereinabove.

In some embodiments, the mitochondrion comprising one or more polypeptides is linked to and/or enveloped in a protective layer, wherein the protective layer is a lipid formulation, optionally wherein the lipid formulation is a cationic lipid formulation, further optionally wherein the cationic lipid formulation is electrostatically linked to the one or more polypeptide(s). In some embodiments, the protective layer is a lipid formulation, optionally wherein the lipid formulation is a cationic lipid formulation, further optionally wherein the cationic lipid formulation is electrostatically linked to the one or more negatively-charged polypeptide(s). Preferably, the lipid formulation comprises DC-cholesterol (3β-[N—(N′,N′-Dimethylaminoethane)-carbamoyl]cholesterol hydrochloride), DLinDMA (1,2-dilinoleyloxy-3-dimethylaminopropane), DLinMC3DMA (dilinoleylmethyl-4-dimethylaminobutyrate), DODMA (1,2-dioleyloxy-3-dimethylaminopropane), DOGS (dioctadecylamidoglycylspermine), DOSPA (2,3-dioleyloxy-N-[2 (sperminecarboxamido)ethyl]-N,N-dimethyl-1-propanaminium), DOTAP (1,2-dioleoyl-3-trimethylammonium-propane chloride), DOTMA (1,2-di-O-octadecenyl-3-trimethylammonium propane chloride), UGG (unsaturated guanidinium glycoside), DOPE (1,2-Dioleoyl-sn-glycerophosphoethanolamine), lipofectamine or a combination thereof. The lipid formulation and cationic lipid formulation is as defined hereinabove. Moreover, the lipid formulation may further comprise another lipid, preferably wherein said lipid is cholesterol, a substituted or unsubstituted cholesterol, a cholesterol derivative, such as a hydroxylated cholesterol derivative (e.g., a hydroxycholesterol), a PEG-lipid, DMPC (1,2-Dimyristoyl-sn-glycero-3-phosphocholine), DSPC (1,2-Distearoyl-sn-glycero-3-phosphocholine), DODAP (1,2-dioleoyl-3-dimethylammonium propane), DDA (dimethyldioctadecylammonium), 1,2-dioleoyl-sn-glycero-3-phosphate, 1,2-dimyristoyl-sn-glycero-3-phosphate, bis(monooleoylglycerol)phosphate or a combination thereof. The further lipid is as defined hereinabove.

In some embodiments, the mitochondrion comprising the protective layer as defined hereinabove may be linked to a targeting moiety, such as an antibody or carbohydrate. The targeting moiety is as described hereinabove. In some embodiments, the protective layer is connected to an antibody, optionally wherein the protective layer connected to an antibody is electrostatically linked to the one or more polypeptide(s). In further embodiments, the protective layer is connected to a carbohydrate, optionally wherein the protective layer connected to a carbohydrate is electrostatically linked to the one or more polypeptide(s).

In another aspect, any of the mitochondria described herein may be incorporated into a composition. Accordingly, the present invention provides a composition comprising a plurality of mitochondria comprising one or more polypeptide(s) attached to the outer membrane of the mitochondrion, wherein the one or more polypeptide(s):
a) is electrostatically attached to the outer membrane of the mitochondrion, optionally via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule.

The present invention provides a mitochondrion, compositions, and pharmaceutical compositions for use in the treatment of a disease that may benefit from the use of healthy mitochondria and the combination of healthy mitochondria and polypeptides. It is envisioned to increase a biological activity, for example through a delivery of polypeptides attached to mitochondria or decrease a biological activity through a delivery of polypeptides attached to mitochondria. Accordingly, the present invention provides a mitochondrion for use as a medicament, comprising one or more polypeptide attached to the outer membrane of the mitochondrion, wherein the one or more polypeptide:
a) is electrostatically attached to the outer membrane of the mitochondrion, optionally via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule.

The present invention provides a composition for use as a medicament comprising a plurality of a mitochondrion, comprising one or more polypeptide attached to the outer membrane of the mitochondrion, wherein the one or more polypeptide:
a) is electrostatically attached to the outer membrane of the mitochondrion, optionally via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule.

The present invention provides a pharmaceutical composition for use as a medicament comprising a plurality of a mitochondrion, comprising one or more polypeptide attached to the outer membrane of the mitochondrion, wherein the one or more polypeptide:
a) is electrostatically attached to the outer membrane of the mitochondrion, optionally via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule.

The mitochondrion, compositions and pharmaceutical compositions of the present invention may be used for gene therapy. The present invention provides a delivery platform for polypeptides which is especially useful for in vivo, ex vivo or in vitro gene therapy. Accordingly, the methods and uses of the present invention may be in vivo, ex vivo or in vitro. Accordingly, the present invention provides a mitochondrion for use in gene therapy, comprising one or more polypeptide attached to the outer membrane of the mitochondrion, wherein the one or more polypeptide:
a) is electrostatically attached to the outer membrane of the mitochondrion, optionally via a positively-charged species; or
b) is covalently linked to the outer membrane of the mitochondrion; or
c) is linked to an antibody that specifically binds to an antigen comprised in the outer membrane of the mitochondrion;
d) is linked to a mitochondria-targeting small molecule.

The present invention provides a composition for use in gene therapy comprising a plurality of a mitochondrion, comprising one or more polypeptide attached to the outer membrane of the mitochondrion, wherein the one or more polypeptide:
a) is electrostatically attached to the outer membrane of the mitochondrion, optionally via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule.

The present invention provides a pharmaceutical composition for use in gene therapy comprising a plurality of a mitochondrion, comprising one or more polypeptide attached to the outer membrane of the mitochondrion, wherein the one or more polypeptide:
a) is electrostatically attached to the outer membrane of the mitochondrion, optionally via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule.

Accordingly, the present invention provides a mitochondrion for use in in vitro, ex vivo, or in vivo genome editing, comprising one or more polypeptide attached to the outer membrane of the mitochondrion, wherein the one or more polypeptide:
a) is electrostatically attached to the outer membrane of the mitochondrion, optionally via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule.

The present invention provides a composition for use in in vitro, ex vivo, or in vivo genome editing comprising a plurality of a mitochondrion, comprising one or more polypeptide attached to the outer membrane of the mitochondrion, wherein the one or more polypeptide:
a) is electrostatically attached to the outer membrane of the mitochondrion, optionally via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule.

The present invention provides a pharmaceutical composition for use in in vitro, ex vivo, or in vivo genome editing comprising a plurality of a mitochondrion, comprising one or more polypeptide attached to the outer membrane of the mitochondrion, wherein the one or more polypeptide:
a) is electrostatically attached to the outer membrane of the mitochondrion, optionally via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule.

The present invention provides a mitochondrion for use in the treatment of a disease, comprising one or more polypeptide attached to the outer membrane of the mitochondrion, wherein the one or more polypeptide:
a) is electrostatically attached to the outer membrane of the mitochondrion, optionally via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule.

In general the mitochondrion comprising one or more polypeptide attached to the outer membrane of the present invention may be used in the treatment of any desired disease. In particular, the polypeptide may be used to increase or decrease a desired biological activity thereby treating a disease associated with said biological activity.

Accordingly, the present invention provides a mitochondrion for use in the treatment of cardiovascular diseases, kidney disease, or aging related diseases, comprising one or more polypeptide attached to the outer membrane of the mitochondrion, wherein the one or more polypeptide:
a) is electrostatically attached to the outer membrane of the mitochondrion, optionally via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule.

Accordingly, the present invention provides a mitochondrion for use in the treatment of cancer, comprising one or more polypeptide attached to the outer membrane of the mitochondrion, wherein the one or more polypeptide:
a) is electrostatically attached to the outer membrane of the mitochondrion, optionally via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule.

The mitochondrion, compositions and pharmaceutical compositions of the present invention may be used in radiation therapy. In particular, the mitochondria of the present invention may be used to deliver a radioactive agent which may be used for radiation therapy. Such a radioactive agent for radiation therapy may be delivered by the delivery system of the present invention into solid tumors. The present invention is not particularly limited to any agent for radiation therapy. Iodine 131 is an exemplary agent for radiation therapy of thyroid cancer. Accordingly, the present invention provides a mitochondrion for use in radiation therapy, comprising one or more peptides attached to the outer membrane of the mitochondrion, wherein the one or more polypeptide:
a) is electrostatically attached to the outer membrane of the mitochondrion, optionally via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule.

Accordingly, the present invention provides a mitochondrion for use in radiation therapy, comprising one or more radioactive agent attached to the outer membrane of the mitochondrion, wherein the one or more radioactive agent:
a) is electrostatically attached to the outer membrane of the mitochondrion, via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule.

In yet another aspect, the present invention provides methods for delivering polypeptides to an organ in a subject by administering the delivery platform of the present invention to a subject. The terms “administering”, “introducing” and “delivering” are used interchangeably in the context of the present invention, e.g., the delivery platform of the present invention, i.e. mitochondrion polypeptide complex may be introduced into a subject by a method or route that results in at least partial localization of the introduced complex at a desired site, such as a site where it is appreciated to produce a desired effect, such as a treatment or therapy. A mitochondrion, composition or pharmaceutical composition of the present invention may be administered into the bloodstream upstream of the target organ. Accordingly, the present invention provides a method for delivering a polypeptide to a target organ, the method comprising a step of administering a pharmaceutical composition comprising a mitochondrion comprising one or more polypeptide attached to the outer membrane of the mitochondrion, wherein the one or more polypeptide:
a) is electrostatically attached to the outer membrane of the mitochondrion, optionally via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule; and a pharmaceutically acceptable carrier, into the bloodstream of a subject in need, wherein the pharmaceutical composition is administered into the bloodstream upstream of the target organ.

The present invention provides a method for delivering a polypeptide to a target organ, the method comprising a step of administering a pharmaceutical composition comprising a mitochondrion comprising one or more polypeptide attached to the outer membrane of the mitochondrion, wherein the one or more polypeptide:
a) is electrostatically attached to the outer membrane of the mitochondrion, optionally via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule; and a pharmaceutically acceptable carrier, into the bloodstream of a subject having a cardiovascular disease, an aging related disease, a kidney disease, or cancer, wherein the pharmaceutical composition is administered into the bloodstream upstream of the target organ.

In certain embodiments, a mitochondrion comprising one or more polypeptide attached to the outer membrane of the mitochondrion, or a composition or pharmaceutical composition comprising a mitochondrion comprising one or more polypeptide attached to the outer membrane of the mitochondrion, is delivered to the kidney of a subject. In certain embodiments, delivery into the kidney is achieved through injection into the renal artery or through direct injection into the kidney. In certain embodiments, delivery into the kidney is achieved through injection into the renal artery or through direct injection into the kidney and the one or more polypeptide is electrostatically attached to the outer membrane of the mitochondrion, optionally via a positively-charged species. In certain embodiments, delivery into the kidney is achieved through injection into the renal artery or through direct injection into the kidney and the one or more polypeptide is covalently linked to the outer membrane of the mitochondrion. In certain embodiments, delivery into the kidney is achieved through injection into the renal artery or through direct injection into the kidney and the one or more polypeptide is linked to an antibody that specifically binds to an antigen comprised in the outer membrane of the mitochondrion. In certain embodiments, delivery into the kidney is achieved through injection into the renal artery or through direct injection into the kidney and the one or more polypeptide is linked to a mitochondria-targeting small molecule.

In certain embodiments, a mitochondrion comprising one or more polypeptide attached to the outer membrane of the mitochondrion, or a composition or pharmaceutical composition comprising a mitochondrion comprising one or more polypeptide attached to the outer membrane of the mitochondrion, is delivered to the heart of a subject. In certain embodiments, delivery into the heart is achieved through injection into the intracoronary or through direct injection into the heart. In certain embodiments, delivery into the heart is achieved through injection into the intracoronary or through direct injection into the heart and the one or more polypeptide is electrostatically attached to the outer membrane of the mitochondrion, optionally via a positively-charged species. In certain embodiments, delivery into the heart is achieved through injection into the intracoronary or through direct injection into the heart and the one or more polypeptide is covalently linked to the outer membrane of the mitochondrion. In certain embodiments, delivery into the heart is achieved through injection into the intracoronary or through direct injection into the heart and the one or more polypeptide is linked to an antibody that specifically binds to an antigen comprised in the outer membrane of the mitochondrion. In certain embodiments, delivery into the heart is achieved through injection into the intracoronary or through direct injection into the heart and the one or more polypeptide is linked to a mitochondria-targeting small molecule.

In certain embodiments, a mitochondrion comprising one or more polypeptide attached to the outer membrane of the mitochondrion, or a composition or pharmaceutical composition comprising a mitochondrion comprising one or more polypeptide attached to the outer membrane of the mitochondrion, is delivered to the liver of a subject. In certain embodiments, delivery into the liver is achieved through injection into the hepatic artery or portal vein or through direct injection into the liver. In certain embodiments, delivery into the liver is achieved through injection into the hepatic artery or portal vein or through direct injection into the liver and the one or more polypeptide is electrostatically attached to the outer membrane of the mitochondrion, optionally via a positively-charged species. In certain embodiments, delivery into the liver is achieved through injection into the hepatic artery or portal vein or through direct injection into the liver and the one or more polypeptide is covalently linked to the outer membrane of the mitochondrion. In certain embodiments, delivery into the liver is achieved through injection into the hepatic artery or portal vein or through direct injection into the liver and the one or more polypeptide is linked to an antibody that specifically binds to an antigen comprised in the outer membrane of the mitochondrion. In certain embodiments, delivery into the liver is achieved through injection into the hepatic artery or portal vein or through direct injection into the liver and the one or more polypeptide is linked to a mitochondria-targeting small molecule.

In certain embodiments, a mitochondrion comprising one or more polypeptide attached to the outer membrane of the mitochondrion, or a composition or pharmaceutical composition comprising a mitochondrion comprising one or more polypeptide attached to the outer membrane of the mitochondrion, is delivered to the pancreas of a subject. In certain embodiments, delivery into the pancreas is achieved through injection into the hepatic artery or through direct injection into the pancreas. In certain embodiments, delivery into the pancreas is achieved through injection into the hepatic artery or through direct injection into the pancreas and the one or more polypeptide is electrostatically attached to the outer membrane of the mitochondrion, optionally via a positively-charged species. In certain embodiments, delivery into the pancreas is achieved through injection into the hepatic artery or through direct injection into the pancreas and the one or more polypeptide is covalently linked to the outer membrane of the mitochondrion. In certain embodiments, delivery into the pancreas is achieved through injection into the hepatic artery or through direct injection into the pancreas and the one or more polypeptide is linked to an antibody that specifically binds to an antigen comprised in the outer membrane of the mitochondrion. In certain embodiments, delivery into the pancreas is achieved through injection into the hepatic artery or through direct injection into the pancreas and the one or more polypeptide is linked to a mitochondria-targeting small molecule.

In certain embodiments, a mitochondrion comprising one or more polypeptide attached to the outer membrane of the mitochondrion, or a composition or pharmaceutical composition comprising a mitochondrion comprising one or more polypeptide attached to the outer membrane of the mitochondrion, is delivered to the duodenum of a subject. In certain embodiments, delivery into the duodenum is achieved through injection into the hepatic artery or through direct injection into the duodenum. In certain embodiments, delivery into the duodenum is achieved through injection into the hepatic artery or through direct injection into the duodenum and the one or more polypeptide is electrostatically attached to the outer membrane of the mitochondrion, optionally via a positively-charged species. In certain embodiments, delivery into the duodenum is achieved through injection into the hepatic artery or through direct injection into the duodenum and the one or more polypeptide is covalently linked to the outer membrane of the mitochondrion. In certain embodiments, delivery into the duodenum is achieved through injection into the hepatic artery or through direct injection into the duodenum and the one or more polypeptide is linked to an antibody that specifically binds to an antigen comprised in the outer membrane of the mitochondrion. In certain embodiments, delivery into the duodenum is achieved through injection into the hepatic artery or through direct injection into the duodenum and the one or more polypeptide is linked to a mitochondria-targeting small molecule.

In certain embodiments, a mitochondrion comprising one or more polypeptide attached to the outer membrane of the mitochondrion, or a composition or pharmaceutical composition comprising a mitochondrion comprising one or more polypeptide attached to the outer membrane of the mitochondrion, is delivered to the spleen of a subject. In certain embodiments, delivery into the spleen is achieved through injection into the splenic artery or through direct injection into the spleen. In certain embodiments, delivery into the spleen is achieved through injection into the splenic artery or through direct injection into the spleen and the one or more polypeptide is electrostatically attached to the outer membrane of the mitochondrion, optionally via a positively-charged species. In certain embodiments, delivery into the spleen is achieved through injection into the splenic artery or through direct injection into the spleen and the one or more polypeptide is covalently linked to the outer membrane of the mitochondrion. In certain embodiments, delivery into the spleen is achieved through injection into the splenic artery or through direct injection into the spleen and the one or more polypeptide is linked to an antibody that specifically binds to an antigen comprised in the outer membrane of the mitochondrion. In certain embodiments, delivery into the spleen is achieved through injection into the splenic artery or through direct injection into the spleen and the one or more polypeptide is linked to a mitochondria-targeting small molecule.

In certain embodiments, a mitochondrion comprising one or more polypeptide attached to the outer membrane of the mitochondrion, or a composition or pharmaceutical composition comprising a mitochondrion comprising one or more polypeptide attached to the outer membrane of the mitochondrion, is delivered to the lung of a subject. In certain embodiments, delivery into the lung is achieved through injection into the pulmonary artery or through direct injection into the lung. In certain embodiments, delivery into the lung is achieved through injection into the pulmonary artery or through direct injection into the lung and the one or more polypeptide is electrostatically attached to the outer membrane of the mitochondrion, optionally via a positively-charged species. In certain embodiments, delivery into the lung is achieved through injection into the pulmonary artery or through direct injection into the lung and the one or more polypeptide is covalently linked to the outer membrane of the mitochondrion. In certain embodiments, delivery into the lung is achieved through injection into the pulmonary artery or through direct injection into the lung and the one or more polypeptide is linked to an antibody that specifically binds to an antigen comprised in the outer membrane of the mitochondrion. In certain embodiments, delivery into the lung is achieved through injection into the pulmonary artery or through direct injection into the lung and the one or more polypeptide is linked to a mitochondria-targeting small molecule.

In certain embodiments, a mitochondrion comprising one or more polypeptide attached to the outer membrane of the mitochondrion, or a composition or pharmaceutical composition comprising a mitochondrion comprising one or more polypeptide attached to the outer membrane of the mitochondrion, is delivered to the intestines of a subject. In certain embodiments, delivery into the intestines is achieved through injection into the superior mesenteric artery or through direct injection into the intestines. In certain embodiments, delivery into the intestines is achieved through injection into the superior mesenteric artery or through direct injection into the intestines and the one or more polypeptide is electrostatically attached to the outer membrane of the mitochondrion, optionally via a positively-charged species. In certain embodiments, delivery into the intestines is achieved through injection into the superior mesenteric artery or through direct injection into the intestines and the one or more polypeptide is covalently linked to the outer membrane of the mitochondrion. In certain embodiments, delivery into the intestines is achieved through injection into the superior mesenteric artery or through direct injection into the intestines and the one or more polypeptide is linked to an antibody that specifically binds to an antigen comprised in the outer membrane of the mitochondrion. In certain embodiments, delivery into the intestines is achieved through injection into the superior mesenteric artery or through direct injection into the intestines and the one or more polypeptide is linked to a mitochondria-targeting small molecule.

In certain embodiments, a mitochondrion comprising one or more polypeptide attached to the outer membrane of the mitochondrion, or a composition or pharmaceutical composition comprising a mitochondrion comprising one or more polypeptide attached to the outer membrane of the mitochondrion, is delivered to the bladder of a subject. In certain embodiments, delivery into the bladder is achieved through injection into the superior and inferior vesical arteries or through direct injection into the bladder. In certain embodiments, delivery into the bladder is achieved through injection into the superior and inferior vesical arteries or through direct injection into the bladder and the one or more polypeptide is electrostatically attached to the outer membrane of the mitochondrion, optionally via a positively-charged species. In certain embodiments, delivery into the bladder is achieved through injection into the superior and inferior vesical arteries or through direct injection into the bladder and the one or more polypeptide is covalently linked to the outer membrane of the mitochondrion. In certain embodiments, delivery into the bladder is achieved through injection into the superior and inferior vesical arteries or through direct injection into the bladder and the one or more polypeptide is linked to an antibody that specifically binds to an antigen comprised in the outer membrane of the mitochondrion. In certain embodiments, delivery into the bladder is achieved through injection into the superior and inferior vesical arteries or through direct injection into the bladder and the one or more polypeptide is linked to a mitochondria-targeting small molecule.

A mitochondrion, composition or pharmaceutical composition of the present invention may be administered by inhalation. Accordingly, the present invention provides a method for delivering a polypeptide to the lung, the method comprising a step of administering a pharmaceutical composition comprising a mitochondrion comprising one or more polypeptide attached to the outer membrane of the mitochondrion, wherein the one or more polypeptide:
a) is electrostatically attached to the outer membrane of the mitochondrion, optionally via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule; and a pharmaceutically acceptable carrier to a subject in need, wherein the pharmaceutical composition is administered by inhalation.

The present invention provides a method for delivering a polypeptide to the lung, the method comprising a step of administering a pharmaceutical composition comprising a mitochondrion comprising one or more polypeptide attached to the outer membrane of the mitochondrion, wherein the one or more polypeptide:
a) is electrostatically attached to the outer membrane of the mitochondrion, optionally via a positively-charged species; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule; and a pharmaceutically acceptable carrier to a subject having a cardiovascular disease, an aging related disease, a kidney disease, or cancer, wherein the pharmaceutical composition is administered by inhalation.

The mitochondrion comprising a polypeptide, the composition or pharmaceutical composition thereof is for use in the treatment of various diseases including cardiovascular diseases, ischemia-reperfusion injury, kidney diseases, cancer, mitochondrial dysfunction disorders, metabolic disorders, autoimmune disorders, infectious diseases, inflammatory diseases, muscular diseases and aging related diseases. Said diseases or disorders have been described hereinabove.

Moreover, the present invention provides methods for delivering polypeptides to an organ in a subject by administering the delivery platform of the present invention to a subject. The method of delivering the mitochondrion comprising a polypeptide is analogous to the methods described hereinabove for the delivery of a mitochondrion comprising a nucleic acid molecule, or the composition or pharmaceutical composition thereof.

In one aspect the invention provides a method for attaching a polypeptide to a mitochondrion thereby producing the mitochondrion of the present invention. In the sense of the present invention “contacting” means bringing a first substance into close physical proximity with a second substance so that both can perform a reaction. For example, the mitochondrion may be contacted with polypeptides and optionally positively-charged species in a solution, such as a buffer.

Accordingly, the present invention provides a method for attaching a polypeptide to the outer membrane of a mitochondrion, the method comprising the steps of:
a) providing a preparation of mitochondria;
b) contacting the mitochondria provided in step (a) with at least one polypeptide, optionally in the presence of a positively-charged species;
c) attaching the at least one polypeptide to the mitochondria via the positively-charged species.

The at least one polypeptide may be contacted with the positively-charged species and the mitochondria simultaneously. In some embodiments, the at least one polypeptide may be first contacted with a positively-charged species to form a positively-charged complex before the positively-charged complex is contacted with the mitochondria. Moreover, in certain embodiments, the mitochondrion is contacted with the positively-charged species and subsequently contacted with the at least one polypeptide. Accordingly, the present invention provides a method for attaching a polypeptide to the outer membrane of a mitochondrion, the method comprising the steps of:
a) providing a preparation of mitochondria;
b) contacting the mitochondria provided in step (a) with at least one polypeptide in the presence of a positively-charged species, wherein the at least one polypeptide is simultaneously contacted with the positively-charged species and the mitochondria; or wherein the at least one polypeptide is contacted with the positively-charged species to form a positively-charged complex before the positively-charged complex is contacted with the mitochondria; or the mitochondrion is contacted with the positively-charged species and subsequently contacted with the at least one polypeptide;
c) attaching the at least one polypeptide to the mitochondria via the positively-charged species.

The step of contacting mitochondria with a plurality of polypeptides and a polycationic species may be performed in a suitable buffer. Accordingly, the present invention provides a method for attaching a polypeptide to the outer membrane of a mitochondrion, the method comprising the steps of:
a) providing a preparation of mitochondria;
b) contacting the mitochondria provided in step (a) with at least one polypeptide in the presence of a positively-charged species;
c) attaching the at least one polypeptide to the mitochondria via the positively-charged species,
   wherein the mitochondria are contacted with the at least one polypeptide and the positively-charged species, in a suitable buffer.

Preferably, the present invention provides a method for attaching a polypeptide to the outer membrane of a mitochondrion, the method comprising the steps of:
a) providing a preparation of mitochondria;
b) contacting the mitochondria provided in step (a) with at least one polypeptide in the presence of a positively-charged species;
c) attaching the at least one polypeptide to the mitochondria via the positively-charged species,
   wherein the mitochondria are contacted with the plurality of polypeptides and the polycationic species in a buffer comprising a 4:1 mixture of Solution X comprising or consisting of 20 mM HEPES, 1 mM EGTA and 300 mM Trehalose (pH 7.2) and Solution Y comprising or consisting of 0.1 M CHES (pH 10) and 0.2 M sodium phosphate dibasic dihydrate.

The contacting step of the present invention is not particularly limited to any reaction conditions or times. In general, any reaction conditions facilitating the attachment of polypeptides to a mitochondrion, optionally via a positively-charged species thereby facilitating the formation of the delivery complex may be used. However, it is preferred, that the mitochondria are contacted with the plurality of polypeptides and optionally the positively-charged species at room temperature for more than 5 minutes, preferably in the dark. Accordingly, the present invention provides a method for attaching a polypeptide to the outer membrane of a mitochondrion, the method comprising the steps of:
a) providing a preparation of mitochondria;
b) contacting the mitochondria provided in step (a) with at least one polypeptide in the presence of a positively-charged species, wherein the mitochondria are contacted with the at least one polypeptide and the positively-charged species at room temperature for at least 5 minutes, such as at least 10 minutes, 20 or 30 minutes;
c) attaching the at least one polypeptide to the mitochondria via the positively-charged species.

The present invention provides a method for attaching a polypeptide to the outer membrane of a mitochondrion, the method comprising the steps of:
a) providing a preparation of mitochondria;
b) contacting the mitochondria provided in step (a) with at least one polypeptide in the presence of a positively-charged species, wherein the mitochondria are contacted with the at least one polypeptide and the positively-charged species in the dark;
c) attaching the at least one polypeptide to the mitochondria via the positively-charged species.

The present invention provides a method for attaching a polypeptide to the outer membrane of a mitochondrion, the method comprising the steps of:
a) providing a preparation of mitochondria;
b) contacting the mitochondria provided in step (a) with at least one polypeptide in the presence of a positively-charged species, wherein the mitochondria are contacted with the at least one polypeptide and the positively-charged species at room temperature for at least 5 minutes, such as at least 10 minutes, 20 or 30 minutes in the dark;
c) attaching the at least one polypeptide to the mitochondria via the positively-charged species.

Preferably, the present invention provides a method for attaching a polypeptide to the outer membrane of a mitochondrion, the method comprising the steps of:
a) providing a preparation of mitochondria;
b) contacting the mitochondria provided in step (a) with at least one polypeptide in the presence of a positively-charged species, wherein the mitochondria are contacted with the at least one polypeptide and the positively-charged species at room temperature for 30 minutes in the dark;
c) attaching the at least one polypeptide to the mitochondria via the positively-charged species.

As described herein above, the present invention provides for attachment of polypeptides to a mitochondrion, optionally via a positively-charged species. Accordingly, the present invention provides a method for attaching a polypeptide to the outer membrane of a mitochondrion, the method comprising the steps of:
a) providing a preparation of mitochondria;
b) contacting the mitochondria provided in step (a) with at least one polypeptide, optionally in the presence of a polycationic species;
c) attaching the at least one polypeptide to the mitochondria, optionally via the polycationic species.

In the sense of the present invention a polycationic species may be a linear or branched polycationic polymer. A linear or branched polycationic polymer may be electrostatically linked to a polypeptide, comprised in the plurality of polypeptides. The present invention is not particularly limited to any polycationic polymers. In general, any polycationic polymers facilitating the attachment of polypeptides to a mitochondrion thereby facilitating the formation of the delivery complex may be used. However, it is preferred that a linear or branched polycationic polymer is polylysine, histidylated polylysine, polyornithine, polyarginine, high-mobility group protein (HMG) 1 and 17, modified chitosan, cationized human serum albumin, polyethyleneimine (PEI), polypropyleneimine (PPI), a cationic dendrimer, poly(2-(dimethylamino)ethyl methacrylate) (PDMAEMA), a polyallylamine derivative, diethylaminoethyl (DEAE)-dextran, poly(N-alkyl-4-vinylpyridinium), a poly(amidoamine), cationic gelatin, cationic cellulose or a combination thereof. Accordingly, the present invention provides a method for attaching a polypeptide to the outer membrane of a mitochondrion, the method comprising the steps of:
a) providing a preparation of mitochondria;
b) contacting the mitochondria provided in step (a) with at least one polypeptide in the presence of a linear or branched polycationic polymer;
c) attaching the at least one polypeptide to the mitochondria via the linear or branched polycationic polymer.

The present invention provides a method for attaching a polypeptide to the outer membrane of a mitochondrion, the method comprising the steps of:
a) providing a preparation of mitochondria;
b) contacting the mitochondria provided in step (a) with at least one polypeptide in the presence of a linear or branched polycationic polymer which is electrostatically linked to a polypeptide;
c) attaching the at least one polypeptide to the mitochondria via the linear or branched polycationic polymer.

The present invention provides a method for attaching a polypeptide to the outer membrane of a mitochondrion, the method comprising the steps of:
a) providing a preparation of mitochondria;
b) contacting the mitochondria provided in step (a) with at least one polypeptide in the presence of polycationic polymer, wherein the polycationic polymer is polylysine, histidylated polylysine, polyornithine, polyarginine, high-mobility group protein (HMG) 1 and 17, modified chitosan, cationized human serum albumin, polyethyleneimine (PEI), polypropyleneimine (PPI), a cationic dendrimer, poly(2-(dimethylamino)ethyl methacrylate) (PDMAEMA), a polyallylamine derivative, diethylaminoethyl (DEAE)-dextran, poly(N-alkyl-4-vinylpyridinium), a poly(amidoamine), cationic gelatin, cationic cellulose or a combination thereof, optionally, wherein the polycationic polymer is covalently linked to the polypeptide;
c) attaching the at least one polypeptide to the mitochondria via the polycationic polymer.

As described herein above, the negative surface charge profile of mitochondria can also be useful for attaching one or more polypeptides electrostatically to the outer membrane of a mitochondrion via a positively-charged nanoparticle. Polypeptides may be attached to the surface of a positively-charged nanoparticle or may be encapsulated in the same. The present invention is not particularly limited to any nanoparticles. In general, any positively-charged nanoparticles facilitating the attachment of polypeptides to a mitochondrion thereby facilitating the formation of the delivery complex may be used. However, it is preferred that a positively-charged nanoparticle is a lipid nanoparticle, a dendrimer nanoparticle, a micelle nanoparticle, a protein nanoparticle, a liposome, a non-porous silica nanoparticle, a mesoporous silica nanoparticle, a silicon nanoparticle, a gold nanoparticle, a gold nanowire, a silver nanoparticle, a platinum nanoparticle, a palladium nanoparticle, a titanium dioxide nanoparticle, a carbon nanotube, a carbon dot nanoparticle, a polymer nanoparticle, a zeolite nanoparticle, an aluminium oxide nanoparticle, a hydroxyapatite nanoparticle, a quantum dot nanoparticle, a zinc oxide nanoparticle, a zirconium oxide nanoparticle, graphene or a graphene oxide nanoparticle. Accordingly, the present invention provides a method for attaching a polypeptide to the outer membrane of a mitochondrion, the method comprising the steps of:
a) providing a preparation of mitochondria;
b) contacting the mitochondria provided in step (a) with at least one polypeptide in the presence of a positively-charged nanoparticle;
c) attaching the at least one polypeptide to the mitochondria via the positively-charged nanoparticle.

The present invention provides a method for attaching a polypeptide to the outer membrane of a mitochondrion, the method comprising the steps of:
a) providing a preparation of mitochondria;
b) contacting the mitochondria provided in step (a) with at least one polypeptide in the presence of a positively-charged nanoparticle;
c) attaching the plurality of polypeptides to the surface of the positively-charged nanoparticle; or encapsulating the polypeptides within the positively-charged nanoparticle;
d) attaching the at least one polypeptide to the mitochondria via the positively-charged nanoparticle.

The present invention provides a method for attaching a polypeptide to the outer membrane of a mitochondrion, the method comprising the steps of:
a) providing a preparation of mitochondria;
b) contacting the mitochondria provided in step (a) with a plurality of polypeptides in the presence of a positively-charged nanoparticle;
c) attaching the plurality of polypeptides to the surface of the positively-charged nanoparticle; or encapsulating the polypeptides within the positively-charged nanoparticle, wherein the positively-charged nanoparticle is a lipid nanoparticle, a dendrimer nanoparticle, a micelle nanoparticle, a protein nanoparticle, a liposome, a non-porous silica nanoparticle, a mesoporous silica nanoparticle, a silicon nanoparticle, a gold nanoparticle, a gold nanowire, a silver nanoparticle, a platinum nanoparticle, a palladium nanoparticle, a titanium dioxide nanoparticle, a carbon nanotube, a carbon dot nanoparticle, a polymer nanoparticle, a zeolite nanoparticle, an aluminium oxide nanoparticle, a hydroxyapatite nanoparticle, a quantum dot nanoparticle, a zinc oxide nanoparticle, a zirconium oxide nanoparticle, graphene or a graphene oxide nanoparticle;
d) attaching the at least one polypeptide to the mitochondria via the positively-charged species.

In another aspect, the present invention provides methods for covalently attaching a polypeptide to the outer membrane of a mitochondrion. As described herein above, the present invention provides polypeptides that may be covalently linked to the outer membrane of a mitochondrion be it directly or indirectly, such as via an intermediate entity. An exemplary intermediate entity comprises an activated ester such as a N-hydroxysuccinimide (NHS) ester. Accordingly, the present invention provides a method for covalently attaching a polypeptide to the outer membrane of a mitochondrion, the method comprising the steps of:
a) providing a preparation of mitochondria;
b) providing a polypeptide that has been modified to comprise an activated ester; and
c) attaching the polypeptide provided in step (b) to an amine comprised in a second polypeptide in the outer membrane of the mitochondria.

Preferably, the present invention provides a method for covalently attaching a polypeptide to the outer membrane of a mitochondrion, the method comprising the steps of:
a) providing a preparation of mitochondria;
b) providing a polypeptide that has been modified to comprise a N-hydroxysuccinimide (NHS) ester; and
c) attaching the polypeptide provided in step (b) to an amine comprised in a second polypeptide in the outer membrane of the mitochondria.

As described herein above, polypeptides may be attached to or encapsulated in a nanoparticle which then may be covalently attached to a mitochondrion via e.g. an amide bond.

The invention provides a method for covalently attaching a polypeptide to the outer membrane of a mitochondrion, the method comprising the steps of:
a) providing a preparation of mitochondria;
b) attaching to or encapsulating a polypeptide in a nanoparticle, wherein the surface of the nanoparticle comprises an activated ester; and
c) attaching the nanoparticle provided in step (b) to an amine comprised in a second polypeptide in the outer membrane of the mitochondria.

Preferably, the invention provides a method for covalently attaching a polypeptide to the outer membrane of a mitochondrion, the method comprising the steps of:
a) providing a preparation of mitochondria;
b) attaching to or encapsulating a polypeptide in a nanoparticle, wherein the surface of the nanoparticle comprises a N-hydroxysuccinimide (NHS) ester; and
c) attaching the nanoparticle provided in step (b) to an amine comprised in a second polypeptide in the outer membrane of the mitochondria.

In the sense of the present invention the polypeptides do not necessarily relate to identical polypeptides, i.e. molecules of identical sequence. Although it is appreciated to deliver identical polypeptides in some aspects, in other aspects of the invention at least two or more different polypeptides may be attached to the outer membrane of a mitochondrion.

The invention also provides a method for attaching a polypeptide to the outer membrane of a mitochondrion, the method comprising the steps of:
a) providing a preparation of mitochondria;
b) contacting the mitochondria provided in step (a) with at least one polypeptide linked to a mitochondria-targeting small molecule; and
c) attaching the at least one polypeptide to the mitochondria via a mitochondria-targeting small molecule.

Preferably, the method for attaching a polypeptide to the outer membrane of the mitochondrion, wherein said polypeptide is attached via said mitochondria-targeting small molecule is a method wherein the mitochondria-targeting small molecule is selected from: triphenylphosphonium (TPP), dequalinium (DQA), E-4-(1H-Indol-3-ylvinyl)-N-Methylpyridineiodide (F16), Rhodamine 19, biguanidine and guanidine.

Within the methods of the present invention for attaching a polypeptide to the outer membrane of a mitochondrion is a method wherein an amount of 50 μg to 200 μg of mitochondria are contacted with 0.1 to 10 μg of the polypeptides and 0.2 to 10 μg of the positively-charged species. Within the present invention, the amount of mitochondria may be determined by the skilled person based on the circumstances and needs in a particular setting based on available methods. Herein, the amount of mitochondria may be within 50 μg to 200 μg, 75 μg to 150 μg, 100 μg to 125 μg. The mitochondria may be contacted with 0.1 to 10 μg of the polypeptide(s), particularly 0.2 to 8 μg, 0.3 to 7 μg, 0.4 to 6 μg, 0.5 to 5 μg, 1 to 2.5 μg, 1.5 to 2 μg. The positively-charged species may be present in an amount of 0.2 to 10 μg, 0.5 to 5 μg, 1 to 2.5 μg, 1.5 to 2 μg.

In a further embodiment, the method of the present invention for attaching a polypeptide to the outer membrane of a mitochondrion is a method wherein an amount of 50 μg to 200 μg of mitochondria are contacted with 0.1 to 10 μg of the polypeptides linked to a mitochondria-targeting small molecule.

In some embodiments, the method of the invention further comprises linking to and/or enveloping the mitochondrion comprising one or more polypeptide(s) in a protective layer. The mitochondrion comprising the one or more polypeptide(s) may be any mitochondrion as described hereinabove and any protective layer as described hereinabove. In preferred embodiments, the invention is a method for attaching a polypeptide to the outer membrane of a mitochondrion, wherein the method comprises the steps of:
a) providing a preparation of mitochondria;
b) contacting the mitochondria provided in step (a) with at least one polypeptide in the presence of a positively-charged species;
c) attaching the at least one polypeptide to the mitochondria via the positively-charged species; and
d) linking and/or enveloping the mitochondrion provided in steps (a) to (c) with a protective layer.

In some embodiments, the protective layer is a protective polymer. The protective polymer is as described herein above.

In some embodiments of the method of the present invention, the protective polymer is a linear or branched cationic polymer, optionally wherein the linear or branched cationic polymer is electrostatically linked to the one or more polypeptide(s), preferably wherein said polypeptide is a negatively-charged polypeptide. Preferably, the linear or branched cationic polymer is polyethyleneimine, RGD-modified polyethyleneimine, polylysine, RGD-modified polylysine, polyornithine, RGD-modified polyornithine, polyarginine, RGD modified polyarginine, polypropyleneimine, RGD-modified polypropyleneimine, polyallylamine, RGD-modified polyallylamine, chitosan, RGD-modified chitosan, poly(2-(dimethylamino)ethyl methacrylate), RGD-modified poly(2-(dimethylamino)ethyl methacrylate), poly(amidoamine)s, RGD-modified poly(amidoamine)s or a combination thereof.

In some embodiments of the method of the present invention, the protective polymer is a linear or branched cationic block copolymer, optionally wherein the linear or branched cationic block copolymer is electrostatically linked to the one or more polypeptide(s), preferably wherein said polypeptide is a negatively-charged polypeptide. Preferably, the cationic block copolymer is poly(ethylene glycol)-block-polyethyleneimine, RGD-modified poly(ethylene glycol)-block-polyethyleneimine, poly(ethylene glycol)-block-polylysine, RGD-modified poly(ethylene glycol)-block-polylysine, poly(ethylene glycol)-block-polyornithine, RGD-modified poly(ethylene glycol)-block-polyornithine, poly(ethylene glycol)-block-polyarginine, RGD-modified poly(ethylene glycol)-block-polyarginine, poly(ethylene glycol)-block-polypropyleneimine, RGD-modified poly(ethylene glycol)-block-polypropyleneimine, poly(ethylene glycol)-block-polyallylamine, RGD-modified poly(ethylene glycol)-block-polyallylamine, poly(ethylene glycol)-block-poly(2-(dimethylamino)ethyl methacrylate), RGD-modified poly(ethylene glycol)-block-poly(2-(dimethylamino)ethyl methacrylate), poly(ethylene glycol)-block-poly(amidoamine)s, RGD-modified poly(ethylene glycol)-block-poly(amidoamine)s or a combination thereof.

In some embodiments of the method of the present invention, the protective polymer is a linear or branched cationic graft (g) copolymer, optionally wherein the linear or branched cationic graft (g) copolymer is electrostatically linked to the one or more polypeptide(s), preferably wherein said polypeptide is a negatively-charged polypeptide. Preferably, the cationic graft (g) copolymer is poly(ethylene glycol)-g-polyethyleneimine, RGD-modified poly(ethylene glycol)-g-polyethyleneimine, poly(ethylene glycol)-g-polylysine, RGD-modified poly(ethylene glycol)-g-polylysine, poly(ethylene glycol)-g-polyornithine, RGD-modified poly(ethylene glycol)-g-polyornithine, poly(ethylene glycol)-g-polyarginine, RGD-modified poly(ethylene glycol)-g-polyarginine, poly(ethylene glycol)-g-polypropyleneimine, RGD-modified poly(ethylene glycol)-g-polypropyleneimine, poly(ethylene glycol)-g-polyallylamine, RGD-modified poly(ethylene glycol)-g-polyallylamine, poly(ethylene glycol)-g-poly(2-(dimethylamino)ethyl methacrylate), RGD-modified poly(ethylene glycol)-g-poly(2-(dimethylamino)ethyl methacrylate), poly(ethylene glycol)-g-poly(amidoamine)s, RGD-modified poly(ethylene glycol)-g-poly(amidoamine)s or a combination thereof.

In some embodiments of the method of the present invention, the protective polymer is a linear or branched pegylated (PEG) cationic polymer, optionally wherein the linear or branched pegylated (PEG) cationic polymer is electrostatically linked to the one or more polypeptide(s), preferably wherein said polypeptide is a negatively-charged polypeptide. Preferably, the pegylated (PEG) cationic polymer is pegylated-polyethyleneimine, RGD-modified pegylated polyethyleneimine, pegylated polylysine, RGD-modified pegylated polylysine, histidylated polylysine, pegylated polyornithine, RGD-modified pegylated polyornithine, pegylated polyarginine, RGD-modified pegylated polyarginine, pegylated polypropyleneimine, RGD-modified pegylated polypropyleneimine, pegylated polyallylamine, RGD-modified pegylated polyallylamine, pegylated chitosan, RGD-modified pegylated chitosan, pegylated poly(2-(dimethylamino)ethyl methacrylate), RGD-modified pegylated poly(2-(dimethylamino)ethyl methacrylate), pegylated poly(amidoamine)s RGD-modified pegylated poly(amidoamine)s or a combination thereof.

In some embodiments of the method of the present invention, the protective layer is a lipid formulation, optionally wherein the lipid formulation is a cationic lipid formulation, further optionally wherein the cationic lipid formulation is electrostatically linked to the one or more polypeptide(s), preferably wherein said polypeptide is a negatively-charged polypeptide. Preferably, wherein the lipid formulation comprises DC-cholesterol (3β-[N—(N′,N′-Dimethylaminoethane)-carbamoyl]cholesterol hydrochloride), DLinDMA (1,2-dilinoleyloxy-3-dimethylaminopropane), DLinMC3DMA (dilinoleylmethyl-4-dimethylaminobutyrate), DODMA (1,2-dioleyloxy-3-dimethylaminopropane), DOGS (dioctadecylamidoglycylspermine), DOSPA (2,3-dioleyloxy-N-[2 (sperminecarboxamido)ethyl]-N,N-dimethyl-1-propanaminium), DOTAP (1,2-dioleoyl-3-trimethylammonium-propane chloride), DOTMA (1,2-di-O-octadecenyl-3-trimethylammonium propane chloride), UGG (unsaturated guanidinium glycoside), DOPE (1,2-Dioleoyl-sn-glycerophosphoethanolamine), lipofectamine or a combination thereof. In further embodiments, the lipid formulation further comprises another lipid, preferably wherein said lipid is cholesterol, a substituted or unsubstituted cholesterol, a cholesterol derivative, such as a hydroxylated cholesterol derivative (e.g., a hydroxycholesterol), a PEG-lipid, DMPC (1,2-Dimyristoyl-sn-glycero-3-phosphocholine), DSPC (1,2-Distearoyl-sn-glycero-3-phosphocholine), DODAP (1,2-dioleoyl-3-dimethylammonium propane), DDA (dimethyldioctadecylammonium), 1,2-dioleoyl-sn-glycero-3-phosphate, 1,2-dimyristoyl-sn-glycero-3-phosphate, bis(monooleoylglycerol)phosphate or a combination thereof.

In some embodiments of the method of the present invention the protective polymer is a zwitterionic protective polymer, optionally wherein the zwitterionic protective polymer is electrostatically linked to the one or more polypeptide(s). Preferably, the zwitterionic protective polymer is selected from: poly(2-methacryloyloxyethyl phosphorylcholine) (PMPC), polyethyleneimine-g-poly(2-methacryloyloxyethyl phosphorylcholine) (PEI-g-PMPC), co-assembly of cationic (carboxyl-functionalized) and anionic (amino-functionalized) copolyesters based on poly(ε-caprolactone)-block-poly(butylene fumarate)-block-poly(8-caprolactone) (PCL-b-PBF-b-PCL), poly(lactic-co-glycolic acid) (PLGA)-PCB block copolymers (PLGA-b-PCB).

In some embodiments of the methods of the present invention, the protective layer is linked to an antibody, optionally wherein the protective layer linked to an antibody is electrostatically linked to the polypeptide(s), preferably wherein said polypeptide is a negatively-charged polypeptide.

In some embodiments of the method of the invention, the protective layer is linked to a carbohydrate, optionally wherein the protective layer linked to a carbohydrate is electrostatically linked to the one or more polypeptide(s), preferably wherein said polypeptide is a negatively-charged polypeptide.

In further embodiments, the method of the present invention may involve a centrifugation step. The centrifugation step, within the context of the present invention enables the removal of the components comprising the mitochondrion delivery vehicle, e.g., unattached payload, such as the polypeptide, the positively-charged species or the protective layer to facilitate the formation of the delivery vehicle. As the skilled person is aware, the centrifugation step may be performed after any step which requires removal of excess components of the delivery vehicle, e.g. excess payload, excess positive-charged species, excess protective layer. The centrifugation step may be added to the methods of attaching one or more polypeptide(s) to the mitochondrion as described hereinabove in an analogous manner as described hereinabove for the embodiments relating to the mitochondrion comprising the nucleic acid molecules.

In another aspect, the invention provides a mitochondrion comprising one or more drug(s) attached to the outer membrane of the mitochondrion, wherein the one or more drug(s):
a) is electrostatically attached to the outer membrane of the mitochondrion; or
b) is covalently linked to the outer membrane of the mitochondrion; or
c) is linked to an antibody that specifically binds to an antigen comprised in the outer membrane of the mitochondrion; or
d) is linked to a mitochondria-targeting small molecule.

Herein, the one or more drug(s) may be a charged drug.

In a more specific embodiment, the drug may be an anionic drug. In a preferred embodiment, the anionic drug may be selected from: potassium iodide, iodide, artesunate, sodium fluoride, carbamide peroxide, sodium zirconium cyclosilicate, nitrite, lithium carbonate, zinc chloride, aluminium hydroxide, magaldrate, aluminium sesquichlorohydrate, hydrotalcite, aluminium glycinate, aloglutamol, dihydroxyaluminium sodium carbonate, cystine, nitroprusside, montelukast, stepronin, prostaglandin G2, pyrophosphoric acid, OXI-4503, tetrachlorodecaoxide, NCX 701, PX-12, nitrous acid, chromic chloride, ferric pyrophosphate, activated charcoal, monopotassium phosphate, dipotassium phosphate, sodium fluorophosphate, potassium nitrate, potassium bicarbonate, sulfur hexafluoride, PF-4191834, allicin, artefenomel, lodenafil carbonate, devimistat, GW-274150, imrecoxib, chlorine dioxide, perflubutane, CHS-828, QGC-001, trabodenoson, magnesium phosphate, TAK-243, dostarlimab, GC-376 free acid, sodium metabisulfite, diquafosol, ammonium carbonate, NCX-1000 and ethyl nitrite, Nitroprusside, Technetium Tc-99m polyphosphate, Sodium phosphate monobasic, Sodium sulfate, Indium, Chromic nitrate, Tetrafluoroborate, Darapladib, PF-03715455 and Umifenovir.

In another embodiment, the drug may be a cationic drug. In a preferred embodiment, the cationic drug may be selected from Methyl-piperidino-pyrazole (MPP), Bretylium, Acetylcarnitine, Fluorocholine F-18, Hexamethonium, Edrophonium, Choline, Succinylcholine, Oxyphenonium, Carbamoylcholine, Gallamine triethiodide, Glycopyrronium, Bethanechol, Ambenonium, Methacholine, Betaine, Benzalkonium, Benzethonium, Emepronium, Benzoxonium, Gallamine, Octenidine, Methantheline, Propantheline, Tubocurarine, Neostigmine, Butylscopolamine, Alcuronium, Metocurine iodide, Levocarnitine, Hexafluronium, Decamethonium, Oxtriphylline, Metocurine, Choline magnesium trisalicylate, Platelet Activating Factor, N,N,N-Trimethyl-2-(phosphonooxy) ethanaminium, Butyrylthiocholine, Betaine aldehyde, C31G, Perifosine, Tetraethylammonium, Miltefosine, Citicoline, Benzododecinium, Choline salicylate, Cetyltrimethylammonium naproxenate and Trimethyltetradecylammonium.

As described above, mitochondria generally have a negative surface charge. Accordingly, an anionic drug, which is negatively-charged at conditions where the mitochondria remain negatively-charged, requires a positively-charged species in order to be attached to the mitochondria. Accordingly, in a further embodiment of the present invention, the anionic drug is electrostatically attached to the outer membrane of the mitochondrion via a positively-charged species.

In contrast, a drug having a positive surface charge at conditions where the mitochondrion is negatively-charged may be electrostatically attached to the outer membrane of the mitochondrion.

In some embodiments, the drug may be a zwitterionic drug.

The positively-charged species may be a polycationic species, wherein the polycationic species is a linear or branched polycationic polymer, optionally wherein the linear or branched polycationic polymer is electrostatically linked to one or more anionic drug(s).

The linear or branched polycationic polymer may be polylysine, histidylated polylysine, polyornithine, polyarginine, high-mobility group protein (HMG) 1 and 17, modified chitosan, cationized human serum albumin, polyethyleneimine (PEI), polypropyleneimine (PPI), a cationic dendrimer, poly(2-(dimethylamino)ethyl methacrylate) (PDMAEMA), a polyallylamine derivative, diethylaminoethyl (DEAE)-dextran, poly(N-alkyl-4-vinylpyridinium), a poly(amidoamine), cationic gelatin, cationic cellulose or a combination thereof.

The positively-charged species may be a positively-charged nanoparticle.

The positively-charged species may be a positively-charged particle.

Within the present invention, the one or more anionic drug(s) may be attached to the surface of the positively-charged nanoparticle or encapsulated in the positively-charged nanoparticle. Within the present invention, the one or more anionic drug(s) may be attached to the surface of the positively-charged particle or encapsulated in the positively-charged particle.

Within the present invention, the positively-charged nanoparticle/particle may be a lipid nanoparticle/particle, a dendrimer nanoparticle/particle, a micelle nanoparticle/particle, a protein nanoparticle/particle, a liposome, a non-porous silica nanoparticle/particle, a mesoporous silica nanoparticle/particle, a silicon nanoparticle/particle, a gold nanoparticle/particle, a gold nanowire, a silver nanoparticle/particle, a platinum nanoparticle/particle, a palladium nanoparticle/particle, a titanium dioxide nanoparticle/particle, a carbon nanotube, a carbon dot nanoparticle/particle, a polymer nanoparticle/particle, a zeolite nanoparticle/particle, an aluminium oxide nanoparticle/particle, a hydroxyapatite nanoparticle/particle, a quantum dot nanoparticle/particle, a zinc oxide nanoparticle/particle, a zirconium oxide nanoparticle/particle, graphene or a graphene oxide nanoparticle/particle.

The one or more drug(s) may be linked to a polypeptide in the outer membrane of the mitochondrion via an amide bond. Accordingly, the one or more drug(s) may be modified to undergo formation of the amide bond with an amine function comprised in the polypeptide in the outer membrane of the mitochondrion.

In another embodiment, the one or more drug(s) may be encapsulated in a nanoparticle, and wherein the nanoparticle comprises a functional group that allows covalent linkage of the nanoparticle to a polypeptide in the outer membrane of the mitochondrion.

In one embodiment where the one or more drug(s) are linked via an antibody, the antibody may specifically bind to an antigen comprised in the outer membrane of the mitochondrion, wherein the antigen is OPA1, TOM70, TOMM20, Mitofusin 1, Mitofusin 2 or VDAC1.

In the present invention, the one or more drug(s) may also be encapsulated in a nanoparticle, and wherein the nanoparticle is covalently linked to an antibody as described herein. Within the present invention, the one or more anionic drug(s) may be electrostatically linked to the antibody as described herein, wherein the antibody is a modified antibody, wherein the modified antibody possesses one or more positive charge(s).

In another embodiment, the one or more drug(s) may be covalently linked to biotin, wherein biotin is linked to the antibody, wherein the antibody is an avidin conjugated antibody. In another embodiment, the one or more drug(s) may be covalently linked to an activated ester, wherein the activated ester is linked to the antibody via an amide bond.

Alternatively or additionally, the one or more drug(s) may be encapsulated in a nanoparticle, wherein the nanoparticle is electrostatically linked to the antibody, wherein the antibody is a modified antibody, wherein the modified antibody possesses one or more positive charge(s). Additionally or alternatively, the one or more drug(s) may be encapsulated in a nanoparticle, wherein the nanoparticle is covalently linked to biotin, wherein biotin is linked the antibody, wherein the antibody is an avidin conjugated antibody.

The one or more drug(s) may be encapsulated in a nanoparticle, wherein the nanoparticle is covalently linked to an activated ester, wherein the activated ester is linked to the antibody via an amide bond.

The mitochondria-targeting small molecule may be selected from the group consisting of triphenylphosphonium (TPP), dequalinium (DQA), E-4-(1H-Indol-3-ylvinyl)-N-Methylpyridineiodide (F16), Rhodamine 19, biguanidine and guanidine.

In one embodiment of the invention, the mitochondrion of the invention may be linked to and/or enveloped in a protective layer. The protective layer may be a protective polymer. Within the present invention, the protective polymer may be a linear or branched cationic polymer. Herein, the linear or branched cationic polymer may be electrostatically linked to the one or more drug(s). In an alternative embodiment, the protective polymer may be a linear or branched cationic block copolymer. Herein, the linear or branched cationic block copolymer may be electrostatically linked to the one or more drug(s).

In a further embodiment, the protective polymer may be a cationic graft (g) copolymer. Herein, the cationic graft (g) copolymer may be electrostatically linked to the one or more drug(s). The protective polymer may be a linear or branched pegylated (PEG) cationic polymer, optionally wherein the linear or branched pegylated (PEG) cationic polymer is electrostatically linked to the one or more drug(s).

The protective layer may be a lipid formulation, optionally wherein the lipid formulation is a cationic lipid formulation, further optionally wherein the cationic lipid formulation is electrostatically linked to the one or more drug(s).

The protective layer may be linked to a targeting moiety, optionally wherein the protective layer linked to a targeting moiety is electrostatically linked to the one or more drugs.

The protective layer may be linked to an antibody, optionally wherein the protective layer linked to an antibody is electrostatically linked to the one or more drug(s). The protective layer may be connected to a carbohydrate, optionally wherein the protective layer connected to a carbohydrate is electrostatically linked to the one or more drug(s).

The linear or branched cationic polymer may be polyethyleneimine, RGD-modified polyethyleneimine, polylysine, RGD-modified polylysine, polyornithine, RGD-modified polyornithine, polyarginine, RGD modified polyarginine, polypropyleneimine, RGD-modified polypropyleneimine, polyallylamine, RGD-modified polyallylamine, chitosan, RGD-modified chitosan, poly(2-(dimethylamino)ethyl methacrylate), RGD-modified poly(2-(dimethylamino)ethyl methacrylate), poly(amidoamine)s, RGD-modified poly(amidoamine) s or a combination thereof.

The cationic block copolymer may be poly(ethylene glycol)-block-polyethyleneimine, RGD-modified poly(ethylene glycol)-block-polyethyleneimine, poly(ethylene glycol)-block-polylysine, RGD-modified poly(ethylene glycol)-block-polylysine, poly(ethylene glycol)-block-polyornithine, RGD-modified poly(ethylene glycol)-block-polyornithine, poly(ethylene glycol)-block-polyarginine, RGD-modified poly(ethylene glycol)-block-polyarginine, poly(ethylene glycol)-block-polypropyleneimine, RGD-modified poly(ethylene glycol)-block-polypropyleneimine, poly(ethylene glycol)-block-polyallylamine, RGD-modified poly(ethylene glycol)-block-polyallylamine, poly(ethylene glycol)-block-poly(2-(dimethylamino)ethyl methacrylate), RGD-modified poly(ethylene glycol)-block-poly(2-(dimethylamino)ethyl methacrylate), poly(ethylene glycol)-block-poly(amidoamine)s, RGD-modified poly(ethylene glycol)-block-poly(amidoamine)s or a combination thereof.

The cationic graft (g) copolymer may be poly(ethylene glycol)-g-polyethyleneimine, RGD-modified poly(ethylene glycol)-g-polyethyleneimine, poly(ethylene glycol)-g-polylysine, RGD-modified poly(ethylene glycol)-g-polylysine, poly(ethylene glycol)-g-polyornithine, RGD-modified poly(ethylene glycol)-g-polyornithine, poly(ethylene glycol)-g-polyarginine, RGD-modified poly(ethylene glycol)-g-polyarginine, poly(ethylene glycol)-g-polypropyleneimine, RGD-modified poly(ethylene glycol)-g-polypropyleneimine, poly(ethylene glycol)-g-polyallylamine, RGD-modified poly(ethylene glycol)-g-polyallylamine, poly(ethylene glycol)-g-poly(2-(dimethylamino)ethyl methacrylate), RGD-modified poly(ethylene glycol)-g-poly(2-(dimethylamino)ethyl methacrylate), poly(ethylene glycol)-g-poly(amidoamine)s, RGD-modified poly(ethylene glycol)-g-poly(amidoamine)s or a combination thereof.

The pegylated (PEG) cationic polymer may be pegylated-polyethyleneimine, RGD-modified pegylated polyethyleneimine, pegylated polylysine, RGD-modified pegylated polylysine, histidylated polylysine, pegylated polyornithine, RGD-modified pegylated polyornithine, pegylated polyarginine, RGD-modified pegylated polyarginine, pegylated polypropyleneimine, RGD-modified pegylated polypropyleneimine, pegylated polyallylamine, RGD-modified pegylated polyallylamine, pegylated chitosan, RGD-modified pegylated chitosan, pegylated poly(2-(dimethylamino)ethyl methacrylate), RGD-modified pegylated poly(2-(dimethylamino)ethyl methacrylate), pegylated poly(amidoamine)s RGD-modified pegylated poly(amidoamine)s or a combination thereof.

The lipid formulation may comprise DC-cholesterol (3β-[N—(N′,N′-Dimethylaminoethane)-carbamoyl]cholesterol hydrochloride), DLinDMA (1,2-dilinoleyloxy-3-dimethylaminopropane), DLinMC3DMA (dilinoleylmethyl-4-dimethylaminobutyrate), DODMA (1,2-dioleyloxy-3-dimethylaminopropane), DOGS (dioctadecylamidoglycylspermine), DOSPA (2,3-dioleyloxy-N-[2 (sperminecarboxamido)ethyl]-N,N-dimethyl-1-propanaminium), DOTAP (1,2-dioleoyl-3-trimethylammonium-propane chloride), DOTMA (1,2-di-O-octadecenyl-3-trimethylammonium propane chloride)),

UGG (unsaturated guanidinium glycoside), DOPE (1,2-Dioleoyl-sn-glycerophosphoethanolamine), lipofectamine or a combination thereof.

The lipid formulation may further comprise another lipid, preferably wherein said lipid is cholesterol, a substituted or unsubstituted cholesterol, a cholesterol derivative, such as a hydroxylated cholesterol derivative (e.g., a hydroxycholesterol), a PEG-lipid, DMPC (1,2-Dimyristoyl-sn-glycero-3-phosphocholine), DSPC (1,2-Distearoyl-sn-glycero-3-phosphocholine), DODAP (1,2-dioleoyl-3-dimethylammonium propane), DDA (dimethyldioctadecylammonium), 1,2-dioleoyl-sn-glycero-3-phosphate, 1,2-dimyristoyl-sn-glycero-3-phosphate, bis(monooleoylglycerol)phosphate or a combination thereof.

The mitochondrion may be linked to and/or enveloped in a zwitterionic protective polymer, optionally wherein the zwitterionic protective polymer is electrostatically linked to the one or more drug(s).

The zwitterionic protective polymer may be selected from: poly(2-methacryloyloxyethyl phosphorylcholine) (PMPC), polyethyleneimine-g-poly(2-methacryloyloxyethyl phosphorylcholine) (PEI-g-PMPC), co-assembly of cationic (carboxyl-functionalized) and anionic (amino-functionalized) copolyesters based on poly(ε-caprolactone)-block-poly(butylene fumarate)-block-poly(ε-caprolactone) (PCL-b-PBF-b-PCL), poly(lactic-co-glycolic acid) (PLGA)-PCB block copolymers (PLGA-b-PCB).

The invention also relates to a composition comprising a plurality of mitochondria according to the invention provided herein, in particular the mitochondrion linked to one or more drug(s) as provided herein above.

In another embodiment, the invention relates to a pharmaceutical composition comprising a plurality of mitochondria according to this invention, in particular the mitochondria linked to one or more drug(s) as provided herein and a pharmaceutically acceptable carrier. In one embodiment, the pharmaceutical composition may be formulated as a solution. The pharmaceutical composition may be formulated as an aerosol.

The present invention also relates to the mitochondrion according to the present invention, the composition according to the present invention and/or the pharmaceutical composition according to the present invention for use as a medicament.

The present invention also relates to the mitochondrion according to the present invention, the composition according to the present invention and/or the pharmaceutical composition according to the present invention for use in gene therapy.

The present invention also relates to the mitochondrion according to the present invention, the composition according to the present invention and/or the pharmaceutical composition according to the present invention for use in the treatment of cardiovascular diseases, in particular for use in the treatment of ischemic heart disease, ischemia-reperfusion injury and/or atherosclerosis.

The present invention also relates to the mitochondrion according to the present invention, the composition according to the present invention and/or the pharmaceutical composition according to the present invention for use in the treatment of aging related diseases, in particular for use in the treatment of, sarcopenia, Parkinson's disease or Hutchinson-Gilford progeria syndrome.

The present invention also relates to the mitochondrion according to the present invention, the composition according to the present invention and/or the pharmaceutical composition according to the present invention for use in the treatment of kidney diseases, in particular for use in the treatment of, autosomal dominant polycystic kidney disease, Alport syndrome, Nephronophthisis, or Fabry disease.

The present invention also relates to the mitochondrion according to the present invention, the composition according to the present invention and/or the pharmaceutical composition according to the present invention for use in the treatment of cancer.

The present invention also relates to the mitochondrion according to the present invention, the composition according to the present invention and/or the pharmaceutical composition according to the present invention for use in in vitro, ex vivo, or in vivo genome editing.

The present invention also relates to the mitochondrion according to the present invention, the composition according to the present invention and/or the pharmaceutical composition according to the present invention for use in radiation therapy.

In a further embodiment, the invention relates to a method for delivering a drug to a target organ, the method comprising a step of administering the pharmaceutical composition according to the present invention into the bloodstream of a subject in need, wherein the pharmaceutical composition is administered into the bloodstream upstream of the target organ.

The invention also relates to a method for delivering a drug to the lung, the method comprising a step of administering the pharmaceutical composition according to the invention to a subject in need, wherein the pharmaceutical composition is administered by inhalation.

In a further embodiment, the invention relates to a method for attaching at least one drug to the outer membrane of a mitochondrion, the method comprising the steps of:
a) providing a preparation of mitochondria;
b) contacting the mitochondria provided in step (a) with at least one drug, optionally in the presence of a positively-charged species; and
c) attaching the at least one drug to the mitochondria, optionally via the positively-charged species.

In a preferred embodiment, in step (b), the at least one drug is contacted with the mitochondrion in the presence of the positively-charged species, wherein:
a) the at least one drug is simultaneously contacted with mitochondria and the positively-charged species; or
b) wherein the at least one drug is contacted with the positively-charged species to form a positively-charged complex before the positively-charged complex is contacted with the mitochondria; or
c) the mitochondrion is contacted with the positively-charged species and subsequently contacted with the at least one drug.

Within the invention, the mitochondria may be contacted with the at least one drug and the positively-charged species in a suitable buffer.

In the method provided herein above, the buffer may comprise or consist of HEPES, EGTA, Trehalose CHES and/or sodium phosphate dibasic dihydrate. It is preferred that the buffer comprises a mixture of a Solution X comprising or consisting of HEPES, EGTA and Trehalose and of a Solution Y comprising or consisting of CHES and sodium phosphate dibasic dihydrate. It is more preferred that the buffer comprises a 4:1 mixture of Solution X comprising or consisting of 20 mM HEPES, 1 mM EGTA and 300 mM Trehalose (pH 7.2) and Solution Y comprising or consisting of 0.1 M CHES (pH 10) and 0.2 M sodium phosphate dibasic dihydrate.

In one embodiment, the mitochondria are contacted with the at least one drug and the positively-charged species at room temperature for at least 5 minutes, such as at least 10 minutes, 20, 30, 40, 50, 60, 70, 80, 90, 100, 110, or 120 minutes.

In one embodiment, the mitochondria are contacted with of the at least one drug and the positively-charged species in the dark.

Within the present invention, the positively-charged species may be a polycationic species, wherein the polycationic species is a linear or branched polycationic polymer, optionally wherein the linear or branched polycationic polymer is electrostatically linked to the at least one drug.

As such, the linear or branched polycationic polymer may be polylysine, histidylated polylysine, polyornithine, polyarginine, high-mobility group protein (HMG) 1 and 17, modified chitosan, cationized human serum albumin, polyethyleneimine (PEI), polypropyleneimine (PPI), a cationic dendrimer, poly(2-(dimethylamino)ethyl methacrylate) (PDMAEMA), a polyallylamine derivative, diethylaminoethyl (DEAE)-dextran, poly(N-alkyl-4-vinylpyridinium), a poly(amidoamine), cationic gelatin, cationic cellulose or a combination thereof.

The positively-charged species may be a positively-charged nanoparticle.

### Accordingly, the Method Provided Herein Above May Comprise a Further Step of

a) attaching the at least one drug to the surface of the positively-charged nanoparticle; or
b) encapsulating the at least one drug within the positively-charged nanoparticle.

In this embodiment, the positively-charged nanoparticle may be a lipid nanoparticle, a dendrimer nanoparticle, a micelle nanoparticle, a protein nanoparticle, a liposome, a non-porous silica nanoparticle, a mesoporous silica nanoparticle, a silicon nanoparticle, a gold nanoparticle, a gold nanowire, a silver nanoparticle, a platinum nanoparticle, a palladium nanoparticle, a titanium dioxide nanoparticle, a carbon nanotube, a carbon dot nanoparticle, a polymer nanoparticle, a zeolite nanoparticle, an aluminium oxide nanoparticle, a hydroxyapatite nanoparticle, a quantum dot nanoparticle, a zinc oxide nanoparticle, a zirconium oxide nanoparticle, graphene or a graphene oxide nanoparticle.

The invention also relates to a method for covalently attaching a drug to the outer membrane of a mitochondrion, the method comprising the steps of:
a) providing a preparation of mitochondria;
b) providing a drug that has been modified to comprise an activated ester; and
c) attaching the drug provided in step (b) to an amine comprised in a polypeptide in the outer membrane of the mitochondria.

Therein, the activated ester may be an N-hydroxysuccinimide (NHS) ester.

A method for covalently attaching a drug to the outer membrane of a mitochondrion, the method comprising the steps of:
a) providing a preparation of mitochondria;
b) encapsulating a drug in a nanoparticle, wherein the surface of the nanoparticle comprises an activated ester; and
c) attaching the nanoparticle provided in step (b) to an amine comprised in a polypeptide in the outer membrane of the mitochondria.

In some embodiments, the activated ester is an NHS ester.

The methods of the present invention may further comprise a step of adding a protective layer. Accordingly, in some embodiments, the method of the invention further comprises linking to and/or enveloping the mitochondrion in a protective layer. The mitochondrion may be any mitochondrion as described hereinabove and any protective layer as described hereinabove. In preferred embodiments, the invention is a method for attaching one or more drug(s) to the outer membrane of a mitochondrion, wherein the method comprises the steps of:
a) providing a preparation of mitochondria;
b) contacting the mitochondria provided in step (a) with at least one drug;
c) attaching the at least one drug to the mitochondria; and
d) contacting the mitochondrion provided in steps (a) to (c) with a protective layer, wherein the protective layer links to and/or envelops the mitochondrion.

In some embodiments, the protective layer is a protective polymer. The protective polymer is as described herein above.

In some embodiments of the method of the present invention, the protective polymer is a linear or branched cationic polymer, optionally wherein the linear or branched cationic polymer is electrostatically linked to the one or more polypeptide(s), preferably wherein said polypeptide is a negatively-charged polypeptide. Preferably, the linear or branched cationic polymer is polyethyleneimine, RGD-modified polyethyleneimine, polylysine, RGD-modified polylysine, polyornithine, RGD-modified polyornithine, polyarginine, RGD modified polyarginine, polypropyleneimine, RGD-modified polypropyleneimine, polyallylamine, RGD-modified polyallylamine, chitosan, RGD-modified chitosan, poly(2-(dimethylamino)ethyl methacrylate), RGD-modified poly(2-(dimethylamino)ethyl methacrylate), poly(amidoamine)s, RGD-modified poly(amidoamine)s or a combination thereof.

In some embodiments of the method of the present invention, the protective polymer is a linear or branched cationic block copolymer, optionally wherein the linear or branched cationic block copolymer is electrostatically linked to the one or more polypeptide(s), preferably wherein said polypeptide is a negatively-charged polypeptide. Preferably, the cationic block copolymer is poly(ethylene glycol)-block-polyethyleneimine, RGD-modified poly(ethylene glycol)-block-polyethyleneimine, poly(ethylene glycol)-block-polylysine, RGD-modified poly(ethylene glycol)-block-polylysine, poly(ethylene glycol)-block-polyornithine, RGD-modified poly(ethylene glycol)-block-polyornithine, poly(ethylene glycol)-block-polyarginine, RGD-modified poly(ethylene glycol)-block-polyarginine, poly(ethylene glycol)-block-polypropyleneimine, RGD-modified poly(ethylene glycol)-block-polypropyleneimine, poly(ethylene glycol)-block-polyallylamine, RGD-modified poly(ethylene glycol)-block-polyallylamine, poly(ethylene glycol)-block-poly(2-(dimethylamino)ethyl methacrylate), RGD-modified poly(ethylene glycol)-block-poly(2-(dimethylamino)ethyl methacrylate), poly(ethylene glycol)-block-poly(amidoamine)s, RGD-modified poly(ethylene glycol)-block-poly(amidoamine)s or a combination thereof.

In some embodiments of the method of the present invention, the protective polymer is a linear or branched cationic graft (g) copolymer, optionally wherein the linear or branched cationic graft (g) copolymer is electrostatically linked to the one or more polypeptide(s), preferably wherein said polypeptide is a negatively-charged polypeptide. Preferably, the cationic graft (g) copolymer is poly(ethylene glycol)-g-polyethyleneimine, RGD-modified poly(ethylene glycol)-g-polyethyleneimine, poly(ethylene glycol)-g-polylysine, RGD-modified poly(ethylene glycol)-g-polylysine, poly(ethylene glycol)-g-polyornithine, RGD-modified poly(ethylene glycol)-g-polyornithine, poly(ethylene glycol)-g-polyarginine, RGD-modified poly(ethylene glycol)-g-polyarginine, poly(ethylene glycol)-g-polypropyleneimine, RGD-modified poly(ethylene glycol)-g-polypropyleneimine, poly(ethylene glycol)-g-polyallylamine, RGD-modified poly(ethylene glycol)-g-polyallylamine, poly(ethylene glycol)-g-poly(2-(dimethylamino)ethyl methacrylate), RGD-modified poly(ethylene glycol)-g-poly(2-(dimethylamino)ethyl methacrylate), poly(ethylene glycol)-g-poly(amidoamine)s, RGD-modified poly(ethylene glycol)-g-poly(amidoamine)s or a combination thereof. In some embodiments of the method of the present invention, the protective polymer is a linear or branched pegylated (PEG) cationic polymer, optionally wherein the linear or branched pegylated (PEG) cationic polymer is electrostatically linked to the one or more polypeptide(s), preferably wherein said polypeptide is a negatively-charged polypeptide. Preferably, the pegylated (PEG) cationic polymer is pegylated-polyethyleneimine, RGD-modified pegylated polyethyleneimine, pegylated polylysine, RGD-modified pegylated polylysine, histidylated polylysine, pegylated polyornithine, RGD-modified pegylated polyornithine, pegylated polyarginine, RGD-modified pegylated polyarginine, pegylated polypropyleneimine, RGD-modified pegylated polypropyleneimine, pegylated polyallylamine, RGD-modified pegylated polyallylamine, pegylated chitosan, RGD-modified pegylated chitosan, pegylated poly(2-(dimethylamino)ethyl methacrylate), RGD-modified pegylated poly(2-(dimethylamino)ethyl methacrylate), pegylated poly(amidoamine)s RGD-modified pegylated poly(amidoamine)s or a combination thereof.

In some embodiments of the method of the present invention, the protective layer is a lipid formulation, optionally wherein the lipid formulation is a cationic lipid formulation, further optionally wherein the cationic lipid formulation is electrostatically linked to the one or more polypeptide(s), preferably wherein said polypeptide is a negatively-charged polypeptide. Preferably, wherein the lipid formulation comprises DC-cholesterol (3β-[N—(N′,N′-Dimethylaminoethane)-carbamoyl]cholesterol hydrochloride), DLinDMA (1,2-dilinoleyloxy-3-dimethylaminopropane), DLinMC3DMA (dilinoleylmethyl-4-dimethylaminobutyrate), DODMA (1,2-dioleyloxy-3-dimethylaminopropane), DOGS (dioctadecylamidoglycylspermine), DOSPA (2,3-dioleyloxy-N-[2 (sperminecarboxamido) ethyl]-N,N-dimethyl-1-propanaminium), DOTAP (1,2-dioleoyl-3-trimethylammonium-propane chloride), DOTMA (1,2-di-O-octadecenyl-3-trimethylammonium propane chloride), UGG (unsaturated guanidinium glycoside), DOPE (1,2-Dioleoyl-sn-glycerophosphoethanolamine), lipofectamine or a combination thereof. In further embodiments, the lipid formulation further comprises another lipid, preferably wherein said lipid is cholesterol, a substituted or unsubstituted cholesterol, a cholesterol derivative, such as a hydroxylated cholesterol derivative (e.g., a hydroxycholesterol), a PEG-lipid, DMPC (1,2-Dimyristoyl-sn-glycero-3-phosphocholine), DSPC (1,2-Distearoyl-sn-glycero-3-phosphocholine), DODAP (1,2-dioleoyl-3-dimethylammonium propane), DDA (dimethyldioctadecylammonium), 1,2-dioleoyl-sn-glycero-3-phosphate, 1,2-dimyristoyl-sn-glycero-3-phosphate, bis(monooleoylglycerol)phosphate or a combination thereof. In some embodiments of the method of the present invention the protective polymer is a zwitterionic protective polymer, optionally wherein the zwitterionic protective polymer is electrostatically linked to the one or more polypeptide(s). Preferably, the zwitterionic protective polymer is selected from: poly(2-methacryloyloxyethyl phosphorylcholine) (PMPC), polyethyleneimine-g-poly(2-methacryloyloxyethyl phosphorylcholine) (PEI-g-PMPC), co-assembly of cationic (carboxyl-functionalized) and anionic (amino-functionalized) copolyesters based on poly(ε-caprolactone)-block-poly(butylene fumarate)-block-poly(ε-caprolactone) (PCL-b-PBF-b-PCL), poly(lactic-co-glycolic acid) (PLGA)-PCB block copolymers (PLGA-b-PCB).

In some embodiments of the methods of the present invention, the protective layer is linked to a targeting moiety, optionally wherein the protective layer linked to a targeting moiety is electrostatically linked to the drug(s), preferably wherein said drug is a negatively-charged drug.

In some embodiments of the methods of the present invention, the protective layer is linked to an antibody, optionally wherein the protective layer linked to an antibody is electrostatically linked to the drug(s), preferably wherein said drug is a negatively-charged drug.

In some embodiments of the method of the invention, the protective layer is linked to a carbohydrate, optionally wherein the protective layer linked to a carbohydrate is electrostatically linked to the one or more drug(s), preferably wherein said drug is a negatively-charged drug.

In further embodiments, the method of the present invention may involve a centrifugation step. The centrifugation step, within the context of the present invention enables the removal of the components comprising the mitochondrion delivery vehicle, e.g., unattached payload, such as the drug, the positively-charged species or the protective layer to facilitate the formation of the delivery vehicle. As the skilled person is aware, the centrifugation step may be performed after any step which requires removal of excess components of the delivery vehicle, e.g. excess payload, excess positive-charged species, excess protective layer. The centrifugation step may be added to the methods of attaching one or more polypeptide(s) to the mitochondrion as described hereinabove in an analogous manner as described hereinabove for the embodiments relating to the mitochondrion comprising the nucleic acid molecules.

### In a Further Aspect, the Invention Relates to a Mitochondrion Comprising Two or More of (a) to (c):

(a) one or more nucleic acid molecule(s) attached to the outer membrane of the mitochondrion
(b) one or more polypeptide(s) attached to the outer membrane of the mitochondrion,
(c) one or more drug(s) attached to the outer membrane of the mitochondrion, wherein the one or more nucleic acid molecule(s), polypeptide(s) and/or drug(s)
i) is/are electrostatically attached to the outer membrane of the mitochondrion, optionally is/are electrostatically attached to the outer membrane of the mitochondrion via a positively-charged species; or
ii) is/are covalently linked to the outer membrane of the mitochondrion; or
iii) is/are linked to an antibody that specifically binds to an antigen comprised in the outer membrane of the mitochondrion; or
iv) is/are linked to a mitochondria-targeting small molecule.

The one or more nucleic acid molecule(s) may be DNA and/or RNA.

The one or more polypeptide(s) may be a charged polypeptide. In particular, the charged polypeptide may be a negatively-charged polypeptide.

Alternatively, the charged polypeptide may be a positively-charged polypeptide.

The one or more drug(s) may be a charged drug. In this respect, the charged drug may be an anionic drug optionally wherein the anionic drug is selected from: potassium iodide, iodide, artesunate, sodium fluoride, carbamide peroxide, sodium zirconium cyclosilicate, nitrite, lithium carbonate, zinc chloride, aluminium hydroxide, magaldrate, aluminium sesquichlorohydrate, hydrotalcite, aluminium glycinate, aloglutamol, dihydroxyaluminium sodium carbonate, cystine, nitroprusside, montelukast, stepronin, prostaglandin G2, pyrophosphoric acid, OXI-4503, tetrachlorodecaoxide, NCX 701, PX-12, nitrous acid, chromic chloride, ferric pyrophosphate, activated charcoal, monopotassium phosphate, dipotassium phosphate, sodium fluorophosphate, potassium nitrate, potassium bicarbonate, sulfur hexafluoride, PF-4191834, allicin, artefenomel, lodenafil carbonate, devimistat, GW-274150, imrecoxib, chlorine dioxide, perflubutane, CHS-828, QGC-001, trabodenoson, magnesium phosphate, TAK-243, dostarlimab, GC-376 free acid, sodium metabisulfite, diquafosol, ammonium carbonate, NCX-1000 and ethyl nitrite, Nitroprusside, Technetium Tc-99m polyphosphate, Sodium phosphate monobasic, Sodium sulfate, Indium, Chromic nitrate, Tetrafluoroborate, Darapladib, PF-03715455 and Umifenovir.

The charged drug may also be a cationic drug, optionally wherein the cationic drug selected from Methyl-piperidino-pyrazole (MPP), Bretylium, Acetylcarnitine, Fluorocholine F-18, Hexamethonium, Edrophonium, Choline, Succinylcholine, Oxyphenonium, Carbamoylcholine, Gallamine triethiodide, Glycopyrronium, Bethanechol, Ambenonium, Methacholine, Betaine, Benzalkonium, Benzethonium, Emepronium, Benzoxonium, Gallamine, Octenidine, Methantheline, Propantheline, Tubocurarine, Neostigmine, Butylscopolamine, Alcuronium, Metocurine iodide, Levocarnitine, Hexafluronium, Decamethonium, Oxtriphylline, Metocurine, Choline magnesium trisalicylate, Platelet Activating Factor, N,N,N-Trimethyl-2-(phosphonooxy) ethanaminium, Butyrylthiocholine, Betaine aldehyde, C31G, Perifosine, Tetraethylammonium, Miltefosine, Citicoline, Benzododecinium, Choline salicylate, Cetyltrimethylammonium naproxenate and Trimethyltetradecylammonium.

In such an embodiment, the positively-charged polypeptide and/or the cationic drug(s) may be electrostatically attached to the outer membrane of the mitochondrion.

Alternatively, the one or more nucleic acid molecule(s), negatively-charged polypeptide(s) and/or anionic drug(s) may be electrostatically attached to the outer membrane of the mitochondrion via a positively-charged species.

The positively-charged species may be a polycationic species, wherein the polycationic species is a linear or branched polycationic polymer, optionally wherein the linear or branched polycationic polymer is electrostatically linked to the one or more nucleic acid molecules, the negatively-charged polypeptide and/or the anionic drug.

The linear or branched polycationic polymer may be polylysine, histidylated polylysine, polyornithine, polyarginine, high-mobility group protein (HMG) 1 and 17, modified chitosan, cationized human serum albumin, polyethyleneimine (PEI), polypropyleneimine (PPI), a cationic dendrimer, poly(2-(dimethylamino)ethyl methacrylate) (PDMAEMA), a polyallylamine derivative, diethylaminoethyl (DEAE)-dextran, poly(N-alkyl-4-vinylpyridinium), a poly(amidoamine), cationic gelatin, cationic cellulose or a combination thereof.

The positively-charged species may be a positively-charged nanoparticle.

The positively-charged species may be a positively-charged particle.

The one or more nucleic acid molecule(s), the one or more negatively-charged polypeptide(s) and/or the one or more anionic drug(s) may be attached to the surface of the positively-charged nanoparticle or encapsulated in the positively-charged nanoparticle.

The one or more nucleic acid molecule(s), the one or more negatively-charged polypeptide(s) and/or the one or more anionic drug(s) may be attached to the surface of the positively-charged particle or encapsulated in the positively-charged particle.

The positively-charged nanoparticle/particle may be a lipid nanoparticle/particle, a dendrimer nanoparticle/particle, a micelle nanoparticle/particle, a protein nanoparticle/particle, a liposome, a non-porous silica nanoparticle/particle, a mesoporous silica nanoparticle/particle, a silicon nanoparticle/particle, a gold nanoparticle/particle, a gold nanowire/wire, a silver nanoparticle/particle, a platinum nanoparticle/particle, a palladium nanoparticle/particle, a titanium dioxide nanoparticle/particle, a carbon nanotube, a carbon dot nanoparticle/particle, a polymer nanoparticle/particle, a zeolite nanoparticle/particle, an aluminium oxide nanoparticle/particle, a hydroxyapatite nanoparticle/particle, a quantum dot nanoparticle/particle, a zinc oxide nanoparticle/particle, a zirconium oxide nanoparticle/particle, graphene or a graphene oxide nanoparticle/particle.

The one or more nucleic acid molecule(s), one or more polypeptide(s) and/or one or more drug(s) may be linked to a second polypeptide in the outer membrane of the mitochondrion via an amide bond. The second polypeptide may be identical, similar or different to the primary polypeptide used in the methods provided herein.

The one or more nucleic acid molecule(s), one or more polypeptide(s) and/or one or more drug(s) may have been modified to undergo formation of the amide bond with an amine function comprised in the second polypeptide in the outer membrane of the mitochondrion.

The one or more nucleic acid molecule(s), the one or more polypeptide(s) and/or the one or more drug(s) may be encapsulated in a nanoparticle, and wherein the nanoparticle comprises a functional group that allows covalent linkage of the nanoparticle to a second polypeptide in the outer membrane of the mitochondrion.

The antibody may specifically bind to an antigen comprised in the outer membrane of the mitochondrion, wherein the antigen is OPA1, TOM70, TOMM20, Mitofusin 1, Mitofusin 2 or VDAC1.

The one or more nucleic acid molecule(s), the one or more polypeptide(s) and/or the one or more drug(s) may be encapsulated in a nanoparticle, and wherein the nanoparticle is covalently linked to the antibody.

The one or more nucleic acid molecule(s), one or more polypeptide(s) and/or one or more anionic drug(s) may be electrostatically linked to the antibody, wherein the antibody is a modified antibody, wherein the modified antibody possesses one or more positive charge(s).

The one or more nucleic acid molecule(s), one or more polypeptide(s) and/or one or more drug(s) may be covalently linked to biotin, wherein biotin is linked to the antibody, wherein the antibody is an avidin conjugated antibody.

The one or more nucleic acid molecule(s), one or more polypeptide(s) and/or one or more drug(s) may be covalently linked to an activated ester, wherein the activated ester is linked to the antibody via an amide bond.

The mitochondrion of the invention may comprise one or more nucleic acid molecule(s), wherein the one or more nucleic acid molecule(s) is a single-stranded nucleic acid molecule (ssDNA or ssRNA), wherein the single-stranded nucleic acid molecule is hybridized with one or more complementary single-stranded nucleic acid molecule attached on or to the antibody.

The one or more nucleic acid molecule(s), one or more polypeptide(s) and/or one or more drug(s) may be encapsulated in a nanoparticle, wherein the nanoparticle is electrostatically linked to the antibody, wherein the antibody is a modified antibody, wherein the modified antibody possesses one or more positive charge(s).

The one or more nucleic acid molecule(s), one or more polypeptide(s) and/or one or more drug(s) may be encapsulated in a nanoparticle, wherein the nanoparticle is covalently linked to biotin, wherein biotin is linked the antibody, wherein the antibody is an avidin conjugated antibody.

The one or more nucleic acid molecule(s), one or more polypeptide(s) and/or one or more drug(s) may be encapsulated in a nanoparticle, wherein the nanoparticle is covalently linked to an activated ester, wherein the activated ester is linked to the antibody via an amide bond.

The mitochondria-targeting small molecule may be selected from the group consisting of triphenylphosphonium (TPP), dequalinium (DQA), E-4-(1H-Indol-3-ylvinyl)-N-Methylpyridineiodide (F16), Rhodamine 19, biguanidine and guanidine.

The mitochondrion may comprise one or more nucleic acid molecule(s) and one or more cationic drug(s), wherein the cationic drug is electrostatically linked to the one or more nucleic acid molecules.

The mitochondrion may be linked to and/or enveloped in a protective layer.

The protective layer may be a protective polymer.

The protective polymer may be a linear or branched cationic polymer, optionally wherein the linear or branched cationic polymer is electrostatically linked to the one or more nucleic acid molecule(s) and/or the one or more negatively-charged polypeptide(s) and/or the one or more anionic drug(s).

The protective polymer may be a linear or branched cationic block copolymer, optionally wherein the linear or branched cationic block copolymer is electrostatically linked to the one or more nucleic acid molecule(s) and/or the one or more negatively-charged polypeptide(s) and/or the one or more anionic drug(s).

The protective polymer may be a cationic graft (g) copolymer, optionally wherein the cationic graft (g) copolymer is electrostatically linked to the one or more nucleic acid molecule(s) and/or the one or more negatively-charged polypeptide(s) and/or the one or more anionic drug(s).

The protective polymer may be a linear or branched pegylated (PEG) cationic polymer, optionally wherein the linear or branched pegylated (PEG) cationic polymer is electrostatically linked to the one or more nucleic acid molecule(s) and/or the one or more negatively-charged polypeptide(s) and/or the one or more anionic drug(s).

The protective layer may be a lipid formulation, optionally wherein the lipid formulation is a cationic lipid formulation, further optionally wherein the cationic lipid formulation is electrostatically linked to the one or more nucleic acid molecule(s) and/or the one or more negatively-charged polypeptide(s) and/or the one or more anionic drug(s).

The protective layer may be linked to a targeting moiety, optionally wherein the protective layer linked to a targeting moiety is electrostatically linked to the one or more nucleic acid molecule(s) and/or the one or more negatively-charged polypeptide(s) and/or the one or more anionic drug(s).

The protective layer may be linked to an antibody, optionally wherein the protective layer linked to an antibody is electrostatically linked to the one or more nucleic acid molecule(s) and/or the one or more negatively-charged polypeptide(s) and/or the one or more anionic drug(s).

The protective layer may be linked to a carbohydrate, optionally wherein the protective layer linked to a carbohydrate is electrostatically linked to the one or more nucleic acid molecule(s) and/or the one or more negatively-charged polypeptide(s) and/or the one or more anionic drug(s).

The linear or branched cationic polymer may be polyethyleneimine, RGD-modified polyethyleneimine, polylysine, RGD-modified polylysine, polyornithine, RGD-modified polyornithine, polyarginine, RGD modified polyarginine, polypropyleneimine, RGD-modified polypropyleneimine, polyallylamine, RGD-modified polyallylamine, chitosan, RGD-modified chitosan, poly(2-(dimethylamino)ethyl methacrylate), RGD-modified poly(2-(dimethylamino)ethyl methacrylate), poly(amidoamine)s, RGD-modified poly(amidoamine) s or a combination thereof.

The cationic block copolymer may be poly(ethylene glycol)-block-polyethyleneimine, RGD-modified poly(ethylene glycol)-block-polyethyleneimine, poly(ethylene glycol)-block-polylysine, RGD-modified poly(ethylene glycol)-block-polylysine, poly(ethylene glycol)-block-polyornithine, RGD-modified poly(ethylene glycol)-block-polyornithine, poly(ethylene glycol)-block-polyarginine, RGD-modified poly(ethylene glycol)-block-polyarginine, poly(ethylene glycol)-block-polypropyleneimine, RGD-modified poly(ethylene glycol)-block-polypropyleneimine, poly(ethylene glycol)-block-polyallylamine, RGD-modified poly(ethylene glycol)-block-polyallylamine, poly(ethylene glycol)-block-poly(2-(dimethylamino)ethyl methacrylate), RGD-modified poly(ethylene glycol)-block-poly(2-(dimethylamino)ethyl methacrylate), poly(ethylene glycol)-block-poly(amidoamine)s, RGD-modified poly(ethylene glycol)-block-poly(amidoamine)s or a combination thereof.

The cationic graft (g) copolymer may be poly(ethylene glycol)-g-polyethyleneimine, RGD-modified poly(ethylene glycol)-g-polyethyleneimine, poly(ethylene glycol)-g-polylysine, RGD-modified poly(ethylene glycol)-g-polylysine, poly(ethylene glycol)-g-polyornithine, RGD-modified poly(ethylene glycol)-g-polyornithine, poly(ethylene glycol)-g-polyarginine, RGD-modified poly(ethylene glycol)-g-polyarginine, poly(ethylene glycol)-g-polypropyleneimine, RGD-modified poly(ethylene glycol)-g-polypropyleneimine, poly(ethylene glycol)-g-polyallylamine, RGD-modified poly(ethylene glycol)-g-polyallylamine, poly(ethylene glycol)-g-poly(2-(dimethylamino)ethyl methacrylate), RGD-modified poly(ethylene glycol)-g-poly(2-(dimethylamino)ethyl methacrylate), poly(ethylene glycol)-g-poly(amidoamine)s, RGD-modified poly(ethylene glycol)-g-poly(amidoamine)s or a combination thereof.

The pegylated (PEG) cationic polymer may be pegylated-polyethyleneimine, RGD-modified pegylated polyethyleneimine, pegylated polylysine, RGD-modified pegylated polylysine, histidylated polylysine, pegylated polyornithine, RGD-modified pegylated polyornithine, pegylated polyarginine, RGD-modified pegylated polyarginine, pegylated polypropyleneimine, RGD-modified pegylated polypropyleneimine, pegylated polyallylamine, RGD-modified pegylated polyallylamine, pegylated chitosan, RGD-modified pegylated chitosan, pegylated poly(2-(dimethylamino)ethyl methacrylate), RGD-modified pegylated poly(2-(dimethylamino)ethyl methacrylate), pegylated poly(amidoamine)s RGD-modified pegylated poly(amidoamine)s or a combination thereof.

The lipid formulation may comprise DC-cholesterol (3β-[N—(N′,N′-Dimethylaminoethane)-carbamoyl]cholesterol hydrochloride), DLinDMA (1,2-dilinoleyloxy-3-dimethylaminopropane), DLinMC3DMA (dilinoleylmethyl-4-dimethylaminobutyrate), DODMA (1,2-dioleyloxy-3-dimethylaminopropane), DOGS (dioctadecylamidoglycylspermine), DOSPA (2,3-dioleyloxy-N-[2 (sperminecarboxamido) ethyl]-N,N-dimethyl-1-propanaminium), DOTAP (1,2-dioleoyl-3-trimethylammonium-propane chloride), DOTMA (1,2-di-O-octadecenyl-3-trimethylammonium propane chloride), UGG (unsaturated guanidinium glycoside), DOPE (1,2-Dioleoyl-sn-glycerophosphoethanolamine), lipofectamine or a combination thereof.

The lipid formulation may further comprise another lipid, preferably wherein said lipid is cholesterol, a substituted or unsubstituted cholesterol, a cholesterol derivative, such as a hydroxylated cholesterol derivative (e.g., a hydroxycholesterol), a PEG-lipid, DMPC (1,2-Dimyristoyl-sn-glycero-3-phosphocholine), DSPC (1,2-Distearoyl-sn-glycero-3-phosphocholine), DODAP (1,2-dioleoyl-3-dimethylammonium propane), DDA (dimethyldioctadecylammonium), 1,2-dioleoyl-sn-glycero-3-phosphate, 1,2-dimyristoyl-sn-glycero-3-phosphate, bis(monooleoylglycerol)phosphate or a combination thereof.

The mitochondrion may be linked to and/or enveloped in a zwitterionic protective polymer, optionally wherein the zwitterionic protective polymer is electrostatically linked to the one or more nucleic acid molecule(s).

The zwitterionic protective polymer may be selected from: poly(2-methacryloyloxyethyl phosphorylcholine) (PMPC), polyethyleneimine-g-poly(2-methacryloyloxyethyl phosphorylcholine) (PEI-g-PMPC), co-assembly of cationic (carboxyl-functionalized) and anionic (amino-functionalized) copolyesters based on poly(ε-caprolactone)-block-poly(butylene fumarate)-block-poly(ε-caprolactone) (PCL-b-PBF-b-PCL), poly(lactic-co-glycolic acid) (PLGA)-PCB block copolymers (PLGA-b-PCB).

Further provided herein is a composition comprising a plurality of mitochondria according to the embodiments provided herein above.

Also provided is a pharmaceutical composition comprising a plurality of mitochondria according to the invention and a pharmaceutically acceptable carrier. The pharmaceutical composition may be formulated as a solution.

The pharmaceutical composition may also be formulated as an aerosol.

Further provided is the mitochondrion of the invention, the composition of the invention or the pharmaceutical composition of the invention for use as a medicament.

The present invention also relates to the mitochondrion according to the present invention, the composition according to the present invention and/or the pharmaceutical composition according to the present invention for use in gene therapy.

The present invention also relates to the mitochondrion according to the present invention, the composition according to the present invention and/or the pharmaceutical composition according to the present invention for use in the treatment of cardiovascular diseases, in particular for use in the treatment of ischemic heart disease, ischemia-reperfusion injury and/or atherosclerosis.

The present invention also relates to the mitochondrion according to the present invention, the composition according to the present invention and/or the pharmaceutical composition according to the present invention for use in the treatment of aging related diseases, in particular for use in the treatment of, sarcopenia, Parkinson's disease or Hutchinson-Gilford progeria syndrome.

The present invention also relates to the mitochondrion according to the present invention, the composition according to the present invention and/or the pharmaceutical composition according to the present invention for use in the treatment of kidney diseases, in particular for use in the treatment of, autosomal dominant polycystic kidney disease, Alport syndrome, Nephronophthisis, or Fabry disease.

The present invention also relates to the mitochondrion according to the present invention, the composition according to the present invention and/or the pharmaceutical composition according to the present invention for use in the treatment of cancer.

The present invention also relates to the mitochondrion according to the present invention, the composition according to the present invention and/or the pharmaceutical composition according to the present invention for use in in vitro, ex vivo, or in vivo genome editing.

The present invention also relates to the mitochondrion according to the present invention, the composition according to the present invention and/or the pharmaceutical composition according to the present invention for use in radiation therapy.

Also provided is a method for delivering an active agent to a target organ, the method comprising a step of administering the pharmaceutical composition of the invention into the bloodstream of a subject in need, wherein the pharmaceutical composition is administered into the bloodstream upstream of the target organ.

A method for delivering an active agent to the lung, the method comprising a step of administering the pharmaceutical composition of the invention to a subject in need, wherein the pharmaceutical composition is administered by inhalation, is also provided.

Further Provided is a Method for Attaching Two or More of (i) to (iii):
i) one or more nucleic acid molecule(s);
ii) one or more polypeptide(s);
iii) and/or one or more drug(s);

### To the Outer Membrane of a Mitochondrion, the Method Comprising the Steps of:

a) providing a preparation of mitochondria;
b) contacting the mitochondria provided in step (a) with one or more nucleic acid molecule(s), one or more polypeptide(s) and/or one or more drug(s), optionally in the presence of a positively-charged species; and
c) attaching the one or more nucleic acid molecule(s), one or more polypeptide(s) and/or one or more drug(s) to the mitochondria, optionally via the positively-charged species.

### The Method May Further be Defined, Wherein

a) the one or more nucleic acid molecule(s), one or more polypeptide(s) and/or one or more drug(s) are contacted with the positively-charged species to form a positively-charged complex before the positively-charged complex is contacted with the mitochondria; or
b) the mitochondrion is contacted with the positively-species and subsequently contacted with the one or more nucleic acid molecule(s), one or more polypeptide(s) and/or one or more drug(s).

Within the present invention, the mitochondria may be contacted with one or more nucleic acid molecule(s), one or more polypeptide(s), and/or one or more drug(s) and the positively-charged species in a suitable buffer.

The buffer may comprise or consist of HEPES, EGTA, Trehalose CHES and sodium phosphate dibasic dihydrate, preferably wherein buffer comprises a mixture of a Solution X comprising or consisting of HEPES, EGTA and Trehalose and of a Solution Y comprising or consisting of CHES and sodium phosphate dibasic dihydrate, more preferably, wherein the buffer comprises a 4:1 mixture of Solution X comprising or consisting of 20 mM HEPES, 1 mM EGTA and 300 mM Trehalose (pH 7.2) and Solution Y comprising or consisting of 0.1 M CHES (pH 10) and 0.2 M sodium phosphate dibasic dihydrate.

The mitochondria may be contacted with the one or more nucleic acid molecule(s), one or more polypeptide(s) and/or one or more drug(s) and the positively-charged species at room temperature for at least 5 minutes, such as at least 10 minutes, 20, 30, 40, 50, 60, 70, 80, 90, 100, 110 or 120 minutes.

The mitochondria may be contacted with the one or more nucleic acid molecule(s), one or more polypeptide(s) and/or one or more drug(s) and the positively-charged species in the dark.

The positively-charged species may be a polycationic species, wherein the polycationic species is a linear or branched polycationic polymer, optionally wherein the linear or branched polycationic polymer is electrostatically linked to the one or more nucleic acid molecule(s), one or more polypeptide(s) and/or one or more drug(s).

The linear or branched polycationic polymer may be polylysine, histidylated polylysine, polyornithine, polyarginine, high-mobility group protein (HMG) 1 and 17, modified chitosan, cationized human serum albumin, polyethyleneimine (PEI), polypropyleneimine (PPI), a cationic dendrimer, poly(2-(dimethylamino)ethyl methacrylate) (PDMAEMA), a polyallylamine derivative, diethylaminoethyl (DEAE)-dextran, poly(N-alkyl-4-vinylpyridinium), a poly(amidoamine), cationic gelatin, cationic cellulose or a combination thereof.

The positively-charged species may be a positively-charged nanoparticle.

### The Method May Comprise a Further Step of

a) attaching the one or more nucleic acid molecule(s), one or more polypeptide(s) and/or one or more drug(s) to the surface of the positively-charged nanoparticle; or
b) encapsulating the one or more nucleic acid molecule(s), one or more polypeptide(s) and/or one or more drug(s) within the positively-charged nanoparticle.

The positively-charged nanoparticle may be a lipid nanoparticle, a dendrimer nanoparticle, a micelle nanoparticle, a protein nanoparticle, a liposome, a non-porous silica nanoparticle, a mesoporous silica nanoparticle, a silicon nanoparticle, a gold nanoparticle, a gold nanowire, a silver nanoparticle, a platinum nanoparticle, a palladium nanoparticle, a titanium dioxide nanoparticle, a carbon nanotube, a carbon dot nanoparticle, a polymer nanoparticle, a zeolite nanoparticle, an aluminium oxide nanoparticle, a hydroxyapatite nanoparticle, a quantum dot nanoparticle, a zinc oxide nanoparticle, a zirconium oxide nanoparticle, graphene or a graphene oxide nanoparticle.

Further Provided is a Method for Covalently Attaching Two or More of (i) to (iii):
i) one or more nucleic acid molecule(s);
ii) one or more polypeptide(s);
iii) and/or one or more drug(s);
to the outer membrane of a mitochondrion, the method comprising the steps of:
a) providing a preparation of mitochondria;
b) providing one or more nucleic acid molecule(s), one or more polypeptide(s) and/or one or more drug(s) that have been modified to comprise an activated ester; and
c) attaching the one or more nucleic acid molecule(s), one or more polypeptide(s) and/or one or more drug(s) provided in step (b) to an amine comprised in a polypeptide in the outer membrane of the mitochondria.

The activated ester may be an N-hydroxysuccinimide (NHS) ester.

Also Provided is a Method for Covalently Attaching Two or More of (i) to (iii):
i) one or more nucleic acid molecule(s);
ii) one or more polypeptide(s);
iii) and/or one or more drug(s);
to the outer membrane of a mitochondrion, the method comprising the steps of:
a) providing a preparation of mitochondria;
b) encapsulating one or more nucleic acid molecule(s), one or more polypeptide(s) and/or one or more drug(s) in a nanoparticle, wherein the surface of the nanoparticle comprises an activated ester; and
c) attaching the nanoparticle provided in step (b) to an amine comprised in a polypeptide in the outer membrane of the mitochondria.

The method according to the invention, wherein the activated ester is an NHS ester.

The methods of the present invention may further comprise a step of adding a protective layer.

Accordingly, in some embodiments, the method of the invention further comprises linking to and/or enveloping the mitochondrion in a protective layer. The mitochondrion may be any mitochondrion as described hereinabove and any protective layer as described hereinabove. In preferred embodiments, the invention is a method for attaching one or more drug(s) to the outer membrane of a mitochondrion, wherein the method comprises the steps of:
a) providing a preparation of mitochondria;
b) contacting the mitochondria provided in step (a) with one or more nucleic acid molecule(s), one or more polypeptide(s) and/or one or more drug(s);
c) attaching the at least one of b) to the mitochondria; and
d) linking and/or enveloping the mitochondrion provided in steps (a) to (c) with a protective layer.

Accordingly, the invention also provides for a method comprising linking covalently the nucleic acid to a protective layer, optionally wherein the protective layer is linked to a targeting moiety (e.g. antibody/carbohydrate) and linking to and/or enveloping a mitochondrion in the protective layer, optionally in presence of positively charged species. In this method, the linking of the nucleic acid to the protective layer may be on the surface of the protective layer opposite to the surface of the same protective layer linked to the targeting moiety. Preferably the nucleic acid is liked on the inner surface of the protective layer.

In some embodiments, the protective layer is a protective polymer. The protective polymer is as described herein above.

In some embodiments of the method of the present invention, the protective polymer is a linear or branched cationic polymer, optionally wherein the linear or branched cationic polymer is electrostatically linked to the one or more polypeptide(s), preferably wherein said polypeptide is a negatively-charged polypeptide. Preferably, the linear or branched cationic polymer is polyethyleneimine, RGD-modified polyethyleneimine, polylysine, RGD-modified polylysine, polyornithine, RGD-modified polyornithine, polyarginine, RGD modified polyarginine, polypropyleneimine, RGD-modified polypropyleneimine, polyallylamine, RGD-modified polyallylamine, chitosan, RGD-modified chitosan, poly(2-(dimethylamino)ethyl methacrylate), RGD-modified poly(2-(dimethylamino)ethyl methacrylate), poly(amidoamine)s, RGD-modified poly(amidoamine)s or a combination thereof. In some embodiments of the method of the present invention, the protective polymer is a linear or branched cationic block copolymer, optionally wherein the linear or branched cationic block copolymer is electrostatically linked to the one or more polypeptide(s), preferably wherein said polypeptide is a negatively-charged polypeptide. Preferably, the cationic block copolymer is poly(ethylene glycol)-block-polyethyleneimine, RGD-modified poly(ethylene glycol)-block-polyethyleneimine, poly(ethylene glycol)-block-polylysine, RGD-modified poly(ethylene glycol)-block-polylysine, poly(ethylene glycol)-block-polyornithine, RGD-modified poly(ethylene glycol)-block-polyornithine, poly(ethylene glycol)-block-polyarginine, RGD-modified poly(ethylene glycol)-block-polyarginine, poly(ethylene glycol)-block-polypropyleneimine, RGD-modified poly(ethylene glycol)-block-polypropyleneimine, poly(ethylene glycol)-block-polyallylamine, RGD-modified poly(ethylene glycol)-block-polyallylamine, poly(ethylene glycol)-block-poly(2-(dimethylamino)ethyl methacrylate), RGD-modified poly(ethylene glycol)-block-poly(2-(dimethylamino)ethyl methacrylate), poly(ethylene glycol)-block-poly(amidoamine)s, RGD-modified poly(ethylene glycol)-block-poly(amidoamine)s or a combination thereof.

In some embodiments of the method of the present invention, the protective polymer is a linear or branched cationic graft (g) copolymer, optionally wherein the linear or branched cationic graft (g) copolymer is electrostatically linked to the one or more polypeptide(s), preferably wherein said polypeptide is a negatively-charged polypeptide. Preferably, the cationic graft (g) copolymer is poly(ethylene glycol)-g-polyethyleneimine, RGD-modified poly(ethylene glycol)-g-polyethyleneimine, poly(ethylene glycol)-g-polylysine, RGD-modified poly(ethylene glycol)-g-polylysine, poly(ethylene glycol)-g-polyornithine, RGD-modified poly(ethylene glycol)-g-polyornithine, poly(ethylene glycol)-g-polyarginine, RGD-modified poly(ethylene glycol)-g-polyarginine, poly(ethylene glycol)-g-polypropyleneimine, RGD-modified poly(ethylene glycol)-g-polypropyleneimine, poly(ethylene glycol)-g-polyallylamine, RGD-modified poly(ethylene glycol)-g-polyallylamine, poly(ethylene glycol)-g-poly(2-(dimethylamino)ethyl methacrylate), RGD-modified poly(ethylene glycol)-g-poly(2-(dimethylamino)ethyl methacrylate), poly(ethylene glycol)-g-poly(amidoamine)s, RGD-modified poly(ethylene glycol)-g-poly(amidoamine)s or a combination thereof. In some embodiments of the method of the present invention, the protective polymer is a linear or branched pegylated (PEG) cationic polymer, optionally wherein the linear or branched pegylated (PEG) cationic polymer is electrostatically linked to the one or more polypeptide(s), preferably wherein said polypeptide is a negatively-charged polypeptide. Preferably, the pegylated (PEG) cationic polymer is pegylated-polyethyleneimine, RGD-modified pegylated polyethyleneimine, pegylated polylysine, RGD-modified pegylated polylysine, histidylated polylysine, pegylated polyornithine, RGD-modified pegylated polyornithine, pegylated polyarginine, RGD-modified pegylated polyarginine, pegylated polypropyleneimine, RGD-modified pegylated polypropyleneimine, pegylated polyallylamine, RGD-modified pegylated polyallylamine, pegylated chitosan, RGD-modified pegylated chitosan, pegylated poly(2-(dimethylamino)ethyl methacrylate), RGD-modified pegylated poly(2-(dimethylamino)ethyl methacrylate), pegylated poly(amidoamine)s RGD-modified pegylated poly(amidoamine)s or a combination thereof.

In some embodiments of the method of the present invention, the protective layer is a lipid formulation, optionally wherein the lipid formulation is a cationic lipid formulation, further optionally wherein the cationic lipid formulation is electrostatically linked to the one or more polypeptide(s), preferably wherein said polypeptide is a negatively-charged polypeptide. Preferably, wherein the lipid formulation comprises DC-cholesterol (3β-[N—(N′,N′-Dimethylaminoethane)-carbamoyl]cholesterol hydrochloride), DLinDMA (1,2-dilinoleyloxy-3-dimethylaminopropane), DLinMC3DMA (dilinoleylmethyl-4-dimethylaminobutyrate), DODMA (1,2-dioleyloxy-3-dimethylaminopropane), DOGS (dioctadecylamidoglycylspermine), DOSPA (2,3-dioleyloxy-N-[2 (sperminecarboxamido)ethyl]-N,N-dimethyl-1-propanaminium), DOTAP (1,2-dioleoyl-3-trimethylammonium-propane chloride), DOTMA (1,2-di-O-octadecenyl-3-trimethylammonium propane chloride), UGG (unsaturated guanidinium glycoside), DOPE (1,2-Dioleoyl-sn-glycerophosphoethanolamine), lipofectamine or a combination thereof. In further embodiments, the lipid formulation further comprises another lipid, preferably wherein said lipid is cholesterol, a substituted or unsubstituted cholesterol, a cholesterol derivative, such as a hydroxylated cholesterol derivative (e.g., a hydroxycholesterol), a PEG-lipid, DMPC (1,2-Dimyristoyl-sn-glycero-3-phosphocholine), DSPC (1,2-Distearoyl-sn-glycero-3-phosphocholine), DODAP (1,2-dioleoyl-3-dimethylammonium propane), DDA (dimethyldioctadecylammonium), 1,2-dioleoyl-sn-glycero-3-phosphate, 1,2-dimyristoyl-sn-glycero-3-phosphate, bis(monooleoylglycerol)phosphate or a combination thereof.

In some embodiments of the method of the present invention the protective polymer is a zwitterionic protective polymer, optionally wherein the zwitterionic protective polymer is electrostatically linked to the one or more polypeptide(s). Preferably, the zwitterionic protective polymer is selected from: poly(2-methacryloyloxyethyl phosphorylcholine) (PMPC), polyethyleneimine-g-poly(2-methacryloyloxyethyl phosphorylcholine) (PEI-g-PMPC), co-assembly of cationic (carboxyl-functionalized) and anionic (amino-functionalized) copolyesters based on poly(ε-caprolactone)-block-poly(butylene fumarate)-block-poly(ε-caprolactone) (PCL-b-PBF-b-PCL), poly(lactic-co-glycolic acid) (PLGA)-PCB block copolymers (PLGA-b-PCB).

In some embodiments of the methods of the present invention, the protective layer is linked to a targeting moiety, optionally wherein the protective layer linked to a targeting moiety is electrostatically linked to the one or more nucleic acid molecule(s) and/or the one or more negatively-charged polypeptide(s) and/or the one or more anionic drug(s).

In some embodiments of the methods of the present invention, the protective layer is linked to an antibody, optionally wherein the protective layer linked to an antibody is electrostatically linked to the polypeptide(s), preferably wherein said polypeptide is a negatively-charged polypeptide.

In some embodiments of the method of the invention, the protective layer is linked to a carbohydrate, optionally wherein the protective layer linked to a carbohydrate is electrostatically linked to the one or more polypeptide(s), preferably wherein said polypeptide is a negatively-charged polypeptide.

In further embodiments, the method of the present invention may involve a centrifugation step. The centrifugation step, within the context of the present invention enables the removal of the components comprising the mitochondrion delivery vehicle, e.g., unattached payload, such as the nucleic acid molecule, polypeptide and/or drug, the positively-charged species or the protective layer to facilitate the formation of the delivery vehicle. As the skilled person is aware, the centrifugation step may be performed after any step which requires removal of excess components of the delivery vehicle, e.g. excess payload, excess positive-charged species, excess protective layer. The centrifugation step may be added to the methods of attaching one or more polypeptide(s) to the mitochondrion as described hereinabove in an analogous manner as described hereinabove for the embodiments relating to the mitochondrion comprising the nucleic acid molecules.

In one aspect, the methods of the present invention are not methods for treatment of the human or animal body by therapy. In a further aspect, the methods of the present invention are not processes for modifying the germ line genetic identity of human beings. In one aspect, the methods of the present invention are in vitro or ex vivo methods.

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art. As appropriate, procedures involving the use of commercially available kits and reagents are generally carried out in accordance with manufacturer-defined protocols and conditions unless otherwise noted.

As used herein, the singular forms “a,” “an,” and “the” include the plural referents unless the context clearly indicates otherwise. The terms “include,” “such as,” and the like are intended to convey inclusion without limitation, unless otherwise specifically indicated.

As used herein, the term “or” is generally employed in its usual sense including “and/or” unless the content clearly dictates otherwise. The term “and/or” means one or all of the listed elements or a combination of any two or more of the listed elements.

As used herein, the term “comprising” also specifically includes embodiments “consisting of” and “consisting essentially of” the recited elements, unless specifically indicated otherwise.

As used herein, the term “about” indicates and encompasses an indicated value and a range above and below that value. In certain embodiments, the term “about” indicates the designated value ±10%, ±5%, or ±1%. In certain embodiments, where applicable, the term “about” indicates the designated value(s)±one standard deviation of that value(s).

As used herein, the term “linked” can mean that a first compound or moiety is attached to a second compound or moiety either directly or indirectly. Linkage of compounds or moieties is not particularly limited in the present invention. In the sense of the present invention a compound or moiety may be e.g. electrostatically linked or linked by a covalent bond.

The invention is illustrated by the following examples.

### BRIEF DESCRIPTION OF THE FIGURES

FIG. 1: Description of mitochondria payload and their potential applications.

FIG. 2: A. Description of attachment of oligonucleotides on mitochondrial surface based on electrostatic interaction via a positively-charged species, B. based on covalent interaction, C. based on nanoparticle attachment via covalent bond/electrostatic interaction, D. via antibody-antigen interaction and E. via mitochondria-targeting small molecule.

FIG. 3: A. Fluorescence micrograph showing the colocalization of MitoTracker™ Red CMXRos and FAM-labeled DNA signals, indicating the successful functionalization of fluorescently labeled DNA molecules (FAM-ssDNA) on the mitochondria surface. B. Flow cytometry (FACS) data also confirms the presence of double staining signals, suggesting the availability of the DNA on mitochondria outer membrane. The sample was stored 5 days at −80° C. before FACS experiment was conducted.

FIG. 4: A. Colocalization of MitoTracker™ Red CMXRos and FAM-labeled DNA signal under fluorescence microscope showing stability of the synthesized mitochondria-DNA complex in cell culture medium (post 22 h). B. Flow cytometry analysis detects the presence of both fluorescence signals in the complex. The complex was previously stored at −80° C. for 5 and 60 days. FACS data suggest that samples are stable upon long term storage.

FIG. 5: Brightfield and fluorescence images showing internalization of mitochondria-DNA complex by human cardiac fibroblast (HCF). Mitochondria-DNA complex is shown by the arrow; left panel). Fluorescence micrographs show integration of mitochondria-DNA complex into existing mitochondrial network in HCF cells. RI denotes refractive index image.

FIG. 6: A. Fluorescence microscopy imaging and particle tracking reveal the internal transport of mitochondria-DNA complex from one mitochondrial network to the next network. B. Preparation steps to engineer a 3D coculture cells consisting of A549 cells and HCF cells. C. Top view and side view of 3D image showing the uptake of mitochondria-ssDNA complex in the 3D cell model. 3D visualization of the coculture revealed that majority of the complex were uptaken by A549 cells (apical side). In addition, the complex was able to penetrate the cellQART membrane insert and reach the basolateral side of the coculture (HCF cells). Oval structures highlight cell nuclei. Arrows denote the penetrated complex in HCF cells.

FIG. 7: Fluorescence microscopy imaging of plasmid DNA (pDNA)-transfected cells shows the presence of fluorescence staining in mitochondria network inside the cells. The cells were transfected with pDNA using Lipofectamine.

FIG. 8: A. Expression of GFP (Green Fluorescent Protein) signals inside HCF cells (white arrow) indicates the successful in vitro delivery of plasmid DNA (pDNA) by mitochondria (bottom panel). B. Naked DNA plasmid (DNA only) is used as a negative control. Time point: after 4 days. The GFP and MitoTracker panel highlights the expressed GFP signal and MitoTracker™ Red CMXRos-labeled mitochondria inside the cells, respectively. Overlay panels show the overlay image between the two channels and the respective bright field images.

FIG. 9: A. Fluorescence micrograph shows the colocalization of MitoTracker™ Red CMXRos and FAM-labeled ssRNA signals, indicating the successful functionalization of fluorescently labeled RNA molecules on the mitochondria surface. B. Transplantation of mitochondria-ssRNA complex in HCF cells was observed after 24 h of incubation (pointed out by arrows).

FIG. 10: Fluorescence micrographs showing successful internalization and translation of StemMACS™ Nuclear EGFP (Enhanced GFP) mRNA carried by mitochondria in HepG2 cancer cells. Translation of StemMACS™ Nuclear EGFP mRNA is seen by the presence of fluorescence signal in nuclei of the cells. The results were confirmed through a positive control experiment using Lipofectamine as delivery agent. Concentration of StemMACS™ Nuclear EGFP mRNA were varied from 3 to 9 μmol (for 40 μg/mL of mitochondria). Negative control shows untreated cells.

FIG. 11: Fluorescence micrographs showing successful internalization of Silencer™ FAM-labeled GAPDH (Glyceraldehyde 3 phosphate dehydrogenase) siRNA carried by mitochondria in HepG2 cancer cells. The results were confirmed through positive control experiment using widely used Lipofectamine as delivery agent. Concentration of Silencer™ FAM-labeled GAPDH siRNA were varied from 3 to 9 μmol (for 40 μg/mL of mitochondria). Negative control shows untreated cells.

FIG. 12: A. Cell counting assay confirmed through a DAPI staining. B. Cell counting reveals HepG2 cells treated with mitochondria-Silencer™ FAM-labeled GAPDH siRNA or mitochondria-Ambion™ MDM2 siRNA (60 μg/mL for 48 h) complex show a significant reduction in cell proliferation. C. The result is further confirmed by MTS assay. Lower absorbance value of cells treated with mitochondria-Ambion™ MDM2 siRNA complex in comparison to untreated cells is observed. In the case of cells treated with mitochondria-Silencer™ FAM-labeled GAPDH siRNA, increase in absorbance is due to the presence of FAM-labeled siRNA. FAM (fluorescein) molecule has a maximum peak of absorbance at 488 nm.

FIG. 13: A. Western Blot as an assay to measure protein knockdown. B. Reduced expression of GAPDH was observed in mitochondria-Silencer™ FAM-labeled GAPDH siRNA complex-treated HepG2 cells, in comparison to untreated cells. Quantification of the intensity was performed using image processing in Fiji.

FIG. 14: A. Description of spheroid (cancer) invasion assay. B. Fluorescence and brightfield micrograph showing the formation of cancer spheroid. Cell nuclei were stained with DAPI for visualization. C. Escaped cells were monitored 3 days after spheroid seeding. Round object indicates the nucleus of a single cell. No significant reduction in cell invasion was observed in all samples.

FIG. 15: A. In vivo study of biodistribution of mitochondria-ssDNA complex inside a heart of a pig. Mitochondria-ssDNA complex (1 mg/mL; in 5 mL of the conjugation buffer) was directly injected into the heart. Post two hours, the pig was sacrificed and a small piece of heart tissue where the injection occurred was cut. B. The tissue was fixed using formaldehyde and a histology cut was performed. The tissue was stained with DAPI and rhodamine phalloidin for visualization of cell nuclei and F-actin networks, respectively. FAM signal represents ssDNA signal. The control experiment was prepared by direct injection of naked ssDNA. 1 mg/mL of mitochondria corresponds to ca. 3 billion mitochondria/mL.

FIG. 16: A. Image showing the collection of the sample on well-plate during nebulization. B. Fluorescence micrographs showing the presence of mitochondria and mitochondria-ssDNA complex in the nebulized sample. Dispersion control denotes the samples which were taken before nebulization at concentration of 1 mg/mL. For nebulization, the concentration of the liquid was reduced to 0.4 mg/mL. Red signal shows fluorescence of MitoTracker™ Red CMXRos (for mitochondria) and green signals shows fluorescently labeled ssDNA.

FIG. 17: Fluorescence micrograph showing internalization of aerosolized mitochondria-ssDNA complex by HepG2 cells. Aerosolized mitochondria-ssDNA complex was introduced to the cells for 30 seconds and after the nebulization, the cells were kept inside incubator for 20 h before imaging experiment was conducted. Red signal shows fluorescence of MitoTracker™ Red CMXRos (for mitochondria) and green signals shows fluorescently labeled ssDNA. Dash line shows cell border.

FIG. 18: Overview location of A. Cortical and B. Medullar kidney biopsies. C. Scheme of sample locations and the respective macroscopy annotations.

FIG. 19: Representative images of HE staining of medulla and cortex acquired using the 20× objective magnification. Veins can be observed as a tubular structure containing a single layer of flattened endothelial cells.

FIG. 20: A. Biodistribution of mitochondria-FAM-ssDNA complex (green dots) in cortex and medulla. B. Thorough microscopy analysis shows the presence of FAM signal in all designated areas, with the highest signal being seen in the medullar sections. C. Distribution of the complex (green, white arrows) in the proximity of cell nuclei (blue), suggesting the presence of the complex inside the cells in the kidney tissue. Objective magnification is 20×.

FIG. 21: Overview of area selection of the heart for the imaging analysis. Black dots show the target area for Left Anterior Descending (LAD) and Left Ventricle (LV).

FIG. 22: Representative images of HE staining of the LAD and LV taken using the 20× objective magnification. Blood vessels consisting of red blood cells (red arrow) were observed. Blue color indicates cell nuclei. The scale bar is 100 μm.

FIG. 23: Fluorescence images showing A. Autofluorescence of heart tissue seen in transverse section. The distribution of FAM-labeled ssDNA-mitochondria complex (green dots, white arrows) in B. the LV (in transverse section) and in C. LAD (in longitudinal section) of the pig's heart was observed. Hearts possess autofluorescence signal in the green (FITC) channel, however distinct dot green structures (Panel B and C) indicating mitochondria could be easily distinguishable from the autofluorescence signal in the images (Panel A). Red arrows show the red blood cells in the blood vessel. The scale bar is 30 μm.

FIG. 24A: Comparison of the 1^{st} generation (1^{st} gen, left) and the 2^{nd} generation mitochondria-complex (2^{nd} gen, right). Functionalization of mitochondria with oligonucleotides is achieved through a layer-by-layer technique. Negatively-charged mitochondria are modified with cationic polymers such as poly-1-lysine (PLL) or polyethyleneimine (PEI) before being conjugated with negatively-charged oligonucleotides such as DNA or RNA.

FIG. 24B: For the 2^{nd} gen system, the complex was further wrapped with a protective layer (e.g., poly(ethylene glycol)-block-polyethyleneimine or PEG/PEI). The use of two different polymers helps not only to attach different oligonucleotides (i.e., mRNA, siRNA, plasmid DNA, etc.) on the mitochondrial surface, but also to protect the oligonucleotides from degradation, increase the uptake of mitochondria-oligonucleotide complex, as well as to avoid any complex aggregation and phagocytosis by immune cells. Description of the 2^{nd} gen mitochondria-complex carrying simultaneously two different oligonucleotides (left) or oligonucleotide and small-molecule anionic drugs (right).

FIG. 25: A. Fluorescence micrograph of MitoTracker™ Red CMXRos (MT Red) labeled mitochondria-FAM ssDNA 2^{nd} gen complex (concentration of 1 mg/mL). The presence of double staining (yellow color) under fluorescence microscope indicates a successful formation of the 2^{nd} gen complex. B. Photograph showing injection of the concentrated mitochondria-ssDNA 2^{nd} gen complex in a smaller volume (20 mg/mL; 50 μL) using a 30G needle. C. Fluorescence image of the injected sample. The colocalization of both fluorescent signals of MitoTracker™ Red CMXRos and FAM-labeled ssDNA was present, indicating the stability of the 2^{nd} complex at higher concentrations and upon injection. Mitochondria were shown in red while ssDNA was shown in green.

FIG. 26: Size characterization of the mitochondria-oligonucleotide 2^{nd} gen complex was performed using a Coulter Counter device. A. The size of the object is expected to increase at each step of functionalization. B. The change of median size of naked mitochondria and mitochondria-oligonucleotide complex from 0.82 μm to 1.05 μm was observed, indicating successful functionalization of CleanCap® EGFP mRNA on the mitochondrial surface. C. Stability study of mitochondria-mRNA complex in different media. Mean complex size was measured at 0 h and 24 h upon incubation in different media.

FIG. 27: A. Brightfield image showing the association of mitochondria-mCherry mRNA 2^{nd} gen complex on A549 cells after 4.5 h. Dot structures found on and in the cells and cell vicinity are the individual complexes (pointed by arrows). B. Fluorescence measurement shows that the signal of mCherry expression started to appear after 4.5 h post complex incubation (white arrow). C. The expression of the mCherry mRNA after 24 h of incubation where more cells possess fluorescence signals.

FIG. 28: Time-lapse fluorescence imaging of EGFP mRNA expression in HCF cells. A live cell imaging was conducted 6 h post mitochondria-EGFP mRNA 2^{nd} gen complex incubation. The increase of the EGFP signal overtime in the cell of interest (pointed by arrow) was observed.

FIG. 29: Intensity analysis of the timelapse imaging data shows the increase of the EGFP signal over time.

FIG. 30: The mitochondria-EGFP mRNA 2^{nd} gen complex was internalized in A549 and the expression of EGFP mRNA in the cells was observed after 24 h. The complex could be stored at −80° C. for 2 days and thawed before the in vitro experiment is conducted. The expression of EGFP mRNA from a frozen sample was observed in the cells in a similar fashion to the fresh sample.

FIG. 31: A comparison study of EGFP expression in A549 cells after 24 h of incubation with the 1^{st} gen complex, 2^{nd} gen complex, Lipofectamine, and naked mRNA. No expression was observed in the naked mRNA sample, and a major improvement in expression efficiency was detected in the 2^{nd} gen compared to the 1^{st} gen complex.

FIG. 32: A comparison study of EGFP mRNA expression carried by Lipofectamine transfection agent vs. mitochondria in different cells: A549 and HCF represent human cells, while MEF and WEHI represent mouse (animal) cells.

FIG. 33: A comparison study of mCherry mRNA expression carried by Lipofectamine transfection agent vs. mitochondria in different cells: A549 and HCF represent human cells, while MEF and WEHI represent mouse (animal) cells.

FIG. 34: A. In vitro experiment of association and expression of fluorescently labeled mitochondria-mCherry mRNA 2^{nd} gen complex in three different cells (22 h of incubation). By using GFP-labeled HepG2 mitochondria, we were able to show that in A549, HCF, and MEF cells where mCherry protein (red) was expressed, the presence of mitochondria (green) was always observed. B. FACS analysis of HCF cells after 48 h of incubation of different samples. The ratio of mCherry signal to GFP signal allows us to calculate the translation efficiency.

FIG. 35: A. FACS analysis of the expression of EGFP mRNA in A549, varying different N/P (positive and negative) ratios between PEI, PEG/PEI polymers, and mRNA after 24 h of incubation. Lipofectamine and polymeric nanoparticles (PEI and PEG/PEI NPs) were used for comparison. B. EGFP expression of the cells per well quantified using image processing. The complex was administered to the cells in two ways: premixed first with culture media (premixed) and mixed in the well plate by gently shaking (standard). C. Fluorescence micrograph showing the expression of EGFP in A549 cells using the complex which was prepared with centrifugation.

FIG. 36: FACS measurement showing the relative expression of EGFP mRNA to Lipofectamine in A549, HCF, and MEF carried by mitochondria after 24 h or 48 h of incubation. Lipofectamine and/or polymeric nanoparticles were used for comparison. Increasing the incubation time, as expected, increases the percentage of the cell population possessing the EGFP signal. In mouse MEF cells, the translation efficiency is low compared to human ones.

FIG. 37: Fluorescence imaging of the mitochondria-EGFP mRNA 2^{nd} gen complex in vitro with changing mRNA concentration (1×-2×). After 24 hours of incubation in A549 cells, FACS analysis was performed to assess EGFP mRNA expression, resulting in more than 75% relative mRNA expression in the cells.

FIG. 38: A. In vitro imaging of mitochondria-EGFP mRNA and mitochondria-mCherry mRNA 2^{nd} gen complex in iCell® Cardiomyocytes² cells after 24 h of incubation. B. FACS measurements show a relative expression of EGFP mRNA carried by mitochondria after 48 h of incubation. Lipofectamine was used as comparison. C. Calculation of beating rate in the iCell® Cardiomyocytes² cells showing increase of the beating rate 48 h after complex incubation.

FIG. 39: In vitro mRNA expression study of stored mitochondria-mRNA 2^{nd} gen complex in HCF and A549 cells. The complex was stored for up to 4 months at −80° C. before being thawed and administered to the cell. The mCherry (mCh) or EGFP signal (EGFP) was observed 24 h post incubation, indicating storage does not alter and destroy the mitochondria-mRNA complex.

FIG. 40: Fluorescence micrograph showing the expression of mitochondria-mCherry 2^{nd} gen complex and MTS assay for measuring potential cytotoxicity of the administered complex in A549 cells. No toxicity was observed in the cells after 24 h of complex incubation at concentrations of 50 μg and 75 μg. The complex was centrifuged beforehand and resuspended, in order to make sure there is no free mRNA nanoparticles in the system. The control sample was cells treated with buffer.

FIG. 41: Formation of mitochondria-siRNA 2^{nd} gen complex (left) and Lipofectamine-siRNA nanoparticles (right) were observed using the 2^{nd} gen conjugation approach. GAPDH siRNA was labeled with FAM (green). The black panel shows magnified images.

FIG. 42: In vitro association of the mitochondria-siRNA 2^{nd} gen complex (green dots) in A549 cells after 3 h of incubation. Mitochondria concentration is 50 μg (ca. 150 million mitochondria), FAM-labeled siRNA concentration is 10 μmol.

FIG. 43: Fluorescence and brightfield microscopy showing the A549 cells after being exposed to mitochondria-GAPDH siRNA 2^{nd} gen complex, mitochondria-MDM2 siRNA 2^{nd} gen complex, Lipofectamine-GAPDH siRNA and Lipofectamine-MDM2 siRNA for 72 h. Mitochondria concentration is 50 μg and siRNA is 10 μmol.

FIG. 44: Cell proliferation analysis after 48 h and 96 h of complex incubation is measured by MTS assay. Successful knockdown of GAPDH and MDM2 by siRNA delivered by mitochondria was observed through reduction of proliferation activity of A549 cells. Mitochondria concentration is 50 μg and siRNA is 10 μmol (1×, 96 h) and 20 μmol (2×, 48 h).

FIG. 45: A. Western Blot protein analysis of GAPDH. B. Knockdown of GAPDH in A549 cells was observed after 72 h post-delivery of GAPDH siRNA by mitochondria. Mitochondria concentration is 50 μg and siRNA is 30 μmol (3×).

FIG. 46: A. Description of mitochondria carrying two types of oligonucleotides. B. Fluorescence micrograph showing the colocation of FAM and Cy3 fluorescence signals indicating successful dual oligonucleotide labeling on the mitochondrial surface.

FIG. 47: Fluorescence micrograph showing in vitro association of mitochondria carrying simultaneously FAM-GAPDH siRNA and EGFP mRNA. A. FAM-GAPDH siRNA appears as dot structures inside A549 cells post 2 h incubation. Presence of the EGFP signal in the cytoplasm of A549 cells shows the successful internalization of EGFP mRNA and EGFP protein expression inside the cells. The first EGFP mRNA expression started after B. 4 h of incubation with the complex and the signal increased when the time was increased to C. 23 h. D. The expression mCherry mRNA (red) and the presence of FAM-GAPDH siRNA were observed simultaneously. E. MTS assay showing the reduce of proliferation of A549 cells after 24 h of incubation with a complex simultaneously carrying FAM-GAPDH siRNA and EGFP mRNA. Mitochondria concentration is 50 μg and siRNA is 20 μmol.

FIG. 48: A. A photograph showing nebulization of the complex to A549 cells using a standard nebulizer. The cells were nebulized with the 2^{nd} gen complex for 30s. B. Fluorescence imaging showing A549 cells expressing EGFP mRNA (green) 24 h post nebulization of mitochondria-EGFP mRNA complex.

FIG. 49: A. Western Blot protein analysis of GAPDH. B. Intensity analysis showing the knockdown of GAPDH in A549 cells was observed after 48 h post simultaneous delivery of GAPDH siRNA and PX-12 by mitochondria. Mitochondria concentration is 50 μg and siRNA is 20 μmol. Analysis of the GAPDH intensity band was performed using Fiji.

FIG. 50: In vitro translation study of EGFP mRNA delivered by Lipofectamine, mitochondria with DOTAP functionalization and mitochondria with PEG/PEI functionalization after 24 h.

FIG. 51: Luciferase activity of mitochondria-Renilla Luciferase mRNA 2^{nd} gen complex compared to Lipofectamine-Renilla Luciferase mRNA complex and negative control samples in A549 cells (72 h).

FIG. 52: A. Illustration of mitochondria carrying nanoparticles that encapsulate oligonucleotides. B. Fluorescence micrograph showing the formation of DOTAP NPs encapsulating FAM-ssDNA. C. The attachment of DOTAP NPs encapsulating FAM-ssDNA on fluorescently labeled mitochondria can be analyzed through the colocalization between the two fluorescence signals (e.g., MitoTracker™ Red CMXRos and FAM-ssDNA). D. An in vitro study of EGFP expression using the complex of mitochondria-DOTAP NPs encapsulating EGFP mRNA in A549 cells after 22 hours of incubation.

### EXAMPLES

### General Methods and Materials

### Cell Culture

Human cardiac fibroblasts (HCF) were cultured in Fibroblast Medium-2 supplemented with fetal bovine serum (FBS), fibroblast growth supplement-2, and an antibiotic solution (penicillin/streptomycin) according to the supplier's directions (ScienCell) until reaching 80-90% cell confluency in a T-150 flask (total cell number: 6-8 million). Human lung epithelial cells (A549) were cultured in RPMI medium supplemented with 10% FBS, 1% Pen/Strep, and 1% L-Glutamine until reaching 80-90% cell confluency (8-10 million cells/flask). Mouse Embryonic Fibroblasts (MEF) were cultured in DMEM medium supplemented with 10% FBS, 1% Pen/Strep, and 1% L-Glutamine until reaching 80-90% cell confluency (8-10 million cells/flask). WEHI 164 cell line from mouse skin was cultured in RPMI medium supplemented with 10% FBS, 1% Pen/Strep, and 1% L-Glutamine until reaching 80-90% cell confluency (2-4 million cells/flask). Mitochondria GFP labeled-HepG2 cells were cultured in RPMI medium supplemented with 10% FBS, 1% Pen/Strep, and 1% L-Glutamine until reaching 80-90% cell confluency (8-10 million cells/flask). iCell® Cardiomyocytes 201434 vials were purchased from FUJIFILM Cellular Dynamics Inc. and cultured in Maintenance medium provided by the manufacturer.

### Mitochondria Isolation

The isolation of mitochondria was carried out using protocols described in-house (NPL8). In brief, HCF cells, GFP-labeled HepG2 cells, or MEF cells were treated with trypsin for 5 minutes at 37° C. and then mixed with fresh medium to neutralize the trypsin. The cell suspension was collected and centrifuged at 300 rpm; the supernatant was removed, and the cell pellet was dispersed in an isolation buffer containing 10 mM HEPES, 1 mM EGTA, 300 mM sucrose, and 2 mg of Subtilisin A (Sigma-Aldrich, Catalog #P5380). The cell suspension was kept on ice at 4° C. for 5 minutes and then vortexed for 2 minutes. Unopened cells were removed by centrifugation at 300 rpm, and the supernatant containing mitochondria was filtered through a three-step filtration process using a 40-micron, a 40-micron (Fisher Scientific, Catalog #352340), and a 10-micron filter (pluriSelect, Catalog #43-50010-03). Mitochondria were pelleted by centrifugation at 9500 rpm and washed three times using an isolation buffer. Mitochondria were then resuspended in isolation buffer at a final concentration of 1 milligram/mL based on the Qubit's protein count following the protocol described by the manufacturer (Thermo Fisher Scientific). Coulter Counter was used to count the mitochondria particles using a protocol developed by the manufacturer (Beckman Coulter Inc.). Typically, 1 mg/mL corresponds to ca. 3 billion mitochondria/mL. The mitochondria were stored at −80° C. before being thawed for the synthesis of the mitochondria-oligonucleotide complex. Mitochondria staining was performed using MitoTracker™ Red CMXRos (Thermo Fisher Scientific).

### Synthesis of Mitochondria-ssDNA 1^{st} Complex

Human cardiac fibroblasts (HCF) were cultured in Fibroblast Medium-2 (ScienCell) until they reached 80-90% cell confluency (2-4 million cells/flask). Thirty minutes to 1 hour prior to isolation, mitochondria were pre-labeled with MitoTracker™ Red CMXRos (Thermo Fisher Scientific, USA) following the protocols described by the manufacturer (Thermo Fisher Scientific). Labeled mitochondria were isolated according to the established Cellvie SOP (NPL8). Isolated mitochondria were then resuspended in a conjugation buffer comprising a mixture of Solution X (20 mM HEPES+1 mM EGTA+300 mM Trehalose, pH 7.2) and Solution Y (0.1 M CHES, pH 10+0.2 M sodium phosphate dibasic dihydrate), at a final concentration of 1 mg/mL, as determined by a Qubit Protein BR Assay following the protocols described by the manufacturer (Thermo Fisher Scientific). For each 50 μL of the mitochondrial solution, 0.2-0.5 μL of poly-L-Lysine (PLL) solution in H₂O (Sigma-Aldrich, Germany) at a concentration of 10 mg/mL was added and gently mixed with the solution. The mixture was kept at room temperature (RT) for 1-5 minutes. Fluorescently labeled ssDNA (Fluorescein-ssDNA or FAM-ssDNA with an oligo sequence 5′ to 3′ GCAACAGTGAAGGAAAGCC) was previously purchased from Thermo Fisher Scientific (USA) and used without any further purification. FAM-ssDNA was dispersed either in DNase/RNase-Free Water or in phosphate-buffered saline or in Solution X at a concentration of 30 μmol. One μL of the fluorescently labeled ssDNA solution was added and gently mixed with the PLL-mitochondria solution. The incubation was performed at RT for 30 minutes in a dark environment.

### Synthesis of Mitochondria-ssRNA 1^{st} Gen Complex

Human cardiac fibroblasts (HCF) were cultured in Fibroblast Medium-2 (ScienCell) until reaching 80-90% cell confluency (2-4 million cells/flask). Thirty minutes to 1 hour prior isolation, mitochondria were pre-labeled with MitoTracker™ Red CMXRos (Thermo Fisher Scientific, USA) following the protocols described by the manufacture (Thermo Fisher Scientific). Labeled mitochondria were isolated according to the established cellvie SOP (NPL8). Isolated mitochondria were resuspended in the conjugation buffer, at a final concentration of 1 mg/mL as determined by a Qubit Protein BR Assay following the protocols described by the manufacture. For each 50 μL of the mitochondria solution, 1 μL of poly-L-Lysine solution (10 mg/mL) was added and gently mixed with the solution. Fluorescently labeled SSRNA (FAM-ssRNA with oligo sequence 5′ to 3′ UUCUCCGAACGUGUCACGUUU) was previously purchased from Thermo Fisher Scientific (USA) and used without any further purification. FAM-ssDNA was dispersed either in DNase/RNase-Free Water or in phosphate buffered saline or in solution X at a concentration of 30 μmol. One μL of the fluorescently labeled ssRNA solution was added and gently mixed with the PLL-mitochondria solution. Incubation was performed at RT for 30 minutes under dark environment.

### Synthesis of Mitochondria-Silencer™ FAM-Labeled GAPDH siRNA 1^{st} Gen Complex

Human cardiac fibroblasts (HCF) were cultured in Fibroblast Medium-2 (ScienCell) until reaching 80-90% cell confluency (2-4 million cells/flask). Mitochondria were isolated according to the established cellvie SOP (NPL8). All mitochondria were resuspended in conjugation buffer at a final concentration of 1 mg/mL based on Qubit's protein count. For each 50 μL of the mitochondria solution, 1 μL of poly-L-Lysine solution (10 mg/mL) was added and gently mixed with the solution. Then, 2.5 μL of Silencer™ FAM-labeled GAPDH siRNA (Thermo Fisher Scientific, US) at a concentration of 50 μM in nuclease-free water was added and gently mixed with the PLL-mitochondria solution. Incubation was performed at RT for 30 minutes in the dark.

For a comparison study using Lipofectamine, in tube A, 50 μL of Opti-MEM™ I Reduced Serum Medium (Thermo Fisher Scientific, Cat. Nr. 31985062) was gently mixed with 6 μL of Lipofectamine™ RNAiMAX Transfection Reagent (Thermo Fisher Scientific, Cat. Nr. 13778100). In tube B, 50 μL of Opti-MEM™ I Reduced Serum Medium was gently mixed with 6 μL of StemMACS™ Nuclear EGFP mRNA. The entire content of Tube A and B were mixed, and the mixture was further incubated for 5 minutes at room temperature in the dark.

### Synthesis of Mitochondria-Ambion™ Silencer™ Pre-Designed MDM2 siRNA 1^{st} Gen Complex

Human cardiac fibroblasts (HCF) were cultured in Fibroblast Medium-2 (ScienCell) until reaching 80-90% cell confluency (2-4 million cells/flask). Thirty minutes to 1 hour prior to isolation, mitochondria were pre-labeled with MitoTracker™ Red CMXRos (Thermo Fisher Scientific, USA) following the protocols described by the manufacturer (Thermo Fisher Scientific). Labeled mitochondria were isolated according to the established cellvie SOP (NPL8). Isolated mitochondria were resuspended in the conjugation buffer, at a final concentration of 1 mg/mL as determined by a Qubit Protein BR Assay following the protocols described by the manufacturer. For each 50 μL of the mitochondria solution, 1 μL of poly-L-Lysine solution (10 mg/mL) was added and gently mixed with the solution. Then, 2.5 μL of Ambion™ Silencer™ Pre-Designed MDM2 siRNA AM51331 (Thermo Fisher Scientific, US), at a concentration of 50 μM in ddH₂O was added and gently mixed with the PLL-mitochondria solution. Incubation was performed at RT for 30 minutes, in the dark.

### Transplantation/Internalization Study

Method 1: Approximately 20000 HepG2 or HCF cells were cultured on 24-well plates or MatTek glass bottom dishes (MatTek Corporation, USA). After 24 hours, 40-60 μg/mL of mitochondria-DNA/RNA complex or Lipofectamine-mRNA complex was added. The cells were incubated for 24-48 hours before performing different assays. For cell counting, DAPI staining was performed following the protocols provided by the manufacturer (Thermo Fisher Scientific). For the proliferation assay, MTS assay was performed as per the manufacturer's instructions.

Method 2: Approximately 10000-25000 A549, HCF, or MEF cells were grown on 48-well plates or Ibidi dishes (Ibidi GmbH, Germany) for 24-48 hours. Afterwards, 50 or 75 μg of the complex composed of mitochondria and ssDNA/mRNA/siRNA or Lipofectamine-mRNA/siRNA or nanoparticles-mRNA was added, and the cells were incubated for 24-96 hours before conducting various tests. The expression of the relevant protein was examined using a fluorescence microscope. Cell proliferation or cytotoxicity was assessed using the MTS assay as per the manufacturer's instructions.

### Cancer Invasion Assay

Mitochondria-siRNA 1^{st} gen complex-treated and untreated cells were harvested using trypsin-EDTA (ScienceCell, USA). The spheroid was engineered by mixing the cell pellet with 1.5% (w/v) sodium alginate (Sigma-Aldrich, USA, Cat. No. 180947) in cell culture medium. The mixture was injected using an Omnican® syringe with a 30-gauge needle (Braun, Germany) into a bath solution containing 75 mM CaCl2 (Sigma-Aldrich, Cat. No. C1016) in cell culture media. The spheroid was allowed to jellify for 5 minutes before being collected and subsequently washed with fresh culture media. The spheroid was cultured on a well-plate, and escaped cells were monitored a few days after seeding using a fluorescence microscope.

Uptake and Translation Study of Mitochondria-FAM ssDNA Complex in 3D Coculture Model

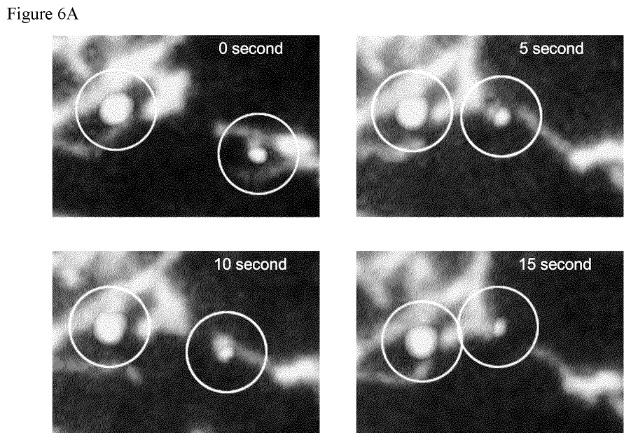

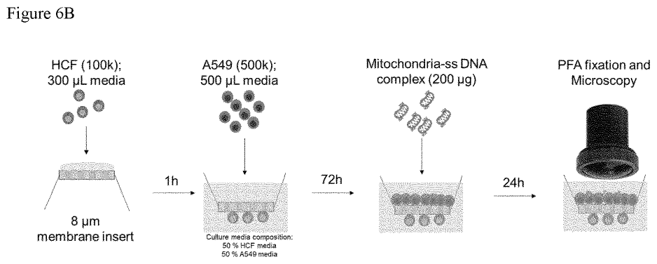

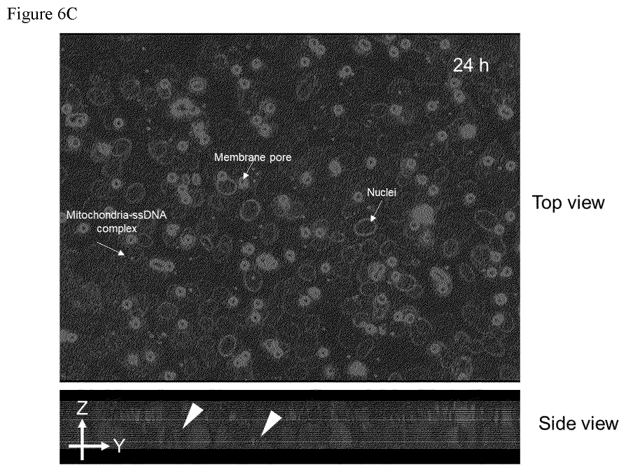

An in vitro 3D coculture was created by combining A549 and HCF cells, following the procedure outlined in FIG. 6B. Initially, 1×10⁵ HCF fibroblast cells were cultured basally on a cellQART membrane insert (cellQART, 6 well plate configuration, with a pore size of 8 μm). Subsequently, 1×10⁵ A549 cells were added, and the cells were allowed to grow for 72 hours until a cell monolayer of A549 cells was formed. The old media was then exchanged with fresh media, after which mitochondria-FAM ssDNA complex (200 μg) was added to the cells. The cells were further incubated with the complex for 24 hours and then fixed with 4% paraformaldehyde. DAPI staining was performed, and the cells were analyzed using z-stack fluorescence imaging. Image analysis and 3D visualization were carried out using Fiji.

### Fluorescence Microscopy

Fluorescence imaging was performed using either a Nanolive fluorescence microscope (Nanolive SA, Switzerland) or a Keyence fluorescence microscope BZ-X800 (Keyence, Japan) with two magnification options: 20× or 40×. These microscopes have various LED light excitations and sets of emission filters that enable the capture of fluorescence in the blue (DAPI), green (GFP), and red emission spectrum (RFP). These microscopes allow for direct visualization of labeled mitochondria samples. The images were automatically processed using Fiji software (NIH, USA).

### Flow Cytometry (FACS)

A flow cytometry experiment of mitochondria-ssDNA complex suspension was performed on a FACSLyric (BD Biosciences), and the recorded fluorescence signals were analyzed using FlowJo software (Tree Star, Ashland, USA). The data are shown as a scatter profile, with the x-axis and γ-axis representing mitochondria and DNA, respectively.

### MTS Assay

Approximately 25000 HepG2 cells were cultured on a 24-well plate dish at 37° C. and 5% CO₂. After 48 hours, mitochondria-Silencer™ FAM-labeled GAPDH siRNA and mitochondria-Ambion™ MDM2 siRNA complex with a concentration of 50 μg/mL was added. The cells were further incubated for 48 hours before the unbound mitochondria-siRNA complex was removed by washing with PBS (Phosphate Buffer Saline). The MTS assay, based on the reduction of the MTS tetrazolium compound by viable cells to generate a colored formazan dye that is soluble in cell culture media, was performed following the protocols described by the manufacturer (Promega). After 1 hour of incubation, the formazan dye was quantified by measuring the absorbance at 490-500 nm using a plate reader.

### SDS-PAGE and Western Blot

Approximately 25000 HepG2 cells were cultured on a 24-well plate dish at 37° C. and 5% CO₂. After 48 hours, a mitochondria-Silencer™ FAM-labeled GAPDH siRNA complex with a concentration of 50 μg/mL was added. The cells were further incubated for 48 hours before the unbound mitochondria-siRNA complex was removed by washing with PBS. Untreated cells were used as controls in the experiments. The cells were washed once with PBS and lysed directly in the wells of a 24-well plate by adding RIPA buffer (Sigma-Aldrich, Cat. Nr. R0278-50 ML) supplemented with 1× Complete EDTA-free protease inhibitor cocktail (Merck, Cat. Nr. 11873580001) and Pierce™ Universal Nuclease for Cell Lysis (Thermo Fisher Scientific, Cat. Nr 88701). After 20 minutes of incubation at 4° C., the lysed cells were collected, and protein content was determined using the Pierce™ BCA Protein Assay Kit (Thermo Fisher Scientific, Cat. Nr. 23225). The samples were adjusted to an equal protein concentration of 2 mg/mL, and 1× Pierce™ LDS Sample Buffer, Non-Reducing (Thermo Fisher Scientific, Cat. Nr. 84788) was added. The samples were stored frozen at −20° C. until use.

For SDS-PAGE, pre-cast NuPAGE™ 12%, Bis-Tris, 1.0 mm, Mini Protein Gels (Thermo Fisher Scientific, Cat. Nr. NP0343BOX) were loaded with samples in LDS Sample buffer (25 μg protein per well) and run in NuPAGE™ MES SDS Running Buffer (Thermo Fisher Scientific, Cat. Nr. NP0002) at 200 V for 1 hour. After that, the gels were blotted on polyvinylidene fluoride (PVDF)/Filter Paper Sandwich, 0.2 μm, 8.3×7.3 cm (Thermo Fisher Scientific, Cat. Nr. LC2002) using a Mini Trans-Blot Electrophoretic Transfer Cell (Bio-Rad Cat. Nr. 1703930) at 200 V for 1 hour.

For western blotting, a PVDF (polyvinylidene fluoride) membrane was blocked overnight with 10% skimmed milk in tris-buffered saline supplemented with 0.1% Tween-20 (TBST). Then, it was incubated with the antibodies of interest (GAPDH Mouse McAb, ProteinTech Cat. Nr. 60004-1-Ig; MDM2 Rabbit PolyAb, ProteinTech Cat. Nr. 27883-1-AP; or P53 Rabbit PolyAb, ProteinTech Cat. Nr. 10442-1-AP) for 1 hour at room temperature, followed by 3×5 min washing in TBST. After that, the membrane was incubated with secondary HRP-conjugated antibodies (ProteinTech Cat. Nr. SA00001-1 for HRP-conjugated Affinipure Goat Anti-Mouse IgG(H+L) and SA00001-2 for HRP-conjugated Affinipure Goat Anti-Rabbit IgG(H+L)) for 1 hour at room temperature. Finally, the membrane was washed three times for 5 minutes in TBST, developed using Pierce™ ECL Western Blotting Substrate (Thermo Fisher Scientific, Cat. Nr. 32106), and exposed on a CL-XPosure™ Film, 8×10 in. (20×25 cm) (Thermo Fisher Scientific, Cat. Nr. 34091).

### Quantification of Mitochondria Numbers by Using Multisizer 4e Coulter Counter

To determine the dosage (or number) of mitochondria, Beckman Coulter Multisizer 4e (Beckman Coulter Inc.) with an aperture tube of 30 μm was used, allowing us to measure particles in a range of 0.6 μm to 18 μm. One microliter of mitochondrial suspension was gently mixed with 10 mL of Isotone solution (Beckman Coulter Inc.). From this solution, the total number of particles in a fifty microliter of the solution was measured.

The analysis of Coulter Counter shows a distribution of particles/complexes based on the size, their corresponding counts and total particle number in a 50 microliter sample solution.

The total number of particles or mitochondria per mL (Nc) was calculated using the following equation (Eq. 1):

$${N}_{c} = \frac{Average particle number}{50} \times Dilution factor \times {10}^{7} \qquad ( Eq . 1 )$$

In Vivo Study of the Direct Injection of Mitochondria-ssDNA 1^{st} Gen Complex into Pig Hearts

Five milliliters of mitochondria-ssDNA suspension (ssDNA is fluorescently labeled with FAM, and the ssDNA oligo sequence 5′ to 3′ is GCAACAGTGAAGGAAAGCC) in conjugation buffer (at a concentration of 1 mg/mL) were directly injected into the heart of the pig. After two hours, the pig was sacrificed, and a small piece of heart tissue where the injection had occurred was cut. The tissue was fixed using formaldehyde, and a histology cut was performed. The tissue was stained with DAPI and rhodamine phalloidin (Thermo Fisher Scientific) to visualize cell nuclei and F-actin networks, respectively. The FAM signal represents the ssDNA signal. A control experiment was prepared by directly injecting naked ssDNA.

### In Vitro Study of Aerosolized Mitochondria and Mitochondria-ssDNA 1^{st} Gen Complexes

MitoTracker™ Red CMXRos-labeled mitochondria or MitoTracker™ Red CMXRos-labeled mitochondria-ssDNA complexes were dispersed in solution X at a concentration of 0.4 mg/mL. Nebulization was performed using a commercially available inhalator, Beurer IH55 (Beurer, Germany), at a speed of 0.25 mL/min. Aerosolized mitochondria were collected on 24-well plates for fluorescence microscopy experiments.

An in vitro study of the internalization of aerosolized mitochondria-ssDNA was performed using HepG2 cells. Briefly, 5000 HepG2 cells were cultured on a 96-well plate overnight. The cells were exposed to aerosolized mitochondria-ssDNA for 30 seconds, and after nebulization, the cells were kept inside an incubator for 20 hours before conducting a fluorescence imaging experiment.

### Example 1: Synthesis, Characterization, and In Vitro Delivery of Fluorescently Labeled ssDNA Using Mitochondria (1^{st} Gen Complex)

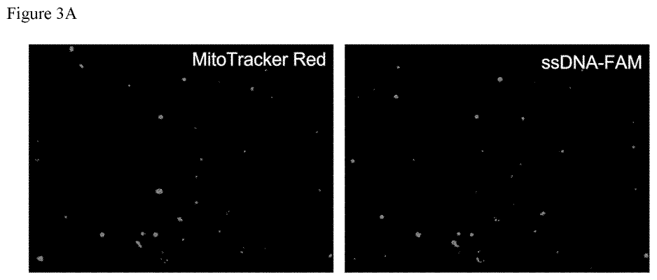

Isolated viable mitochondria possess a negatively-charged surface; therefore, they can be functionalized with cationic molecules, which turn the surface charge of the mitochondria's outer membrane to a more positive value. Subsequently, positively-charged mitochondria can be conjugated with negatively-charged DNA (FIG. 2A). To easily characterize the systems under a fluorescence microscope, isolated mitochondria were first pre-labeled with MitoTracker™ Red CMXRos, and single-stranded DNA (ssDNA) with an oligo sequence of 5′ to 3′ GCAACAGTGAAGGAAAGCC (NPL9) was modified with fluorescein dyes (FAM). The choice of different absorption/emission wavelengths for MitoTracker™ Red CMXRos and FAM was carefully considered to avoid any signal overlapping during microscopy analysis. To perform the electrostatic interaction, isolated (labeled) mitochondria were first dispersed in a conjugation buffer consisting of a 4:1 mixture of Solution X made from a mixture of 20 mM HEPES+1 mM EGTA+300 mM Trehalose (pH 7.2) and Solution Y, comprising 0.1 M CHES (pH 10)+0.2 M sodium phosphate dibasic dihydrate. The mitochondria suspension was first gently mixed with poly-L-lysine for 1-5 minutes at room temperature, protected from the light, before FAM-labeled ssDNA was added to the mixture. The complex was then mixed and incubated for the next 30 minutes at RT and in a dark environment. A fluorescence microscopy experiment was conducted to characterize the complex.

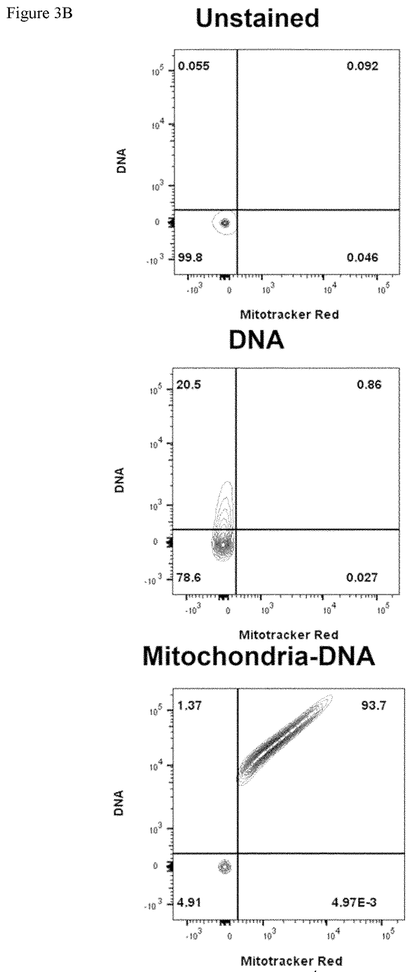

Fluorescence microscopy data shows the formation of spherical objects with a diameter in the range of 1.2 to 4 micrometers under appropriate light excitation. The presence of both fluorescence signals in the same spot indicates the successful functionalization of labeled-DNA molecules on the mitochondria surface. Flow cytometry (FACS) data also confirms the existence of double staining signals in the suspension containing mitochondria-ssDNA complex, suggesting the availability of DNA on the mitochondria outer membrane. More importantly, more than 93% of the population possesses a double staining profile, indicating a high yield of the conjugation process (FIG. 3).

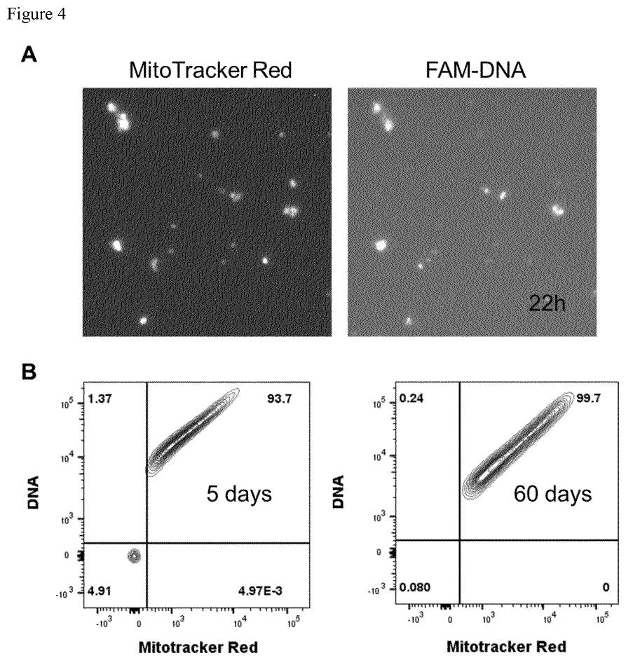

High colloidal stability of vectors inside the body is crucial for the successful delivery of DNA/RNA. In particular, aggregation or agglomeration and disintegration of the system should be avoided. To test the colloidal stability of the systems in biological media, the mitochondria-ssDNA complex was incubated in cell culture medium at 37° C. and 5% CO₂ for 22 hours. Fluorescence microscopy data shows that there was no disintegration or agglomeration of the complex, indicating great stability of the complex in a protein-rich environment (FIG. 4). The complex was also stored at −80° C. in the conjugation buffer for up to 2 months. FACS data shows the continued presence of a double staining profile after brief thawing, suggesting the stability of the complex upon long-term storage (FIG. 4).

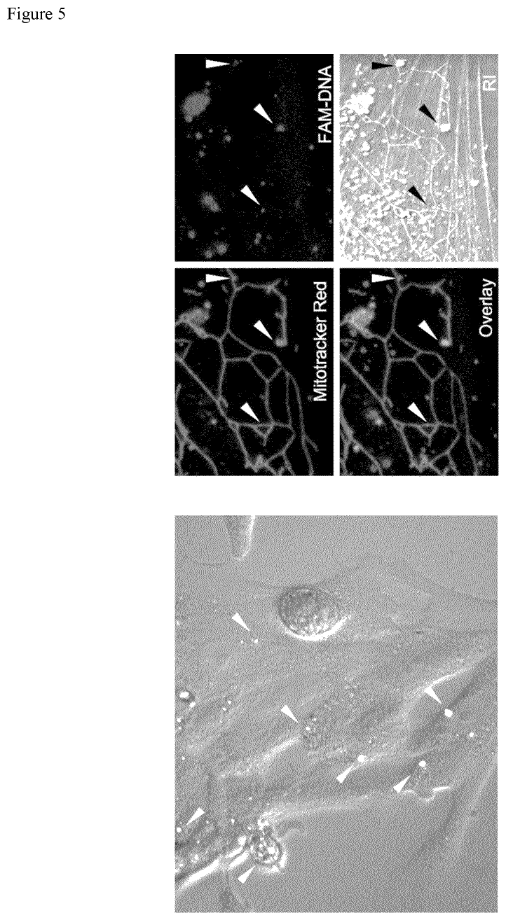

To understand the in vitro behavior of the complex, mitochondria-ssDNA complex was administered to human cardiac fibroblasts as a cell model at a concentration of 50 μg/mL. The cells were incubated at 37° C. and 5% CO₂ for 24 hours, and after the incubation was finished, unbound mitochondria-ssDNA complex was removed by intensive washing using PBS. Fluorescence microscopy data confirms the internalization of the complex by HCF cells (FIG. 5). In addition, no signs of cell toxicity were observed, indicating that the complex was biocompatible and nontoxic. By using a live cell imaging approach, the distribution of mitochondria-ssDNA complex was intensively monitored. Mitochondria-ssDNA complex was observed both in the cytoplasm of the cells, as well as attached to the network of endogenous mitochondria, suggesting the integration of the mitochondria complex into the existing mitochondrion network inside the cells. This biological integration can also be seen through active transport of mitochondria-ssDNA complex from one mitochondrial network to the next network, analyzed by particle tracking analysis (FIG. 6A). Moreover, the presence of single staining of either mitochondria or ssDNA inside the cells was detected (FIG. 5), indicating disintegration and release of the ssDNA from the mitochondria inside the cells. Intracellular release property itself is indeed crucial in a drug delivery system.

Uptake and translocation of mitochondria-ssDNA complex in the 3D model were investigated using fluorescence imaging. Following this objective, an in vitro 3D coculture was created by combining A549 and HCF cells, following the procedure outlined in FIG. 6B. The complex was added to the A549 and HCF coculture, and the cells were incubated for 24 hours. After this incubation period, the cells were fixed and imaged using confocal microscopy. The results showed that the complex was taken up by both A549 and HCF cells and were distributed throughout the coculture (FIG. 6C). Furthermore, 3D visualization of the coculture revealed that the complex (shown by arrows) was able to penetrate the cellQART membrane insert and reach the basolateral side of the coculture (HCF cells). This suggests that the complex could potentially cross biological barriers, which is an important consideration for drug delivery applications. Overall, these results demonstrate the potential of the 3D coculture model for studying mitochondria-oligonucleotide complex uptake and translocation in a more physiologically relevant environment.

### Example 2: Intracellular Delivery of Plasmid DNA Using Mitochondria (1^{st} Gen Complex)

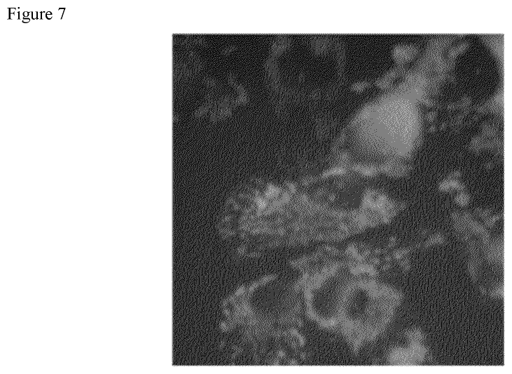

Example 2 has shown the successful functionalization, high colloidal stability of the resulting complex, as well as its interesting in vitro properties (internalization, biocompatibility, distribution, transport, and importantly, disintegration/DNA release). As the selected FAM-labeled ssDNA does not have any subtle biological activity, it was chosen merely as an example of DNA model because of its fluorescence property, so tracking the DNA's biological activity after release inside the cells is hindered. A new system was therefore designed to test whether the released DNA is still biologically active and able to perform its transcription/translational function. Here, a plasmid DNA (pDNA) encoding the mitochondria GFP protein (pTurboGFP-mito) was used. pDNA was attached to the mitochondria surface following a similar procedure described previously in Example 1. pTurboGFP-mito is a commercially available vector that encodes green fluorescent protein TurboGFP that can be fused to the mitochondrial targeting sequence derived from subunit VIII of human cytochrome C oxidase. Upon translation, the presence of a fluorescent signal inside endogenous mitochondria of living cells is detected. The successful translational activity of this pDNA was previously tested with the Lipofectamine system (FIG. 7).

The mitochondria-pDNA complex was then incubated in HCF cells for 96 hours following washing with PBS to remove the unbound complex. Fluorescence microscopy data confirmed the presence of MitoTracker™ Red staining, indicating successful internalization of the mitochondria-pDNA complex, as well as GFP staining in the mitochondria of the cells (white arrow), demonstrating the successful internalization and release of the pDNA followed by pDNA translation (FIG. 8). No signs of cell toxicity were observed through the observation of cell shape under the fluorescence microscope.

### Example 3: Synthesis, Characterization, and Intracellular Delivery of Fluorescently Labeled ssRNA Using Mitochondria (1^{st} Gen Complex)

In addition to DNA, RNA is of great interest in gene therapy and can be used as a candidate for our delivery system. The following example demonstrates that RNA, like DNA, can also be bound to the surface of mitochondria through electrostatic interaction. To easily characterize the systems under a fluorescence microscope, mitochondria were pre-labeled with MitoTracker™ Red, and ssRNA (with an oligo sequence of 5′ to 3′ UUCUCCGAACGUGUCACGUUU (NPL10)) was modified with fluorescein dyes (FAM). It is important to note that, similarly to FAM-ssDNA, FAM-ssRNA does not have any subtle biological activity but is an example of an RNA model chosen due to its fluorescence property. To perform the electrostatic interaction, isolated mitochondria were first dispersed in a conjugation buffer consisting of a 4:1 mixture of Solution X and Solution Y. The mitochondria suspension was then gently mixed with poly-L-lysine for 1-5 minutes at room temperature before FAM-labeled ssRNA was added to the mixture. The complex was then mixed and incubated for the next 30 minutes before a fluorescence microscopy experiment was conducted. Fluorescence microscopy data showed the formation of spherical objects with a diameter in the range of 1.2 to 3 micrometers, with the presence of both fluorescence signals in the same spot, indicating the successful functionalization of labeled-RNA molecules on the mitochondria surface (FIG. 9).

In vitro studies using HCF cells were performed following similar procedures performed using the mitochondria-ssDNA complex. After 24 hours of incubation followed by an extensive washing process, the presence of the mitochondria-ssRNA complex inside the cells was observed. In addition, no sign of any cytotoxicity was detected (FIG. 9).

### Example 4: Intracellular Delivery of Messenger RNA Using Mitochondria and Protein Expression Study (1^{st} Gen Complex)

As Example 3 has already shown the successful internalization of mitochondria-RNA by HCF cells, the use of fully functional RNA, such as messenger RNA (mRNA) for protein expression or small interference RNA (siRNA) for gene silencing, was emphasized.

StemMACS™ Nuclear EGFP mRNA was selected as a candidate because successful delivery and translation of StemMACS™ Nuclear EGFP mRNA could be visually observed by the presence of fluorescence staining inside cell nuclei, indicating the expression of green fluorescent protein (EGFP) linked to a nuclear localization signal.

To perform the electrostatic interaction, 50 μg of isolated mitochondria were first dispersed in 50 μL of conjugation buffer consisting of a 4:1 mixture of Solution X made from a mixture of 20 mM HEPES+1 mM EGTA+300 mM Trehalose (pH 7.2) and Solution Y comprising 0.1 M CHES (pH 10)+0.2 M sodium phosphate dibasic dihydrate. The mitochondria suspension was first gently mixed with poly-L-lysine for 1-5 minutes at room temperature before adding StemMACS™ Nuclear EGFP mRNA to the mixture. The complex was then mixed and incubated for the next 30 minutes before in vitro incubation into HepG2 cells for 24 hours. In addition, a comparison study using conventional RNA delivery using Lipofectamine was performed.

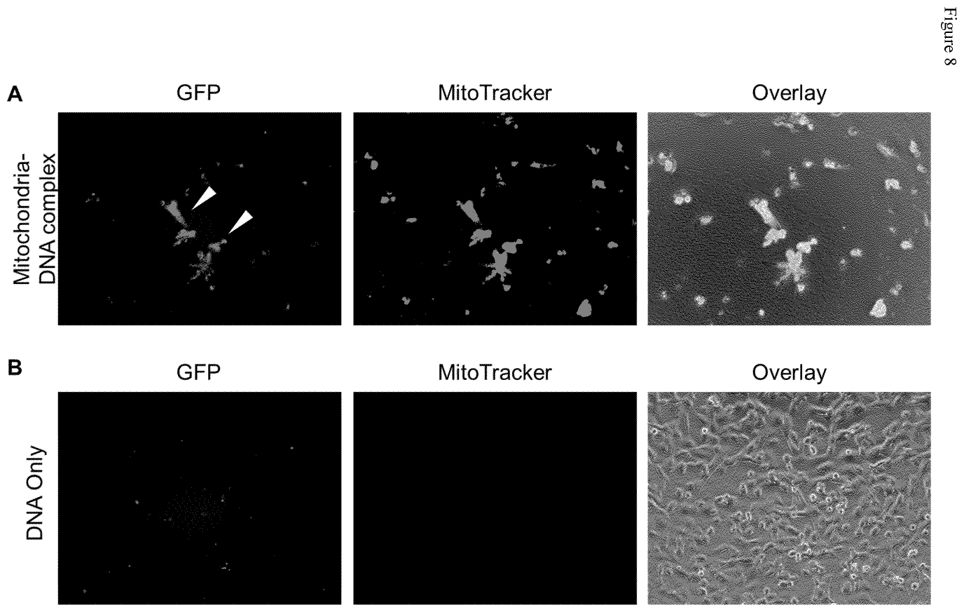

After 24 hours, the presence of fluorescence staining inside cell nuclei was observed for both samples: mitochondria-StemMACS™ Nuclear EGFP mRNA or Lipofectamine-StemMACS™ Nuclear EGFP mRNA (FIG. 10). A co-localization study using DAPI, fluorescence molecules that stain the nucleus, confirmed the nuclear localisation of the EGFP-dependent fluorescence.

### Example 5: Intracellular Delivery of Small Interference RNA and Gene Silencing Study Using Mitochondria-siRNA Complex (1^{st} Gen Complex)

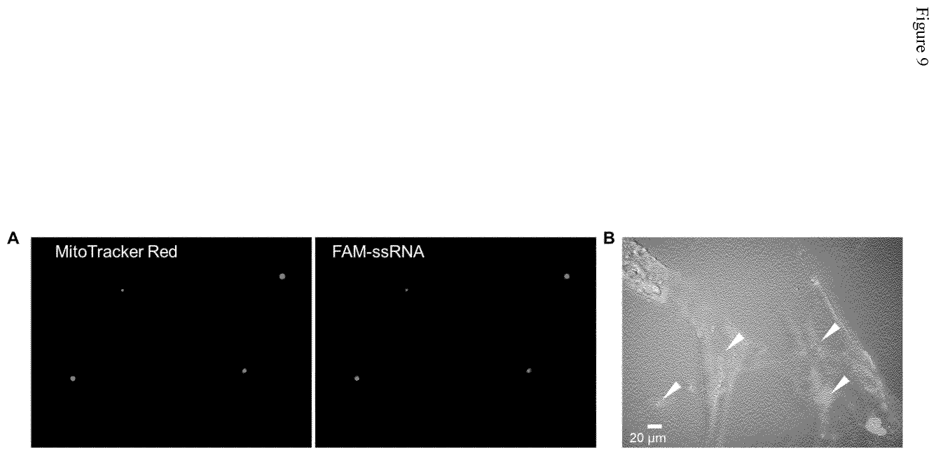

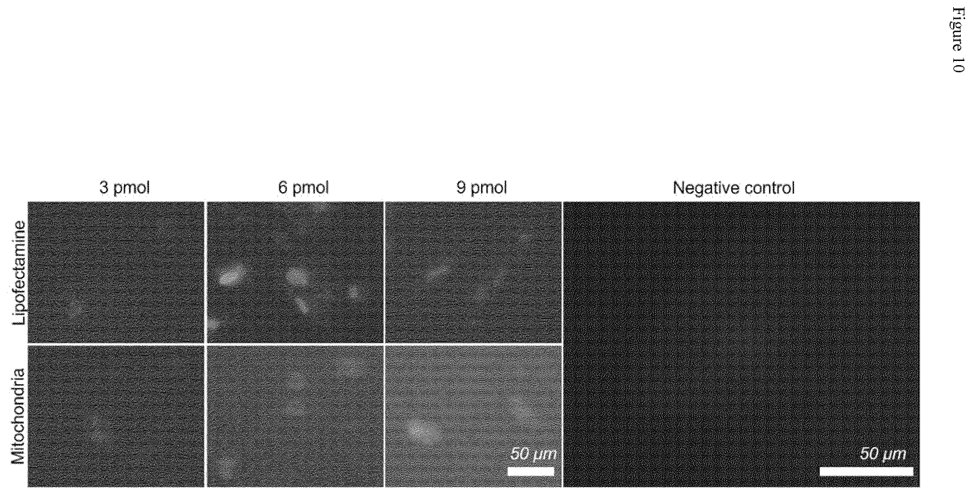

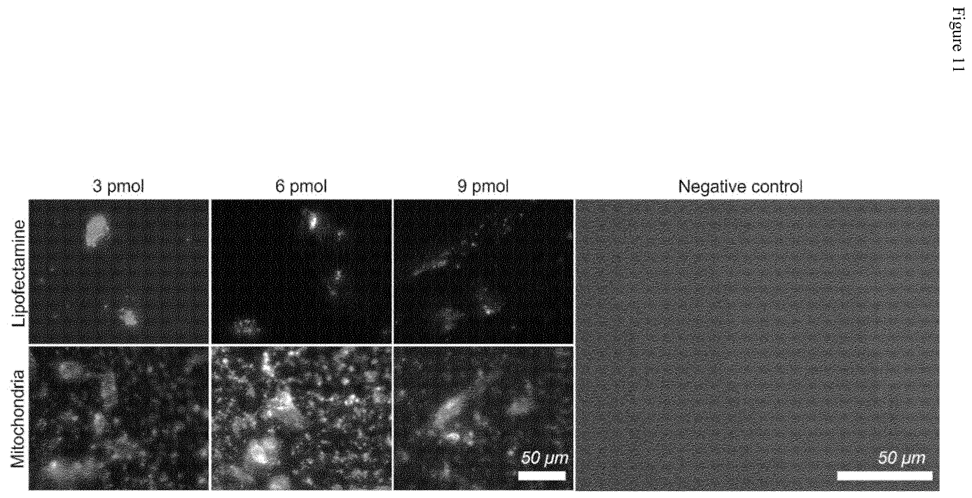

The following example demonstrates the use of mitochondria to transport small interference RNA, specifically Silencer™ FAM-labeled GAPDH siRNA and Ambion™ MDM2 siRNA, into living cancer cells. Glyceraldehyde 3-phosphate dehydrogenase (GAPDH) is an enzyme that catalyzes the sixth step of glycolysis, serving to break down glucose for energy and carbon molecules. Knockdown of GAPDH expression by siRNA will disturb glycolysis, a pathway necessary for cancer cells to produce approximately 60% of their ATP (NPL11), resulting in a reduction in cancer cell viability. MDM2 is an important negative regulator of the p 53 tumor suppressor protein, which is crucial in preventing cancer formation. Inhibition of MDM2 leads to downregulation of tumor suppressive p 53 pathways (NPL12), reducing cancer cell viability. Isolated mitochondria were conjugated with Silencer™ FAM-labeled GAPDH siRNA or Ambion™ MDM2 siRNA using a similar procedure to that previously described. Additionally, Silencer™ FAM-labeled GAPDH siRNA was modified with a fluorescence reporter to allow for easy visualization under a fluorescence microscope. In agreement with the results of a positive control experiment using the widely used Lipofectamine as a delivery agent, fluorescence micrographs show successful internalization of Silencer™ FAM-labeled GAPDH siRNA by mitochondria (with siRNA concentration ranging from 3 to 9 μmol, for 40 μg/mL of mitochondria) in HepG2 cells (FIG. 11). The complex was shown to be distributed throughout the cell cytoplasm, with no major signs of agglomeration of the complex inside the cells after 48 hours of incubation. Moreover, there were no signs of cell toxicity after treatment of mitochondria-Silencer™ FAM-labeled GAPDH siRNA or mitochondria-Ambion™ MDM2 siRNA at concentrations up to 60 μg/mL (FIG. 12).

To understand whether the effect of delivery and release of Silencer™ FAM-labeled GAPDH siRNA from the mitochondrial surface inside the cells can be seen in the reduction of cancer activity (e.g., cell proliferation), different assays such as cell number counting, MTS assay, Western Blot, as well as spheroid cancer invasion assay were performed. The cell count assay was confirmed through DAPI staining and nucleus counting. The counting revealed that HepG2 cells treated with mitochondria-Silencer™ FAM-labeled GAPDH siRNA or mitochondria-Ambion™ MDM2 siRNA (60 g/mL for 48 h) show a significant reduction in cell proliferation compared to untreated cells (FIG. 12). The result is further confirmed by the MTS assay. A lower absorbance value of cells treated with mitochondria-Ambion™ MDM2 siRNA also suggests a reduction in the proliferation of formazan compared to untreated cells. In the case of cells treated with mitochondria-Silencer™ FAM-labeled GAPDH siRNA, an increase in absorbance is expected due to the presence of FAM labeled siRNA, as the FAM molecule has a maximum peak of absorbance at 488 nm (FIG. 12).

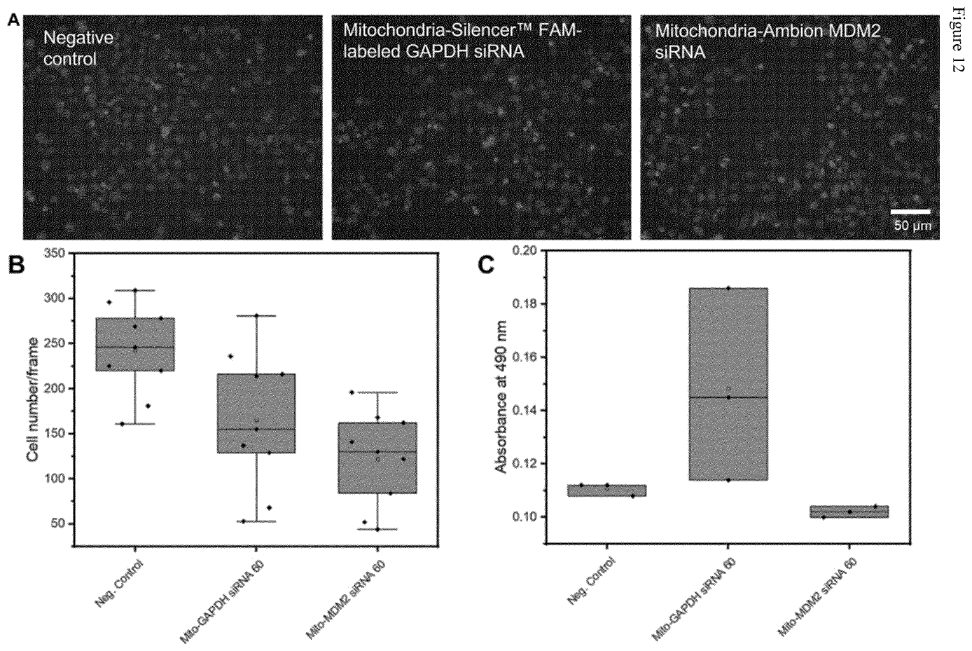

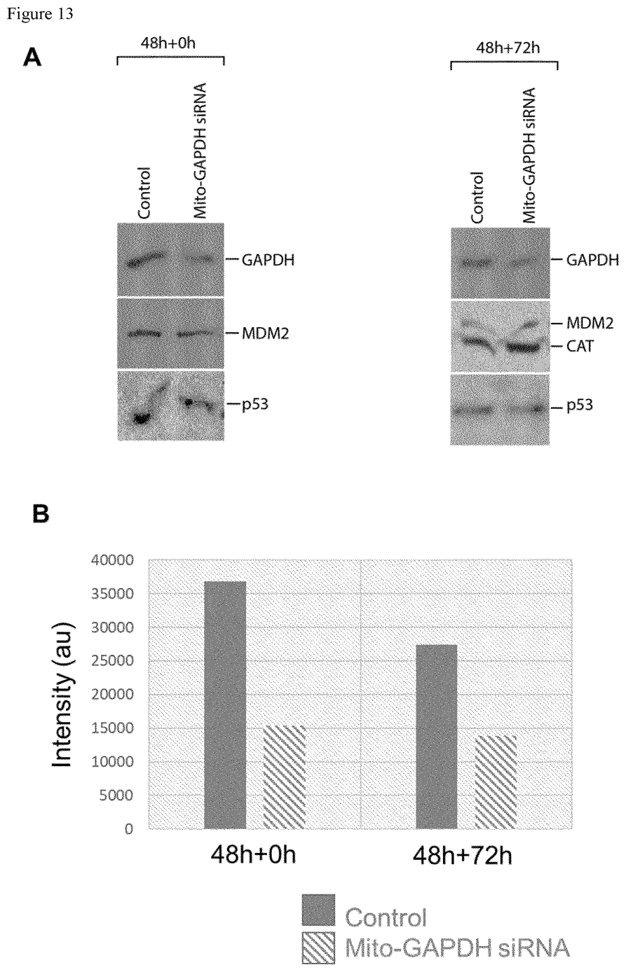

To quantify the protein activity of the cells after being treated with mitochondria-Silencer™ FAM-labeled GAPDH siRNA, SDS Page-Western Blot was performed. HepG2 cells treated with the complex had a lower protein content compared to untreated cells. Additionally, the cells were allowed to grow for 72 hours post-treatment, and SDS Page-Western Blot was performed (FIG. 13A). The result further confirms that the daughter cells treated with the complex still maintain their low GAPDH content compared with untreated cells (FIG. 13B). Finally, a spheroid (cancer) invasion assay was conducted. siRNA-treated cells and untreated cells were harvested, and a spheroid encapsulating the cells, which is made of calcium alginate, was formed (FIG. 14A, 14B). Escaped cells from the spheroid were monitored three days after the spheroid seeding. Nonetheless, no significant reduction in cell invasion was observed in all samples (FIG. 14C).

### Example 6: In Vivo Delivery of Fluorescently Labeled ssDNA Using Mitochondria (1^{st} Gen Complex)

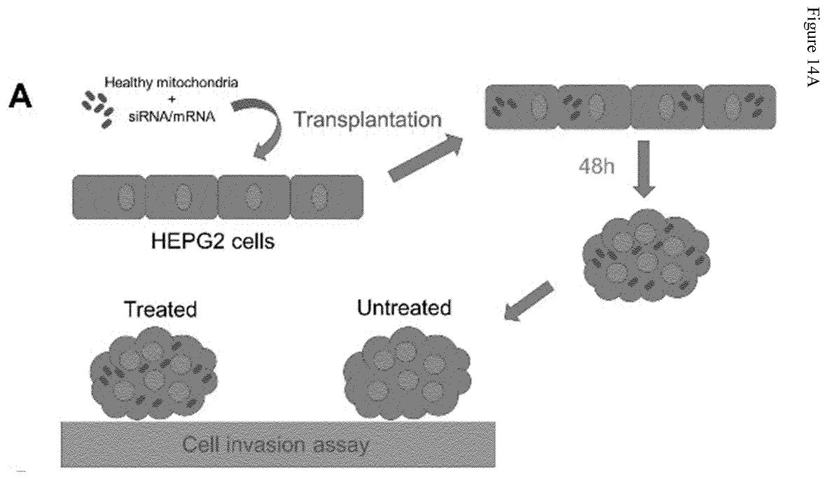

An in vivo experiment was conducted to understand the stability of the mitochondria-ssDNA complex inside a tissue or organ after injection. Using a syringe, either the mitochondria-ssDNA complex or naked ssDNA (control) were directly injected into the heart of a pig. Two hours post-injection, the tissue was processed and fluorescently stained using DAPI and rhodamine phalloidin to stain cell nuclei and F-actins, respectively. The samples were analyzed using a fluorescence microscope to visualize nuclei, actin and the FAM-labeled ssDNA. FIG. 15 shows the distribution of mitochondria-ssDNA inside the heart tissue, near the injection site. Imaging data confirms not only the presence of the complex inside the tissue but also the stability of the complex 2 hours post-injection (FIG. 15). No ssDNA signal was observed in the control experiment (FIG. 15).

It is also worth noting that the serial sections from an injected heart contained different numbers of mitochondria in various positions, attributable to the thickness of the paraffin sections (approximately 5 μm). The image of an injected heart illustrated the presence of most mitochondria in the heart tissue, within the interstitial spaces between cardiomyocytes, as reported previously (NPL13).

### Example 7: Biodistribution of Mitochondria-Oligonucleotide by Magnetic Resonance Imaging (1^{st} Gen Complex)

10 nm N-succinimidyl ester-functionalized (NHS)-modified gold nanoparticles or 30 nm NHS-modified iron oxide nanoparticles are attached on amino groups present on mitochondria membrane-associated proteins through covalent interaction (e.g. peptide bond) (NPL13). Gold nanoparticles-labeled mitochondria are then functionalized with poly-L-lysine to allow the attachment with oligonucleotides. Approximately 1×10⁸ iron oxide nanoparticles-labeled mitochondria-oligonucleotide are introduced into the left ventricular area at risk (AAR) or through injection in renal artery, and magnetic resonance imaging (MRI) experiment is conducted.

### Example 8: Nebulization of Mitochondria and Mitochondria-ssDNA Complex (1^{st} Gen Complex)

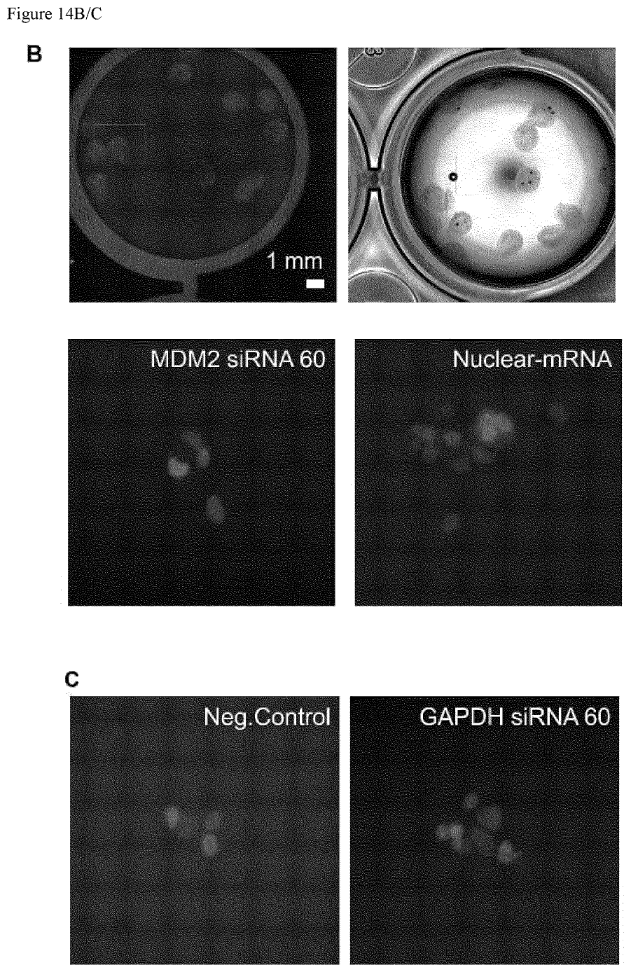

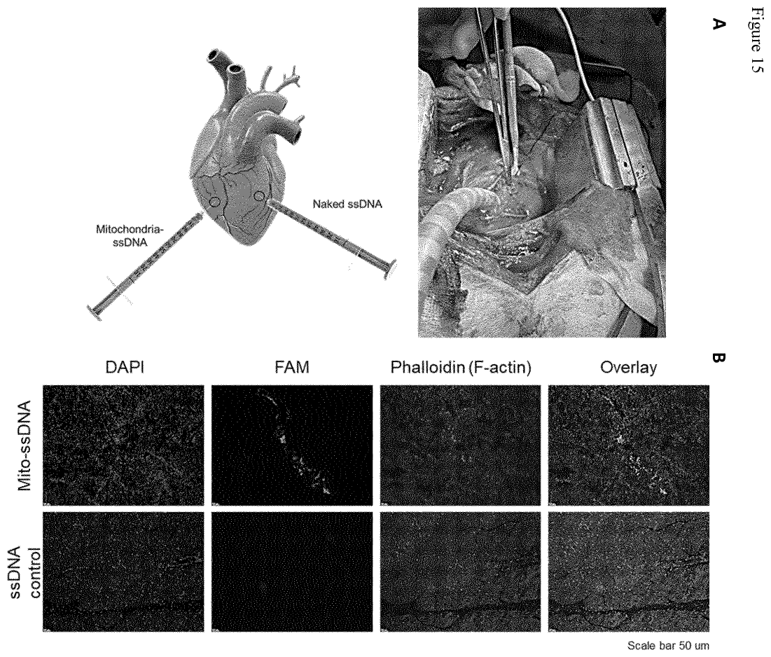

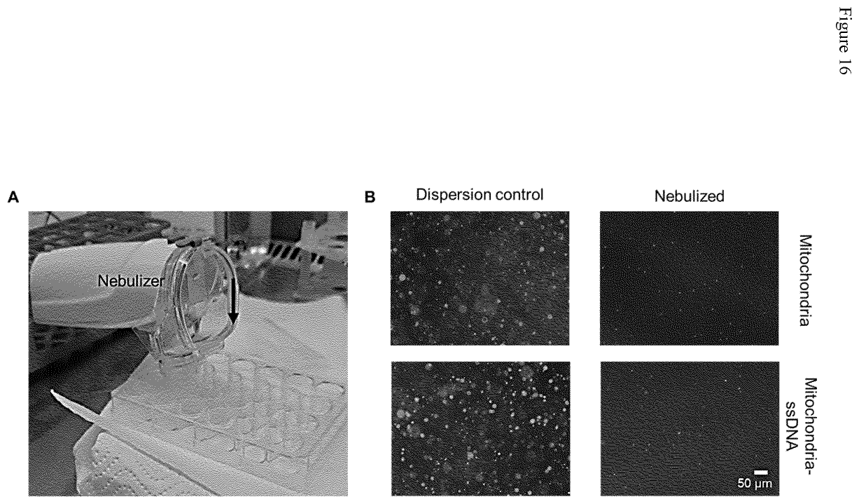

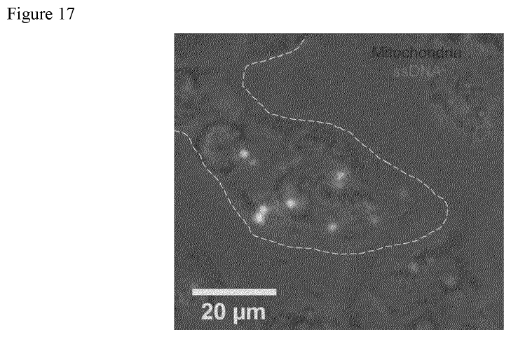

To test the hypothesis that mitochondria and mitochondria carrying oligonucleotides can be may be nebulized (or aerosolized) for possible aerosol delivery treatment through inhalation, solutions containing mitochondria or mitochondria-ssDNA dispersed in solution X at a concentration of 0.4 mg/mL were placed inside a container of a commercially available inhaler. The nebulization was conducted at a speed of 0.25 mL/min, and the resulting aerosol was collected on a well plate for analysis under a fluorescence microscope. Fluorescence microscopy analysis revealed the presence of mitochondria and mitochondria-ssDNA in the collected aerosolized samples, indicating that isolated mitochondria and mitochondria-ssDNA complex can indeed be aerosolized (FIG. 16). Furthermore, the nebulized mitochondria-ssDNA was introduced to the cells for 30 seconds, and the cells were incubated inside an incubator for 20 hours before imaging experiments were conducted. The imaging data showed the presence of mitochondria-ssDNA inside the cells (FIG. 17). No signs of toxicity were observed. These results emphasize the potential delivery pathways involving aerosolized mitochondria carrying oligonucleotides, in addition to direct injection and intravenous injection. Future work is devoted to the in vivo study of delivery of mitochondria-oligonucleotides via aerosolization/nebulization.

### Example 9: Conjugation of Mitochondria to Oligonucleotide which is Modified with an Activated Ester (Covalent Interaction)

Illustration of a conjugation of mitochondria to oligonucleotide which is modified with an activated ester is shown in FIG. 2B. Isolated mitochondria are resuspended in the conjugation buffer at a final concentration of 1 mg/mL. For each 50 μL of the mitochondria solution, 0.2-2 μL of NHS-ester modified oligonucleotide (concentration of 0.1 to 1 mM in DNase/RNase-Free Water or in phosphate-buffered saline or in 0.1 M sodium butyrate at pH 8.5) are gently added and mixed. The mixture is then incubated for 10-20 minutes, followed by three washes in the same buffer containing 1 mg/mL bovine serum albumin.

### Example 10: Conjugation of Mitochondria to Oligonucleotide which is Linked to a Mitochondria Antibody

Illustration of a conjugation of mitochondria to oligonucleotide which is linked to a mitochondria antibody is shown in FIG. 2D. NHS-ester modified oligonucleotides (concentration of 0.1 to 1 mM in DNase/RNase-Free Water or in phosphate-buffered saline or in 0.1 M sodium butyrate at pH 8.5) are gently mixed with an antibody targeting mitochondria TOM20 in the conjugation buffer, followed by incubation for 10-20 minutes and three washes in the same buffer. Isolated mitochondria are resuspended in the conjugation buffer at a final concentration of 1 mg/mL. For each 50 μL of the mitochondrial solution, 0.2-2 μL of oligonucleotide-antibody is added and gently mixed with the solution. The mixture is kept at room temperature for 5-30 minutes.

### Example 11: Conjugation of Mitochondria to Oligonucleotide which is Linked to a Mitochondria-Targeting Small Molecule

Illustration of a conjugation of mitochondria to oligonucleotide which is linked to a mitochondria-targeting small molecule is shown in FIG. 2E. Triphenylphosphonium-labeled oligonucleotides are synthesized as follows. Briefly, NHS-ester modified triphenylphosphonium (concentration of 5 to 20 mM in dimethyl sulfoxide) is mixed with the amino-labeled oligonucleotide (concentration of 0.1 to 1 mM in 0.1 M sodium butyrate at pH 8.5) at a ratio of 1:4 to 1:8, followed by gentle vortexing. The tubes are shaken for 1 to 2 hours at room temperature, and the tube is protected from the light using aluminum foil. An EtOH/3M sodium acetate (9:1 V/V) solution is added to the conjugation reaction, and the solution is vortexed until the conjugate precipitates. The tube is spun at 3000 rpm for 15-25 minutes at 4° C., and the supernatant is removed using a pipette. The precipitate is dispersed in DNase/RNase-Free Water or in phosphate-buffered saline. Isolated mitochondria are resuspended in the conjugation buffer at a final concentration of 1 mg/mL. For each 50 μL of the mitochondrial solution, 0.2-2 μL of triphenylphosphonium-labeled oligonucleotide is added and gently mixed with the solution. The mixture is kept at room temperature for 5-30 minutes.

### Example 12: Conjugation of Mitochondria to Positively-Charged Amino Acids

Isolated mitochondria are resuspended in the conjugation buffer at a final concentration of 1 mg/mL. For each 50 μL of the mitochondrial solution, 0.2-0.5 μL of lysine, arginine, or histidine (concentration of 10 mg/mL in water) is added and gently mixed with the solution. The mixture is kept at room temperature for 5-30 minutes.

### Example 13: In Vivo Delivery of Fluorescently Labeled ssDNA Using Mitochondria into Kidney (1^{st} Gen Complex)

### Methodology

The isolated mitochondria were first dispersed in a conjugation buffer consisting of a 4:1 mixture of Solution X, made from a mixture of 20 mM HEPES+1 mM EGTA+300 mM Trehalose (pH 7.2), and Solution Y, consisting of 0.1 M CHES (pH 10)+0.2 M sodium phosphate dibasic dihydrate. The mitochondrial suspension was then gently mixed with poly-L-lysine for 1-5 minutes at room temperature, protected from light, before FAM-labeled ssDNA was added to the mixture. The complex was then mixed and incubated for the next 30 minutes at room temperature and in a dark environment.

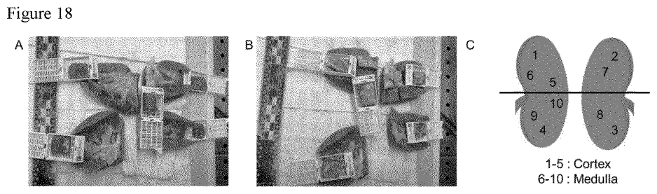

An in vivo experiment was conducted to understand the specific biodistribution of the mitochondria-FAM ssDNA complex inside a tissue or organ after injection. Using a syringe connected to a catheter, 2 mg of the mitochondria-FAM ssDNA complex (in 5 mL of 20 mM HEPES+1 mM EGTA+300 mM Sucrose (pH 7.2)) were directly injected into the renal artery of a pig. Six hours post-injection, the pig was sacrificed, and the kidney tissue was harvested. The kidney was fixed using formalin and stored at 4° C. for a few days before being processed. Five cortical and five medullary biopsies were taken (FIG. 18A-18B). Macroscopically relevant pictures of the tissue samples were taken, and the tissue was immediately transferred to a 10% neutral phosphate-buffered formalin solution (4% w/v formaldehyde solution) for preservation purposes. Histotechnique and histopathological evaluation were performed at C-path (Lier, Belgium). Samples were trimmed, embedded in paraffin, and cut at an approximate thickness of 4 μm. A scheme of the sample location and macroscopy annotations can be seen in the images below (FIG. 18C). Overall, slides 1-5 are cortical sections, while slides 6-10 are medullary sections.

The sections were processed for either H & E staining or DAPI staining. In particular, 20 slides were processed for H & E imaging, while 4 slides (2 cortical, 2 medullary) were processed for DAPI staining.

Images were captured using a Leica DM400 B LED fluorescent microscope. For the H & E-processed sections, images were taken using brightfield (BF) or the fluorescent FITC channel. Furthermore, three images were taken per section (both BF and FITC or DAPI and FITC) using the 40× objective, while one image per section (both BF and FITC or DAPI and FITC) was taken using the 20× objective. Images taken using the 20× objective have a size of 496×447 μm, while images taken using the 40× objective have a size of 298 μm×223 μm.

### Results and Discussion

Representative images highlighting the main structural features of the kidney are shown in FIG. 19. The structures in the cortex and medulla sections include the glomerulus, interstitium, blood vessels, and convoluted tubules. H & E staining also allows visualization of the nuclei of the cells in the tissue under the microscope.

Based on the imaging data, the mitochondria-ssDNA complex was distributed in all areas of the cortex and medulla (FIG. 20) after 6 hours of renal artery injection. In particular, the mitochondria-FAM ssDNA complex was found in the interstitium as well as inside the glomerulus and blood vessels, with the highest signal seen in the medullar sections. Mitochondria were found to be in close proximity to the cell's nucleus, which suggests the presence of the mitochondria-FAM ssDNA complex in individual cells (FIG. 20C).

The results show an important organ-specific biodistribution property of the mitochondria-ssDNA complex post-renal artery injection, allowing specific delivery of mitochondria and the payloads (such as oligonucleotides) in the kidney of the animal.

### Finally, Based on the Observations, the Following Points can be Concluded:

1. The kidney tissue and its structure are preserved after formalin fixation, as shown in the histology images.
2. Mitochondria-FAM ssDNA complex is specifically distributed inside the cortex and medulla of all samples (20 slides: 5 areas cortex, 5 areas medulla, in different regions of the tissue).
3. Mitochondria-FAM ssDNA complex is found inside the glomerulus, interstitium, and blood vessels in the kidney.
4. Mitochondria-FAM ssDNA complex is located in close proximity to the cell nuclei, suggesting its presence inside the cells.

### Example 14: In Vivo Delivery of Fluorescently Labeled ssDNA Using Mitochondria into Heart (1^{st} Gen Complex)

### Methodology

Isolated mitochondria were first dispersed in a conjugation buffer consisting of a 4:1 mixture of Solution X, made from a mixture of 20 mM HEPES, 1 mM EGTA, and 300 mM Trehalose (pH 7.2), and Solution Y, consisting of 0.1 M CHES (pH 10) and 0.2 M sodium phosphate dibasic dihydrate. The mitochondrial suspension was then gently mixed with poly-L-lysine for 1-5 minutes at room temperature, protected from the light, before FAM-labeled ssDNA was added to the mixture. The complex was then mixed and incubated for the next 30 minutes at room temperature and in a dark environment.

An in vivo experiment was conducted to understand the specific biodistribution of the mitochondria-FAM ssDNA complex inside a tissue or organ after injection. By using a syringe connected to a catheter, 1.5 mg of the mitochondria-ssDNA complex (in 5 mL of 20 mM HEPES, 1 mM EGTA, and 300 mM Sucrose (pH 7.2)) was directly injected intracoronary into a pig. Two hours post-injection, the pig was sacrificed, and the heart tissue was harvested. The heart was fixed using formalin and stored at 4° C. for a few days before processing.

Four chosen areas for the left anterior descending artery (LAD) and four for the left ventricle (LV) were taken. Next, the heart tissue was processed for paraffin embedding and sectioned into 16 slides of approximately 5 microns in thickness and ca. 1 to 1.5 cm² in size. H & E staining was performed to visualize tissue architecture.

A total of four fluorescent images were taken per slide at the Leica DM400 B LED fluorescent microscope using a 20× or 40× objective lens. Brightfield was used to visualize the H & E staining and FITC (495/519 ex/em) for fluorescence imaging. FITC allows the visualization of the mitochondria-FAM ssDNA complex.

### Results and Discussion

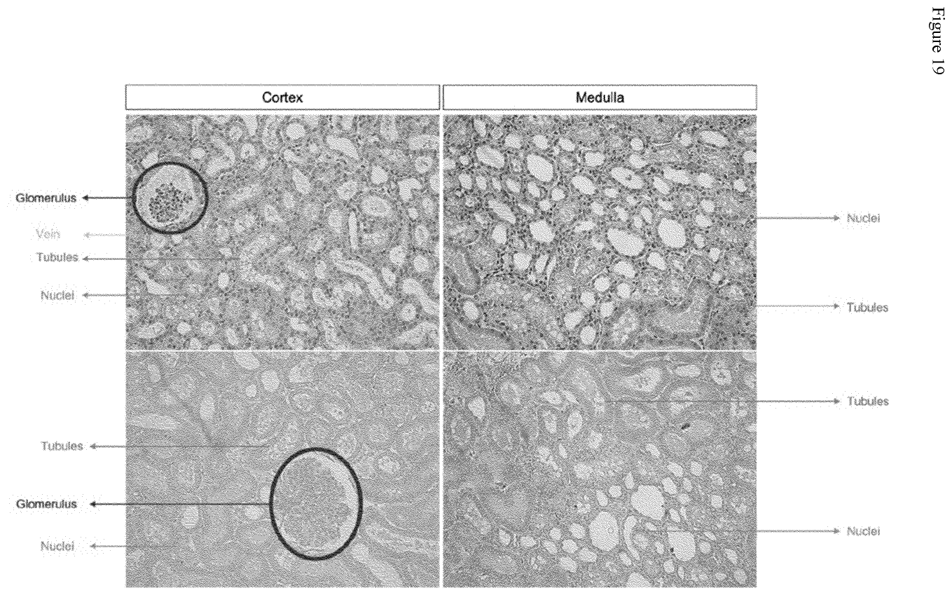

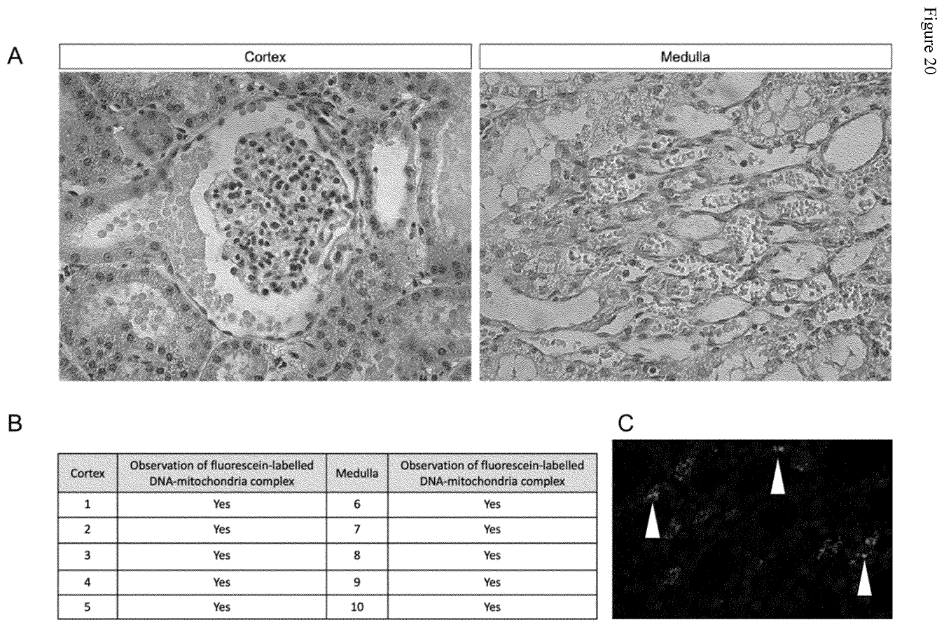

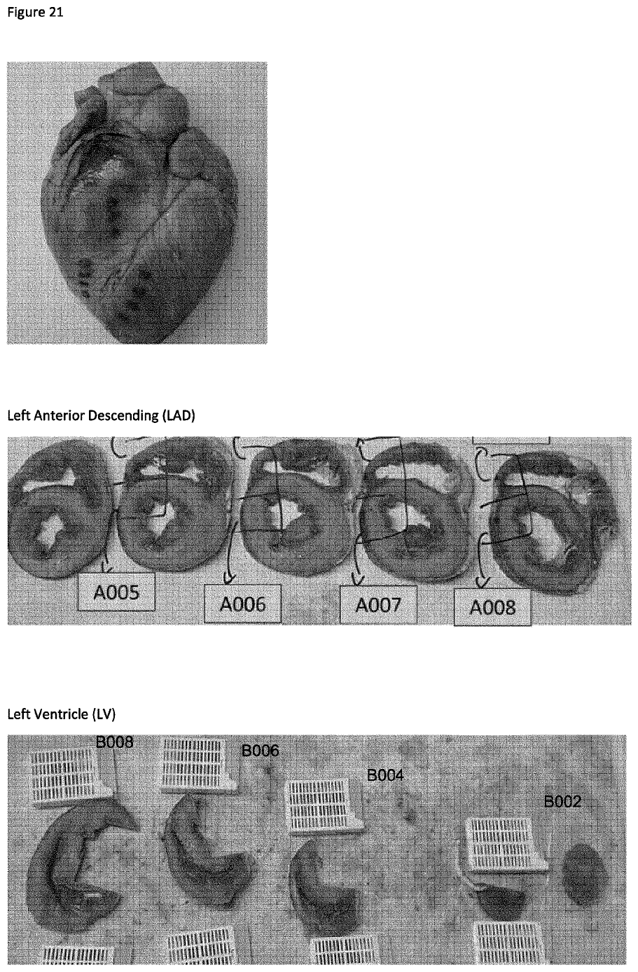

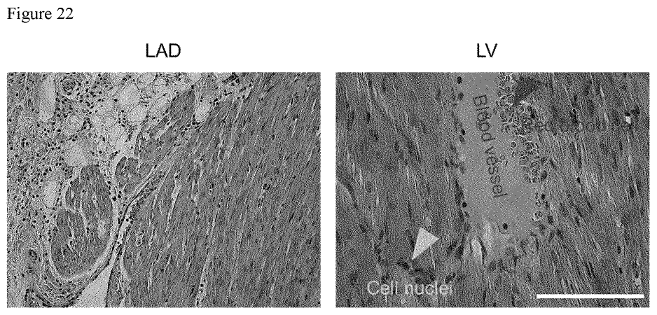

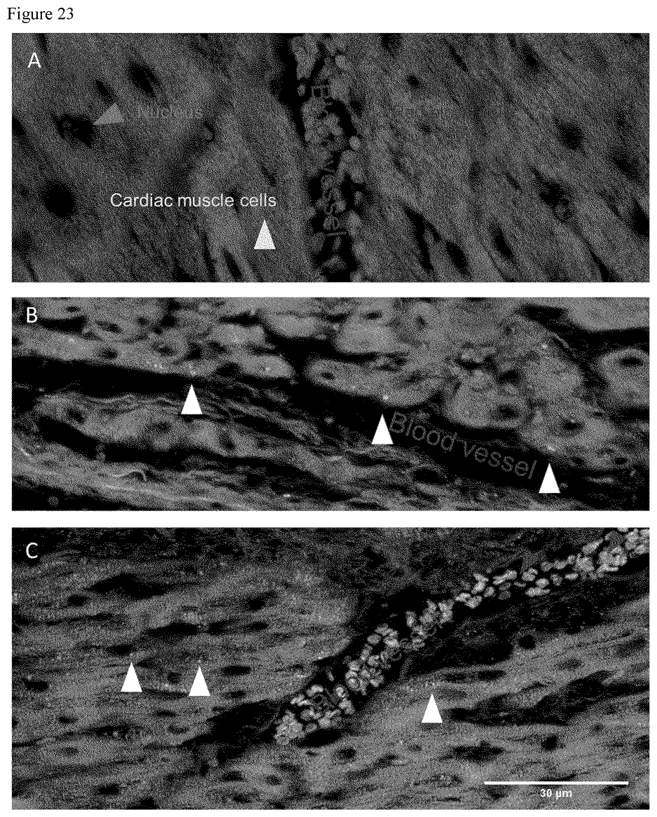

Representative images highlighting the main structural features of the left anterior descending artery (LAD) and the left ventricle (LV) of the pig's heart are shown in FIG. 21. The presence of blood vessels containing red blood cells and cardiomyocytes were observed in the histology images (FIG. 22-23). It is important to note that the heart tissue and its structure are well-preserved after formalin fixation, as shown in the histology images.

Fluorescence imaging using the FITC channel reveals the presence of strong autofluorescence of the heart tissue. Nonetheless, this autofluorescence was useful to see the details of heart structure, such as cardiac muscle cells or red blood cells (FIG. 23A). Two hours post intracoronary injection, mitochondria-FAM ssDNA complex (green dots, white arrows) could already be found distributed in the left ventricle and left anterior descending artery (LAD). In particular, the presence of the dot-like structure inside the cells (e.g., cardiomyocytes/cardiac muscle cells) next to blood vessels was detected, indicating successful internalization of the complex inside the heart post intracoronary injection (FIG. 23).

The results here show the important organ-specific biodistribution of the mitochondria-ssDNA complex post 2 h of intracoronary injection, allowing specific delivery of mitochondria and the payloads (such as oligonucleotides) to the heart of the animal.

### Finally, Based on the Observations, the Following Points can be Concluded:

1. The heart tissue and its structure were preserved after formalin fixation, as shown from histology and fluorescence images.
2. The mitochondria-FAM ssDNA complex was specifically distributed inside the LAD and LV of the heart post two hours of intracoronary injection.
3. The mitochondria-FAM ssDNA complex was found inside the cardiac muscle cells.

### Example 15. In Vivo Delivery of Oligonucleotides Using Mitochondria into Liver

Isolated mitochondria are first dispersed in a conjugation buffer consisting of 4:1 mixture of Solution X made from mixture of 20 mM HEPES+1 mM EGTA+300 mM Trehalose (pH 7.2) and Solution Y consisting of 0.1 M CHES (pH 10)+0.2 M sodium phosphate dibasic dihydrate. Mitochondria suspension are first gently mixed with poly-L-lysine or polyethyleneimine (PEI) for 1-5 minutes at room temperature, protected from the light, before FAM-labeled ssDNA or GFP mRNA or mCherry mRNA or Luciferase mRNA is added into the mixture. The complex is then mixed and incubated for the next 30 minutes at RT and in dark environment.

In vivo experiment is conducted to understand the specific biodistribution of mitochondria-oligonucleotide complex inside a tissue or organ after injection. By using a syringe connected to a catheter, 2 mg of mitochondria-ssDNA complex (in 5 mL of 20 mM HEPES+1 mM EGTA+300 mM Sucrose (pH 7.2)) are directly injected into the hepatic artery or the portal vein of a pig. Six hours post injection, the pig is sacrificed, and the liver tissue is harvested. The liver is fixed using formalin and stored at 4° C. for a few days before processed.

The samples are trimmed, embedded in paraffin, and cut at approximate thickness of 4 μm. The sections are processed for either H & E staining or DAPI staining. Images are processed using a Leica DM400 B Led fluorescent microscope.

### Example 16. In Vivo Delivery of Oligonucleotides Using Mitochondria into Pancreas

Isolated mitochondria are first dispersed in a conjugation buffer consisting of 4:1 mixture of Solution X made from mixture of 20 mM HEPES+1 mM EGTA+300 mM Trehalose (pH 7.2) and Solution Y consisting of 0.1 M CHES (pH 10)+0.2 M sodium phosphate dibasic dihydrate. Mitochondria suspension are first gently mixed with poly-L-lysine or polyethyleneimine for 1-5 minutes at room temperature, protected from the light, before FAM-labeled ssDNA or GFP mRNA or mCherry mRNA or Luciferase mRNA is added into the mixture. The complex is then mixed and incubated for the next 30 minutes at RT and in dark environment.

In vivo experiment is conducted to understand the specific biodistribution of mitochondria-oligonucleotide complex inside a tissue or organ after injection. By using a syringe connected to a catheter, 2 mg of mitochondria-ssDNA complex (in 5 mL of 20 mM HEPES+1 mM EGTA+300 mM Sucrose (pH 7.2)) are directly injected into the hepatic artery of a pig. Six hours post injection, the pig is sacrificed, and the pancreas tissue is harvested. The pancreas is fixed using formalin and stored at 4° C. for a few days before processed.

The samples are trimmed, embedded in paraffin, and cut at approximate thickness of 4 μm. The sections are processed for either H & E staining or DAPI staining. Images are processed using a Leica DM400 B Led fluorescent microscope.

### Example 17. In Vivo Delivery of Oligonucleotides Using Mitochondria into Duodenum

Isolated mitochondria are first dispersed in a conjugation buffer consisting of 4:1 mixture of Solution X made from mixture of 20 mM HEPES+1 mM EGTA+300 mM Trehalose (pH 7.2) and Solution Y consisting of 0.1 M CHES (pH 10)+0.2 M sodium phosphate dibasic dihydrate. Mitochondria suspension are first gently mixed with poly-L-lysine or polyethyleneimine for 1-5 minutes at room temperature, protected from the light, before FAM-labeled ssDNA or GFP mRNA or mCherry mRNA or Luciferase mRNA is added into the mixture. The complex is then mixed and incubated for the next 30 minutes at RT and in dark environment.

In vivo experiment is conducted to understand the specific biodistribution of mitochondria-oligonucleotide complex inside a tissue or organ after injection. By using a syringe connected to a catheter, 2 mg of mitochondria-ssDNA complex (in 5 mL of 20 mM HEPES+1 mM EGTA+300 mM Sucrose (pH 7.2)) are directly injected into the hepatic artery of a pig. Six hours post injection, the pig is sacrificed, and the duodenum tissue is harvested. The duodenum is fixed using formalin and stored at 4° C. for a few days before processed.

The samples are trimmed, embedded in paraffin, and cut at approximate thickness of 4 μm. The sections are processed for either H & E staining or DAPI staining. Images are processed using a Leica DM400 B Led fluorescent microscope.

### Example 18. In Vivo Delivery of Oligonucleotides Using Mitochondria into Spleen

Isolated mitochondria are first dispersed in a conjugation buffer consisting of 4:1 mixture of Solution X made from mixture of 20 mM HEPES+1 mM EGTA+300 mM Trehalose (pH 7.2) and Solution Y consisting of 0.1 M CHES (pH 10)+0.2 M sodium phosphate dibasic dihydrate. Mitochondria suspension are first gently mixed with poly-L-lysine or polyethyleneimine for 1-5 minutes at room temperature, protected from the light, before FAM-labeled ssDNA or GFP mRNA or mCherry mRNA or Luciferase mRNA is added into the mixture. The complex is then mixed and incubated for the next 30 minutes at RT and in dark environment.

In vivo experiment is conducted to understand the specific biodistribution of mitochondria-oligonucleotide complex inside a tissue or organ after injection. By using a syringe connected to a catheter, 2 mg of mitochondria-ssDNA complex (in 5 mL of 20 mM HEPES+1 mM EGTA+300 mM Sucrose (pH 7.2)) are directly injected into the splenic artery of a pig. Six hours post injection, the pig is sacrificed, and the spleen tissue is harvested. The spleen is fixed using formalin and stored at 4° C. for a few days before processed.

The samples are trimmed, embedded in paraffin, and cut at approximate thickness of 4 μm. The sections are processed for either H & E staining or DAPI staining. Images are processed using a Leica DM400 B Led fluorescent microscope.

### Example 19. In Vivo Delivery of Oligonucleotides Using Mitochondria into Lung

Isolated mitochondria are first dispersed in a conjugation buffer consisting of 4:1 mixture of Solution X made from mixture of 20 mM HEPES+1 mM EGTA+300 mM Trehalose (pH 7.2) and Solution Y consisting of 0.1 M CHES (pH 10)+0.2 M sodium phosphate dibasic dihydrate. Mitochondria suspension are first gently mixed with poly-L-lysine or polyethyleneimine for 1-5 minutes at room temperature, protected from the light, before FAM-labeled ssDNA or GFP mRNA or mCherry mRNA or Luciferase mRNA is added into the mixture. The complex is then mixed and incubated for the next 30 minutes at RT and in dark environment.

In vivo experiment is conducted to understand the specific biodistribution of mitochondria-oligonucleotide complex inside a tissue or organ after injection. By using a syringe connected to a catheter, 2 mg of mitochondria-ssDNA complex (in 5 mL of 20 mM HEPES+1 mM EGTA+300 mM Sucrose (pH 7.2)) are directly injected into the pulmonary artery of a pig. Six hours post injection, the pig is sacrificed, and the lung tissue is harvested. The lung is fixed using formalin and stored at 4° C. for a few days before processed.

The samples are trimmed, embedded in paraffin, and cut at approximate thickness of 4 μm. The sections are processed for either H & E staining or DAPI staining. Images are processed using a Leica DM400 B Led fluorescent microscope.

### Example 20. In Vivo Delivery of Oligonucleotides Using Mitochondria into Intestines

Isolated mitochondria are first dispersed in a conjugation buffer consisting of 4:1 mixture of Solution X made from mixture of 20 mM HEPES+1 mM EGTA+300 mM Trehalose (pH 7.2) and Solution Y consisting of 0.1 M CHES (pH 10)+0.2 M sodium phosphate dibasic dihydrate. Mitochondria suspension are first gently mixed with poly-L-lysine or polyethyleneimine for 1-5 minutes at room temperature, protected from the light, before FAM-labeled ssDNA or GFP mRNA or mCherry mRNA or Luciferase mRNA is added into the mixture. The complex is then mixed and incubated for the next 30 minutes at RT and in dark environment.

In vivo experiment is conducted to understand the specific biodistribution of mitochondria-oligonucleotide complex inside a tissue or organ after injection. By using a syringe connected to a catheter, 2 mg of mitochondria-ssDNA complex (in 5 mL of 20 mM HEPES+1 mM EGTA+300 mM Sucrose (pH 7.2)) are directly injected into the superior mesenteric artery of a pig. Six hours post injection, the pig is sacrificed, and the intestine tissue is harvested. The intestine is fixed using formalin and stored at 4° C. for a few days before processed. The samples are trimmed, embedded in paraffin, and cut at approximate thickness of 4 μm. The sections are processed for either H & E staining or DAPI staining. Images are processed using a Leica DM400 B Led fluorescent microscope.

### Example 21. In Vivo Delivery of Oligonucleotides Using Mitochondria into Bladder

Isolated mitochondria are first dispersed in a conjugation buffer consisting of 4:1 mixture of Solution X made from mixture of 20 mM HEPES+1 mM EGTA+300 mM Trehalose (pH 7.2) and Solution Y consisting of 0.1 M CHES (pH 10)+0.2 M sodium phosphate dibasic dihydrate. Mitochondria suspension are first gently mixed with poly-L-lysine or polyethyleneimine for 1-5 minutes at room temperature, protected from the light, before FAM-labeled ssDNA or GFP mRNA or mCherry mRNA or Luciferase mRNA is added into the mixture. The complex is then mixed and incubated for the next 30 minutes at RT and in dark environment.

In vivo experiment is conducted to understand the specific biodistribution of mitochondria-oligonucleotide complex inside a tissue or organ after injection. By using a syringe connected to a catheter, 2 mg of mitochondria-ssDNA complex (in 5 mL of 20 mM HEPES+1 mM EGTA+300 mM Sucrose (pH 7.2)) are directly injected into the superior and inferior vesical arteries of a pig. Six hours post injection, the pig is sacrificed, and the bladder tissue is harvested. The bladder is fixed using formalin and stored at 4° C. for a few days before processed.

The samples are trimmed, embedded in paraffin, and cut at approximate thickness of 4 μm. The sections are processed for either H & E staining or DAPI staining. Images are processed using a Leica DM400 B Led fluorescent microscope.

### Example 22. In Vivo Delivery of Oligonucleotides Using Mitochondria into the Kidney or Bladder or Intestines or Pancreas or Duodenum or Liver or Lung or Spleen Through Direct Organ Injection

Isolated mitochondria are first dispersed in a conjugation buffer consisting of 4:1 mixture of Solution X made from mixture of 20 mM HEPES+1 mM EGTA+300 mM Trehalose (pH 7.2) and Solution Y consisting of 0.1 M CHES (pH 10)+0.2 M sodium phosphate dibasic dihydrate. Mitochondria suspension are first gently mixed with poly-1-lysine or polyethyleneimine for 1-5 minutes at room temperature, protected from the light, before FAM-labeled ssDNA or GFP mRNA or mCherry mRNA or Luciferase mRNA is added into the mixture. The complex is then mixed and incubated for the next 30 minutes at RT and in dark environment.

In vivo experiment is conducted to understand the specific biodistribution of mitochondria-oligonucleotide complex inside a tissue or organ after injection. By using a syringe connected to a catheter, 2 mg of mitochondria-ssDNA complex (in 5 mL of 20 mM HEPES+1 mM EGTA+300 mM Sucrose (pH 7.2)) are directly injected into the kidney or bladder or intestines or pancreas or duodenum or liver or lung or spleen of a pig. Six hours post injection, the pig is sacrificed, and the organ of interest is harvested. The organ is fixed using formalin and stored at 4° C. for a few days before processed.

The samples are trimmed, embedded in paraffin, and cut at approximate thickness of 4 μm. The sections are processed for either H & E staining or DAPI staining. Images are processed using a Leica DM400 B Led fluorescent microscope.

### Example 23. In Vivo Delivery of Amino Acids Using Mitochondria into Kidney

Mitochondria GFP-tagged human cardiac fibroblasts (GFP-HCF) are cultured in Fibroblast Medium-2 (ScienCell) until reaching 80-90% cell confluency (2-4 million cells/flask). Labeled mitochondria are isolated according to the established cellvie SOP (NPL8). Isolated mitochondria are resuspended in the conjugation buffer at a final concentration of 1 mg/mL. For each 50 μL of the mitochondria solution, 0.2-0.5 μL of lysine or arginine or histidine (concentration of 10 mg/mL in water) is added and gently mixed with the solution. The solution is kept under room temperature for 5-30 minutes.

By using a syringe connected to a catheter, 2 mg of mitochondria-amino acids complex (in 5 mL of 20 mM HEPES+1 mM EGTA+300 mM Sucrose (pH 7.2)) are directly injected into the renal artery of a pig. 6 to 24 hours post injection, the pig is sacrificed, and the kidney tissue is harvested. The kidney is fixed using formalin and stored at 4° C. for a few days before processed.

Five cortical and five medullar biopsies are taken and the tissues are immediately transferred to 10% neutral phosphate-buffered formalin solution (4% w/v formaldehyde solution) for preservation purposes. Samples are trimmed, embedded in paraffin, and cut at approximate thickness of 4 μm.

The sections are processed for either H & E staining or DAPI staining. In particular, 20 slides are processed for H & E imaging, while 4 slides (2 cortical, 2 medullar) are processed for DAPI staining.

The presence of mitochondria-amino acids complex (GFP signal) in the kidney is visualized using a Leica DM400 B Led fluorescent microscope. For H & E processed sections, images are taken using brightfield (BF) or the fluorescent FITC (GFP) channel. Furthermore, 3 images are taken per section (both BF and FITC or DAPI and FITC) using the 40× objective, while one image per section (both BF and FITC or DAPI and FITC) is taken using the 20× objective.

### Example 24: Synthesis, Characterization and In Vitro Study of Mitochondria-ssDNA Complex and Mitochondria-mRNA Complex (2^{nd} Gen Complex)

This example illustrates the procedure for the synthesis, examination of physical and chemical properties, and in vitro examination of (i) a 2^{nd} gen complex consisting of mitochondria, polycationic polymer, single-stranded DNA (ssDNA) and protective polymer as well as a (ii) 2^{nd} gen complex consisting of mitochondria, polycationic polymer, and EGFP/mCherry mRNA and protective polymer in various cell types.

### Materials

Two fluorescently labeled ssDNA (FAM-ssDNA and Cy3-ssDNA), with oligo sequence 5′ to 3′ GCAACAGTGAAGGAAAGCC (NPL9), were purchased from Thermo Fisher Scientific (USA) and used without any further purification. CleanCap® EGFP mRNA, 100 μg (Ref: 040L-7601-100) and CleanCap® mCherry mRNA, 100 μg (Ref: L-7203-100) were purchased from Trilink Biotechnologies. Polyethyleneimine (PEI), branched, molecular weight (MW) 10000, 99% (Ref: 040331.14) were purchased from Thermo Fisher Scientific. Poly(ethylene glycol)-block-polyethyleneimine (PEG/PEI), MW: 15000 (Ref: 910791) was purchased from Sigma-Aldrich. Lipofectamine™ RNAiMAX Transfection Reagent (Ref: 13778100) was purchased from Thermo Fisher Scientific.

### Methodology

### Synthesis of Mitochondria-ssDNA Complex

The frozen HCF mitochondria were thawed at room temperature for a few minutes. The isolated mitochondria were then mixed with a conjugation buffer made up of a 4:1 combination of Solution X, which is a mixture of 20 mM HEPES+1 mM EGTA+300 mM Trehalose (pH 7.2) and Solution Y, which is 0.1 M CHES (pH 10)+0.2 M sodium phosphate dibasic dihydrate. The mitochondrial concentration was kept at 2 mg/mL, equivalent to ca. 6 billion particles/mL.

500 μL of the isolated mitochondrial suspension was then combined with 9 μL of polyethyleneimine with a molecular weight of 10000 (1 mg/mL in deionized water) for 5 minutes at room temperature, in the absence of light, before 10 μL of FAM-labeled ssDNA (0.7 μg/μL in PBS) was added to the mixture. The complex was then mixed and incubated for an additional 25 minutes at room temperature and in the dark. 17 μL of poly(ethylene glycol)-block-polyethyleneimine (1 mg/mL in deionized water) was then added to the mixture, mixed, and incubated for an additional 5 minutes.

For higher concentration and injection studies using a 30G needle (Braun), the complex (2 mg/mL) was centrifuged and the supernatant was discarded. The pellet was resuspended in 50 μL of conjugation buffer, mixed gently using a pipette. The final concentration of the concentrated complex is 20 mg/mL. The complex after injection was analyzed using a fluorescence microscope.

### Synthesis of Mitochondria-EGFP mRNA Complex

The isolated HCF or MEF mitochondria were mixed with the conjugation buffer. The mitochondrial concentration was kept at 2 or 4 mg/mL. 500 μL of the isolated mitochondrial suspension was then combined with 26 μL of polyethyleneimine (PEI, 1 mg/mL in deionized water) for 5 minutes at room temperature, in the absence of light, before 10-20 μL of CleanCap® EGFP mRNA (1 μg/μL in 1 mM Sodium Citrate pH 6.4) was added to the mixture. The complex was then mixed and incubated for an additional 25 minutes at room temperature and in the dark. 52 μL of poly(ethylene glycol)-block-polyethyleneimine (PEG/PEI, 1 mg/mL in deionized water) was then added to the mixture, mixed, and incubated for an additional 5 minutes.

### Synthesis of Mitochondria-EGFP mRNA Complex with Centrifugation Step in Between

The isolated MEF mitochondria were mixed with the conjugation buffer inside Protein LoBind® Tubes (Eppendorf). The mitochondrial concentration was kept at 4 mg/mL. 50 μL of the isolated mitochondrial suspension was then combined with 2.6 μL of polyethyleneimine (PEI, 1 mg/mL in deionized water) for 5 minutes at room temperature, in the absence of light. After, the mixture was centrifuged at 9500 rpm for 5 mins and the supernatant was removed and the pellet was resuspended in 50 μL of conjugation buffer. This washing process was repeated once more. After, 1.5 μL of CleanCap® EGFP mRNA (1 μg/μL in 1 mM Sodium Citrate pH 6.4) was added to the mixture. The complex was then mixed and incubated for an additional 25 minutes at room temperature and in the dark. Next, the mixture was centrifuged at 9500 rpm for 5 mins and the supernatant was removed and the pellet was resuspended in 50 μL of conjugation buffer. 5.2 μL of poly(ethylene glycol)-block-polyethyleneimine (PEG/PEI, 1 mg/mL in deionized water) was then added to the mixture, mixed, and incubated for an additional 10 minutes.

### Synthesis of Mitochondria-mCherry mRNA Complex

The isolated HCF or HepG2 or MEF mitochondria were first dispersed in the conjugation buffer at 2 or 4 mg/mL. 500 μL of isolated mitochondria suspension were first gently mixed with 26 μL polyethyleneimine (1 mg/mL in deionized water), for 5 minutes at room temperature, protected from the light, before 10-20 μL CleanCap® mCherry mRNA (1 μg/μL in 1 mM Sodium Citrate pH 6.4) was added into the mixture. The complex was then mixed and incubated for the next 25 minutes at RT and in a dark environment. 52 μL poly(ethylene glycol)-block-polyethyleneimine (1 mg/mL in deionized water) was added to the mixture and the solution was mixed and incubated for another 5 minutes.

In Vitro Administration: Standard Vs. Premix

The influence of experimental design upon administration of the complex in A549 cells was studied. Two conditions were applied: in one experiment (standard), mitochondria-EGFP mRNA complex was added directly to the well plate containing the cells and cell medium, and then the complex was gently mixed by shaking the plate. In the second experiment, culture medium in the well plate was removed and the complex, which was first premixed in culture medium, was added to the cells. The cells were incubated for 24 h-48 h before fluorescence imaging experiments were conducted. By using image processing, the surface area containing EGFP positive cells were quantified and normalized to untreated cells. The result is shown as EGFP expression per well.

### Synthesis of Mitochondria-EGFP mRNA Complex Varying N/P Ratio

The isolated HCF mitochondria were first dispersed in the conjugation buffer at 2 mg/mL. For sample with N/P ratio 20, 250 μL of isolated mitochondria suspension were first gently mixed with 13 μL polyethyleneimine (1 mg/mL in deionized water), for 5 minutes at room temperature, protected from the light, before 5 μL CleanCap® EGFP mRNA (1 μg/μL in 1 mM Sodium Citrate pH 6.4) was added into the mixture. The complex was then mixed and incubated for the next 25 minutes at RT and in a dark environment.

The solution was then split into 5×50 μL tubes. For sample 20_1, 20_2, 20_3, 20_4, 20_5 each tube was mixed with 1.3, 2.6, 3.9, 5.2, and 6.5 μL poly(ethylene glycol)-block-polyethyleneimine (1 mg/mL in deionized water). The solutions were incubated for another 5 minutes.

For sample with N/P ratio 30, 250 μL of isolated mitochondria suspension were first gently mixed with 19.5 μL polyethyleneimine (1 mg/mL in deionized water), for 5 minutes at room temperature, protected from the light, before 5 μL CleanCap® EGFP mRNA (1 μg/μL in 1 mM Sodium Citrate pH 6.4) was added into the mixture. The complex was then mixed and incubated for the next 25 minutes at RT and in a dark environment.

The solution was then split into 5×50 μL tubes. For sample 30_1, 30_2, 30_3, 30_4, 30_5 each tube was mixed with 1.95, 3.9, 5.85, 7.8, and 9.75 μL poly(ethylene glycol)-block-polyethyleneimine (1 mg/mL in deionized water). The solution incubated for another 5 minutes.

### Synthesis of Lipofectamine-EGFP/mCherry mRNA

In mixture A, 500 μL of OptiMEM was mixed with 50 μL of Lipofectamine RNAiMAX and incubated at RT for 10 minutes. In mixture B, 500 μL of OptiMEM was mixed with either 10 μL of CleanCap® EGFP mRNA or CleanCap® mCherry mRNA and incubated at RT for 10 minutes. The two mixtures were mixed and incubated at RT for an additional 5 minutes.

### Synthesis of Polymeric Nanoparticles Encapsulating EGFP mRNA

To synthesize PEI nanoparticles, 500 μL of conjugation buffer were gently mixed with 26 μL polyethyleneimine MW: 10000 (1 mg/mL in deionized water and 10 μL CleanCap® EGFP mRNA (1 μg/μL in 1 mM Sodium Citrate pH 6.4). The complex was then incubated for the next 30 minutes at RT and in a dark environment.

To synthesize PEG/PEI nanoparticles, 500 μL of conjugation buffer were gently mixed with 52 μL poly(ethylene glycol)-block-polyethyleneimine MW: 15000 (1 mg/mL in deionized water and 10 μL CleanCap® EGFP mRNA (1 μg/μL in 1 mM Sodium Citrate pH 6.4). The complex was then incubated for the next 30 minutes at RT and in a dark environment.

Quantification of mRNA Concentration Using Qubit RNA HS Assay Kits

The isolated HCF mitochondria were mixed with the conjugation buffer at a concentration of 4 mg/mL. 100 μL of the isolated mitochondrial suspension was then combined with 5.2 μL of polyethyleneimine (1 mg/mL in deionized water) for 5 minutes at room temperature, in the absence of light. The mixture was then centrifuged at 9500 rpm for 5 minutes and the supernatant was discarded. The pellet was resuspended in 50 μL of conjugation buffer. The mixture was then recentrifuged at 9500 rpm for 5 minutes and the supernatant was discarded. The pellet was resuspended in 50 μL of conjugation buffer and next, 2 μL of CleanCap® EGFP mRNA (1 μg/μL in 1 mM Sodium Citrate pH 6.4) was added to the mixture. The complex was then mixed and incubated for an additional 25 minutes at room temperature and in the dark. The mixture was then centrifuged at 9500 rpm for 5 minutes and the supernatant was collected for the Qubit™ RNA HS measurement. The measurement assay was performed following the protocol developed by the manufacture (Thermo Fisher Scientific). Briefly, Qubit™ working solution was prepared by diluting the Qubit™ RNA HS reagent 1:200 in Qubit™ RNA HS buffer. Calibration was performed by measuring standards consisting of a mixture of 10 μL of Qubit™ standard and 190 μL of Qubit™ working solution using Qubit™ 3 Fluorometer (RNA High Sensitivity). For mitochondria-mRNA complex measurement, 2 μL of the supernatant sample was mixed with 198 μL of Qubit™ working solution, and the sample was measured. For control sample, 2 μL of CleanCap® EGFP mRNA (1 μg/μL in 1 mM Sodium Citrate pH 6.4) was added to the conjugation buffer and incubated for 25 minutes. 2 μL of the control sample was mixed with 198 μL of Qubit™ working solution, and the sample was measured. The total amount of mRNA (in ng/mL) attached on the mitochondria was calculated based on the Qubit™ read out value, using the following equation:

$$mRNA attached = Control sample value - Supernatant value \qquad ( Eq . 2 )$$

The Amount of mRNA Per Mitochondrion was Calculated by the Following Equation:

$$mRNA per mitochondrion = \frac{number of mRNA attached}{number of mitochondria} \qquad ( Eq . 3 )$$

Where Number of mRNA Molecules was Calculated Using the Following Equation:

$$number of mRNA molecule = number of moles \times Avogadro constant \qquad ( Eq . 3 )$$

### Stability of Mitochondria-mRNA Complex in Different Media

In this study, we aimed to evaluate the stability of nanoparticles in different media, including in conjugation buffer, FBS and cell culture medium. To accomplish this, we prepared mitochondria-EGFP mRNA working solution by mixing equal volumes of mitochondria-EGFP mRNA stock solution (12B/mL) with each media at a ratio of 1:1 (v/v). After the mixing (0 h), size analysis was performed using Coulter Counter. The solutions were then incubated at 37° C. for 24 hours to allow for any potential changes in stability (i.e. change in size) and the sample was analyzed. All experiments were performed in triplicate. One-way ANOVA followed by Tukey's post hoc test was used to analyze the statistical significance of the differences among the groups.

Overall, this experimental method allowed us to assess the stability of the complex in different media and determine whether they can maintain their integrity and properties after being exposed to different environments.

### Long Term Storage Study

Mitochondria-mCherry mRNA and mitochondria-EGFP mRNA complex were stored at −80° C. for 1 or 2 or 4 months before being used for the in vitro imaging experiment. Approximately 10000-25000 A549 or HCF cells were grown on 48 well plates for 24-48 h. The cells were incubated with the complex at concentration of 50 and 100 μg for 24 h, before fluorescence imaging experiment was conducted.

### Flow Cytometry (FACS)

Post mitochondria-mRNA complex or Lipofectamine-mRNA or nanoparticle-mRNA incubation, cells were washed with PBS and trypsinized, then collected, and washed with FACS buffer (1×PBS, 2% FBS, 0.5 mM EDTA). The cells were resuspended in FACS buffer. A flow cytometry experiment to measure EGFP/mCherry positive cells after complex transplantation was performed on FACS machine Symphony A1 using the FACS Diva software (BD, USA) and recorded fluorescence signals were analyzed using FlowJo software (Tree Star, Ashland, USA). The result was also represented as a relative value to Lipofectamine (Lipofectamine as 100%).

In Vitro Study of Protein Expression in iCell Cardiomyocytes²

Approximately 50000 iCell Cardiomyocytes² cells were grown on 96 well plates for 5 days. The medium of the cells was changed every two days. Afterwards, 40 μg of the complex composed of mitochondria-mCherry mRNA or mitochondria-EGFP mRNA complex or 10 μL of Lipofectamine-mCherry mRNA and Lipofectamine-EGFP mRNA was added, and the cells were incubated for 24-48 hours before fluorescence microscopy or FACS was conducted. Beating rate measurement was performed by manually counting the beat rate of the cells per minute after the treatment with all samples. The experiments were performed in triplicate.

### Results and Discussion

(Isolated) viable mitochondria possess a negatively-charged surface, therefore may be functionalized with cationic molecules or polymers, turning the surface charge of mitochondria's outer membrane toward a positive value. Subsequently, positively-charged mitochondria may be conjugated with negatively-charged oligonucleotides.

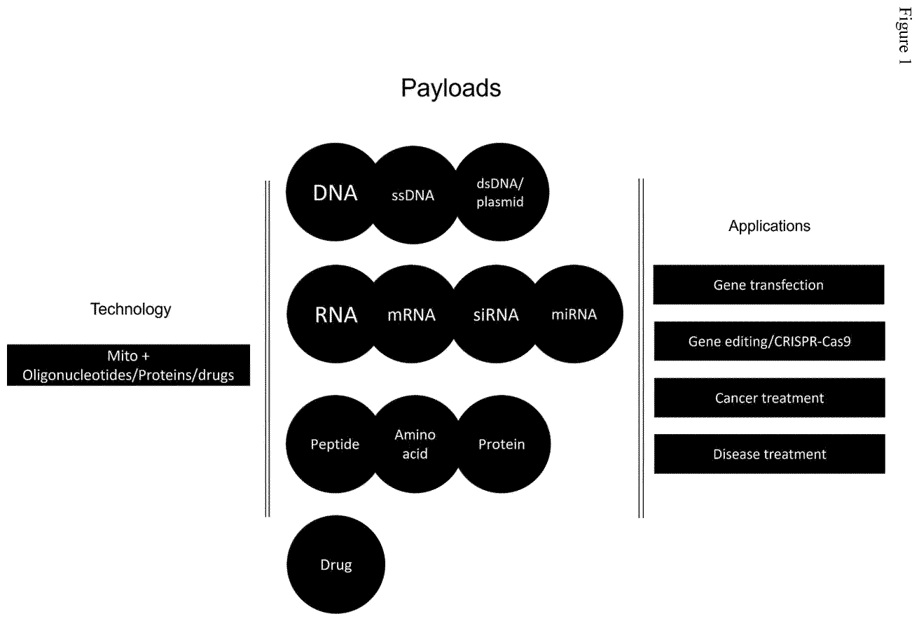

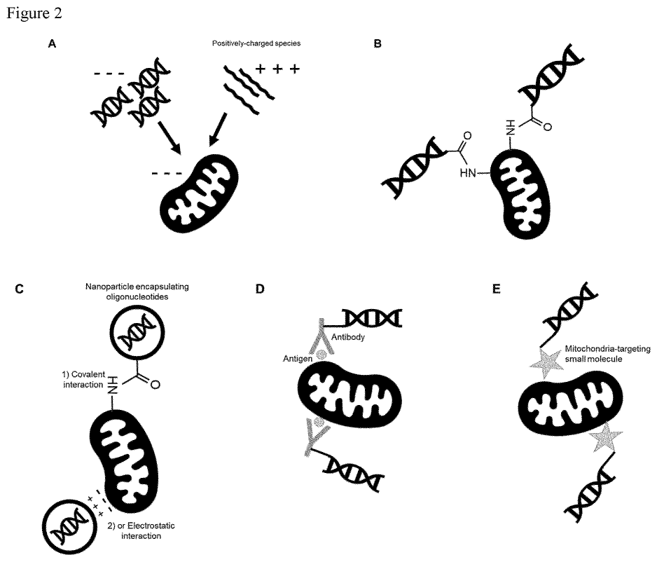

In our previous system, mitochondria were conjugated first with cationic polymers and then, with oligonucleotides (e.g. ssDNA, pDNA, ssRNA, mRNA and siRNA) creating the overall mitochondria-oligonucleotide complex (FIGS. 24 and 25, 1^{st} generation (1^{st} gen)). The uptake of the complex was observed in various cells. However, the overall uptake and mRNA protein expression efficiency is rather low. We attribute this to any possible degradation of the oligonucleotides upon transport or during interaction with the cellular environments, as oligonucleotides are directly exposed to the environment because they sit on the outer part of the complex. Therefore, providing protection to the complex by adding an extra layer of molecules/polymers could be used to overcome the mentioned limitations. PEG-modified polymers (such PEG/PEI) are indeed a good candidate for this, as a previous example in the nanotechnology field has allowed various nanoparticles to be efficiently internalized by cells and more importantly, to escape the digestive organelles (i.e. lysosomes) upon internalization (NPL14).

Hereby, an improved design is proposed where an additional polymer layer (protective layer) in the preform mitochondria-oligonucleotide complex is added (FIGS. 24 and 25, 2^{nd} generation (2^{nd} gen)). We employ electrostatic interaction to conjugate oligonucleotides and polymer-functionalized mitochondria, by using a layer-by-layer technique (lbl). The chemical reaction can only be performed under special conditions. For this, we used a similar conjugation buffer made up of a 4:1 combination of Solution X, which is a mixture of 20 mM HEPES+1 mM EGTA+300 mM Trehalose (pH 7.2) and Solution Y, which is 0.1 M CHES (pH 10)+0.2 M sodium phosphate dibasic dihydrate, which allows the reaction to take place and avoids any mitochondria and oligonucleotide degradation/aggregation during the reaction. The use of different polymers helps not only to attach different oligonucleotides (i.e., mRNA, siRNA, ssDNA, etc.) on the mitochondrial surface, but also to increase the uptake of the mitochondria-oligo complex as well as to avoid any complex aggregation and phagocytosis by immune cells. Modes of action:
Cationic polymers such as polyethyleneimine (PEI) modify negatively-charged mitochondrion's surface, allowing the attachment of negatively-charged oligonucleotides (e.g., mRNA, siRNA, pDNA, etc.) or negatively-charged polypeptides.
Protective layer such as poly(ethylene glycol)-block-polyethyleneimine (PEG/PEI) protect the attached oligonucleotides from degradation, and they allow the mitochondria-oligo complex to be efficiently internalized and more importantly, to escape the digestive organelles (i.e., lysosomes) upon internalization
Inside the cells, the polymer is degraded, and the oligonucleotides can be released and subsequently processed (transcription, translation or knockdown)

To test our hypothesis of formation of the 2^{nd} gen of mitochondria-oligonucleotide complex, fluorescently labeled mitochondria was conjugated with fluorescently labeled ssDNA (FAM labeled-ssDNA). MitoTracker™ Red CMXRos labeled mitochondria were first functionalized with polyethyleneimine before being conjugated with ssDNA. Afterward, the overall complex was functionalized with cationic block copolymers (i.e. poly(ethylene glycol)-block-polyethyleneimine). The ratio of the two polymers was kept at 1:1 or 1:2. The presence of fluorescent labels is important, as it allows visualization of the complex under a microscope. Fluorescence microscopy data reveals the formation of the complex, with the overall size of >1 μm (FIG. 25A). The presence of double staining (in yellow color) at the same pixel locations indicates a successful attachment of ssDNA on the mitochondria's surface. The resulting complex can be further centrifuged and concentrated at a higher concentration. Photograph showing injection of the concentrated ssDNA-mitochondria complex in a smaller volume (20 mg/mL; 50 μL) using a 30G needle was shown in FIG. 25B-25C. The colocalization of both fluorescent signals of MitoTracker™ Red CMXRos and FAM-ssDNA was presence, indicating the stability of the complex at higher concentration and upon injection.

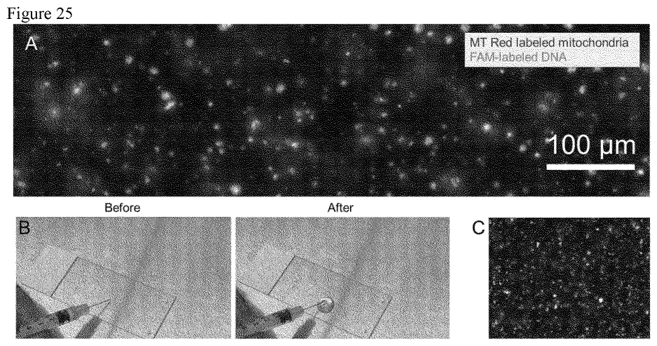

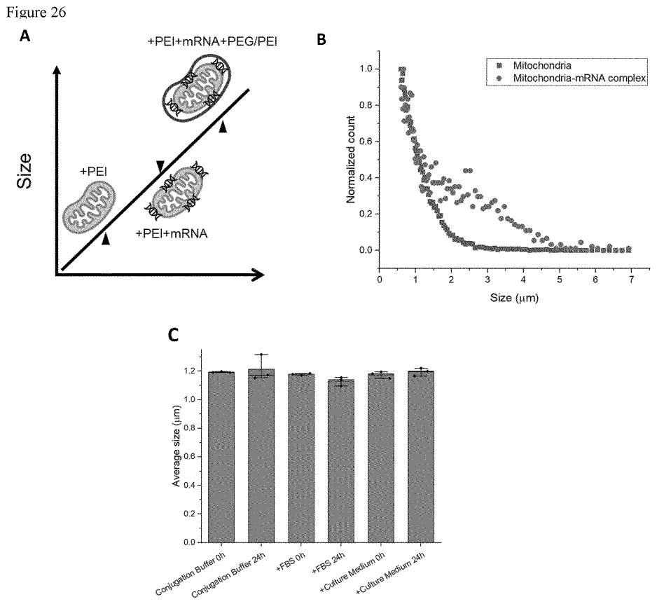

We then tested the same conjugation technique to attach messenger RNA (mRNA) to mitochondria's surface. Two different mRNAs, namely CleanCap® EGFP mRNA or CleanCap® mCherry mRNA, expressing EGFP or mCherry protein in the cells, were used, respectively. We used the 1:2 ratio of the polyethyleneimine and poly(ethylene glycol)-block-polyethyleneimine. The size of mitochondria before and after functionalization was accessed by means of a Coulter Counter device (i.e. particle counter). The size of the mitochondrion is expected to increase in each step of functionalization (FIG. 26A). The change of mean size of a single naked mitochondrion and mitochondria-oligonucleotide complex from 0.981±0.006 μm to 1.193±0.0034 μm was observed (FIG. 26B), suggesting successful functionalization of CleanCap® EGFP mRNA on the mitochondrial surface.

Quantification of the amount of mRNA attached to the single mitochondrion was performed using a well-known Qubit™ RNA HS Assay. However, in the presence of cationic polymers such as polyethyleneimine and poly(ethylene glycol)-block-polyethyleneimine, direct Qubit based quantification is not feasible. Therefore, indirect quantification (i.e., quantification of free mRNA in the supernatant) was done. Quantification of the supernatant of the complex solution after centrifugation at high speed (9500 rpm), indicating the presence of non-binding mRNA, yields ca. 60% of mRNA concentration, suggesting the attachment efficiency of the system closer to 40%. The calculation of number of mRNA molecules, therefore, yields the presence of ca. 2442 mRNA per single mitochondrion. As a comparison, the number of mRNA encapsulated inside a 100-200 nm single lipid nanoparticle was between 2 to 8 mRNA (NPL15). In this regard, our system possesses a thousand folds higher in terms of mRNA encapsulated per unit compared to standard LNPs (Lipid nanoparticles).

The stability of mitochondria-mRNA complex in different media was evaluated by Coulter Counter. The mean particle size of the complex in conjugation buffer, FBS and cell culture medium at 0 h and after 24 hours of incubation was shown in FIG. 26C. There was no significant change in the mean size of the complex in conjugation buffer, FBS and cell culture medium indicating that the complex did not aggregate/agglomerate and did not release the cargo in the media. These findings suggest that mitochondria-mRNA complex can be used in different media without compromising their stability, which is crucial for their biological and biomedical applications.

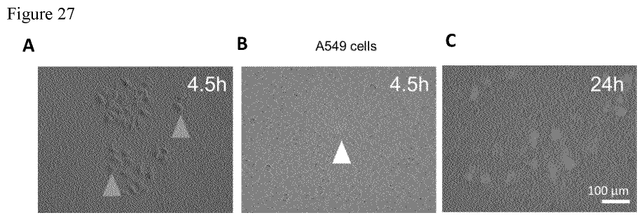

The first in vitro experiment on the developed complex was performed using A549 human epithelial carcinoma cells cultured on 48 well plates. The cells were exposed to the complex at a concentration of 50 μg of mitochondria-mRNA complex (which corresponds to exposure of 150 million mitochondria and 500 ng of mCherry mRNA in a single 48-well plate). After 4.5 h, the cells were monitored under a fluorescence microscope. Brightfield image (FIG. 27A) showing the association of mitochondria-mCherry mRNA complex on A549 cells after 4.5 h of exposure. Dot structures found on the cell surface, inside the cells, and in the cell vicinity were the complexes (FIG. 27A, pointed by the arrow). Fluorescence imaging measurement shows that the signal of mCherry expression started to appear after 4.5 h post complex incubation (FIG. 27B, white arrow). Increasing the incubation time to 24 hours as expected, increases the fluorescence intensity and the number of the cells expressing mCherry protein (FIG. 27C). A similar 24-hour experiment was conducted using the mitochondria-EGFP mRNA complex. The result shows high EGFP expression inside most of the A549 cells.

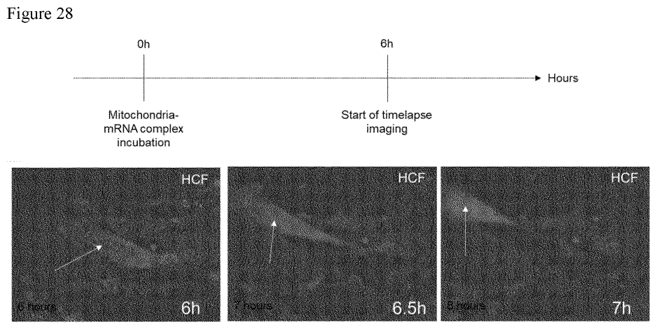

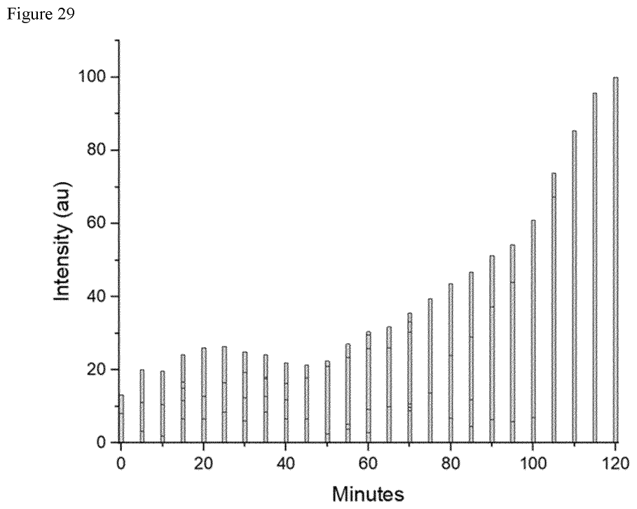

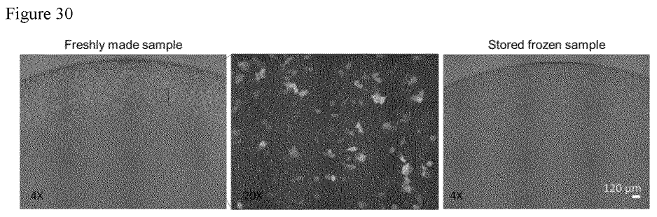

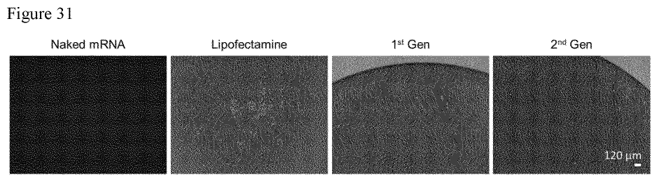

Time-lapse imaging was then performed to visualize the increase of the intensity of EGFP unraveling the evolution of protein expression in a single cell. After 5 hours of exposure to the complex, a few individual cells were monitored for the next 2 hours using a live cell imaging option. Intensity analysis based on image processing (FIGS. 28 and 29) on the EGFP positive cells shows the increase of EGFP signal overtime.

Comparison of the expression of the complexes which were prepared freshly and stored frozen at −80° C. for 2 days was conducted. The expression of the EGFP in A549 cells was observed in both samples. However, a slight reduction in intensity was observed in the −80° C. stored sample. The experiment concludes that the complex can be stored at −80° C. and maintain stability upon thawing.

Direct comparison with a widely-used Lipofectamine delivery system and a previously developed complex (1^{st} gen) was performed. Exposure to naked EGFP mRNA was also studied. The concentration of EGFP mRNA was kept constant in all experiments. Post 24 hours of exposure, as expected, yields a negative EGFP signal in a naked EGFP mRNA sample. The highest signal was detected in the Lipofectamine sample, followed by the current system (2^{nd} gen) and 1^{st} gen complex. A noticeable increase in the numbers of the A549 cells expressing EGFP in 2^{nd} gen compared to 1^{st} gen, confirms the ability of protective layer (e.g., poly(ethylene glycol)-block-polyethyleneimine) to indeed improve the cellular uptake of the complex. Therefore, increasing the percentage share of EGFP cells. PEG-modified PEI (PEG/PEI) itself has been used to increase the uptake of nanoparticles as well as to avoid any nanoparticle aggregation and phagocytosis by immune cells.

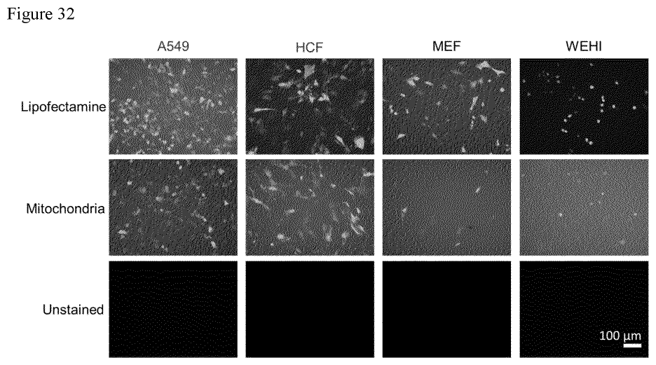

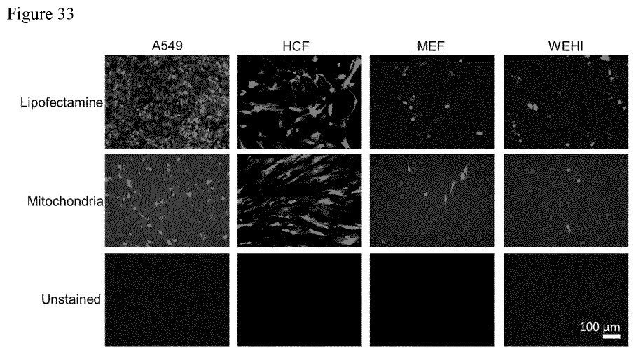

The overall systems (e.g. mitochondria-EGFP mRNA and mitochondria-mCherry mRNA complex) were exposed to different human and mouse cells for 24 hours. Lipofectamine was used as the benchmark for the efficiency study. FIGS. 32 and 33 show the expression of EGFP and mCherry in all tested human and animal cells. In HCF and A549 cells, the number of cells expressing the fluorescent proteins was relatively high compared to in mouse cells (e.g., MEF and WEHI) after being exposed to the complex. Lipofectamine samples yield the highest number of fluorescent positive cells.

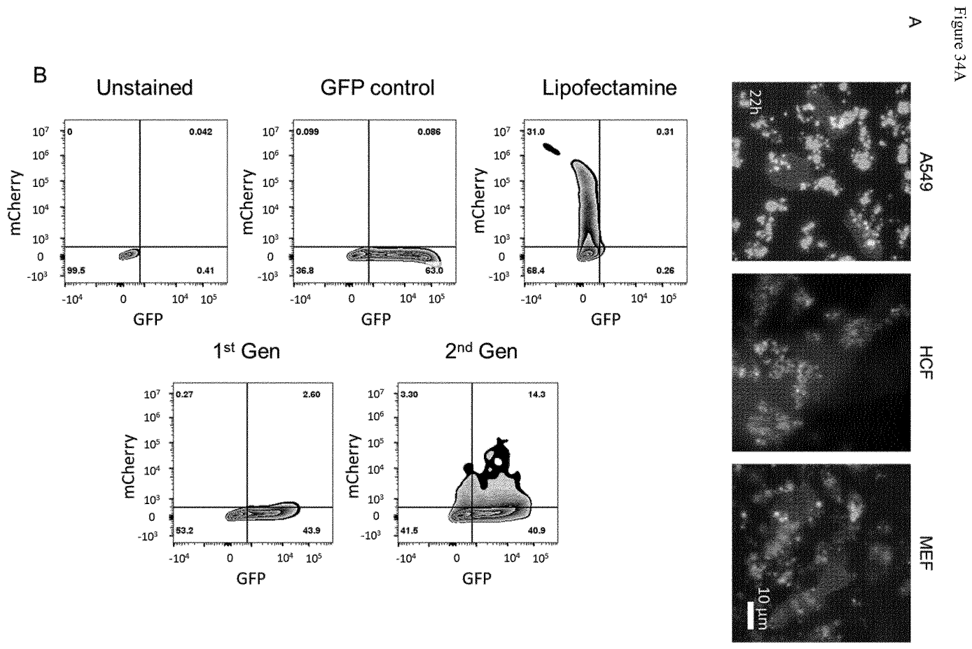

Using fluorescently labeled mitochondria, the presence of the complex inside the cells was tracked. First, GFP-labeled mitochondria were isolated from HepG2 cells possessing GFP-labeled mitochondria. The complex with mCherry mRNA was then formed following a similar procedure described above. A549, HCF and MEF cells were exposed to the complex for 22 h, before being analyzed using a fluorescence microscope. By using GFP-labeled HepG2 mitochondria, we were able to show that in A549, HCF and MEF cells where mCherry protein (red) was expressed, the presence of mitochondria (green) was always observed (FIG. 34A). Further experiment using FACS in HCF cells post 48 h of incubation with a GFP-labeled mitochondria-mCherry mRNA complex was performed to understand the translation efficiency of the internalized complex in the cells. Comparison with the 1^{st} gen complex (without protective layer), the GFP-labeled mitochondria and the Lipofectamine-mCherry mRNA complex was also performed. Analysis of the green GFP signal revealed that 63% of the total cell population internalized GFP-labeled mitochondria, 43.9% internalized the 1^{st} gen complex and 40.9% internalized the 2^{nd} gen complex. As the size of the complexes increased due to functionalization, a reduce in cellular uptake of functionalized mitochondria in comparison to naked mitochondria was indeed expected. Analysis of the red fluorescent signal yielded 2.87%, 17.6% and 31.31% of the total cell population possessed mCherry signal after the incubation with 1^{st} gen complex, 2^{nd} gen complex and Lipofectamine-mCherry mRNA complex, respectively. Comparison of the mCherry over GFP positive cells allowed us to calculate mRNA translation efficiency of the internalized complex, which was found at 6.53% and 43% for the 1^{st} and 2^{nd} gen complex. Here we observed a 6-fold improvement in the efficiency from 1^{st} gen to the 2^{nd} gen complex, in HCF cells (FIG. 34B).

In the field of polymeric nanoparticles, the ratio of positive (typically due to the presence of the nitrogen atom) of polymers and negative charge (typically from the phosphate) of oligonucleotides, which is translated to the amount (weight) of corresponding polymers and oligonucleotides, play an important role in dictating the formation of polyplex nanoparticles affecting the internalization of the polyplex and expression of oligonucleotides in vitro. Positive N/P ratio ((N/P)>4)), as an example, allows the nanoparticles to be efficiently internalized while possessing the highest mRNA expression (NPL16). We argue that in our case, by varying the N/P ratio of the polyethyleneimine, mRNA and poly(ethylene glycol)-block-polyethyleneimine, improvement in the internalization efficiency and mRNA translation can be achieved.

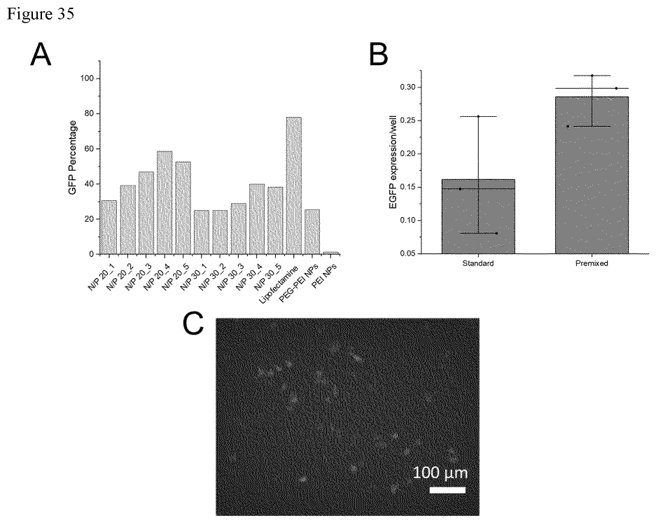

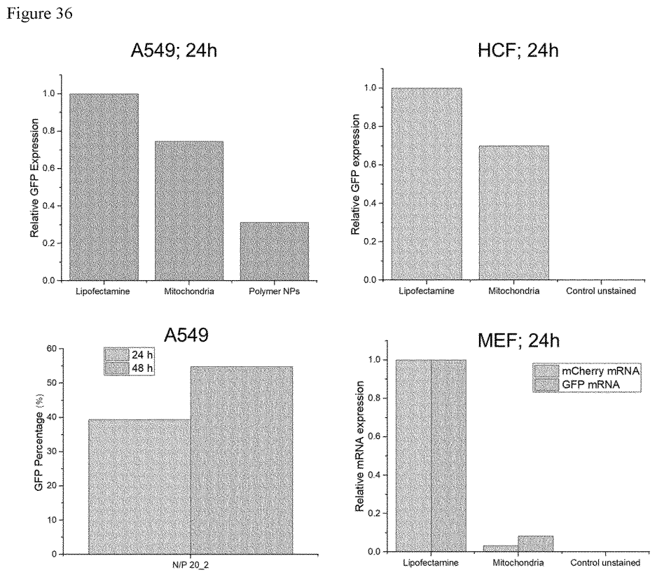

Two N/P ratios of polyethyleneimine and EGFP mRNA, e.g., N/P 20 and 30, were selected while the amount of poly(ethylene glycol)-block-polyethyleneimine were kept at 0.5, 1, 1.5, 2 and 2.5 times of polyethyleneimine. In vitro experiments in A549 cells and in HCF cells were conducted and the cells were collected to be analyzed by means of FACS. As comparison, Lipofectamine, polyplex polyethyleneimine nanoparticles (PEI NPs) and polyplex poly(ethylene glycol)-block-polyethyleneimine nanoparticles (PEG/PEI NPs) were used. FACS analysis shows that the best N/P ratio is N/P 20 with twice the ratio of protective layer (N/P 20_4), yielding ca. 60% cell population expressing the EGFP signal (FIG. 35A). In this respect, relative comparison to Lipofectamine (100%) shows >70% of A549 and HCF cells were positive (FIG. 36). Increasing the incubation/exposure from 24 h to 48 h in A549 cells, as expected, increases the percentage of the cell population possessing the EGFP signal (FIG. 36). However, low EGFP expression (10%) was observed in MEF cells (FIG. 36), which was likely attributed to a low endocytosis profile of the cells.

The influence of experimental design during administration of the complex was studied. Here, two conditions were applied. In one experiment, mitochondria-EGFP mRNA complex was added directly to the well plate containing the cell medium and gently mixed by shaking the plate and in the other experiment, the complex was premixed in culture medium before the complex-cell medium mixture was added to the cells. The cells were incubated for 24 h-48 h before fluorescence imaging experiments were conducted. Image processing analysis shows that premixed in culture medium before complex administration to the cells was shown to improve the overall amount EGFP positive cells compared to mixed in the well plate (standard, FIG. 35B).

To remove any potential unbound polymers and mRNA and formation of mRNA-polymers nanoparticles, centrifugation was introduced during each step of the synthesis. The complex was then administered to A549 cells for 24 h before the imaging experiment was conducted. The expression of EGFP was observed 24 h inside the cells (FIG. 35C), confirming that mitochondria play the only role to carry the oligonucleotides inside the cells.

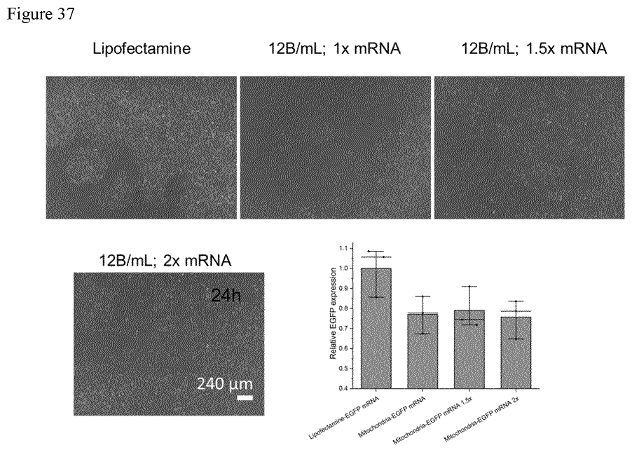

The effect of number of mitochondria and mRNA concentration on the EGFP mRNA translation efficiency was also studied. The concentration of mitochondria was increased from 6 billion/mL to 12 billion/mL, while the mRNA concentration was increased from 10 μg to 15 and 20 μg upon the synthesis (for a mitochondria suspension of 500 μL). In vitro experiment was conducted in A549 cells and fluorescence imaging data shows an increase in the number of cells expressing EGFP signal after 24 h of incubation (FIG. 37). FACS analysis detects a 79.11% and 75.72% of relative EGFP expression in mitochondria-EGFP mRNA complex with 15 μg mRNA (Mito-mRNA 1.5×) and mitochondria-EGFP mRNA complex with 20 μg mRNA (Mito-mRNA 2×) after 48 h in culture, respectively (FIG. 37). The findings affirm that an increase in the quantity of mitochondria and mRNA leads to enhanced internalization and higher translation.

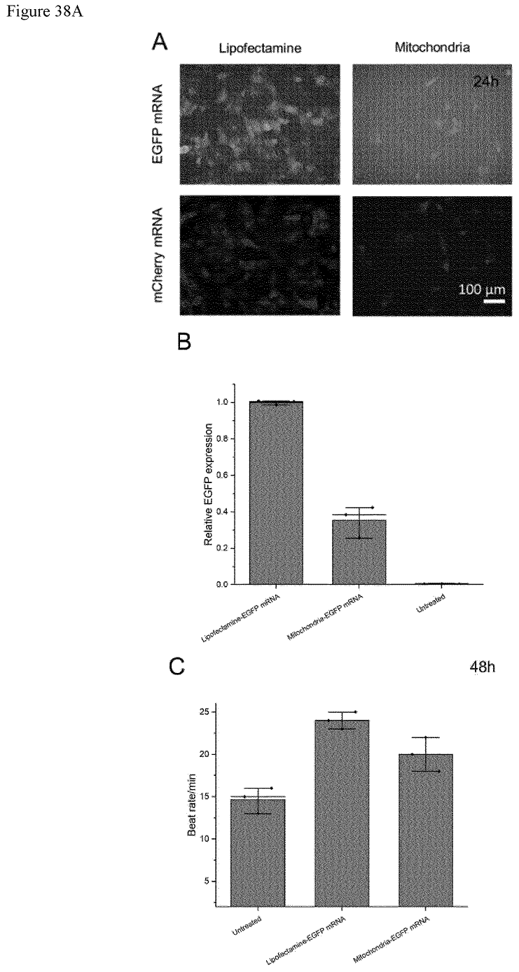

In vitro imaging of mitochondria-mRNA complex (both EGFP and mCherry mRNA), in iCell® Cardiomyocytes² cells after 24 h of incubation was performed. Cardiomyocytes are commonly used in biomedical research due to their crucial role in the functioning of the heart. Studying the behavior of mitochondria-mRNA complex within cardiomyocytes can provide insights into potential therapeutic strategies for treating heart-related conditions, such as genetic disorders or cardiovascular diseases. In vitro fluorescence imaging indicating translation of the mRNA was observed inside cardiomyocytes upon 24 h delivery of the complex (FIG. 38A). FACS measurements show relative expression of mRNA carried by mitochondria (Mito-mRNA 2×) after 48 h of incubation, showing ca. 39% of translation efficiency compared to Lipofectamine as a positive control (FIG. 38B). Calculation of beating rate in the iCell® Cardiomyocytes² cells interestingly shows an increase of the beating rate 48 h after complex or Lipofectamine incubation, compared to untreated cells (FIG. 38C).

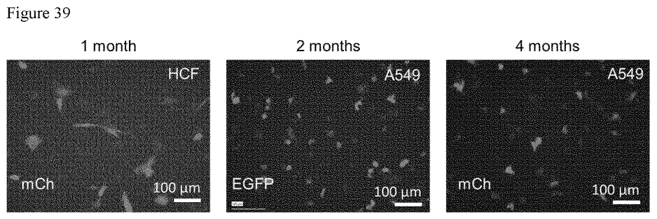

A long term storage study was performed by storing the mitochondria-mCherry mRNA complex and/or mitochondria-EGFP mRNA complex at −80° C. for 1 to 4 month. After thawing, the complex was exposed to HCF or A549 cells. The in vitro mRNA expression study of the stored mitochondria-mRNA complex in HCF was shown in FIG. 39. The mCherry signal was observed 24 h post incubation, indicating long term storage (4 months) at low temperature does not alter and destroy the mitochondria-mRNA complex.

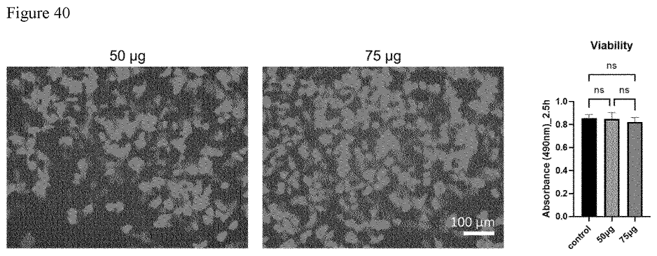

Finally, an MTS assay was performed to understand the cytotoxicity profile of the synthesized complex at two different concentrations. A fluorescent micrograph showing the expression of the mitochondria-mCherry complex and MTS absorbance value for measuring potential cytotoxicity of the administered complex in A549 cells is shown in FIG. 40. Similar absorbance values with negative control (i.e. cells treated only with buffer) were measured, indicating no toxicity was observed in the cells after 24 h of complex incubation at concentrations of 50 μg and 75 μg.

In conclusion, mitochondria-mRNA complex containing protective layer were successfully synthesized and characterized with a significant improvement in the biological activity of the cells (i.e., internalization and mRNA translation) than the 1^{st} gen complex. The uptake and translation of EGFP and mCherry mRNA carried by mitochondria were observed in different cells including A549, HCF, MEF, WEHI and cardiomyocytes. More than 70% of relative mRNA translation efficiency (in comparison to Lipofectamine) was successfully achieved in HCF and A549, without any sign of cytotoxicity.

### Example 25. Synthesis, Characterization and In Vitro Study of Mitochondria-siRNA Complex (2^{nd} Gen Complex)

This example illustrates that the 2^{nd} gen system can also be used to deliver small interference RNA (siRNA) in the same way as mRNA.

### Materials

Silencer™ FAM-labeled GAPDH siRNA and Ambion™ MDM2 siRNA were purchased from Thermo Fisher Scientific. Polyethyleneimine, branched, molecular weight (MW) 10000, 99% (Ref: 040331.14) was purchased from Thermo Fisher Scientific. Poly(ethylene glycol)-block-polyethyleneimine, MW: 15000 (Ref: 910791) was purchased from Sigma-Aldrich. Lipofectamine™ RNAiMAX Transfection Reagent (Ref: 13778100) was purchased from Thermo Fisher Scientific.

### Methodology

### Synthesis of Mitochondria-GAPDH siRNA Complex

The isolated HCF mitochondria were first dispersed in the conjugation buffer at 2 mg/ml (6B/mL). 500 μL of isolated mitochondria suspension were gently mixed with either 4.3 μL, 8 μL, or 12 μL polyethyleneimine (1 mg/mL in deionized water), for 5 minutes at room temperature, protected from the light, before either 4 μL, 8 μL, or 12 μL Silencer™ FAM-labeled GAPDH siRNA (50 M in RNAse/DNAse free water) was added into the mixture. The complex was then mixed and incubated for the next 25 minutes at RT and in a dark environment. 8.6 μL, 12 μL, or 24 μL poly(ethylene glycol)-block-polyethyleneimine (1 mg/mL in deionized water) was added to the mixture and the solution was mixed and incubated for another 5 minutes. The resulting complexes correspond to 50 μg mitochondria (150 million mitochondria) carrying different concentrations of siRNA, e.g. 10, 20, and 30 μmol, respectively.

### Synthesis of Mitochondria-MDM2 siRNA Complex

The isolated HCF mitochondria were first dispersed in the conjugation buffer at 2 mg/mL. 500 μL of isolated mitochondria suspension were gently mixed with 4.3 μL or 8 μL polyethyleneimine MW: 10000 (1 mg/mL in deionized water), for 5 minutes at room temperature, protected from the light, before 4 μL or 8 μL Ambion™ MDM2 siRNA (50 μM in RNAse/DNAse free water) was added into the mixture. The complex was then mixed and incubated for the next 25 minutes at RT and in a dark environment. 8.6 μL or 17.2 μL poly(ethylene glycol)-block-polyethyleneimine (1 mg/mL in deionized water) was added to the mixture and the solution was mixed and incubated for another 5 minutes. The resulting complexes correspond to 50 μg mitochondria (150 million mitochondria) carrying different concentrations of siRNA, e.g. 10 and 20 μmol, respectively.

### Synthesis of Lipofectamine-GAPDH/MDM2 siRNA

In mixture A, 150 μL of OptiMEM was mixed with 4 μL, 8 μL, 12 μL or 16 μL of Lipofectamine RNAiMAX and incubated at RT for 10 minutes. In mixture B, 150 μL of OptiMEM was mixed with either 2.4 μL, 4.8 μL, 7.2 or 9.6 μL of Silencer™ FAM-labeled GAPDH siRNA or Ambion™ MDM2 siRNA and incubated at RT for 10 minutes. The two mixtures were mixed and incubated at RT for an additional 5 minutes. The resulting complexes correspond to 25 μL of Lipofectamine-siRNA complex carrying different concentrations of siRNA, e.g. 10, 20, 30, and 40 μmol, respectively.

### MTS Assay

Approximately 10000-25000 A549 cells were cultured on 24 well-plate at 37° C. and 5% CO₂. After 48 hours, 50 or 75 μg of mitochondria-Silencer™ FAM-labeled GAPDH siRNA or mitochondria-Ambion™ MDM2 siRNA complex (10-40 μmol of siRNA concentration) was added. The cells were further incubated for 96 hours before the unbound mitochondria-siRNA complex was removed through washing with PBS. The MTS assay based on the reduction of the MTS tetrazolium compound by viable cells to generate a colored formazan dye that is soluble in cell culture media was performed following the protocols described by the manufacture (Promega). After 1 h of incubation, the formazan dye is quantified by measuring the absorbance at 490-500 nm using a plate reader.

### SDS-PAGE and Western Blot

Approximately 25000 A549 cells were cultured on 48 well-plate at 37° C. and 5% CO₂. After 48 hours, 50 μg of mitochondria-Silencer™ FAM-labeled GAPDH siRNA complex (10-40 μmol of siRNA concentration) was added. The cells were further incubated for 72 hours before the unbound mitochondria-siRNA complex was removed through washing with PBS. Untreated cells and cells treated with conjugation buffer were used as control in the experiments.

The cells were washed once with PBS, collected by trypsinization, washed twice with PBS supplemented with a 1× Complete EDTA-free protease inhibitor cocktail (Merck, Cat. Nr. 11873580001) (PBS+PIC) and frozen in 30 μl of PBS+PIC at −20° C. After thawing, 0.2 μl of Pierce™ Universal Nuclease for Cell Lysis (Thermo Fisher Scientific, Cat. Nr 88701) was added per sample, and samples were incubated for 20 minutes on ice. Protein content was determined using Pierce™ BCA Protein Assay Kit (Thermo Fisher Scientific, Cat. Nr. 23225) and adjusted to approximately 2 mg/ml for all samples. The samples were mixed with 4× Pierce™ LDS Sample Buffer, Non-Reducing (Thermo Fisher Scientific, Cat. Nr. 84788) to obtain the final 1× concentration. The samples were stored frozen at −20° C. until use. For SDS-PAGE, pre-cast NuPAGE™ 12%, Bis-Tris, 1.0 mm, Mini Protein Gels (Thermo Fisher Scientific, Cat. Nr. NP0343BOX) were loaded with samples in LDS Sample buffer (25 μg protein per well) and run in NuPAGE™ MES SDS Running Buffer (Thermo Fisher Scientific, Cat. Nr. NP0002) at 200 V for 30 min. After that, the gels were blotted on polyvinylidene fluoride (PVDF)/Filter Paper Sandwich, 0.2 μm, 8.3×7.3 cm (Thermo Fisher Scientific, Cat. Nr. LC2002) using Mini Trans-Blot Electrophoretic Transfer Cell (Bio-Rad Cat. Nr. 1703930) at 200 V for 1 hour.

For western blotting, PVDF membrane was blocked for at least 1 h with 10% skimmed milk in phosphate-buffered saline supplemented with 0.1% Tween-20 (PBST) and incubated with antibodies of interest (GAPDH Mouse McAb, ProteinTech Cat. Nr. 60004-1-Ig or Histone-H3 Polyclonal antibody, Proteintech Cat No. 17168-1-AP) for 1 hour at room temperature, followed by 3×5 min washes in PBST and incubation with secondary alkaline phosphatase-conjugated antibodies (Alkaline Phosphatase-conjugated Affinipure Goat Anti-Mouse IgG(H+L), Proteintech Cat. Nr. SA00002-1 or Alkaline Phosphatase-conjugated Affinipure Goat Anti-Rabbit IgG(H+L), Proteintech Cat. Nr. SA00002-2) for 1 hour at room temperature. The membrane was washed three times for 5 minutes in PBST, and developed using Bio-Rad AP Conjugate Substrate Kit (Cat. No. 1706432).

### Results and Discussion

We used Silencer™ FAM-labeled Glyceraldehyde 3-phosphate dehydrogenase (GAPDH) siRNA and Ambion™ MDM2 siRNA as siRNA models. GAPDH plays a role in catalyzing the sixth step of glycolysis and breaking down glucose for energy and carbon molecules. Reduced expression or knockdown of GAPDH expression by siRNA will disrupt glycolysis, a route needed by cancer cells to produce ca. 60% of their ATP (NPL11). Therefore, a reduction in cancer cell viability is expected. Fluorescent labeling with FAM molecules is important, as it allows us to track the complex inside the cells by using a fluorescence microscope. MDM2 provides a function as an important negative regulator of the p 53 tumor suppressor protein, which is vital in inhibiting cancer formation. Reduction of MDM2 protein leads to downregulation of tumor suppressive p 53 pathways (NPL17). Similar to the biological effect of the knockdown of GAPDH, reduction in cancer cell viability is also expected.

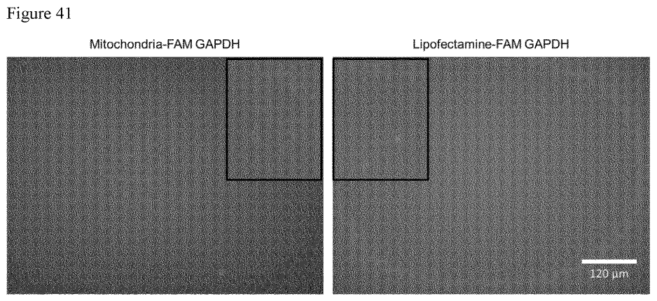

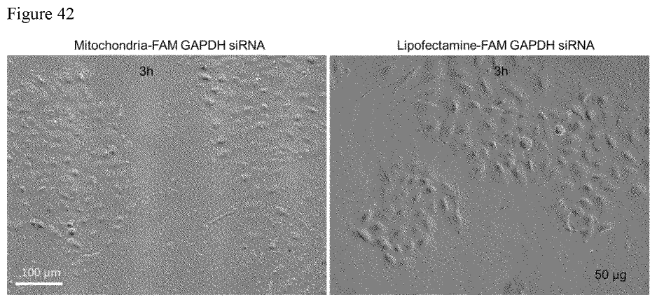

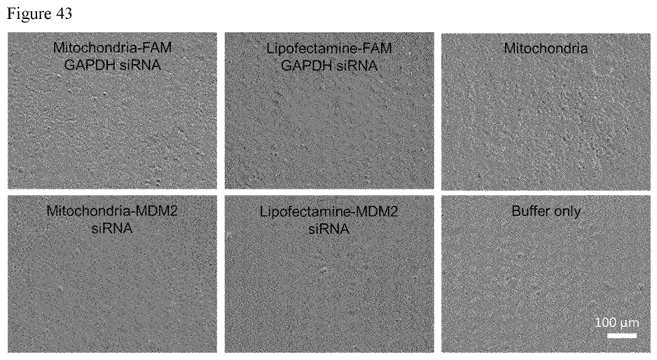

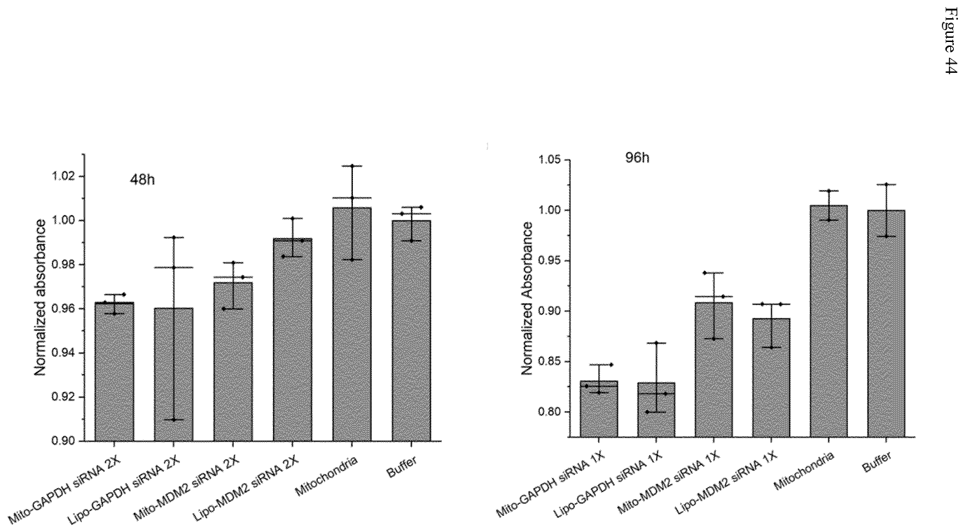

Isolated HCF mitochondria were attached with Silencer™ FAM-labeled GAPDH siRNA and Ambion™ MDM2 siRNA following a similar procedure described previously. The formation of the mitochondria-GAPDH siRNA complex was observed under fluorescence microscope (FIG. 41). In agreement to the results of positive control experiment using Lipofectamine as a delivery agent, fluorescence micrographs show a successful in vitro association of Silencer™ FAM-labeled GAPDH siRNA by mitochondria in A549 cells after 3 h of exposure (FIG. 42). Under the microscope, the complex was shown scattered over the cell cytoplasm, and more importantly without any major sign of agglomeration of the complex inside the cells after 72 h of incubation (FIG. 43). In vitro data further confirmed there was no sign of cell toxicity (no sign of apoptosis/necrosis) after treatment of mitochondria-Silencer™ FAM-labeled GAPDH siRNA or mitochondria-Ambion™ MDM2 siRNA for concentration up to 50 μg (ca. 150 million mitochondria; 10-20 μmol of siRNA) (FIGS. 43 and 44).

The therapeutic effect of the delivery and release of Silencer™ FAM-labeled GAPDH siRNA from the mitochondrial surface inside the cancer cells which can be seen from reduction of cell proliferation in MTS assay. Cell proliferation analysis after 48 h and 96 h of complex incubation reveals a successful knockdown of GAPDH and MDM2 by siRNA delivered by mitochondria. Lower absorbance value of cells treated with mitochondria-siRNA in comparison to untreated cells or mitochondria-treated cells was detected (FIG. 44). At siRNA concentration of ca. 20 μmol, similar absorbance values were found on positive controls using Lipofectamine.

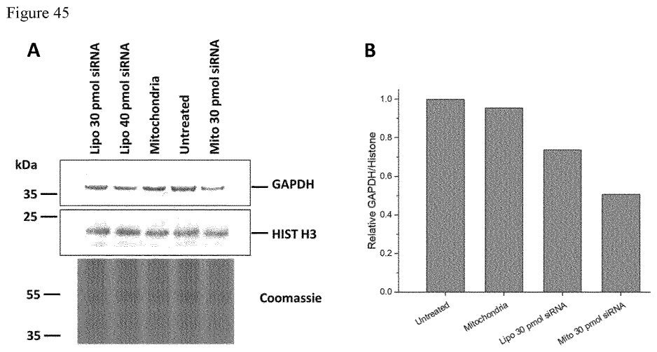

Finally, to quantify protein activity of the cells after being treated with mitochondria-Silencer™ FAM-labeled GAPDH siRNA, SDS Page-Western Blot was performed. A549 cells which were treated with the complex had the lowest GAPDH protein content in comparison to all samples. At similar siRNA concentration (30 μmol), the Western Blot result shows that our 2^{nd} gen system outperforms Lipofectamine to knockdown the protein of interest (FIG. 45).

In conclusion, mitochondria-carrying siRNA was successfully synthesized. We showed that the 2^{nd} gen system outperforms Lipofectamine to knockdown of GAPDH in cancer cells. The result provide a potential therapeutic strategy in the field of oncology.

### Example 26. In Vitro Simultaneous Delivery of Two Different Oligonucleotides (2^{nd} Gen Complex)

This example provides an information regarding simultaneous delivery of different types of oligonucleotides and their combinations.

### Methodology

### Synthesis of Mitochondria Carrying Simultaneously Two Fluorescently Labeled ssDNA

The isolated mitochondria were first dispersed in the conjugation buffer at 1 mg/mL. One mL of isolated mitochondria suspension were first gently mixed with 10 μL poly-1-lysine MW: 70000 (10 mg/mL in deionized water), for 5 minutes at room temperature, protected from the light, before 10 UL FAM-labeled ssDNA and 10 μL Cy3-labeled ssDNA (0.7 μg/μL in water) were added into the mixture. The complex was then mixed and incubated for the next 25 minutes at RT and in a dark environment. Next, 52 μL poly(ethylene glycol)-block-polyethyleneimine was added to the mixture and the solution was incubated further for 5 minutes.

### Synthesis of Mitochondria Simultaneously Carrying EGFP mRNA and FAM-GAPDH siRNA

The isolated mitochondria were first dispersed in the conjugation buffer at 2 mg/mL. 100 μL of isolated mitochondria suspension were first gently mixed with 5.2 μL polyethyleneimine (1 mg/mL in deionized water), for 5 minutes at room temperature, protected from the light, before 1 μL CleanCap® EGFP mRNA (1 μg/μL) and 1 μL Silencer™ FAM-labeled GAPDH siRNA (50 μM in RNAse/DNAse free water) were added into the mixture. The complex was then mixed and incubated for the next 25 minutes at RT and in a dark environment. 10.4 μL poly(ethylene glycol)-block-polyethyleneimine was added to the mixture and the solution was incubated further for 5 minutes.

### Synthesis of Mitochondria Simultaneously Carrying mCherry mRNA and FAM-GAPDH siRNA

The isolated mitochondria were first dispersed in the conjugation buffer at 2 mg/mL. 100 μL of isolated mitochondria suspension were first gently mixed with 5.2 μL polyethyleneimine (1 mg/mL in deionized water), for 5 minutes at room temperature, protected from the light, before 1 μL CleanCap® mCherry mRNA (1 μg/μL) and 1 μL Silencer™ FAM-labeled GAPDH siRNA (50 μM in RNAse/DNAse free water) were added into the mixture. The complex was then mixed and incubated for the next 25 minutes at RT and in a dark environment. 10.4 μL poly(ethylene glycol)-block-polyethyleneimine was added to the mixture and the solution was incubated further for 5 minutes.

### Results and Discussion

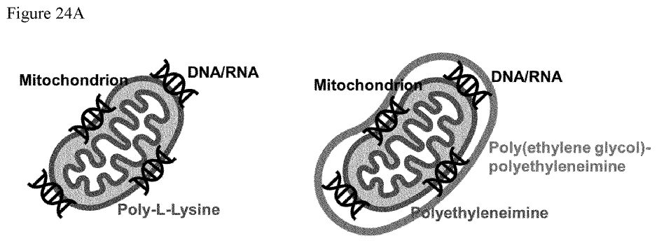

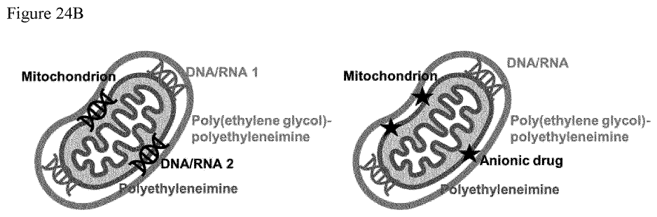

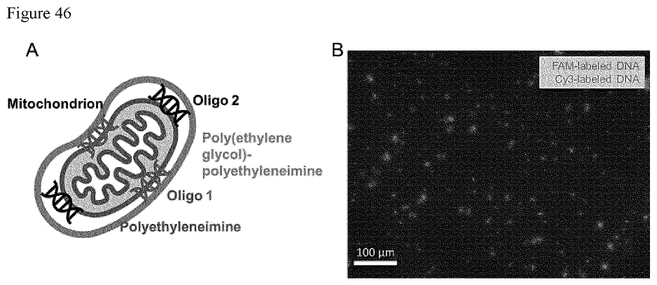

Isolated viable mitochondria possess a negatively-charged surface and therefore, can be functionalized with cationic molecules such as polycations, turning the surface charge of mitochondria's outer membrane to a more positive value. Subsequently, positively-charged mitochondria can be conjugated with negatively-charged oligonucleotides. Following this argument, a mixture of different oligonucleotides, such as two different ssDNA, could be attached simultaneously (FIGS. 24A, 24B and 46). The ratio of the two nucleotides will determine the amount of each nucleotide that can be attached to the mitochondrial surface. To perform the electrostatic interaction, isolated mitochondria were first dispersed in the conjugation buffer. Mitochondria suspension was then first gently mixed with poly-1-lysine for 1-5 minutes at room temperature, protected from the light, before FAM-labeled ssDNA and Cy3-labeled ssDNA (1:1 weight ratio) were simultaneously added into the mixture. The choice of fluorophores was dictated by the difference in the emission profile, avoiding any signal mixing during the analysis. The complex was then mixed and incubated for the next 30 minutes at RT and in a dark environment. A fluorescent microscopy experiment was conducted to characterize the complex.

Fluorescence microscopy data shows a formation of spherical objects with a diameter of in the range of 1.2 to 4 micrometer, under appropriate light excitation (FIG. 46). The presence of both fluorescence signals in the same spot was observed, indicating the successful functionalization of both labeled-DNA molecules on the mitochondrial surface.

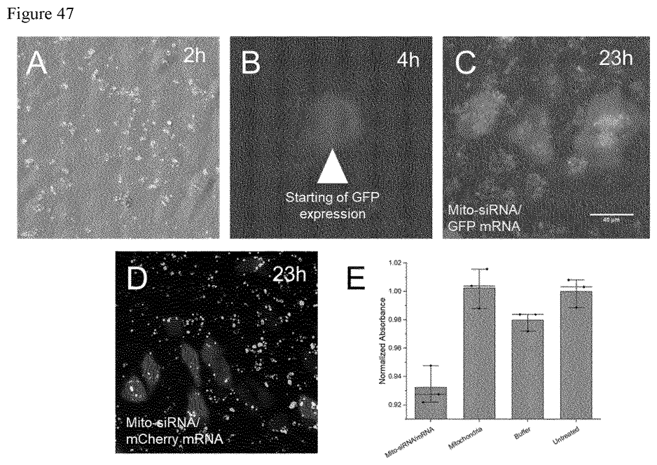

To study whether simultaneous expression and knockdown of protein could be observed, mitochondria carrying EGFP mRNA or mCherry mRNA and FAM-GAPDH siRNA were synthesized. The synthesized complex was then incubated to A549 cells and the cells were monitored at 2 h, 4 h and 23 h post incubation (FIG. 47). The presence of fluorescence signal from FAM-GAPDH associated with the cells was observed after 2 h. However, EGFP protein expression was not yet detected. Increasing the incubation time to 4 h allows the first signal of the EGFP expression to be detected with the presence of the low intensity fluorescent signal in the entire cytoplasm of the cells. This fluorescence intensity increased more when the incubation was increased to 23 h. Similar response was also observed by using mitochondria carrying mCherry mRNA and FAM-GAPDH siRNA, the presence of fluorescence mCherry was detected in the cytoplasm of the cells possessing the FAM-GAPDH siRNA signal. The knockdown expression of GAPDH in the cells was assessed using MTS assay. Reduction in the proliferation of A549 cells was observed in the cells treated with mitochondria carrying the mCherry mRNA and the FAM-GAPDH siRNA complex.

This result suggests that mitochondria could indeed simultaneously carry different oligonucleotides, opening a possibility to perform dual delivery to achieve a synergistic effect between the two oligonucleotides.

### Example 27. In Vitro Nebulization Study of Mitochondria-mRNA Complex in Lung Cell Model Mimicking Inhalation (2^{nd} Gen Complex)

### Methodology

### Synthesis and In Vitro Nebulization Study on A549 Cells

HCF mitochondria-EGFP mRNA complex were dispersed in the conjugation buffer at a concentration of 2 mg/mL. In vitro study of internalization of aerosolized mitochondria-ssDNA were performed using A549 cells. Briefly, 10000 cells were cultured on 48 well plate for 48 h. Nebulization was performed using a commercially available inhalator Beurer IH55 (Beurer, Germany) at the speed of 0.25 mL/min. The cells (four wells) were introduced to aerosolized complex for 30 seconds. After the nebulization, the cells were kept inside incubator for 24 h before a fluorescence imaging experiment to see EGFP expression inside the cells was conducted.

### Results and Discussion

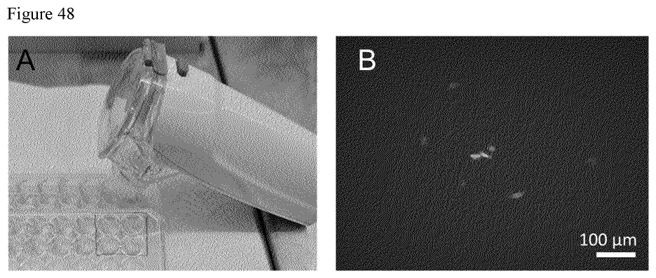

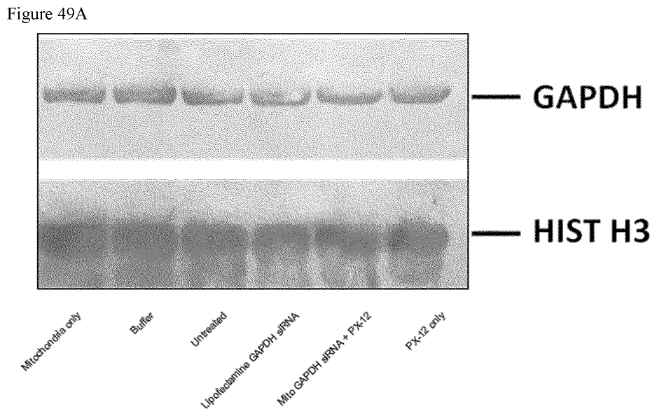

The hypothesis that mitochondria-EGFP mRNA complex could be delivered via aerosol inhalation was tested by nebulizing a solution containing 2 mg/mL of the complex using a commercially available inhalator. The nebulized complex was introduced to A549 lung epithelial cells for 30 seconds (FIG. 48A). After a 24 hour incubation period, the expression of EGFP in the cells were detected in all four wells (FIG. 48B), without any signs of cellular toxicity. This suggests that aerosolized delivery of mitochondria-oligonucleotides may be a viable alternative to direct injection or intravenous injection, and further research is conducted to apply this method in vivo.

### Example 28. In Vitro and In Vivo Study Simultaneous Delivery of siRNA and Anionic Drugs (2^{nd} Gen Complex)

### Methodology

In the following example, combination of Silencer™ FAM-labeled GAPDH siRNA and 2-[(1-methylpropyl)dithio]-1H-imidazole (PX-12) anionic drug were used. PX-12 is a small-molecule inhibitor of Trx-1 (thioredoxin-1), which can stimulate apoptosis, down-regulate HIF-la and vascular endothelial growth factor (VEGF) and inhibits tumor growth in animal models. To synthesize mitochondria carrying both Silencer™ FAM-labeled GAPDH siRNA and 2-[(1-methylpropyl)dithio]-1H-imidazole (PX-12), firstly isolated HCF mitochondria were suspended in the conjugation buffer at a final concentration of 2 mg/mL (corresponds to 6 billion mitochondria/mL). 500 μL of isolated mitochondria suspension were gently mixed with 16 μL polyethyleneimine MW: 10000 (1 mg/mL in deionized water), for 5 minutes at room temperature, protected from the light, before 8 μL Silencer™ FAM-labeled GAPDH siRNA (50 μM in RNAse/DNAse free water) and 0.4 μL of PX-12 (100 mM in DMSO) were added into the mixture. This yields a final concentration of siRNA ca. 20 μmol and PX-12 ca. 20 μM (for 50 μg of mitochondria), respectively. The complex was then mixed and incubated for the next 25 minutes at RT and in a dark environment. 32 μL poly(ethylene glycol)-block-polyethyleneimine (1 mg/mL in deionized water) was added to the mixture and the solution is mixed and incubated for another 5 minutes.

Lipofectamine-GAPDH siRNA sample was prepared as the following. In mixture A, 150 μL of OptiMEM was mixed with 16 μL of Lipofectamine RNAiMAX and incubated at RT for 10 minutes. In mixture B, 150 μL of OptiMEM was mixed with 9.6 μL of Silencer™ FAM-labeled GAPDH siRNA and incubated at RT for 10 minutes. The two mixtures were mixed and incubated at RT for an additional 5 minutes. The resulting complexes correspond to 25 μL of Lipofectamine-siRNA complex carrying 40 μmol of GAPDH siRNA, respectively.

In vitro study was performed using A549 cells. 10000-20000 cells are grown on 48 well plates for 48 hours before 50 μg of mitochondria-siRNA/PX-12 complex were added to the cells. Naked mitochondria, buffer, PX-12 drug and untreated cells were added as the controls. Comparison with Lipofectamine-encapsulating GAPDH siRNA was also performed. Post 48 h incubation, GAPDH protein knockdown was accessed using Western Blot.

### Results and Discussion

Combination therapy involves the administration of multiple drugs/agents to treat a medical condition. siRNA, or small interfering RNA, is a type of RNA molecule used to specifically target and reduce expression of specific genes, such as GAPDH which plays important role in oncology. The combination of these two treatments can lead to improved outcomes as multiple pathways can be targeted through drugs and the genes contributing to the disease can be targeted with siRNA.

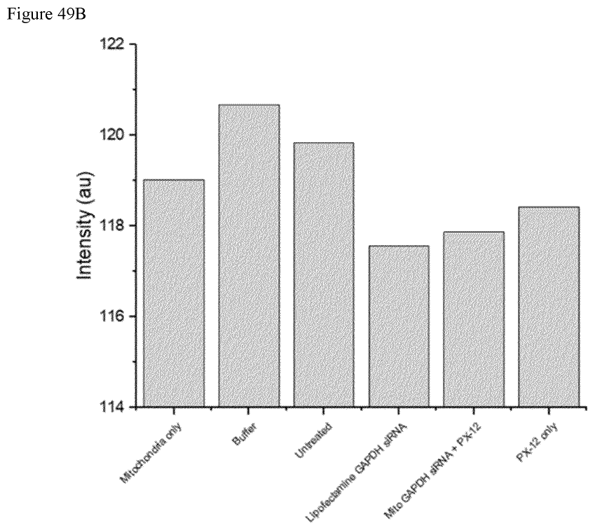

Mitochondria can be engineered to simultaneously carry both anionic drugs and siRNA, potentially increasing the efficiency and specificity of drug and siRNA delivery to target organs/tissues. We show that mitochondria were modified to carry PX-12 and GAPDH siRNA. After 24 hours of interaction with A549 lung cancer cells, a reduction of GAPDH expression was detected by Western blot, compared to control experiments including cells treated with naked mitochondria, buffer and PX-12 drug, or untreated cells. A direct comparison with cells treated with Lipofectamine-GAPDH siRNA (at double the concentration) produced a comparable response in GAPDH knockdown (FIG. 49). These results highlight a potential treatment for cancer.

Future in vivo study in an immunocompetent mouse model having a syngeneic lung tumor is performed by injecting the mitochondria-siRNA/PX-12 complex (100-2000 μg in 20-100 μL) through pulmonary artery or through inhalation-based delivery. Post complex exposure of 48-72 h, the tumor growth is monitored for a few days and up to a few weeks, using a MRI technique, according to local veterinary law.

### Example 29. Synthesis and In Vitro Study of Mitochondria-EGFP mRNA Using Cationic Lipid as Protective Layer (2^{nd} Gen Complex)

### Synthesis

The isolated HCF mitochondria were first dispersed in the conjugation buffer at 2 or 4 mg/mL. 500 μL of isolated mitochondria suspension were first gently mixed with 26 μL polyethyleneimine (1 mg/mL in deionized water), for 5 minutes at room temperature, protected from the light, before 10 μL CleanCap® mCherry mRNA (1 μg/μL in 1 mM Sodium Citrate pH 6.4) was added into the mixture. The complex was then mixed and incubated for the next 25 minutes at RT and in a dark environment. 52 μL DOTAP (1,2-dioleoyl-3-trimethylammonium-propane chloride; Avanti Polar Lipids) (1 mg/mL in PBS) was added to the mixture and the solution was mixed and incubated for another 5 minutes.

### In Vitro Transfection Study

Approximately 20000 A549 cells were grown on 48 well plates for 2 days. 25 μg of the complex composed of mitochondria-EGFP mRNA-DOTAP or Lipofectamine-EGFP mRNA or mitochondria-EGFP mRNA-poly(ethylene glycol)-block-polyethyleneimine was added and the cells were incubated for 24 hours before fluorescence microscopy was conducted.

### Results and Discussion

DOTAP is an example of cationic lipid commonly used as transfection agent to deliver DNA or RNA into mammalian cells. The positively-charged DOTAP molecules can interact with the negatively-charged phosphate groups of nucleic acids, forming complexes that can enter the cell through endocytosis or direct fusion with the cell membrane. In our purpose, DOTAP is used as the protective layer of preformed mitochondria-EGFP mRNA complex.

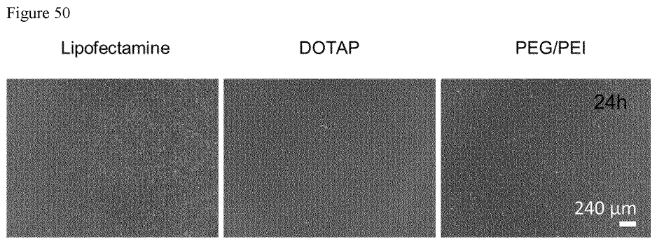

In vitro experiment was performed in A549 cells. After 24 hours of incubation, the expression of EGFP was accessed using a fluorescence microscope. The comparison between Lipofectamine sample and mitochondria-EGFP mRNA complex with poly(ethylene glycol)-block-polyethyleneimine was performed. The expression of EGFP of all three samples in the cells was shown in FIG. 50. We confirmed that DOTAP can be used as protective layer, however, the efficiency was lower in comparison to Lipofectamine and mitochondria-EGFP mRNA complex with poly(ethylene glycol)-block-polyethyleneimine.

### Example 30. In Vivo Delivery and Organ Biodistribution Study of Mitochondria-mRNA Complex (2^{nd} Gen Complex)

In vivo biodistribution study in a pig model is performed to study the expression of EGFP or mCherry mRNA carried by mitochondria in major organs such as kidney, lung, heart and liver. The mitochondria-EGFP/mCherry mRNA complexes with protective layer are injected via renal artery, pulmonary artery, intracoronary artery and hepatic artery route. The main experiment consists of injection of the samples into 1 treatment group (three pigs) and 1 control group (three pigs). For the treatment group, the mitochondria-EGFP/mCherry mRNA complexes with poly(ethylene glycol)-block-polyethyleneimine as protective layer (1-4 mg of mitochondria) are injected using a syringe/needle/catheter. For the control group, one pig is injected with the conjugation buffer, and two pigs are injected with Lipofectamine/nanoparticle carrying mRNA, using similar routes as previously described.

After the injections, the pigs are kept alive for 48 or 72 hours before being sacrificed. The four organs of interest (i.e., kidney, lung, heart and liver) are harvested and processed for bioanalysis. First, tissues are cut in few smaller pieces with the individual size of 1 cm×1 cm×1 cm. In the first evaluation, Western Blot protein analysis is performed. The expression of EGFP or mCherry protein as a successful proof of mRNA translation inside the organs is studied.

In the second evaluation, fluorescence microscopy analysis is performed. The tissues are fixed using 4% formaldehyde solution. For each organ, six random areas (size of 1 cm×1 cm×1 cm) are selected, prepared for a thin histology cut (5-10 micron thick) and then stained. In particular, for the kidney, the areas of interest consist of the cortex and the medulla. For the heart, the areas of left ventricle and left anterior descending are of interest. Nuclei of the cells are stained with DAPI while F-actins are stained with rhodamine phalloidin. The presence of EGFP/mCherry staining under fluorescence microscope is considered as successful delivery and mRNA translation.

### Example 31. Synthesis and In Vitro Wound Healing Study of Mitochondria-Polypeptide Complex (2^{nd} Gen Complex)

This example shows the use of mitochondria to transport positively-charged amino acids (e.g., lysine, arginine, or histidine). We use arginine as an example, an amino acid which is shown to play an important role in wound healing (NPL18). Isolated mitochondria are first dispersed in the conjugation buffer at 2 mg/mL. 500 μL of isolated mitochondria suspension is gently mixed with 26 μL polyethyleneimine MW: 10000 (1 mg/mL in deionized water), for 5 minutes at room temperature, protected from the light, before 10 μL arginine solution (1 mg/mL in water) is added into the mixture. The complex is then mixed and incubated for the next 25 minutes at RT and in a dark environment. 52 μL poly(ethylene glycol)-block-polyethyleneimine (1 mg/mL in deionized water) is added to the mixture and the solution is mixed and incubated for another 5 minutes.

In vitro study is performed using A549 cells. 10000-20000 cells are grown on 48 well plates for 48 hours until forming a cell monolayer. Wound healing assay is performed by scratching the cell layer using a sterile 10 μm pipette tip. After, 50-100 μg of mitochondria-arginine complex are added to the cells. Cell migration and wound healing capacity is monitored under a fluorescence microscope. The negative control experiment is cells treated only with mitochondria and untreated cells.

### Example 32. Synthesis and In Vitro Study of Mitochondria-Anionic Drug Complex (2^{nd} Gen Complex)

This example shows the use of mitochondria to transport anionic drug (PX-12) for an oncology application. Isolated HCF mitochondria are suspended in the conjugation buffer at a final concentration of 2 to 4 mg/mL (corresponds to 6 to 12 billion mitochondria/mL). 500 μL of isolated mitochondria suspension are gently mixed with 16 μL polyethyleneimine MW: 10000 (1 mg/mL in deionized water), for 5 minutes at room temperature, protected from the light, before 0.4-2 μL of PX-12 (100 mM in DMSO) is added into the mixture. This yields a final concentration of PX-12 ca. 20-100 μM (for 50 μg of mitochondria), respectively. The complex is then mixed and incubated for the next 25 minutes at RT and in a dark environment. 32 μL poly(ethylene glycol)-block-polyethyleneimine (1 mg/mL in deionized water) is added to the mixture and the solution is mixed and incubated for another 5 minutes.

In vitro study is performed using A549 carcinoma cells. 10000-20000 cells are grown on 48 well plates for 48 hours until forming a cell monolayer. MTS proliferation assay is performed after incubating the cells with the complex (50-100 μg of mitochondria-complex) for 24-48 hours. In addition, wound healing assay is performed by scratching the cell layer using a sterile 10 μm pipette tip. After, 50-100 μg of mitochondria-arginine complex are added to the cells. Cell migration and wound healing capacity is monitored under a fluorescence microscope. The negative control experiment is cells treated only with mitochondria and untreated cells.

### Example 33. Synthesis and In Vitro Study of Mitochondria-Cationic Drug Complex (2^{nd} Gen Complex)

This example shows the use of mitochondria to transport cationic drug (doxorubicin) for an oncology application. Doxorubicin is positively-charged at pH 7.0 due to its protonatable amino group (NPL19, NPL20), therefore it can engage in electrostatic interactions with negatively-charged mitochondria. Isolated HCF mitochondria are suspended in the conjugation buffer at a final concentration of 2 to 4 mg/mL (corresponds to 6 to 12 billion mitochondria/mL). 500 μL of isolated mitochondria suspension are gently mixed with 0.4-2 μL of doxorubicin (100 mM in DMSO or water) for 5-10 minutes. To remove unbound drugs, the mixture is centrifuged at 9500 rpm for 5 minutes and then the supernatant is removed. The mitochondria-doxorubicin are resuspended in 500 μL of conjugation buffer. 32 μL poly(ethylene glycol)-block-polyethyleneimine (1 mg/mL in deionized water) is added to the mixture and the solution is mixed and incubated for another 5 minutes.

In vitro study is performed using A549 carcinoma cells. 10000-20000 cells are grown on 48 well plates for 48 hours until forming a cell monolayer. MTS proliferation assay is performed after incubating the cells with the complex (50-100 μg of mitochondria-complex) for 24-48 hours. In addition, wound healing assay is performed by scratching the cell layer using a sterile 10 μm pipette tip. After, 50-100 μg of mitochondria-arginine complex are added to the cells. Cell migration and wound healing capacity is monitored under a fluorescence microscope. The negative control experiment is cells treated only with mitochondria and untreated cells.

### Example 34. Synthesis and In Vitro Study of Mitochondria Carrying Simultaneously Cationic Drug and Oligonucleotides (2^{nd} Gen Complex)

This example shows the use of mitochondria to transport simultaneously cationic drug and oligonucleotide (siRNA) for an oncology application. To synthesize mitochondria carrying Silencer™ FAM-labeled GAPDH siRNA and doxorubicin, firstly isolated HCF mitochondria are suspended in the conjugation buffer at a final concentration of 2-4 mg/mL (corresponds to 6-12 billion mitochondria/mL). 500 μL of isolated mitochondria suspension are gently mixed with 16 μL polyethyleneimine MW: 10000 (1 mg/mL in deionized water), for 5 minutes at room temperature, protected from the light, before 8-16 μL Silencer™ FAM-labeled GAPDH siRNA (50 μM in RNAse/DNAse free water) is added into the mixture. The complex is mixed and incubated for the next 25 minutes at RT and in a dark environment. After, 0.4-2 μL of doxorubicin (100 mM in DMSO) is added to the mixture and the solution is incubated for 5-10 minutes. 32 μL poly(ethylene glycol)-block-polyethyleneimine (1 mg/mL in deionized water) is added to the mixture and the solution is mixed and incubated for another 5 minutes.

In vitro study is performed using A549 carcinoma cells. 10000-20000 cells are grown on 48 well plates for 48 hours until forming a cell monolayer. MTS proliferation assay is performed after incubating the cells with the complex (50-100 μg of mitochondria-complex) for 24-48 hours. In addition, wound healing assay is performed by scratching the cell layer using a sterile 10 μm pipette tip. After, 50-100 μg of mitochondria-arginine complex are added to the cells. Cell migration and wound healing capacity is monitored under a fluorescence microscope. The negative control experiment is cells treated only with mitochondria and untreated cells.

### Example 35. In Vitro Study of Mitochondria-Renilla Luciferase mRNA Complex (2^{nd} Gen Complex)

### Materials

CleanCap® Renilla Luciferase mRNA (Ref. L-7204) were purchased from Trilink Biotechnologies. Polyethyleneimine, branched, molecular weight (MW) 10000, 99% (Ref: 040331.14) was purchased from Thermo Fisher Scientific. Poly(ethylene glycol)-block-polyethyleneimine, MW: 15000 (Ref: 910791) was purchased from Sigma-Aldrich. Lipofectamine™ RNAiMAX Transfection Reagent (Ref: 13778100) was purchased from Thermo Fisher Scientific.

### Synthesis of Mitochondria-Renilla Luciferase mRNA Complex

The isolated HCF mitochondria were mixed with the conjugation buffer. The mitochondrial concentration was kept at 4 mg/mL. 500 μL of the isolated mitochondrial suspension was then combined with 26 μL of polyethyleneimine (1 mg/mL in deionized water) for 5 minutes at room temperature, in the absence of light, before 15 μL of CleanCap® Renilla Luciferase mRNA (1 μg/μL in 1 mM Sodium Citrate pH 6.4) was added to the mixture. The complex was then mixed and incubated for an additional 25 minutes at room temperature and in the dark. 52 μL of poly(ethylene glycol)-block-polyethyleneimine (1 mg/mL in deionized water) was then added to the mixture, mixed, and incubated for an additional 5 minutes.

### Synthesis of Lipofectamine-Renilla Luciferase mRNA Complex

In mixture A, 250 μL of OptiMEM was mixed with 5 μL of Lipofectamine RNAiMAX and incubated at RT for 10 minutes. In mixture B, 250 μL of OptiMEM was mixed with 5 μL of CleanCap® Renilla Luciferase mRNA (1 μg/μL in 1 mM Sodium Citrate pH 6.4) and incubated at RT for 10 minutes. The two mixtures were mixed and incubated at RT for an additional 5 minutes.

### In Vitro Study of Luciferase Expression in A549 Cells

Approximately 20000 A549 cells were cultured on 96 well plates for 3 days. The medium of the cells was changed before the experiment. Afterwards, 40 μg of the mitochondria-Renilla Luciferase mRNA complex or 10 μL of Lipofectamine-Renilla Luciferase was added to each well and the cells were incubated for 72 hours before luciferase assay was conducted. Mitochondria only and untreated were used as negative controls. The Renilla Luciferase Assay System developed by Promega (Promega, Catalog number: E2810) was used to detect sea pansy (Renilla reniformis) luciferase. The experiments were minimum performed in triplicate.

### Results and Discussion

In this study, we aimed to investigate the potential of mitochondria carrying Renilla luciferase mRNA with poly(ethylene glycol)-block-polyethyleneimine as protective layer, to induce gene expression in A549 cells. Renilla luciferase is a bioluminescent reporter protein, which is derived from Renilla reniformis (sea pansy). In the presence of the cofactor, coelenterazine, Renilla luciferase produces light.

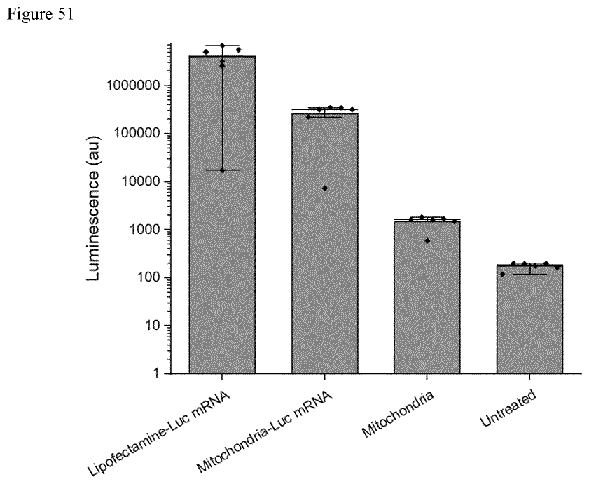

Our results demonstrate that the use of mitochondria as a carrier for luciferase mRNA can effectively induce luciferase expression in A549 cells. This was evident from the significant increase in luciferase activity observed in cells treated with mitochondria carrying luciferase mRNA compared to the control group (i.e., untreated cells and naked mitochondria, FIG. 51). Although the luciferase activity was 10 times lower than Lipofectamine as control, these findings suggest that the mitochondria successfully delivered the mRNA to the cells, resulting in positive translation and expression of the luciferase protein.

### Example 36. Synthesis and In Vitro Study of Mitochondria Carrying Lipid Nanoparticles Encapsulating Oligonucleotides

Illustration of a conjugation of mitochondria carrying lipid nanoparticles encapsulating oligonucleotides is shown in FIG. 2C.

### Methodology

Human cardiac fibroblasts (HCF) were cultured in Fibroblast Medium-2 (ScienCell) until they reached 80-90% cell confluency (2-4 million cells/flask). Thirty minutes to one hour prior to isolation, mitochondria were pre-labeled with MitoTracker™ Red CMXRos (Thermo Fisher Scientific, USA) following the protocols described by the manufacturer (Thermo Fisher Scientific). Labeled mitochondria were isolated according to the established Cellvie SOP (NPL8). The isolated mitochondria were then resuspended in the conjugation buffer at a final concentration of 2 mg/mL (6 billion mitochondria/mL).

To create a DOTAP stock solution (1 mg/mL), 1 mg of DOTAP (1,2-dioleoyl-3-trimethylammonium-propane chloride; Avanti Polar Lipids) was dissolved in 1 mL of PBS.

### Synthesis of Mitochondria-DOTAP NPs with ssDNA Complex

To synthesize DOTAP nanoparticles (NPs) encapsulating ssDNA, 45 μL of DOTAP stock solution was first mixed with 45 μL of conjugation buffer, and then, the mixture was mixed with 1.45 μL of FAM-ssDNA solution in PBS (0.7 μg/μL). The mixture was incubated at room temperature in the dark for 10 minutes.

To create the mitochondria-DOTAP NPs complex, labeled mitochondria were mixed with the DOTAP NPs solution at a (v/v) ratio of 2:1. The mixture was further incubated for 30 minutes. The attachment of DOTAP NPs on mitochondria was visualized using a fluorescence microscope.

### Synthesis of and In Vitro Study of Mitochondria-DOTAP NPs EGFP mRNA

To synthesize DOTAP nanoparticles (NPs) encapsulating CleanCap® EGFP mRNA, 128 μL of DOTAP stock solution was first mixed with 2 μL of CleanCap® EGFP mRNA (1 μg/μL). The mixture was incubated at room temperature in the dark for 10 minutes.

To create the mitochondria-DOTAP NPs complex, 100 μL of HCF mitochondria was mixed with 66 μL of DOTAP NPs encapsulating EGFP mRNA. The mixture was then incubated for a further 30 minutes.

An in vitro study was performed using A549 cells. Briefly, 82 μL of the complex was incubated with 20,000 A549 cells cultured in a 48-well plate for 22 hours. The expression of EGFP, as a successful proof of delivery and mRNA translation, was studied using a fluorescence microscope.

### Results and Discussion

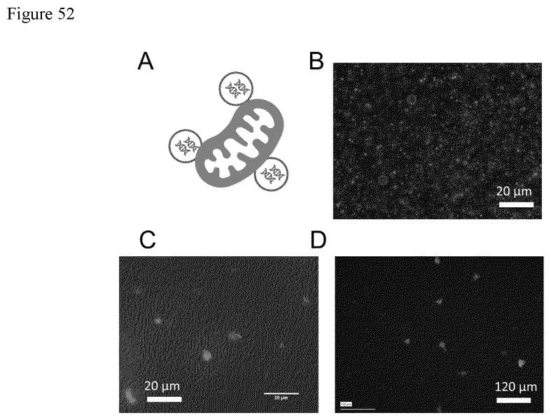

We explored the possibility of attaching positively-charged nanoparticles (NPs) that encapsulate oligonucleotides to mitochondria (FIG. 52A). As described in the literature (NPL21), the formation of DOTAP NPs encapsulating oligonucleotides was observed when cationic DOTAP was mixed with negatively-charged DNA or RNA molecules. In the following example, the formation of DOTAP NPs encapsulating ssDNA was monitored using fluorescent FAM-ssDNA (FIG. 52B).

DOTAP NPs were then mixed with MitoTracker™ Red CMXRos-labeled mitochondria, allowing for the attachment of NPs to the mitochondria surface through electrostatic interaction. The formation of the complex was successfully observed under the fluorescence microscope through colocalization of the ssDNA signal on the mitochondria (FIG. 52C).

Furthermore, mitochondria-DOTAP NPs encapsulating EGFP mRNA were synthesized, and in vitro EGFP expression was studied in A549 cells. After 22 hours of incubation, EGFP expression was observed in A549 cells (FIG. 52D). Analysis of the brightfield image showed no signs of toxicity.

### LIST OF REFERENCES

[NPL1] Al-Dosari, M. S. and Gao, X., 2009. Nonviral gene delivery: principle, limitations, and recent progress. The AAPS journal, 11(4), pp. 671-681.
[NPL2] Büning, H. and Schmidt, M., 2015. Adeno-associated vector toxicity—to be or not to be?. Molecular Therapy, 23(11), pp. 1673-1675.
[NPL3] Das, J. et al., 2016. Potential toxicity of engineered nanoparticles in mammalian germ cells and developing embryos: treatment strategies and anticipated applications of nanoparticles in gene delivery. Human reproduction update, 22(5), pp. 588-619.
[NPL4] Bai, X. et al., 2021. In vivo Protein Corona Formation: Characterizations, Effects on Engineered Nanoparticles' Biobehaviors, and Applications. Frontiers in Bioengineering and Biotechnology, 9, p. 263.
[NPL5] Pacak, C. A. et al., 2015. Actin-dependent mitochondrial internalization in cardiomyocytes: evidence for rescue of mitochondrial function. Biology open, 4(5), pp. 622-626.
[NPL6] McCully, J. D. et al., 2017. Mitochondrial transplantation: from animal models to clinical use in humans. Mitochondrion, 34, pp. 127-134.
[NPL7] Masuzawa, A. et al., 2013. Transplantation of autologously derived mitochondria protects the heart from ischemia-reperfusion injury. American Journal of Physiology—Heart and Circulatory Physiology, 304(7), pp. H966-H982.
[NPL8] Preble, J. M. et al., 2014. Rapid isolation and purification of mitochondria for transplantation by tissue dissociation and differential filtration. JoVE (Journal of Visualized Experiments), (91), p. e51682.
[NPL9] Lülf, H. et al., 2014. Multifunctional inorganic nanocontainers for DNA and drug delivery into living cells. Chemistry—A European Journal, 20(35), pp. 10900-10904.
[NPL10] Huang, R. et al., 2019. Herpes simplex virus 1 microRNA miR-H28 exported to uninfected cells in exosomes restricts cell-to-cell virus spread by inducing gamma interferon mRNA. Journal of virology, 93(21), pp. e01005-19.
[NPL11] Nakashima, R. A. et al., 1984. Contributions of glycolysis and oxidative phosphorylation to adenosine 5′-triphosphate production in AS-30D hepatoma cells. Cancer research, 44(12 Part 1), pp. 5702-5706.
[NPL12] Shi, D. and Gu, W., 2012. Dual roles of MDM2 in the regulation of p 53: ubiquitination dependent and ubiquitination independent mechanisms of MDM2 repression of p 53 activity. Genes & cancer, 3(3-4), pp. 240-248.
[NPL13] Cowan, D. B. et al., 2016. Intracoronary delivery of mitochondria to the ischemic heart for cardioprotection. PloS one, 11 (8), p. e0160889.
[NPL14] Veronese F. M. and Mero A., 2008, BioDrugs, volume 22, pp 315-329, The Impact of PEGylation on Biological Therapies; Suk J., et al., 2016, Adv Drug Deliv Rev. April 1; 99 (Pt A): 28-51. PEGylation as a strategy for improving nanoparticle-based drug and gene delivery.
[NPL15] Carrasco J. M. et al., 2021. Ionization and structural properties of mRNA lipid nanoparticles influence expression in intramuscular and intravascular administration. Comm. Biol., 4:956.
[NPL16] Hamada E. et al., 2021. Anionic Complex with Efficient Expression and Good Safety Profile for mRNA Delivery. Pharmaceutics, 13, 126.
[NPL17] Shi, D. and Gu, W., 2012. Dual roles of MDM2 in the regulation of p 53: ubiquitination dependent and ubiquitination independent mechanisms of MDM2 repression of p 53 activity. Genes & cancer, 3(3-4), pp. 240-248.
[NPL18] Tong B. C. and Barbul A., 2004. Cellular and physiological effects of arginine. Mini Rev Med Chem, October; 4(8):823-32
[NPL19] Li W, Yi X, Liu X. et al. Hyaluronic acid ion-pairing nanoparticles for targeted tumor therapy. J Control Release 2016; 225:170-82.
[NPL20] Manocha B, Margaritis A. Controlled release of doxorubicin from doxorubicin/γ-polyglutamic acid ionic complex. J Nanomater 2010; 2010:1-9.
[NPL21] Felgner, J., et al., 1993. Cationic lipid-mediated transfection in mammalian cells: “Lipofection”. Journal of tissue culture methods, 15(2), pp. 63-68.

The invention described herein, inter alia, relates to the following embodiments.
1. A mitochondrion comprising one or more nucleic acid molecule(s) attached to the outer membrane of the mitochondrion, wherein the one or more nucleic acid molecule(s)
   a) is electrostatically attached to the outer membrane of the mitochondrion via a positively-charged species; or
   b) is covalently linked to the outer membrane of the mitochondrion; or
   c) is linked to an antibody that specifically binds to an antigen comprised in the outer membrane of the mitochondrion; or
   d) is linked to a mitochondria-targeting small molecule.
2. The mitochondrion according to embodiment 1, wherein the nucleic acid molecule is DNA or RNA.
3. The mitochondrion according to embodiment 1 or 2, wherein the positively-charged species is a polycationic species, optionally wherein the polycationic species is a linear or branched polycationic polymer, optionally wherein the linear or branched polycationic polymer is electrostatically linked to the one or more nucleic acid molecule(s).
4. The mitochondrion according to embodiment 3, wherein the linear or branched polycationic polymer is polylysine, histidylated polylysine, polyornithine, polyarginine, high-mobility group protein (HMG) 1 and 17, modified chitosan, cationized human serum albumin, polyethyleneimine (PEI), polypropyleneimine (PPI), a cationic dendrimer, poly(2-(dimethylamino)ethyl methacrylate) (PDMAEMA), a polyallylamine derivative, diethylaminoethyl (DEAE)-dextran, poly(N-alkyl-4-vinylpyridinium), a poly(amidoamine), cationic gelatin, cationic cellulose or a combination thereof.
5. The mitochondrion according to embodiment 1 or 2, wherein the positively-charged species is a positively-charged nanoparticle.
6. The mitochondrion according to embodiment 1 or 2, wherein the positively-charged species is a positively-charged particle.
7. The mitochondrion according to embodiment 5, wherein the one or more nucleic acid molecule(s) is attached to the surface of the positively-charged nanoparticle or encapsulated in the positively-charged nanoparticle.
8. The mitochondrion according to embodiment 6, wherein the one or more nucleic acid molecule(s) is attached to the surface of the positively-charged particle or encapsulated in the positively-charged particle.
9. The mitochondrion according to any one of embodiments 5 to 8, wherein the positively-charged nanoparticle and/or particle is a lipid nanoparticle/particle, a dendrimer nanoparticle/particle, a micelle nanoparticle/particle, a protein nanoparticle/particle, a liposome, a non-porous silica nanoparticle/particle, a mesoporous silica nanoparticle/particle, a silicon nanoparticle/particle, a gold nanoparticle/particle, a gold nanowire, a silver nanoparticle/particle, a platinum nanoparticle/particle, a palladium nanoparticle/particle, a titanium dioxide nanoparticle/particle, a carbon nanotube, a carbon dot nanoparticle/particle, a polymer nanoparticle/particle, a zeolite nanoparticle/particle, an aluminium oxide nanoparticle/particle, a hydroxyapatite nanoparticle/particle, a quantum dot nanoparticle/particle, a zinc oxide nanoparticle/particle, a zirconium oxide nanoparticle/particle, graphene or a graphene oxide nanoparticle/particle.
10. The mitochondrion according to embodiment 1 or 2, wherein the one or more nucleic acid molecule(s) is covalently linked to a polypeptide in the outer membrane of the mitochondrion via an amide bond.
11. The mitochondrion according to embodiment 10, wherein the one or more nucleic acid molecule(s) has been modified to undergo formation of the amide bond with an amine function comprised in the polypeptide in the outer membrane of the mitochondrion.
12. The mitochondrion according to embodiment 1, 2, 5, 7, or 9, or 10, wherein the one or more nucleic acid molecule(s) is encapsulated in a nanoparticle, and wherein the nanoparticle comprises a functional group that allows covalent linkage of the nanoparticle to a polypeptide in the outer membrane of the mitochondrion.
13. The mitochondrion according to embodiment 1 or 2, wherein the antibody specifically binds to an antigen comprised in the outer membrane of the mitochondrion, wherein the antigen is OPA1, TOM70, TOMM20, Mitofusin 1, Mitofusin 2 or VDAC1.
14. The mitochondrion according to any one of embodiments 1, 2 or 13, wherein the one or more nucleic acid molecule(s) is encapsulated in a nanoparticle, and wherein the nanoparticle is covalently linked to the antibody.
15. The mitochondrion according to any one of embodiments 1, 2 or 13, wherein the one or more nucleic acid molecule(s) is electrostatically linked to the antibody, wherein the antibody is a modified antibody, wherein the modified antibody possesses one or more positive charges.
16. The mitochondrion according to any one of embodiments 1, 2 or 13, wherein the one or more nucleic acid molecule(s) is covalently linked to biotin, wherein biotin is linked to the antibody, wherein the antibody is an avidin conjugated antibody.
17. The mitochondrion according to any one of embodiments 1, 2 or 13, wherein the one or more nucleic acid molecule(s) is covalently linked to an activated ester, wherein the activated ester is linked to the antibody via an amide bond.
18. The mitochondrion according to any one of embodiments 1, 2 or 13, wherein the one or more nucleic acid molecule(s) is a single-stranded nucleic acid molecule (ssDNA or ssRNA), wherein the single-stranded nucleic acid molecule is hybridized with one or more complementary single-stranded nucleic acid molecule attached on or to the antibody.
19. The mitochondrion according to embodiment 1 or 2 or 13, wherein the one or more nucleic acid molecule(s) is encapsulated in a nanoparticle, wherein the nanoparticle is electrostatically linked to the antibody, wherein the antibody is a modified antibody, wherein the modified antibody possesses one or more positive charges.
20. The mitochondrion according to any one of embodiments 1, 2 or 13, wherein the one or more nucleic acid molecule(s) is encapsulated in a nanoparticle, wherein the nanoparticle is covalently linked to biotin, wherein biotin is linked to the antibody, wherein the antibody is an avidin conjugated antibody.
21. The mitochondrion according to any one of embodiments 1, 2 or 13, wherein the one or more nucleic acid molecule(s) is encapsulated in a nanoparticle, wherein the nanoparticle is covalently linked to activated ester, wherein activated ester is linked to the antibody via amide bond.
22. The mitochondrion according to any one of embodiment 1 or 2, wherein the mitochondria-targeting small molecule is selected from the group consisting of triphenylphosphonium (TPP), dequalinium (DQA), E-4-(1H-Indol-3-ylvinyl)-N-Methylpyridineiodide (F16), Rhodamine 19, biguanidine and guanidine.
23. The mitochondrion according to any one of embodiments 1 to 22, wherein the mitochondrion is linked to and/or enveloped in a protective layer.
24. The mitochondrion according to embodiment 23, wherein the protective layer is a protective polymer.
25. The mitochondrion according to embodiment 24, wherein the protective polymer is a linear or branched cationic polymer, optionally wherein the linear or branched cationic polymer is electrostatically linked to the one or more nucleic acid molecule(s) or wherein the linear or branched cationic polymer is covalently linked to the one or more nucleic acid molecule(s).
26. The mitochondrion according to embodiment 24, wherein the protective polymer is a linear or branched cationic block copolymer, optionally wherein the linear or branched cationic block copolymer is electrostatically linked to the one or more nucleic acid molecule(s) or wherein the linear or branched cationic block copolymer is covalently linked to the one or more nucleic acid molecule(s).
27. The mitochondrion according to embodiment 24, wherein the protective polymer is a cationic graft (g) copolymer, optionally wherein the cationic graft (g) copolymer is electrostatically linked to the one or more nucleic acid molecule(s) or wherein the cationic graft (g) copolymer is covalently linked to the one or more nucleic acid molecule(s).
28. The mitochondrion according to embodiment 24, wherein the protective polymer is a linear or branched pegylated (PEG) cationic polymer, optionally wherein the linear or branched pegylated (PEG) cationic polymer is electrostatically linked to the one or more nucleic acid molecule(s) or wherein the linear or branched pegylated (PEG) cationic polymer is covalently linked to the one or more nucleic acid molecule(s).
29. The mitochondrion according to embodiment 23, wherein the protective layer is a lipid formulation, optionally wherein the lipid formulation is a cationic lipid formulation, further optionally wherein the cationic lipid formulation is electrostatically linked to the one or more nucleic acid molecule(s) or wherein the cationic lipid formulation is covalently linked to the one or more nucleic acid molecule(s).
30. The mitochondrion according to any one of embodiments 23 to 29, wherein the protective layer is linked to an antibody, optionally wherein the protective layer linked to an antibody is electrostatically linked to the one or more nucleic acid molecule(s) or wherein the protective layer linked to an antibody is covalently linked to the one or more nucleic acid molecule(s).
31. The mitochondrion according to any one of embodiments 23 to 29, wherein the protective layer is linked to a carbohydrate, optionally wherein the protective layer linked to a carbohydrate is electrostatically linked to the one or more nucleic acid molecule(s) or wherein the protective layer linked to a carbohydrate is covalently linked to the one or more nucleic acid molecule(s).
32. The mitochondrion according to embodiment 25, wherein the linear or branched cationic polymer is polyethyleneimine, RGD-modified polyethyleneimine, polylysine, RGD-modified polylysine, polyornithine, RGD-modified polyornithine, polyarginine, RGD modified polyarginine, polypropyleneimine, RGD-modified polypropyleneimine, polyallylamine, RGD-modified polyallylamine, chitosan, RGD-modified chitosan, poly(2-(dimethylamino)ethyl methacrylate), RGD-modified poly(2-(dimethylamino)ethyl methacrylate), poly(amidoamine)s, RGD-modified poly(amidoamine)s or a combination thereof.
33. The mitochondrion according to embodiment 26, wherein the cationic block copolymer is poly(ethylene glycol)-block-polyethyleneimine, RGD-modified poly(ethylene glycol)-block-polyethyleneimine, poly(ethylene glycol)-block-polylysine, RGD-modified poly(ethylene glycol)-block-polylysine, poly(ethylene glycol)-block-polyornithine, RGD-modified poly(ethylene glycol)-block-polyornithine, poly(ethylene glycol)-block-polyarginine, RGD-modified poly(ethylene glycol)-block-polyarginine, poly(ethylene glycol)-block-polypropyleneimine, RGD-modified poly(ethylene glycol)-block-polypropyleneimine, poly(ethylene glycol)-block-polyallylamine, RGD-modified poly(ethylene glycol)-block-polyallylamine, poly(ethylene glycol)-block-poly(2-(dimethylamino)ethyl methacrylate), RGD-modified poly(ethylene glycol)-block-poly(2-(dimethylamino)ethyl methacrylate), poly(ethylene glycol)-block-poly(amidoamine)s, RGD-modified poly(ethylene glycol)-block-poly(amidoamine)s or a combination thereof.
34. The mitochondrion according to embodiment 27, wherein the cationic graft (g) copolymer is poly(ethylene glycol)-g-polyethyleneimine, RGD-modified poly(ethylene glycol)-g-polyethyleneimine, poly(ethylene glycol)-g-polylysine, RGD-modified poly(ethylene glycol)-g-polylysine, poly(ethylene glycol)-g-polyornithine, RGD-modified poly(ethylene glycol)-g-polyornithine, poly(ethylene glycol)-g-polyarginine, RGD-modified poly(ethylene glycol)-g-polyarginine, poly(ethylene glycol)-g-polypropyleneimine, RGD-modified poly(ethylene glycol)-g-polypropyleneimine, poly(ethylene glycol)-g-polyallylamine, RGD-modified poly(ethylene glycol)-g-polyallylamine, poly(ethylene glycol)-g-poly(2-(dimethylamino)ethyl methacrylate), RGD-modified poly(ethylene glycol)-g-poly(2-(dimethylamino)ethyl methacrylate), poly(ethylene glycol)-g-poly(amidoamine)s, RGD-modified poly(ethylene glycol)-g-poly(amidoamine)s or a combination thereof.
35. The mitochondrion according to embodiment 28, wherein the pegylated (PEG) cationic polymer is pegylated-polyethyleneimine, RGD-modified pegylated polyethyleneimine, pegylated polylysine, RGD-modified pegylated polylysine, histidylated polylysine, pegylated polyornithine, RGD-modified pegylated polyornithine, pegylated polyarginine, RGD-modified pegylated polyarginine, pegylated polypropyleneimine, RGD-modified pegylated polypropyleneimine, pegylated polyallylamine, RGD-modified pegylated polyallylamine, pegylated chitosan, RGD-modified pegylated chitosan, pegylated poly(2-(dimethylamino)ethyl methacrylate), RGD-modified pegylated poly(2-(dimethylamino)ethyl methacrylate), pegylated poly(amidoamine) s RGD-modified pegylated poly(amidoamine)s or a combination thereof.
36. The mitochondrion according to embodiment 29, wherein the lipid formulation comprises DC-cholesterol (3β-[N—(N′,N′-Dimethylaminoethane)-carbamoyl]cholesterol hydrochloride), DLinDMA (1,2-dilinoleyloxy-3-dimethylaminopropane), DLinMC3DMA (dilinoleylmethyl-4-dimethylaminobutyrate), DODMA (1,2-dioleyloxy-3-dimethylaminopropane), DOGS (dioctadecylamidoglycylspermine), DOSPA (2,3-dioleyloxy-N-[2 (sperminecarboxamido)ethyl]-N,N-dimethyl-1-propanaminium), DODAP (1,2-dioleoyl-3-dimethylammonium propane), DOTMA (1,2-di-O-octadecenyl-3-trimethylammonium propane chloride)), UGG (unsaturated guanidinium glycoside), DOPE (1,2-Dioleoyl-sn-glycerophosphoethanolamine), lipofectamine or a combination thereof.
37. The mitochondrion according to embodiment 36, wherein the lipid formulation further comprises another lipid, preferably wherein said lipid is cholesterol, a substituted or unsubstituted cholesterol, a cholesterol derivative, such as a hydroxylated cholesterol derivative (e.g., a hydroxycholesterol), a PEG-lipid, DMPC (1,2-Dimyristoyl-sn-glycero-3-phosphocholine), DSPC (1,2-Distearoyl-sn-glycero-3-phosphocholine), DODAP (1,2-dioleoyl-3-dimethylammonium propane), DDA (dimethyldioctadecylammonium), 1,2-dioleoyl-sn-glycero-3-phosphate, 1,2-dimyristoyl-sn-glycero-3-phosphate, bis(monooleoylglycerol)phosphate or a combination thereof.
38. The mitochondrion according to embodiment 24 wherein the mitochondrion is linked to and/or enveloped in a zwitterionic protective polymer, optionally wherein the zwitterionic protective polymer is electrostatically linked to the one or more nucleic acid molecule(s).
39. The mitochondrion according to embodiment 38, wherein the zwitterionic protective polymer is selected from: poly(2-methacryloyloxyethyl phosphorylcholine) (PMPC), polyethyleneimine-g-poly(2-methacryloyloxyethyl phosphorylcholine) (PEI-g-PMPC), co-assembly of cationic (carboxyl-functionalized) and anionic (amino-functionalized) copolyesters based on poly(ε-caprolactone)-block-poly(butylene fumarate)-block-poly(ε-caprolactone) (PCL-b-PBF-b-PCL), poly(lactic-co-glycolic acid) (PLGA)-PCB block copolymers (PLGA-b-PCB).
40. A composition comprising a plurality of mitochondria according to any one of embodiments 1 to 39.
41. A pharmaceutical composition comprising a plurality of mitochondria according to any one of embodiments 1 to 39 and a pharmaceutically acceptable carrier.
42. The pharmaceutical composition according to embodiment 41, wherein the pharmaceutical composition is formulated as a solution.
43. The pharmaceutical composition according to embodiment 41, wherein the pharmaceutical composition is formulated as an aerosol.
44. The mitochondrion according to any one of embodiments 1 to 39, the composition according to embodiment 40 or the pharmaceutical composition according to any one of embodiments 41 to 43 for use as a medicament.
45. The mitochondrion according to any one of embodiments 1 to 39, the composition according to embodiment 40 or the pharmaceutical composition according to any one of embodiments 41 to 43 for use in gene therapy.
46. The mitochondrion according to any one of embodiments 1 to 39, the composition according to embodiment 40 or the pharmaceutical composition according to any one of embodiments 41 to 43 for use in the treatment of cardiovascular diseases, in particular for use in the treatment of ischemic heart disease, ischemia-reperfusion injury, or atherosclerosis.
47. The mitochondrion according to any one of embodiments 1 to 39, the composition according to embodiment 40 or the pharmaceutical composition according to any one of embodiments 41 to 43 for use in the treatment of aging related diseases, in particular for use in the treatment of, sarcopenia, Parkinson's disease or Hutchinson-Gilford progeria syndrome.
48. The mitochondrion according to any one of embodiments 1 to 39, the composition according to embodiment 40 or the pharmaceutical composition according to any one of embodiments 41 to 43 for use in the treatment of cancer.
49. The mitochondrion according to any one of embodiments 1 to 39, the composition according to embodiment 40 or the pharmaceutical composition according to any one of embodiments 41 to 43 for use in in vitro, ex vivo, or in vivo genome editing.
50. The mitochondrion according to any one of embodiments 1 to 39, the composition according to embodiment 40 or the pharmaceutical composition according to any one of embodiments 41 to 43 for use in radiation therapy.
51. A method for delivering a nucleic acid molecule to a target organ, the method comprising a step of administering the pharmaceutical composition according to embodiment 40 or 41 into the bloodstream of a subject in need, wherein the pharmaceutical composition is administered into the bloodstream upstream of the target organ.
52. A method for delivering a nucleic acid molecule to the lung, the method comprising a step of administering the pharmaceutical composition according to embodiment 43 to a subject in need, wherein the pharmaceutical composition is administered by inhalation.
53. A method for attaching a nucleic acid molecule to the outer membrane of a mitochondrion, the method comprising the steps of:
   a) providing a preparation of mitochondria;
   b) contacting the mitochondria provided in step (a) with at least one nucleic acid molecule in the presence of a positively-charged species; and
   c) attaching the at least one nucleic acid molecule(s) to the mitochondria via the positively-charged species.
54. The method according to embodiment 53, wherein
   a) the at least one nucleic acid molecule, is simultaneously contacted with the positively-charged species and the mitochondria;
   b) the at least one nucleic acid molecule, is contacted with the positively-charged species to form a positively-charged complex before the positively-charged complex is contacted with the mitochondria; or
   c) the mitochondrion is contacted with the positively-charged species and subsequently contacted with the at least one nucleic acid molecule.
55. The method according to embodiment 53 or 54, wherein the mitochondria are contacted with the at least one nucleic acid molecule and the positively-charged species in a suitable buffer.
56. The method according to embodiment 55, wherein the buffer comprises consists of HEPES, EGTA, Trehalose, CHES and sodium phosphate dibasic dihydrate, preferably wherein buffer comprises a mixture of a Solution X comprising or consisting of HEPES, EGTA and Trehalose and of a Solution Y comprising or consisting of CHES and sodium phosphate dibasic dihydrate, more preferably, wherein the buffer comprises a 4:1 mixture of Solution X comprising or consisting of 20 mM HEPES, 1 mM EGTA and 300 mM Trehalose (pH 7.2) and Solution Y comprising or consisting of 0.1 M CHES (pH 10) and 0.2 M sodium phosphate dibasic dihydrate.
57. The method according to any one of embodiments 53 to 56, wherein the mitochondria are contacted with the at least one nucleic acid molecule, and the positively-charged species at room temperature for at least 5 minutes, such as at least 10 minutes, 20, 30, 40, 50, 60 or 120 minutes.
58. The method according to any one of embodiments 53 to 57, wherein the mitochondria are contacted with the at least one nucleic acid molecule, and the positively-charged species in the dark.
59. The method according to any one of embodiments 53 to 58, wherein the nucleic acid molecule is DNA or RNA.
60. The method according to any one of embodiments 53 to 59, wherein the positively-charged species is a polycationic species, wherein the polycationic species is a linear or branched polycationic polymer, optionally wherein the linear or branched polycationic polymer is electrostatically linked to the at least one nucleic acid molecules.
61. The method according to embodiment 60, wherein the linear or branched polycationic polymer is polylysine, histidylated polylysine, polyornithine, polyarginine, high-mobility group protein (HMG) 1 and 17, a modified chitosan, cationized human serum albumin, polyethyleneimine (PEI), polypropyleneimine (PPI), a cationic dendrimer, poly(2-(dimethylamino)ethyl methacrylate) (PDMAEMA), a polyallylamine derivative, diethylaminoethyl (DEAE)-dextran poly(N-alkyl-4-vinylpyridinium), a poly(amidoamine), cationic gelatin, cationic cellulose or a combination thereof.
62. The method according to any one of embodiments 53 to 59, wherein the positively-charged species is a positively-charged nanoparticle.
63. The method according to embodiment 62, wherein the method comprises a further step of
   a) attaching the at least one nucleic acid molecule to the surface of the positively-charged nanoparticle; or
   b) encapsulating the at least one nucleic acid molecule within the positively-charged nanoparticle.
64. The method according to embodiment 62 or 63, wherein the positively-charged nanoparticle is a lipid nanoparticle, a dendrimer nanoparticle, a micelle nanoparticle, a protein nanoparticle, a liposome, a non-porous silica nanoparticle, a mesoporous silica nanoparticle, a silicon nanoparticle, a gold nanoparticle, a gold nanowire, a silver nanoparticle, a platinum nanoparticle, a palladium nanoparticle, a titanium dioxide nanoparticle, a carbon nanotube, a carbon dot nanoparticle, a polymer nanoparticle, a zeolite nanoparticle, an aluminium oxide nanoparticle, a hydroxyapatite nanoparticle, a quantum dot nanoparticle, a zinc oxide nanoparticle, a zirconium oxide nanoparticle, graphene or a graphene oxide nanoparticle.
65. A method for covalently attaching at least one nucleic acid molecule to the outer membrane of a mitochondrion, the method comprising the steps of:
   a) providing a preparation of mitochondria;
   b) providing a nucleic acid molecule that has been modified to comprise an activated ester; and
   c) attaching the nucleic acid molecule provided in step (b) to an amine comprised in a polypeptide in the outer membrane of the mitochondria.
   66. The method according to embodiment 65, wherein the activated ester is an N-hydroxysuccinimide (NHS) ester.
67. A method for covalently attaching at least one nucleic acid molecule to the outer membrane of a mitochondrion, the method comprising the steps of:
   a) providing a preparation of mitochondria;
   b) encapsulating at least one nucleic acid molecule in a nanoparticle, wherein the surface of the nanoparticle comprises an activated ester; and
   c) attaching the nanoparticle provided in step (b) to an amine comprised in a polypeptide in the outer membrane of the mitochondria.
68. The method according to embodiment 67, wherein the activated ester is an NHS ester.
69. A method for attaching at least one nucleic acid molecule to the outer membrane of a mitochondrion, the method comprising the steps of:
   a) providing a preparation of mitochondria;
   b) contacting the mitochondria provided in step (a) comprising an antigen in their outer membrane with at least one nucleic acid molecule linked to an antibody; and
   c) attaching the at least one nucleic acid molecule to the mitochondria via the antibody, wherein the antibody specifically binds to the antigen comprised in the outer membrane of the mitochondrion.
70. The method according to embodiment 69, wherein the antibody specifically binds to an antigen comprised in the outer membrane of the mitochondrion, wherein the antigen is OPA1, TOM70, TOMM20, Mitofusin 1, Mitofusin 2 or VDAC1.
71. The method according to embodiment 69 or 70, wherein the one or more nucleic acid molecule(s) is encapsulated in a nanoparticle, and wherein the nanoparticle is covalently linked to the antibody.
72. The method according to embodiment 69 or 70, wherein the one or more nucleic acid molecule(s) is electrostatically linked to the antibody, wherein the antibody is a modified antibody possessing one or more positive charge(s).
73. The method according to embodiment 69 or 70, wherein the one or more nucleic acid molecule(s) is covalently linked to biotin, wherein biotin is linked to the antibody, wherein the antibody is an avidin conjugated antibody.
74. The method according to embodiment 69 or 70, wherein the one or more nucleic acid molecule(s) is covalently linked to an activated ester, wherein the activated ester is linked to the antibody via an amide bond.
75. The method according to embodiment 69 or 70, wherein the one or more nucleic acid molecule(s) is a single-stranded nucleic acid molecule (ssDNA or ssRNA), wherein the single-stranded nucleic acid molecule is hybridized with one or more complementary single-stranded nucleic acid molecule(s) attached on or to the antibody.
76. The method according to embodiment 69 or 70, wherein the one or more nucleic acid molecule(s) is encapsulated in a nanoparticle, wherein the nanoparticle is electrostatically linked to the antibody, wherein the antibody is a modified antibody, wherein the modified antibody possesses one or more positive charge(s).
77. The method according to embodiment 69 or 70, wherein the one or more nucleic acid molecule(s) is encapsulated in a nanoparticle, wherein the nanoparticle is covalently linked to biotin, wherein biotin is linked to the antibody, wherein the antibody is an avidin conjugated antibody.
78. The method according to embodiment 69 or 70, wherein the one or more nucleic acid molecule(s) is encapsulated in a nanoparticle, wherein the nanoparticle is covalently linked to an activated ester, wherein the activated ester is linked to the antibody via amide bond.
79. A method for attaching a nucleic acid molecule to the outer membrane of a mitochondrion, the method comprising the steps of:
   a) providing a preparation of mitochondria;
   b) contacting the mitochondria provided in step (a) with at least one nucleic acid molecule linked to a mitochondria-targeting small molecule; and
   c) attaching the at least one nucleic acid molecule to the mitochondria via a mitochondria-targeting small molecule.
80. The method according to embodiment 79, wherein the mitochondria-targeting small molecule is selected from: triphenylphosphonium (TPP), dequalinium (DQA), E-4-(1H-Indol-3-ylvinyl)-N-Methylpyridineiodide (F16), Rhodamine 19, biguanidine and guanidine.
81. The method of any one of embodiments 53 to 64, wherein an amount of 50 μg to 200 μg of mitochondria are contacted with 0.1 to 50 μmol of the nucleic acid molecules and 0.02 to 10 μg, preferably 0.02 to 5 μg, of the positively-charged species.
82. The method of embodiment 79 or 80, wherein an amount of 50 μg to 200 μg of mitochondria are contacted with 0.1 to 50 μmol of the nucleic acid molecules linked to a mitochondria-targeting small molecule.
83. The method according to any one of embodiments 53 to 82, wherein the method further comprises linking to and/or enveloping the mitochondrion in a protective layer.
84. The method according to embodiment 83, wherein the protective layer is a protective polymer.
85. The method according to embodiment 84, wherein the protective polymer is a linear or branched cationic polymer, optionally wherein the linear or branched cationic polymer is electrostatically linked to the one or more nucleic acid molecule(s).
86. The method according to embodiment 84, wherein the protective polymer is a linear or branched cationic block copolymer, optionally wherein the linear or branched cationic block copolymer is electrostatically linked to the one or more nucleic acid molecule(s).
87. The method according to embodiment 84, wherein the protective polymer is a cationic graft (g) copolymer, optionally wherein the cationic graft (g) copolymer is electrostatically linked to the one or more nucleic acid molecule(s).
88. The method according to embodiment 84, wherein the protective polymer is a linear or branched pegylated (PEG) cationic polymer, optionally wherein the linear or branched pegylated (PEG) cationic polymer is electrostatically linked to the one or more nucleic acid molecule(s).
89. The method according to embodiment to 83, wherein the protective layer is a lipid formulation, optionally wherein the lipid formulation is a cationic lipid formulation, further optionally wherein the cationic lipid formulation is electrostatically linked to the one or more nucleic acid molecule(s).
90. The method according to any one of embodiments 83 to 89, wherein the protective layer is linked to an antibody, optionally wherein the protective layer linked to an antibody is electrostatically linked to the one or more nucleic acid molecule(s).
91. The method according to any one of embodiments 83 to 89, wherein the protective layer is linked to a carbohydrate, optionally wherein the protective layer linked to a carbohydrate is electrostatically linked to the one or more nucleic acid molecule(s).
92. The method according to embodiment 85, wherein the linear or branched cationic polymer is polyethyleneimine, RGD-modified polyethyleneimine, polylysine, RGD-modified polylysine, polyornithine, RGD-modified polyornithine, polyarginine, RGD modified polyarginine, polypropyleneimine, RGD-modified polypropyleneimine, polyallylamine, RGD-modified polyallylamine, chitosan, RGD-modified chitosan, poly(2-(dimethylamino)ethyl methacrylate), RGD-modified poly(2-(dimethylamino)ethyl methacrylate), poly(amidoamine)s, RGD-modified poly(amidoamine)s or a combination thereof.
93. The method according to embodiment 86, wherein the cationic block copolymer is poly(ethylene glycol)-block-polyethyleneimine, RGD-modified poly(ethylene glycol)-block-polyethyleneimine, poly(ethylene glycol)-block-polylysine, RGD-modified poly(ethylene glycol)-block-polylysine, poly(ethylene glycol)-block-polyornithine, RGD-modified poly(ethylene glycol)-block-polyornithine, poly(ethylene glycol)-block-polyarginine, RGD-modified poly(ethylene glycol)-block-polyarginine, poly(ethylene glycol)-block-polypropyleneimine, RGD-modified poly(ethylene glycol)-block-polypropyleneimine, poly(ethylene glycol)-block-polyallylamine, RGD-modified poly(ethylene glycol)-block-polyallylamine, poly(ethylene glycol)-block-poly(2-(dimethylamino)ethyl methacrylate), RGD-modified poly(ethylene glycol)-block-poly(2-(dimethylamino)ethyl methacrylate), poly(ethylene glycol)-block-poly(amidoamine)s, RGD-modified poly(ethylene glycol)-block-poly(amidoamine)s or a combination thereof.
94. The method according to embodiment 87, wherein the cationic graft (g) copolymer is poly(ethylene glycol)-g-polyethyleneimine, RGD-modified poly(ethylene glycol)-g-polyethyleneimine, poly(ethylene glycol)-g-polylysine, RGD-modified poly(ethylene glycol)-g-polylysine, poly(ethylene glycol)-g-polyornithine, RGD-modified poly(ethylene glycol)-g-polyornithine, poly(ethylene glycol)-g-polyarginine, RGD-modified poly(ethylene glycol)-g-polyarginine, poly(ethylene glycol)-g-polypropyleneimine, RGD-modified poly(ethylene glycol)-g-polypropyleneimine, poly(ethylene glycol)-g-polyallylamine, RGD-modified poly(ethylene glycol)-g-polyallylamine, poly(ethylene glycol)-g-poly(2-(dimethylamino)ethyl methacrylate), RGD-modified poly(ethylene glycol)-g-poly(2-(dimethylamino)ethyl methacrylate), poly(ethylene glycol)-g-poly(amidoamine)s, RGD-modified poly(ethylene glycol)-g-poly(amidoamine)s or a combination thereof.
95. The method according to embodiments 88, wherein the pegylated (PEG) cationic polymer is pegylated-polyethyleneimine, RGD-modified pegylated polyethyleneimine, pegylated polylysine, RGD-modified pegylated polylysine, histidylated polylysine, pegylated polyornithine, RGD-modified pegylated polyornithine, pegylated polyarginine, RGD-modified pegylated polyarginine, pegylated polypropyleneimine, RGD-modified pegylated polypropyleneimine, pegylated polyallylamine, RGD-modified pegylated polyallylamine, pegylated chitosan, RGD-modified pegylated chitosan, pegylated poly(2-(dimethylamino)ethyl methacrylate), RGD-modified pegylated poly(2-(dimethylamino)ethyl methacrylate), pegylated poly(amidoamine) s RGD-modified pegylated poly(amidoamine)s or a combination thereof.
96. The method according to embodiment 89, wherein the lipid formulation comprises DC-cholesterol (3β-[N—(N′,N′-Dimethylaminoethane)-carbamoyl]cholesterol hydrochloride), DLinDMA (1,2-dilinoleyloxy-3-dimethylaminopropane), DLinMC3DMA (dilinoleylmethyl-4-dimethylaminobutyrate), DODMA (1,2-dioleyloxy-3-dimethylaminopropane), DOGS (dioctadecylamidoglycylspermine), DOSPA (2,3-dioleyloxy-N-[2 (sperminecarboxamido)ethyl]-N,N-dimethyl-1-propanaminium), DOTAP (1,2-dioleoyl-3-trimethylammonium-propane chloride), DOTMA (1,2-di-O-octadecenyl-3-trimethylammonium propane chloride), UGG (unsaturated guanidinium glycoside), DOPE (1,2-Dioleoyl-sn-glycerophosphoethanolamine), lipofectamine or a combination thereof.
97. The method according to embodiment 96, wherein the lipid formulation further comprises another lipid, preferably wherein said lipid is cholesterol, a substituted or unsubstituted cholesterol, a cholesterol derivative, such as a hydroxylated cholesterol derivative (e.g., a hydroxycholesterol), a PEG-lipid, DMPC (1,2-Dimyristoyl-sn-glycero-3-phosphocholine), DSPC (1,2-Distearoyl-sn-glycero-3-phosphocholine), DODAP (1,2-dioleoyl-3-dimethylammonium propane), DDA (dimethyldioctadecylammonium), 1,2-dioleoyl-sn-glycero-3-phosphate, 1,2-dimyristoyl-sn-glycero-3-phosphate, bis(monooleoylglycerol)phosphate or a combination thereof.
98. The method according to embodiment 84, wherein the protective polymer is a zwitterionic protective polymer, optionally wherein the zwitterionic protective polymer is electrostatically linked to the one or more nucleic acid molecule(s).
99. The method according to embodiment 98, wherein the zwitterionic protective polymer is selected from: poly(2-methacryloyloxyethyl phosphorylcholine) (PMPC), polyethyleneimine-g-poly(2-methacryloyloxyethyl phosphorylcholine) (PEI-g-PMPC), co-assembly of cationic (carboxyl-functionalized) and anionic (amino-functionalized) copolyesters based on poly(ε-caprolactone)-block-poly(butylene fumarate)-block-poly(ε-caprolactone) (PCL-b-PBF-b-PCL), poly(lactic-co-glycolic acid) (PLGA)-PCB block copolymers (PLGA-b-PCB).
100. The method according to any one of embodiments 83 to 99, wherein the mitochondrion comprises a positively-charged species, wherein the positively-charged species is a polycationic polymer according to embodiment 60 or 61, and wherein the ratio of the polycationic polymer to the protective layer is about 1:2.
101. The method according to embodiment 100, wherein 50 μg to 200 μg of mitochondria are contacted with 0.1 to 50 μmol of nucleic acid molecules and 0.02 to 10 μg of the components of protective layer.
102. A mitochondrion comprising one or more polypeptide(s) attached to the outer membrane of the mitochondrion, wherein the one or more polypeptide(s):
   a) is electrostatically attached to the outer membrane of the mitochondrion; or
   b) is covalently linked to the outer membrane of the mitochondrion; or
   c) is linked to an antibody that specifically binds to an antigen comprised in the outer membrane of the mitochondrion; or
   d) is linked to a mitochondria-targeting small molecule.
103. The mitochondrion according to embodiment 102, wherein the one or more polypeptide(s) is a charged polypeptide.
104. The mitochondrion according to embodiment 102 or 103, wherein the polypeptide is negatively-charged.
105. The mitochondrion according to embodiment 102 or 103, wherein the polypeptide is positively-charged.
106. The mitochondrion according to embodiment 104, wherein the polypeptide is electrostatically attached to the outer membrane of the mitochondrion via a positively-charged species.
107. The mitochondrion according to embodiment 105, wherein the polypeptide is electrostatically attached to the outer membrane of the mitochondrion.
108. The mitochondrion according to embodiment 106, wherein the positively-charged species is a polycationic species, wherein the polycationic species is a linear or branched polycationic polymer, optionally wherein the linear or branched polycationic polymer is electrostatically linked to the one or more polypeptide.
109. The mitochondrion according to embodiment 108, wherein the linear or branched polycationic polymer is: polylysine, histidylated polylysine, polyornithine, polyarginine, high-mobility group protein (HMG) 1 and 17, modified chitosan, cationized human serum albumin, polyethyleneimine (PEI), polypropyleneimine (PPI), a cationic dendrimer, poly(2-(dimethylamino)ethyl methacrylate) (PDMAEMA), a polyallylamine derivative, diethylaminoethyl (DEAE)-dextran, poly(N-alkyl-4-vinylpyridinium), a poly(amidoamine), cationic gelatin, cationic cellulose or a combination thereof.
110. The mitochondrion according to embodiment 106, wherein the positively-charged species is a positively-charged nanoparticle.
111. The mitochondrion according to embodiments 106, wherein the positively-charged species is a positively-charged particle.
112. The mitochondrion according to embodiment 110, wherein the one or more polypeptide(s) is attached to the surface of the positively-charged nanoparticle or encapsulated in the positively-charged nanoparticle.
113. The mitochondrion according to embodiment 111, wherein the one or more polypeptide(s) is attached to the surface of the positively-charged particle or encapsulated in the positively-charged particle.
114. The mitochondrion according to embodiments 110 or 112, wherein the positively-charged nanoparticle is a lipid nanoparticle, a dendrimer nanoparticle, a micelle nanoparticle, a protein nanoparticle, a liposome, a non-porous silica nanoparticle, a mesoporous silica nanoparticle, a silicon nanoparticle, a gold nanoparticle, a gold nanowire, a silver nanoparticle, a platinum nanoparticle, a palladium nanoparticle, a titanium dioxide nanoparticle, a carbon nanotube, a carbon dot nanoparticle, a polymer nanoparticle, a zeolite nanoparticle, an aluminium oxide nanoparticle, a hydroxyapatite nanoparticle, a quantum dot nanoparticle, a zinc oxide nanoparticle, a zirconium oxide nanoparticle, graphene or a graphene oxide nanoparticle.
115. The mitochondrion according to any one of embodiments 102 to 105, wherein the one or more polypeptide(s) is linked to a second polypeptide in the outer membrane of the mitochondrion via an amide bond.
116. The mitochondrion according to embodiment 115, wherein the one or more polypeptide(s) has been modified to undergo formation of the amide bond with an amine function comprised in the second polypeptide in the outer membrane of the mitochondrion.
117. The mitochondrion according to any one of embodiments 102 to 105, wherein the one or more polypeptide(s) is encapsulated in a nanoparticle, and wherein the nanoparticle comprises a functional group that allows covalent linkage of the nanoparticle to a second polypeptide in the outer membrane of the mitochondrion.
118. The mitochondrion according to embodiments 102 or 105, wherein the antibody specifically binds to an antigen comprised in the outer membrane of the mitochondrion, wherein the antigen is OPA1, TOM70, TOMM20, Mitofusin 1, Mitofusin 2 or VDAC1.
119. The mitochondrion according to any one of embodiments 102 to 105 or 118, wherein the one or more polypeptide(s) is encapsulated in a nanoparticle, and wherein the nanoparticle is covalently linked to the antibody.
120. The mitochondrion according to any one of embodiments 104, 106 or 118, wherein the one or more polypeptide(s) is electrostatically linked to the antibody, wherein the antibody is a modified antibody, wherein the modified antibody possesses one or more positive charge(s).
121. The mitochondrion according to any one of embodiments 102 to 105 or 118, wherein the one or more polypeptide(s) is covalently linked to biotin, wherein biotin is linked the antibody, wherein the antibody is an avidin conjugated antibody.
122. The mitochondrion according to any one of embodiments 102 to 105 or 118, wherein the one or more polypeptide(s) is covalently linked to an activated ester, wherein the activated ester is linked to the antibody via an amide bond.
123. The mitochondrion according to any one of embodiments 102 to 105 or 118, wherein the one or more polypeptide(s) is encapsulated in a nanoparticle, wherein the nanoparticle is electrostatically linked to the antibody, wherein the antibody is a modified antibody, wherein the modified antibody possesses one or more positive charge(s).
124. The mitochondrion according to any one of embodiments 102 to 105 or 118, wherein the one or more polypeptide(s) is encapsulated in a nanoparticle, wherein the nanoparticle is covalently linked to biotin, wherein biotin is linked the antibody, wherein the antibody is an avidin conjugated antibody.
125. The mitochondrion according to any one of embodiments 102 to 105 or 118, wherein the one or more polypeptide(s) is encapsulated in a nanoparticle, wherein the nanoparticle is covalently linked to an activated ester, wherein the activated ester is linked to the antibody via an amide bond.
126. The mitochondrion according to any one of embodiments 102 to 105, wherein the mitochondria-targeting small molecule is selected from the group consisting of triphenylphosphonium (TPP), dequalinium (DQA), E-4-(1H-Indol-3-ylvinyl)-N-Methylpyridineiodide (F16), Rhodamine 19, biguanidine and guanidine.
127. The mitochondrion according to any one of embodiments 102 to 126, wherein the mitochondrion is linked to and/or enveloped in a protective layer.
128. The mitochondrion according to embodiment 128, wherein the protective layer is a protective polymer.
129. The mitochondrion according to embodiment 128, wherein the protective polymer is a linear or branched cationic polymer, optionally wherein the linear or branched cationic polymer is electrostatically linked to the one or more polypeptide(s).
130. The mitochondrion according to embodiment 128, wherein the protective polymer is a linear or branched cationic block copolymer, optionally wherein the linear or branched cationic block copolymer is electrostatically linked to the one or more polypeptides(s).
131. The mitochondrion according to embodiment 128, wherein protective polymer is a cationic graft (g) copolymer, optionally wherein the cationic graft (g) copolymer is electrostatically linked to the one or more polypeptide(s).
132. The mitochondrion according to embodiment 128, wherein the protective polymer is a linear or branched pegylated (PEG) cationic polymer, optionally wherein the linear or branched pegylated (PEG) cationic polymer is electrostatically linked to the one or more polypeptides(s).
133. The mitochondrion according to embodiment 127, wherein the protective layer is a lipid formulation, optionally wherein the lipid formulation is a cationic lipid formulation, further optionally wherein the cationic lipid formulation is electrostatically linked to the one or more polypeptide(s).
134. The mitochondrion according to any one of embodiments 127 to 133, wherein the protective layer is linked to an antibody, optionally wherein the protective layer linked to an antibody is electrostatically linked to the one or more polypeptide(s).
135. The mitochondrion according to any one of embodiments 127 to 133, wherein the protective layer is linked to a carbohydrate, optionally wherein the protective layer linked to a carbohydrate is electrostatically linked to the one or more polypeptide(s).
136. The mitochondrion according to embodiment 129, wherein the linear or branched cationic polymer is polyethyleneimine, RGD-modified polyethyleneimine, polylysine, RGD-modified polylysine, polyornithine, RGD-modified polyornithine, polyarginine, RGD modified polyarginine, polypropyleneimine, RGD-modified polypropyleneimine, polyallylamine, RGD-modified polyallylamine, chitosan, RGD-modified chitosan, poly(2-(dimethylamino)ethyl methacrylate), RGD-modified poly(2-(dimethylamino)ethyl methacrylate), poly(amidoamine)s, RGD-modified poly(amidoamine)s or a combination thereof.
137. The mitochondrion according to embodiment 130, wherein the cationic block copolymer is poly(ethylene glycol)-block-polyethyleneimine, RGD-modified poly(ethylene glycol)-block-polyethyleneimine, poly(ethylene glycol)-block-polylysine, RGD-modified poly(ethylene glycol)-block-polylysine, poly(ethylene glycol)-block-polyornithine, RGD-modified poly(ethylene glycol)-block-polyornithine, poly(ethylene glycol)-block-polyarginine, RGD-modified poly(ethylene glycol)-block-polyarginine, poly(ethylene glycol)-block-polypropyleneimine, RGD-modified poly(ethylene glycol)-block-polypropyleneimine, poly(ethylene glycol)-block-polyallylamine, RGD-modified poly(ethylene glycol)-block-polyallylamine, poly(ethylene glycol)-block-poly(2-(dimethylamino)ethyl methacrylate), RGD-modified poly(ethylene glycol)-block-poly(2-(dimethylamino)ethyl methacrylate), poly(ethylene glycol)-block-poly(amidoamine)s, RGD-modified poly(ethylene glycol)-block-poly(amidoamine)s or a combination thereof.
138. The mitochondrion according to embodiments 131, wherein the cationic graft (g) copolymer is poly(ethylene glycol)-g-polyethyleneimine, RGD-modified poly(ethylene glycol)-g-polyethyleneimine, poly(ethylene glycol)-g-polylysine, RGD-modified poly(ethylene glycol)-g-polylysine, poly(ethylene glycol)-g-polyornithine, RGD-modified poly(ethylene glycol)-g-polyornithine, poly(ethylene glycol)-g-polyarginine, RGD-modified poly(ethylene glycol)-g-polyarginine, poly(ethylene glycol)-g-polypropyleneimine, RGD-modified poly(ethylene glycol)-g-polypropyleneimine, poly(ethylene glycol)-g-polyallylamine, RGD-modified poly(ethylene glycol)-g-polyallylamine, poly(ethylene glycol)-g-poly(2-(dimethylamino)ethyl methacrylate), RGD-modified poly(ethylene glycol)-g-poly(2-(dimethylamino)ethyl methacrylate), poly(ethylene glycol)-g-poly(amidoamine)s, RGD-modified poly(ethylene glycol)-g-poly(amidoamine)s or a combination thereof.
139. The mitochondrion according to embodiments 132, wherein the pegylated (PEG) cationic polymer is pegylated-polyethyleneimine, RGD-modified pegylated polyethyleneimine, pegylated polylysine, RGD-modified pegylated polylysine, histidylated polylysine, pegylated polyornithine, RGD-modified pegylated polyornithine, pegylated polyarginine, RGD-modified pegylated polyarginine, pegylated polypropyleneimine, RGD-modified pegylated polypropyleneimine, pegylated polyallylamine, RGD-modified pegylated polyallylamine, pegylated chitosan, RGD-modified pegylated chitosan, pegylated poly(2-(dimethylamino)ethyl methacrylate), RGD-modified pegylated poly(2-(dimethylamino)ethyl methacrylate), pegylated poly(amidoamine) s RGD-modified pegylated poly(amidoamine)s or a combination thereof.
140. The mitochondrion according to embodiment 133, wherein the lipid formulation comprises DC-cholesterol (3β-[N—(N′,N′-Dimethylaminoethane)-carbamoyl]cholesterol hydrochloride), DLinDMA (1,2-dilinoleyloxy-3-dimethylaminopropane), DLinMC3DMA (dilinoleylmethyl-4-dimethylaminobutyrate), DODMA (1,2-dioleyloxy-3-dimethylaminopropane), DOGS (dioctadecylamidoglycylspermine), DOSPA (2,3-dioleyloxy-N-[2 (sperminecarboxamido)ethyl]-N,N-dimethyl-1-propanaminium), DOTAP (1,2-dioleoyl-3-trimethylammonium-propane chloride), DOTMA (1,2-di-O-octadecenyl-3-trimethylammonium propane chloride), UGG (unsaturated guanidinium glycoside), DOPE (1,2-Dioleoyl-sn-glycerophosphoethanolamine), lipofectamine or a combination thereof.
141. The mitochondrion according to embodiment 140, wherein the lipid formulation further comprises another lipid, preferably wherein said lipid is cholesterol, a substituted or unsubstituted cholesterol, a cholesterol derivative, such as a hydroxylated cholesterol derivative (e.g., a hydroxycholesterol), a PEG-lipid, DMPC (1,2-Dimyristoyl-sn-glycero-3-phosphocholine), DSPC (1,2-Distearoyl-sn-glycero-3-phosphocholine), DODAP (1,2-dioleoyl-3-dimethylammonium propane), DDA (dimethyldioctadecylammonium), 1,2-dioleoyl-sn-glycero-3-phosphate, 1,2-dimyristoyl-sn-glycero-3-phosphate, bis(monooleoylglycerol)phosphate or a combination thereof.
142. The mitochondrion according to embodiment 128, wherein the mitochondrion is linked to and/or enveloped in a zwitterionic protective polymer, optionally wherein the zwitterionic protective polymer is electrostatically linked to the one or more polypeptide(s).
143. The mitochondrion according to embodiment 142, wherein the zwitterionic protective polymer is selected from: poly(2-methacryloyloxyethyl phosphorylcholine) (PMPC), polyethyleneimine-g-poly(2-methacryloyloxyethyl phosphorylcholine) (PEI-g-PMPC), co-assembly of cationic (carboxyl-functionalized) and anionic (amino-functionalized) copolyesters based on poly(ε-caprolactone)-block-poly(butylene fumarate)-block-poly(ε-caprolactone) (PCL-b-PBF-b-PCL), poly(lactic-co-glycolic acid) (PLGA)-PCB block copolymers (PLGA-b-PCB).
144. A composition comprising a plurality of mitochondria according to any one of embodiments 102 to 143.
145. A pharmaceutical composition comprising a plurality of mitochondria according to any one of embodiments 102 to 143 and a pharmaceutically acceptable carrier.
146. The pharmaceutical composition according to embodiment 145, wherein the pharmaceutical composition is formulated as a solution.
147. The pharmaceutical composition according to embodiment 145, wherein the pharmaceutical composition is formulated as an aerosol.
148. The mitochondrion according to any one of embodiments 102 to 143, the composition according to embodiment 144 or the pharmaceutical composition according to any one of embodiments 145 to 147 for use as a medicament.
149. The mitochondrion according to any one of embodiments 102 to 143, the composition according to embodiment 144 or the pharmaceutical composition according to any one of embodiments 145 to 147 for use in gene therapy.
150. The mitochondrion according to any one of embodiments 102 to 143, the composition according to embodiment 144 or the pharmaceutical composition according to any one of embodiments 145 to 147 for use in the treatment of cardiovascular diseases, in particular for use in the treatment of ischemic heart disease, ischemia-reperfusion injury or atherosclerosis.
151. The mitochondrion according to any one of embodiments 102 to 143, the composition according to embodiment 144 or the pharmaceutical composition according to any one of embodiments 145 to 147 for use in the treatment of aging related diseases, in particular for use in the treatment of, sarcopenia, Parkinson's disease or Hutchinson-Gilford progeria syndrome.
152. The mitochondrion according to any one of embodiments 102 to 143, the composition according to embodiment 144 or the pharmaceutical composition according to any one of embodiments 145 to 147 for use in the treatment of cancer.
153. The mitochondrion according to any one of embodiments 102 to 143, the composition according to embodiment 144 or the pharmaceutical composition according to any one of embodiments 145 to 147 for use in in vitro, ex vivo, or in vivo genome editing.
154. The mitochondrion according to any one of embodiments 102 to 143, the composition according to embodiment 144 or the pharmaceutical composition according to any one of embodiments 145 to 147 for use in radiation therapy.
155. A method for delivering a polypeptide to a target organ, the method comprising a step of administering the pharmaceutical composition according to embodiment 145 or 146 into the bloodstream of a subject in need, wherein the pharmaceutical composition is administered into the bloodstream upstream of the target organ.
156. A method for delivering a polypeptide to the lung, the method comprising a step of administering the pharmaceutical composition according to embodiment 147 to a subject in need, wherein the pharmaceutical composition is administered by inhalation.
157. A method for attaching at least one polypeptide to the outer membrane of a mitochondrion, the method comprising the steps of:
   a) providing a preparation of mitochondria;
   b) contacting the mitochondria provided in step (a) with at least one polypeptide, optionally in the presence of a positively-charged species; and
   c) attaching the at least one polypeptide to the mitochondria, optionally via the positively-charged species.
158. The method according to embodiment 157, wherein in step (b), the at least one polypeptide is contacted with the mitochondrion in the presence of the positively-charged species, wherein:
   a) the at least one polypeptide is simultaneously contacted with the mitochondria and the positively-charged species;
   b) wherein the at least one polypeptide is contacted with the positively-charged species to form a positively-charged complex before the positively-charged complex is contacted with the mitochondria; or
   c) the mitochondrion is contacted with the positively-charged species and subsequently contacted with the at least one polypeptide.
159. The method according to embodiment 157 or 158, wherein the mitochondria are contacted with the at least one polypeptide and optionally the positively-charged species, in a suitable buffer.
160. The method according to embodiment 159, wherein the buffer comprises or consists of HEPES, EGTA, Trehalose CHES and sodium phosphate dibasic dihydrate, preferably wherein buffer comprises a mixture of a Solution X comprising or consisting of HEPES, EGTA and Trehalose and of a Solution Y comprising or consisting of CHES and sodium phosphate dibasic dihydrate, more preferably, wherein the buffer comprises a 4:1 mixture of Solution X comprising or consisting of 20 mM HEPES, 1 mM EGTA and 300 mM Trehalose (pH 7.2) and Solution Y comprising or consisting of 0.1 M CHES (pH 10) and 0.2 M sodium phosphate dibasic dihydrate.
161. The method according to any one of embodiments 157 to 160, wherein the mitochondria are contacted with the at least one polypeptide and the positively-charged species at room temperature for at least 5 minutes, such as at least 10 minutes, 20 or 30 minutes.
162. The method according to any one of embodiments 157 to 161, wherein the mitochondria are contacted with the at least one polypeptide and the positively-charged species in the dark.
163. The mitochondrion according to any one of embodiments 102, 103, 105, 106, or 108 to 154, or the method according to any one of embodiments 157 to 162, wherein the polypeptide comprises lysine, arginine or histidine.
164. The mitochondrion according to any one of embodiments 102 to 104, 107 to 154, or the method according to any one of embodiments 157 to 162, wherein the polypeptide comprises: aspartate or glutamate.
165. The method according to any one of embodiments 157 to 164, wherein the positively-charged species is a polycationic species, wherein the polycationic species is a linear or branched polycationic polymer, optionally wherein the linear or branched polycationic polymer is electrostatically linked to a polypeptide comprised in the at least one polypeptide.
166. The method according to embodiment 165, wherein the linear or branched polycationic polymer is: polylysine, histidylated polylysine, polyornithine, polyarginine, high-mobility group protein (HMG) 1 and 17, modified chitosan, cationized human serum albumin, polyethyleneimine (PEI), polypropyleneimine (PPI), a cationic dendrimer, poly(2-(dimethylamino)ethyl methacrylate) (PDMAEMA), a polyallylamine derivative, diethylaminoethyl (DEAE)-dextran, poly(N-alkyl-4-vinylpyridinium), a poly(amidoamine), cationic gelatin, cationic cellulose or a combination thereof.
167. The method according to any one of embodiments 157 to 164, wherein the positively-charged species is a positively-charged nanoparticle.
168. The method according to embodiment 167, wherein the method comprises a further step of
   a) attaching the at least one polypeptide to the surface of the positively-charged nanoparticle; or
   b) encapsulating the at least one polypeptide within the positively-charged nanoparticle.
169. The method according to embodiment 167 or 169, wherein the positively-charged nanoparticle is a lipid nanoparticle, a dendrimer nanoparticle, a micelle nanoparticle, a protein nanoparticle, a liposome, a non-porous silica nanoparticle, a mesoporous silica nanoparticle, a silicon nanoparticle, a gold nanoparticle, a gold nanowire, a silver nanoparticle, a platinum nanoparticle, a palladium nanoparticle, a titanium dioxide nanoparticle, a carbon nanotube, a carbon dot nanoparticle, a polymer nanoparticle, a zeolite nanoparticle, an aluminium oxide nanoparticle, a hydroxyapatite nanoparticle, a quantum dot nanoparticle, a zinc oxide nanoparticle, a zirconium oxide nanoparticle, graphene or a graphene oxide nanoparticle.
170. A method for covalently attaching a polypeptide to the outer membrane of a mitochondrion, the method comprising the steps of:
   a) providing a preparation of mitochondria;
   b) providing a polypeptide that has been modified to comprise an activated ester; and
   c) attaching the polypeptide provided in step (b) to an amine comprised in a polypeptide in the outer membrane of the mitochondria.
171. The method according to embodiment 170, wherein the activated ester is an N-hydroxysuccinimide (NHS) ester.
172. A method for covalently attaching a polypeptide to the outer membrane of a mitochondrion, the method comprising the steps of:
   a) providing a preparation of mitochondria;
   b) encapsulating a polypeptide in a nanoparticle, wherein the surface of the nanoparticle comprises an activated ester; and
   c) attaching the nanoparticle provided in step (b) to an amine comprised in a polypeptide in the outer membrane of the mitochondria.
173. The method according to embodiment 172, wherein the activated ester is an NHS ester.
174. A method for attaching at least one polypeptide to the outer membrane of a mitochondrion, the method comprising the steps of:
   a) providing a preparation of mitochondria;
   b) contacting the mitochondria provided in step (a) comprising an antigen in their outer membrane with at least one polypeptide linked to an antibody; and
   c) attaching the at least one polypeptide to the mitochondria via an antibody that specifically binds to an antigen comprised in the outer membrane of the mitochondrion.
175. The mitochondrion according to embodiment 174, wherein the one or more polypeptide(s) is a charged polypeptide.
176. The mitochondrion according to embodiment 175, wherein the one or more polypeptide(s) is negatively-charged.
177. The mitochondrion according to embodiment 175, wherein the one or more polypeptide(s) is positively-charged.
178. The method according to embodiment 174, wherein the antibody specifically binds to an antigen comprised in the outer membrane of the mitochondrion, wherein the antigen is OPA1, TOM70, TOMM20, Mitofusin 1, Mitofusin 2 or VDAC1.
179. The method according to any one of embodiments 174 to 176 or 178, wherein the one or more polypeptide(s) is electrostatically linked to the antibody, wherein the antibody is a modified antibody, wherein the modified antibody possesses one or more positive charge(s).
180. The method according to any one of embodiments 174, 175 or 177 or 178, wherein the one or more polypeptide(s) is electrostatically linked to the antibody, wherein the antibody is a modified antibody, wherein the modified antibody possesses one or more negative charge(s).
181. The method according to any one of embodiments 174 to 178 wherein the one or more polypeptide(s) is covalently linked to biotin, wherein biotin is linked to the antibody, wherein the antibody is an avidin conjugated antibody.
182. The method according to any one of embodiments 174 to 178 wherein the one or more polypeptide(s) is covalently linked to an activated ester, wherein the activated ester is linked to the antibody via an amide bond.
183. The method according to any one of embodiments 174, 175 or 178, wherein the one or more polypeptide(s) is encapsulated in a nanoparticle, wherein the nanoparticle is electrostatically linked to the antibody, wherein the antibody is a modified antibody, wherein the modified antibody possesses one or more positive charge(s).
184. The method according to any one of embodiments 174 to 176, wherein the one or more polypeptide(s) is encapsulated in a nanoparticle, wherein the nanoparticle is electrostatically linked to the antibody, wherein the antibody is a modified antibody, wherein the modified antibody possesses one or more negative charge(s).
185. The method according to embodiment 174 or 175, wherein the one or more polypeptide(s) is encapsulated in a nanoparticle, wherein the nanoparticle is covalently linked to biotin, wherein biotin is linked the antibody, wherein the antibody is an avidin conjugated antibody.
186. The method according to embodiment 174 or 175, wherein the one or more polypeptide(s) is encapsulated in a nanoparticle, wherein the nanoparticle is covalently linked to an activated ester, wherein the activated ester is linked to the antibody via amide bond.
187. A method for attaching a polypeptide to the outer membrane of a mitochondrion, the method comprising the steps of:
   a) providing a preparation of mitochondria;
   b) contacting the mitochondria provided in step (a) with at least one polypeptide linked to a mitochondria-targeting small molecule; and
   c) attaching the at least one polypeptide to the mitochondria via a mitochondria-targeting small molecule.
188. The method according to embodiment 187, wherein the mitochondria-targeting small molecule is selected from: triphenylphosphonium (TPP), dequalinium (DQA), E-4-(1H-Indol-3-ylvinyl)-N-Methylpyridineiodide (F16), Rhodamine 19, biguanidine and guanidine.
189. The method according to any one of embodiments 157 to 169, wherein an amount of 50 μg to 200 μg of mitochondria are contacted with 0.1 to 10 μg of the polypeptides and 0.2 to 10 μg of the positively-charged species.
190. The method according to embodiment 187 or 188, wherein an amount of 50 μg to 200 μg of mitochondria are contacted with 0.1 to 10 μg of the polypeptides linked to a mitochondria-targeting small molecule.
191. A method for delivering a nucleic acid molecule to the kidney, the method comprising a step of administering the pharmaceutical composition according to embodiment 41 or 42 into the renal artery of a subject in need.
192. A method for delivering a nucleic acid molecule to the heart, the method comprising a step of administering the pharmaceutical composition according to embodiment 41 or 42 into the intracoronary of a subject in need.
193. A method for delivering a nucleic acid molecule to the liver, the method comprising a step of administering the pharmaceutical composition according to embodiment 41 or 42 into the hepatic artery or portal vein of a subject in need.
194. A method for delivering a nucleic acid molecule to the pancreas, the method comprising a step of administering the pharmaceutical composition according to embodiment 41 or 42 into the hepatic artery of a subject in need.
195. A method for delivering a nucleic acid molecule to the duodenum, the method comprising a step of administering the pharmaceutical composition according to embodiment 41 or 42 into the hepatic artery of a subject in need.
196. A method for delivering a nucleic acid molecule to the spleen, the method comprising a step of administering the pharmaceutical composition according to embodiment 41 or 42 into the splenic artery of a subject in need.
197. A method for delivering a nucleic acid molecule to the lung, the method comprising a step of administering the pharmaceutical composition according to embodiment 41 or 42 into the pulmonary artery of a subject in need.
198. A method for delivering a nucleic acid molecule to the intestines, the method comprising a step of administering the pharmaceutical composition according to embodiment 41 or 42 into the superior mesenteric artery of a subject in need.
199. A method for delivering a nucleic acid molecule to the bladder, the method comprising a step of administering the pharmaceutical composition according to embodiment 41 or 42 into the superior and inferior vesical arteries of a subject in need.
200. A method for delivering a nucleic acid molecule to a target organ, the method comprising a step of administering the pharmaceutical composition according to embodiment 41 or 42 into a subject in need, wherein the pharmaceutical composition is administered into the kidney or bladder or intestines or pancreas or duodenum or liver or lung or spleen through direct injection.
201. A method for delivering a polypeptide to the kidney, the method comprising a step of administering the pharmaceutical composition according to embodiment 145 or 146 into the renal artery of a subject in need.
202. A method for delivering a polypeptide to the heart, the method comprising a step of administering the pharmaceutical composition according to embodiment 145 or 146 into the intracoronary of a subject in need.
203. A method for delivering a polypeptide to the liver, the method comprising a step of administering the pharmaceutical composition according to embodiment 145 or 146 into the hepatic artery or portal vein of a subject in need.
204. A method for delivering a polypeptide to the pancreas, the method comprising a step of administering the pharmaceutical composition according to embodiment 145 or 146 into the hepatic artery of a subject in need.
205. A method for delivering a polypeptide to the duodenum, the method comprising a step of administering the pharmaceutical composition according to embodiment 145 or 146 into the hepatic artery of a subject in need.
206. A method for delivering a polypeptide to is the spleen, the method comprising a step of administering the pharmaceutical composition according to embodiment 145 or 146 into the splenic artery of a subject in need.
207. A method for delivering a polypeptide to the lung, the method comprising a step of administering the pharmaceutical composition according to embodiment 145 or 146 into the pulmonary artery of a subject in need.
208. A method for delivering a polypeptide to the intestines, the method comprising a step of administering the pharmaceutical composition according to embodiment 145 or 146 into the superior mesenteric artery of a subject in need.
209. A method for delivering a polypeptide to the bladder, the method comprising a step of administering the pharmaceutical composition according to embodiment 145 or 146 into the superior and inferior vesical arteries of a subject in need.
210. A method for delivering a polypeptide to a target organ, the method comprising a step of administering the pharmaceutical composition according to embodiment 145 or 146 into a subject in need, wherein the pharmaceutical composition is administered into the kidney or bladder or intestines or pancreas or duodenum or liver or lung or spleen through direct injection.
211. A mitochondrion comprising one or more drug(s) attached to the outer membrane of the mitochondrion, wherein the one or more drug(s):
   a) is electrostatically attached to the outer membrane of the mitochondrion; or
   b) is covalently linked to the outer membrane of the mitochondrion; or
   c) is linked to an antibody that specifically binds to an antigen comprised in the outer membrane of the mitochondrion; or
   d) is linked to a mitochondria-targeting small molecule.
212. The mitochondrion according to embodiment 211, wherein the one or more drug(s) is a charged drug.
213. The mitochondrion according to embodiment 211 or 212, wherein the drug is an anionic drug, optionally wherein the anionic drug is selected from: potassium iodide, iodide, artesunate, sodium fluoride, carbamide peroxide, sodium zirconium cyclosilicate, nitrite, lithium carbonate, zinc chloride, aluminium hydroxide, magaldrate, aluminium sesquichlorohydrate, hydrotalcite, aluminium glycinate, aloglutamol, dihydroxyaluminium sodium carbonate, cystine, nitroprusside, montelukast, stepronin, prostaglandin G2, pyrophosphoric acid, OXI-4503, tetrachlorodecaoxide, NCX 701, PX-12, nitrous acid, chromic chloride, ferric pyrophosphate, activated charcoal, monopotassium phosphate, dipotassium phosphate, sodium fluorophosphate, potassium nitrate, potassium bicarbonate, sulfur hexafluoride, PF-4191834, allicin, artefenomel, lodenafil carbonate, devimistat, GW-274150, imrecoxib, chlorine dioxide, perflubutane, CHS-828, QGC-001, trabodenoson, magnesium phosphate, TAK-243, dostarlimab, GC-376 free acid, sodium metabisulfite, diquafosol, ammonium carbonate, NCX-1000 and ethyl nitrite, Nitroprusside, Technetium Tc-99m polyphosphate, Sodium phosphate monobasic, Sodium sulfate, Indium, Chromic nitrate, Tetrafluoroborate, Darapladib, PF-03715455 and Umifenovir.
214. The mitochondrion according to embodiment 211 or 212, wherein the drug is a cationic drug, optionally wherein the cationic drug selected from Methyl-piperidino-pyrazole (MPP), Bretylium, Acetylcarnitine, Fluorocholine F-18, Hexamethonium, Edrophonium, Choline, Succinylcholine, Oxyphenonium, Carbamoylcholine, Gallamine triethiodide, Glycopyrronium, Bethanechol, Ambenonium, Methacholine, Betaine, Benzalkonium, Benzethonium, Emepronium, Benzoxonium, Gallamine, Octenidine, Methantheline, Propantheline, Tubocurarine, Neostigmine, Butylscopolamine, Alcuronium, Metocurine iodide, Levocarnitine, Hexafluronium, Decamethonium, Oxtriphylline, Metocurine, Choline magnesium trisalicylate, Platelet Activating Factor, N,N,N-Trimethyl-2-(phosphonooxy) ethanaminium, Butyrylthiocholine, Betaine aldehyde, C31G, Perifosine, Tetraethylammonium, Miltefosine, Citicoline, Benzododecinium, Choline salicylate, Cetyltrimethylammonium naproxenate and Trimethyltetradecylammonium.
215. The mitochondrion according to embodiment 213, wherein the anionic drug is electrostatically attached to the outer membrane of the mitochondrion via a positively-charged species.
216. The mitochondrion according to embodiment 214, wherein the cationic drug is electrostatically attached to the outer membrane of the mitochondrion.
217. The mitochondrion according to embodiment 215, wherein the positively-charged species is a polycationic species, wherein the polycationic species is a linear or branched polycationic polymer, optionally wherein the linear or branched polycationic polymer is electrostatically linked to one or more anionic drug(s).
218. The mitochondrion according to embodiment 217, wherein the linear or branched polycationic polymer is polylysine, histidylated polylysine, polyornithine, polyarginine, high-mobility group protein (HMG) 1 and 17, modified chitosan, cationized human serum albumin, polyethyleneimine (PEI), polypropyleneimine (PPI), a cationic dendrimer, poly(2-(dimethylamino)ethyl methacrylate) (PDMAEMA), a polyallylamine derivative, diethylaminoethyl (DEAE)-dextran, poly(N-alkyl-4-vinylpyridinium), a poly(amidoamine), cationic gelatin, cationic cellulose or a combination thereof.
219 The mitochondrion according to embodiment 215, wherein the positively-charged species is a positively-charged nanoparticle.
220. The mitochondrion according to embodiment 215, wherein the positively-charged species is a positively-charged particle.
221. The mitochondrion according to embodiment 219, wherein the one or more anionic drug(s) is attached to the surface of the positively-charged nanoparticle or encapsulated in the positively-charged nanoparticle.
222. The mitochondrion according to embodiment 220, wherein the one or more anionic drug(s) is attached to the surface of the positively-charged particle or encapsulated in the positively-charged particle.
223. The mitochondrion according to embodiments 219 or 221, wherein the positively-charged nanoparticle is a lipid nanoparticle, a dendrimer nanoparticle, a micelle nanoparticle, a protein nanoparticle, a liposome, a non-porous silica nanoparticle, a mesoporous silica nanoparticle, a silicon nanoparticle, a gold nanoparticle, a gold nanowire, a silver nanoparticle, a platinum nanoparticle, a palladium nanoparticle, a titanium dioxide nanoparticle, a carbon nanotube, a carbon dot nanoparticle, a polymer nanoparticle, a zeolite nanoparticle, an aluminium oxide nanoparticle, a hydroxyapatite nanoparticle, a quantum dot nanoparticle, a zinc oxide nanoparticle, a zirconium oxide nanoparticle, graphene or a graphene oxide nanoparticle.
224. The mitochondrion according to any one of embodiments 211 to 214, wherein the one or more drug(s) is linked to a polypeptide in the outer membrane of the mitochondrion via an amide bond.
225. The mitochondrion according to embodiment 224, wherein the one or more drug(s) has been modified to undergo formation of the amide bond with an amine function comprised in the polypeptide in the outer membrane of the mitochondrion.
226. The mitochondrion according to any one of embodiments 211 to 214, wherein the one or more drug(s) is encapsulated in a nanoparticle, and wherein the nanoparticle comprises a functional group that allows covalent linkage of the nanoparticle to a polypeptide in the outer membrane of the mitochondrion.
227. The mitochondrion according to any one of embodiments 211 to 214, wherein the antibody specifically binds to an antigen comprised in the outer membrane of the mitochondrion, wherein the antigen is OPA1, TOM70, TOMM20, Mitofusin 1, Mitofusin 2 or VDAC1.
228. The mitochondrion according to any one of embodiments 211 to 214 or 227, wherein the one or more drug(s) is encapsulated in a nanoparticle, and wherein the nanoparticle is covalently linked to the antibody.
229. The mitochondrion according to embodiments 211 to 213, 215 or 227, wherein the one or more anionic drug(s) is electrostatically linked to the antibody, wherein the antibody is a modified antibody, wherein the modified antibody possesses one or more positive charge(s).
230. The mitochondrion according to any one of embodiments 211 to 214 or 227, wherein the one or more drug(s) is covalently linked to biotin, wherein biotin is linked to the antibody, wherein the antibody is an avidin conjugated antibody.
231. The mitochondrion according to any one of embodiments 211 to 214 or 227, wherein the one or more drug(s) is covalently linked to an activated ester, wherein the activated ester is linked to the antibody via an amide bond.
232. The mitochondrion according to any one of embodiments 211 to 214 or 227, wherein the one or more drug(s) is encapsulated in a nanoparticle, wherein the nanoparticle is electrostatically linked to the antibody, wherein the antibody is a modified antibody, wherein the modified antibody possesses one or more positive charge(s).
233. The mitochondrion according to any one of embodiments 211 to 214 or 227, wherein the one or more drug(s) is encapsulated in a nanoparticle, wherein the nanoparticle is covalently linked to biotin, wherein biotin is linked the antibody, wherein the antibody is an avidin conjugated antibody.
234. The mitochondrion according to any one of embodiments 211 to 214 or 227, wherein the one or more drug(s) is encapsulated in a nanoparticle, wherein the nanoparticle is covalently linked to an activated ester, wherein the activated ester is linked to the antibody via an amide bond.
235. The mitochondrion according to embodiments 211, wherein the mitochondria-targeting small molecule is selected from the group consisting of triphenylphosphonium (TPP), dequalinium (DQA), E-4-(1H-Indol-3-ylvinyl)-N-Methylpyridineiodide (F16), Rhodamine 19, biguanidine and guanidine.
236. The mitochondrion according to any one of embodiments 211 to 235, wherein the mitochondrion is linked to and/or enveloped in a protective layer.
237. The mitochondrion according to embodiment 236, wherein the protective layer is a protective polymer.
238. The mitochondrion according to embodiment 236, wherein the protective polymer is a linear or branched cationic polymer, optionally wherein the linear or branched cationic polymer is electrostatically linked to the one or more drug(s).
239. The mitochondrion according to embodiment 236, wherein the protective polymer is a linear or branched cationic block copolymer, optionally wherein the linear or branched cationic block copolymer is electrostatically linked to the one or more drug(s).
240. The mitochondrion according to embodiment 236, wherein protective polymer is a cationic graft (g) copolymer, optionally wherein the cationic graft (g) copolymer is electrostatically linked to the one or more drug(s).
241. The mitochondrion according to embodiment 236, wherein the protective polymer is a linear or branched pegylated (PEG) cationic polymer, optionally wherein the linear or branched pegylated (PEG) cationic polymer is electrostatically linked to the one or more drug(s).
242. The mitochondrion according to embodiment 235, wherein the protective layer is a lipid formulation, optionally wherein the lipid formulation is a cationic lipid formulation, further optionally wherein the cationic lipid formulation is electrostatically linked to the one or more drug(s).
243. The mitochondrion according to any one of embodiments 235 to 242, wherein the protective layer is linked to an antibody, optionally wherein the protective layer linked to an antibody is electrostatically linked to the one or more drug(s).
244. The mitochondrion according to any one of embodiments 235 to 242, wherein the protective layer is linked to a carbohydrate, optionally wherein the protective layer linked to a carbohydrate is electrostatically linked to the one or more drug(s).
245. The mitochondrion according to embodiment 237 or 238, wherein the linear or branched cationic polymer is polyethyleneimine, RGD-modified polyethyleneimine, polylysine, RGD-modified polylysine, polyornithine, RGD-modified polyornithine, polyarginine, RGD modified polyarginine, polypropyleneimine, RGD-modified polypropyleneimine, polyallylamine, RGD-modified polyallylamine, chitosan, RGD-modified chitosan, poly(2-(dimethylamino)ethyl methacrylate), RGD-modified poly(2-(dimethylamino)ethyl methacrylate), poly(amidoamine)s, RGD-modified poly(amidoamine)s or a combination thereof.
246. The mitochondrion according to embodiment 239, wherein the cationic block copolymer is poly(ethylene glycol)-block-polyethyleneimine, RGD-modified poly(ethylene glycol)-block-polyethyleneimine, poly(ethylene glycol)-block-polylysine, RGD-modified poly(ethylene glycol)-block-polylysine, poly(ethylene glycol)-block-polyornithine, RGD-modified poly(ethylene glycol)-block-polyornithine, poly(ethylene glycol)-block-polyarginine, RGD-modified poly(ethylene glycol)-block-polyarginine, poly(ethylene glycol)-block-polypropyleneimine, RGD-modified poly(ethylene glycol)-block-polypropyleneimine, poly(ethylene glycol)-block-polyallylamine, RGD-modified poly(ethylene glycol)-block-polyallylamine, poly(ethylene glycol)-block-poly(2-(dimethylamino)ethyl methacrylate), RGD-modified poly(ethylene glycol)-block-poly(2-(dimethylamino)ethyl methacrylate), poly(ethylene glycol)-block-poly(amidoamine)s, RGD-modified poly(ethylene glycol)-block-poly(amidoamine)s or a combination thereof.
247. The mitochondrion according to embodiments 240, wherein the cationic graft (g) copolymer is poly(ethylene glycol)-g-polyethyleneimine, RGD-modified poly(ethylene glycol)-g-polyethyleneimine, poly(ethylene glycol)-g-polylysine, RGD-modified poly(ethylene glycol)-g-polylysine, poly(ethylene glycol)-g-polyornithine, RGD-modified poly(ethylene glycol)-g-polyornithine, poly(ethylene glycol)-g-polyarginine, RGD-modified poly(ethylene glycol)-g-polyarginine, poly(ethylene glycol)-g-polypropyleneimine, RGD-modified poly(ethylene glycol)-g-polypropyleneimine, poly(ethylene glycol)-g-polyallylamine, RGD-modified poly(ethylene glycol)-g-polyallylamine, poly(ethylene glycol)-g-poly(2-(dimethylamino)ethyl methacrylate), RGD-modified poly(ethylene glycol)-g-poly(2-(dimethylamino)ethyl methacrylate), poly(ethylene glycol)-g-poly(amidoamine)s, RGD-modified poly(ethylene glycol)-g-poly(amidoamine)s or a combination thereof.
248. The mitochondrion according to embodiments 241, wherein the pegylated (PEG) cationic polymer is pegylated-polyethyleneimine, RGD-modified pegylated polyethyleneimine, pegylated polylysine, RGD-modified pegylated polylysine, histidylated polylysine, pegylated polyornithine, RGD-modified pegylated polyornithine, pegylated polyarginine, RGD-modified pegylated polyarginine, pegylated polypropyleneimine, RGD-modified pegylated polypropyleneimine, pegylated polyallylamine, RGD-modified pegylated polyallylamine, pegylated chitosan, RGD-modified pegylated chitosan, pegylated poly(2-(dimethylamino)ethyl methacrylate), RGD-modified pegylated poly(2-(dimethylamino)ethyl methacrylate), pegylated poly(amidoamine) s RGD-modified pegylated poly(amidoamine)s or a combination thereof.
249. The mitochondrion according to embodiment 242, wherein the lipid formulation comprises DC-cholesterol (3β-[N—(N′,N′-Dimethylaminoethane)-carbamoyl]cholesterol hydrochloride), DLinDMA (1,2-dilinoleyloxy-3-dimethylaminopropane), DLinMC3DMA (dilinoleylmethyl-4-dimethylaminobutyrate), DODMA (1,2-dioleyloxy-3-dimethylaminopropane), DOGS (dioctadecylamidoglycylspermine), DOSPA (2,3-dioleyloxy-N-[2 (sperminecarboxamido)ethyl]-N,N-dimethyl-1-propanaminium), DOTAP (1,2-dioleoyl-3-trimethylammonium-propane chloride), DOTMA (1,2-di-O-octadecenyl-3-trimethylammonium propane chloride), UGG (unsaturated guanidinium glycoside), DOPE (1,2-Dioleoyl-sn-glycerophosphoethanolamine), lipofectamine or a combination thereof.
250. The mitochondrion according to embodiment 249, wherein the lipid formulation further comprises another lipid, preferably wherein said lipid is cholesterol, a substituted or unsubstituted cholesterol, a cholesterol derivative, such as a hydroxylated cholesterol derivative (e.g., a hydroxycholesterol), a PEG-lipid, DMPC (1,2-Dimyristoyl-sn-glycero-3-phosphocholine), DSPC (1,2-Distearoyl-sn-glycero-3-phosphocholine), DODAP (1,2-dioleoyl-3-dimethylammonium propane), DDA (dimethyldioctadecylammonium), 1,2-dioleoyl-sn-glycero-3-phosphate, 1,2-dimyristoyl-sn-glycero-3-phosphate, bis(monooleoylglycerol)phosphate or a combination thereof.
251. The mitochondrion according to embodiment 236 or 237, wherein the mitochondrion is linked to and/or enveloped in a zwitterionic protective polymer, optionally wherein the zwitterionic protective polymer is electrostatically linked to the one or more drug(s).
252. The mitochondrion according to embodiment 251, wherein the zwitterionic protective polymer is selected from: poly(2-methacryloyloxyethyl phosphorylcholine) (PMPC), polyethyleneimine-g-poly(2-methacryloyloxyethyl phosphorylcholine) (PEI-g-PMPC), co-assembly of cationic (carboxyl-functionalized) and anionic (amino-functionalized) copolyesters based on poly(ε-caprolactone)-block-poly(butylene fumarate)-block-poly(ε-caprolactone) (PCL-b-PBF-b-PCL), poly(lactic-co-glycolic acid) (PLGA)-PCB block copolymers (PLGA-b-PCB).
253. A composition comprising a plurality of mitochondria according to any one of embodiments 211 to 252.
254. A pharmaceutical composition comprising a plurality of mitochondria according to any one of embodiments 211 to 252 and a pharmaceutically acceptable carrier.
255. The pharmaceutical composition according to embodiment 254, wherein the pharmaceutical composition is formulated as a solution.
256. The pharmaceutical composition according to embodiment 254, wherein the pharmaceutical composition is formulated as an aerosol.
257. The mitochondrion according to any one of embodiments 211 to 252, the composition according to embodiment 253 or the pharmaceutical composition according to any one of embodiments 254 to 255 for use as a medicament.
258. The mitochondrion according to any one of embodiments 211 to 252, the composition according to embodiment 253 or the pharmaceutical composition according to any one of embodiments 254 to 255 for use in gene therapy.
259. The mitochondrion according to any one of embodiments 211 to 252 the composition according to embodiment 253 or the pharmaceutical composition according to any one of embodiments 254 to 255 for use in the treatment of cardiovascular diseases, in particular for use in the treatment of ischemic heart disease, ischemia-reperfusion injury or atherosclerosis.
260. The mitochondrion according to any one of embodiments 211 to 252, the composition according to embodiment 253 or the pharmaceutical composition according to any one of embodiments 254 to 255 for use in the treatment of aging related diseases, in particular for use in the treatment of, sarcopenia, Parkinson's disease or Hutchinson-Gilford progeria syndrome.
261. The mitochondrion according to any one of embodiments 211 to 252, the composition according to embodiment 253 or the pharmaceutical composition according to any one of embodiments 253 to 255 for use in the treatment of cancer.
262. The mitochondrion according to any one of embodiments 211 to 251, the composition according to embodiment 252 or the pharmaceutical composition according to any one of embodiments 254 to 255 for use in in vitro, ex vivo, or in vivo genome editing.
263. The mitochondrion according to any one of embodiments 211 to 252, the composition according to embodiment 253 or the pharmaceutical composition according to any one of embodiments 254 to 255 for use in radiation therapy.
264. A method for delivering a drug to a target organ, the method comprising a step of administering the pharmaceutical composition according to embodiment 254 or 255 into the bloodstream of a subject in need, wherein the pharmaceutical composition is administered into the bloodstream upstream of the target organ.
265. A method for delivering a drug to the lung, the method comprising a step of administering the pharmaceutical composition according to embodiment 256 to a subject in need, wherein the pharmaceutical composition is administered by inhalation.
266. A method for attaching at least one drug to the outer membrane of a mitochondrion, the method comprising the steps of:
   a) providing a preparation of mitochondria;
   b) contacting the mitochondria provided in step (a) with at least one drug, optionally in the presence of a positively-charged species; and
   c) attaching the at least one drug to the mitochondria, optionally via the positively-charged species.
267. The method according to embodiment 266, wherein in step (b), the at least one drug is contacted with the mitochondrion in the presence of the positively-charged species, wherein:
   a) the at least one drug is simultaneously contacted with mitochondria and the positively-charged species; or
   b) wherein the at least one drug is contacted with the positively-charged species to form a positively-charged complex before the positively-charged complex is contacted with the mitochondria; or
   c) the mitochondrion is contacted with the positively-charged species and subsequently contacted with the at least one drug.
268. The method according to embodiment 266 or 267, wherein the mitochondria are contacted with the at least one drug and the positively-charged species in a suitable buffer.
269. The method according to embodiment 268, wherein the buffer comprises or consists of HEPES, EGTA, Trehalose CHES and sodium phosphate dibasic dihydrate, preferably wherein buffer comprises a mixture of a Solution X comprising or consisting of HEPES, EGTA and Trehalose and of a Solution Y comprising or consisting of CHES and sodium phosphate dibasic dihydrate, more preferably, wherein the buffer comprises a 4:1 mixture of Solution X comprising or consisting of 20 mM HEPES, 1 mM EGTA and 300 mM Trehalose (pH 7.2) and Solution Y comprising or consisting of 0.1 M CHES (pH 10) and 0.2 M sodium phosphate dibasic dihydrate.
270. The method according to any one of embodiments 266 to 269, wherein the mitochondria are contacted with the at least one drug and the positively-charged species at room temperature for at least 5 minutes, such as at least 10 minutes, 20 or 30 minutes.
271. The method according to any one of embodiments 266 to 270, wherein the mitochondria are contacted with of the at least one drug and the positively-charged species in the dark.
272. The method according to any one of embodiments 266 to 271, wherein the positively-charged species is a polycationic species, wherein the polycationic species is a linear or branched polycationic polymer, optionally wherein the linear or branched polycationic polymer is electrostatically linked to the at least one drug.
273. The method according to embodiment 273, wherein the linear or branched polycationic polymer is polylysine, histidylated polylysine, polyornithine, polyarginine, high-mobility group protein (HMG) 1 and 17, modified chitosan, cationized human serum albumin, polyethyleneimine (PEI), polypropyleneimine (PPI), a cationic dendrimer, poly(2-(dimethylamino)ethyl methacrylate) (PDMAEMA), a polyallylamine derivative, diethylaminoethyl (DEAE)-dextran, poly(N-alkyl-4-vinylpyridinium), a poly(amidoamine), cationic gelatin, cationic cellulose or a combination thereof.
274. The method according to any one of embodiments 266 to 271, wherein the positively-charged species is a positively-charged nanoparticle.
275. The method according to embodiment 274, wherein the method comprises a further step of
   a) attaching the at least one drug to the surface of the positively-charged nanoparticle; or
   b) encapsulating the at least one drug within the positively-charged nanoparticle.
276. The method according to embodiment 274 or 275, wherein the positively-charged nanoparticle is a lipid nanoparticle, a dendrimer nanoparticle, a micelle nanoparticle, a protein nanoparticle, a liposome, a non-porous silica nanoparticle, a mesoporous silica nanoparticle, a silicon nanoparticle, a gold nanoparticle, a gold nanowire, a silver nanoparticle, a platinum nanoparticle, a palladium nanoparticle, a titanium dioxide nanoparticle, a carbon nanotube, a carbon dot nanoparticle, a polymer nanoparticle, a zeolite nanoparticle, an aluminium oxide nanoparticle, a hydroxyapatite nanoparticle, a quantum dot nanoparticle, a zinc oxide nanoparticle, a zirconium oxide nanoparticle, graphene or a graphene oxide nanoparticle.
277. A method for covalently attaching a drug to the outer membrane of a mitochondrion, the method comprising the steps of:
   a) providing a preparation of mitochondria;
   b) providing a drug that has been modified to comprise an activated ester; and
   c) attaching the drug provided in step (b) to an amine comprised in a polypeptide in the outer membrane of the mitochondria.
278. The method according to embodiment 277, wherein the activated ester is an N-hydroxysuccinimide (NHS) ester.
279. A method for covalently attaching a drug to the outer membrane of a mitochondrion, the method comprising the steps of:
   a) providing a preparation of mitochondria;
   b) encapsulating a drug in a nanoparticle, wherein the surface of the nanoparticle comprises an activated ester; and
   c) attaching the nanoparticle provided in step (b) to an amine comprised in a polypeptide in the outer membrane of the mitochondria.
280. The method according to embodiment 279, wherein the activated ester is an NHS ester.
281. A mitochondrion comprising two or more of (a) to (c):
   (a) one or more nucleic acid molecule(s) attached to the outer membrane of the mitochondrion
   (b) one or more polypeptide(s) attached to the outer membrane of the mitochondrion,
   (c) one or more drug(s) attached to the outer membrane of the mitochondrion, wherein the one or more nucleic acid molecule(s), polypeptide(s) and/or drug(s)
   i) is/are electrostatically attached to the outer membrane of the mitochondrion, optionally is/are electrostatically attached to the outer membrane of the mitochondrion via a positively-charged species; or
   ii) is/are covalently linked to the outer membrane of the mitochondrion; or
   iii) is/are linked to an antibody that specifically binds to an antigen comprised in the outer membrane of the mitochondrion; or
   iv) is/are linked to a mitochondria-targeting small molecule.
282. The mitochondrion according to embodiment 281, wherein the one or more nucleic acid molecule(s) is DNA and/or RNA.
283. The mitochondrion according to embodiment 281, wherein the one or more polypeptide(s) is charged a charged polypeptide.
284. The mitochondrion according to embodiment 283, wherein the charged polypeptide is a negatively-charged polypeptide.
285. The mitochondrion according to embodiment 283, wherein the charged polypeptide is a positively-charged polypeptide.
286. The mitochondrion according to embodiment 281, wherein the one or more drug(s) is a charged drug.
287. The mitochondrion according to embodiment 286, wherein the charged drug is an anionic drug optionally wherein the anionic drug is selected from: potassium iodide, iodide, artesunate, sodium fluoride, carbamide peroxide, sodium zirconium cyclosilicate, nitrite, lithium carbonate, zinc chloride, aluminium hydroxide, magaldrate, aluminium sesquichlorohydrate, hydrotalcite, aluminium glycinate, aloglutamol, dihydroxyaluminium sodium carbonate, cystine, nitroprusside, montelukast, stepronin, prostaglandin G2, pyrophosphoric acid, OXI-4503, tetrachlorodecaoxide, NCX 701, PX-12, nitrous acid, chromic chloride, ferric pyrophosphate, activated charcoal, monopotassium phosphate, dipotassium phosphate, sodium fluorophosphate, potassium nitrate, potassium bicarbonate, sulfur hexafluoride, PF-4191834, allicin, artefenomel, lodenafil carbonate, devimistat, GW-274150, imrecoxib, chlorine dioxide, perflubutane, CHS-828, QGC-001, trabodenoson, magnesium phosphate, TAK-243, dostarlimab, GC-376 free acid, sodium metabisulfite, diquafosol, ammonium carbonate, NCX-1000 and ethyl nitrite, Nitroprusside, Technetium Tc-99m polyphosphate, Sodium phosphate monobasic, Sodium sulfate, Indium, Chromic nitrate, Tetrafluoroborate, Darapladib, PF-03715455 and Umifenovir.
288. The mitochondrion according to embodiment 286, wherein the charged drug is a cationic drug, optionally wherein the cationic drug selected from Methyl-piperidino-pyrazole (MPP), Bretylium, Acetylcarnitine, Fluorocholine F-18, Hexamethonium, Edrophonium, Choline, Succinylcholine, Oxyphenonium, Carbamoylcholine, Gallamine triethiodide, Glycopyrronium, Bethanechol, Ambenonium, Methacholine, Betaine, Benzalkonium, Benzethonium, Emepronium, Benzoxonium, Gallamine, Octenidine, Methantheline, Propantheline, Tubocurarine, Neostigmine, Butylscopolamine, Alcuronium, Metocurine iodide, Levocarnitine, Hexafluronium, Decamethonium, Oxtriphylline, Metocurine, Choline magnesium trisalicylate, Platelet Activating Factor, N,N,N-Trimethyl-2-(phosphonooxy) ethanaminium, Butyrylthiocholine, Betaine aldehyde, C31G, Perifosine, Tetraethylammonium, Miltefosine, Citicoline, Benzododecinium, Choline salicylate, Cetyltrimethylammonium naproxenate and Trimethyltetradecylammonium.
289. The mitochondrion according to embodiment 285 or 288, wherein the positively-charged polypeptide and/or the cationic drug(s) is electrostatically attached to the outer membrane of the mitochondrion.
290. The mitochondrion according to embodiment 281, 282, 284 or 287, wherein the one or more nucleic acid molecule(s), negatively-charged polypeptide(s) and/or anionic drug(s) is electrostatically attached to the outer membrane of the mitochondrion via a positively-charged species.
291. The mitochondrion according to embodiment 290, wherein the positively-charged species is a polycationic species, wherein the polycationic species is a linear or branched polycationic polymer, optionally wherein the linear or branched polycationic polymer is electrostatically linked to the one or more nucleic acid molecules, the negatively-charged polypeptide and/or the anionic drug.
292. The mitochondrion according to embodiment 291, wherein the linear or branched polycationic polymer is polylysine, histidylated polylysine, polyornithine, polyarginine, high-mobility group protein (HMG) 1 and 17, modified chitosan, cationized human serum albumin, polyethyleneimine (PEI), polypropyleneimine (PPI), a cationic dendrimer, poly(2-(dimethylamino)ethyl methacrylate) (PDMAEMA), a polyallylamine derivative, diethylaminoethyl (DEAE)-dextran, poly(N-alkyl-4-vinylpyridinium), a poly(amidoamine), cationic gelatin, cationic cellulose or a combination thereof.
293. The mitochondrion according to embodiment 290, wherein the positively-charged species is a positively-charged nanoparticle.
294. The mitochondrion according to embodiment 290, wherein the positively-charged species is a positively-charged particle.
295. The mitochondrion according to embodiment 293, wherein the one or more nucleic acid molecule(s), the one or more negatively-charged polypeptide(s) and/or the one or more anionic drug(s) is attached to the surface of the positively-charged nanoparticle or encapsulated in the positively-charged nanoparticle.
296. The mitochondrion according to embodiment 294, wherein the one or more nucleic acid molecule(s), the one or more negatively-charged polypeptide(s) and/or the one or more anionic drug(s) is attached to the surface of the positively-charged particle or encapsulated in the positively-charged particle.
297. The mitochondrion according to embodiment 293 or 295, wherein the positively-charged nanoparticle is a lipid nanoparticle, a dendrimer nanoparticle, a micelle nanoparticle, a protein nanoparticle, a liposome, a non-porous silica nanoparticle, a mesoporous silica nanoparticle, a silicon nanoparticle, a gold nanoparticle, a gold nanowire, a silver nanoparticle, a platinum nanoparticle, a palladium nanoparticle, a titanium dioxide nanoparticle, a carbon nanotube, a carbon dot nanoparticle, a polymer nanoparticle, a zeolite nanoparticle, an aluminium oxide nanoparticle, a hydroxyapatite nanoparticle, a quantum dot nanoparticle, a zinc oxide nanoparticle, a zirconium oxide nanoparticle, graphene or a graphene oxide nanoparticle.
298. The mitochondrion according to any one of embodiments 281 to 288, wherein the one or more nucleic acid molecule(s), one or more polypeptide(s) and/or one or more drug(s) is linked to a second polypeptide in the outer membrane of the mitochondrion via an amide bond.
299. The mitochondrion according to any one of embodiments 281 to 288 or 298, wherein the one or more nucleic acid molecule(s), one or more polypeptide(s) and/or one or more drug(s) has been modified to undergo formation of the amide bond with an amine function comprised in the second polypeptide in the outer membrane of the mitochondrion.
300. The mitochondrion according to any one of embodiments 281 to 288 or 298, wherein the one or more nucleic acid molecule(s), the one or more polypeptide(s) and/or the one or more drug(s) is encapsulated in a nanoparticle, and wherein the nanoparticle comprises a functional group that allows covalent linkage of the nanoparticle to a second polypeptide in the outer membrane of the mitochondrion.
301. The mitochondrion according to embodiments 281 to 288, wherein the antibody specifically binds to an antigen comprised in the outer membrane of the mitochondrion, wherein the antigen is OPA1, TOM70, TOMM20, Mitofusin 1, Mitofusin 2 or VDAC1.
302. The mitochondrion according to any one of embodiments 281 to 288 or 301, wherein the one or more nucleic acid molecule(s), the one or more polypeptide(s) and/or the one or more drug(s) is encapsulated in a nanoparticle, and wherein the nanoparticle is covalently linked to the antibody.
303. The mitochondrion according to any one of embodiments 281, 282, 284 or 287, wherein the one or more nucleic acid molecule(s), one or more polypeptide(s) and/or one or more anionic drug(s) is electrostatically linked to the antibody, wherein the antibody is a modified antibody, wherein the modified antibody possesses one or more positive charge(s).
304. The mitochondrion according to any one of embodiments 281 to 288 or 301, wherein the one or more nucleic acid molecule(s), one or more polypeptide(s) and/or one or more drug(s) is covalently linked to biotin, wherein biotin is linked to the antibody, wherein the antibody is an avidin conjugated antibody.
305. The mitochondrion according to any one of embodiments 281 to 288 or 301, wherein the one or more nucleic acid molecule(s), one or more polypeptide(s) and/or one or more drug(s) is covalently linked to an activated ester, wherein the activated ester is linked to the antibody via an amide bond.
306. The mitochondrion according to any one of embodiments 281 to 288 or 301, wherein the mitochondrion comprises one or more nucleic acid molecule(s), wherein the one or more nucleic acid molecule(s) is a single-stranded nucleic acid molecule (ssDNA or ssRNA), wherein the single-stranded nucleic acid molecule is hybridized with one or more complementary single-stranded nucleic acid molecule attached on or to the antibody.
307. The mitochondrion according to any one of embodiments 281 to 288 or 301, wherein the one or more nucleic acid molecule(s), one or more polypeptide(s) and/or one or more drug(s) is encapsulated in a nanoparticle, wherein the nanoparticle is electrostatically linked to the antibody, wherein the antibody is a modified antibody, wherein the modified antibody possesses one or more positive charge(s).
308. The mitochondrion according to any one of embodiments 281 to 288 or 302, wherein the one or more nucleic acid molecule(s), one or more polypeptide(s) and/or one or more drug(s) is encapsulated in a nanoparticle, wherein the nanoparticle is covalently linked to biotin, wherein biotin is linked the antibody, wherein the antibody is an avidin conjugated antibody.
309. The mitochondrion according to any one of embodiments 281 to 288 or 302, wherein the one or more nucleic acid molecule(s), one or more polypeptide(s) and/or one or more drug(s) is encapsulated in a nanoparticle, wherein the nanoparticle is covalently linked to an activated ester, wherein the activated ester is linked to the antibody via an amide bond.
310. The mitochondrion according to any one of embodiments 281 to 288, wherein the mitochondria-targeting small molecule is selected from the group consisting of triphenylphosphonium (TPP), dequalinium (DQA), E-4-(1H-Indol-3-ylvinyl)-N-Methylpyridineiodide (F16), Rhodamine 19, biguanidine and guanidine.
311. The mitochondrion according to any one of embodiments 281, 282, 288, wherein the mitochondrion comprises one or more nucleic acid molecule(s) and one or more cationic drug(s), wherein the cationic drug is electrostatically linked to the one or more nucleic acid molecules.
312. The mitochondrion according to any one of embodiments 281 to 311, wherein the mitochondrion is linked to and/or enveloped in a protective layer.
313. The mitochondrion according to embodiment 311, wherein the protective layer is a protective polymer.
314. The mitochondrion according to embodiment 313, wherein the protective polymer is a linear or branched cationic polymer, optionally wherein the linear or branched cationic polymer is electrostatically linked to the one or more nucleic acid molecule(s) and/or the one or more negatively-charged polypeptide(s) and/or the one or more anionic drug(s).
315. The mitochondrion according to embodiment 313, wherein the protective polymer is a linear or branched cationic block copolymer, optionally wherein the linear or branched cationic block copolymer is electrostatically linked to the one or more nucleic acid molecule(s) and/or the one or more negatively-charged polypeptide(s) and/or the one or more anionic drug(s).
316. The mitochondrion according to embodiment 313, wherein protective polymer is a cationic graft (g) copolymer, optionally wherein the cationic graft (g) copolymer is electrostatically linked to the one or more nucleic acid molecule(s) and/or the one or more negatively-charged polypeptide(s) and/or the one or more anionic drug(s).
317. The mitochondrion according to embodiment 313, wherein the protective polymer is a linear or branched pegylated (PEG) cationic polymer, optionally wherein the linear or branched pegylated (PEG) cationic polymer is electrostatically linked to the one or more nucleic acid molecule(s) and/or the one or more negatively-charged polypeptide(s) and/or the one or more anionic drug(s).
318. The mitochondrion according to embodiment 312, wherein the protective layer is a lipid formulation, optionally wherein the lipid formulation is a cationic lipid formulation, further optionally wherein the cationic lipid formulation is electrostatically linked to the one or more nucleic acid molecule(s) and/or the one or more negatively-charged polypeptide(s) and/or the one or more anionic drug(s).
319. The mitochondrion according to any one of embodiments 312 to 318, wherein the protective layer is linked to an antibody, optionally wherein the protective layer linked to an antibody is electrostatically linked to the one or more nucleic acid molecule(s) and/or the one or more negatively-charged polypeptide(s) and/or the one or more anionic drug(s).
320. The mitochondrion according to any one of embodiments 312 to 318, wherein the protective layer is linked to a carbohydrate, optionally wherein the protective layer linked to a carbohydrate is electrostatically linked to the one or more nucleic acid molecule(s) and/or the one or more negatively-charged polypeptide(s) and/or the one or more anionic drug(s).
321. The mitochondrion according to embodiment 314, wherein the linear or branched cationic polymer is polyethyleneimine, RGD-modified polyethyleneimine, polylysine, RGD-modified polylysine, polyornithine, RGD-modified polyornithine, polyarginine, RGD modified polyarginine, polypropyleneimine, RGD-modified polypropyleneimine, polyallylamine, RGD-modified polyallylamine, chitosan, RGD-modified chitosan, poly(2-(dimethylamino)ethyl methacrylate), RGD-modified poly(2-(dimethylamino)ethyl methacrylate), poly(amidoamine)s, RGD-modified poly(amidoamine)s or a combination thereof.
322. The mitochondrion according to embodiment 315, wherein the cationic block copolymer is poly(ethylene glycol)-block-polyethyleneimine, RGD-modified poly(ethylene glycol)-block-polyethyleneimine, poly(ethylene glycol)-block-polylysine, RGD-modified poly(ethylene glycol)-block-polylysine, poly(ethylene glycol)-block-polyornithine, RGD-modified poly(ethylene glycol)-block-polyornithine, poly(ethylene glycol)-block-polyarginine, RGD-modified poly(ethylene glycol)-block-polyarginine, poly(ethylene glycol)-block-polypropyleneimine, RGD-modified poly(ethylene glycol)-block-polypropyleneimine, poly(ethylene glycol)-block-polyallylamine, RGD-modified poly(ethylene glycol)-block-polyallylamine, poly(ethylene glycol)-block-poly(2-(dimethylamino)ethyl methacrylate), RGD-modified poly(ethylene glycol)-block-poly(2-(dimethylamino)ethyl methacrylate), poly(ethylene glycol)-block-poly(amidoamine)s, RGD-modified poly(ethylene glycol)-block-poly(amidoamine)s or a combination thereof.
323. The mitochondrion according to embodiments 316, wherein the cationic graft (g) copolymer is poly(ethylene glycol)-g-polyethyleneimine, RGD-modified poly(ethylene glycol)-g-polyethyleneimine, poly(ethylene glycol)-g-polylysine, RGD-modified poly(ethylene glycol)-g-polylysine, poly(ethylene glycol)-g-polyornithine, RGD-modified poly(ethylene glycol)-g-polyornithine, poly(ethylene glycol)-g-polyarginine, RGD-modified poly(ethylene glycol)-g-polyarginine, poly(ethylene glycol)-g-polypropyleneimine, RGD-modified poly(ethylene glycol)-g-polypropyleneimine, poly(ethylene glycol)-g-polyallylamine, RGD-modified poly(ethylene glycol)-g-polyallylamine, poly(ethylene glycol)-g-poly(2-(dimethylamino)ethyl methacrylate), RGD-modified poly(ethylene glycol)-g-poly(2-(dimethylamino)ethyl methacrylate), poly(ethylene glycol)-g-poly(amidoamine)s, RGD-modified poly(ethylene glycol)-g-poly(amidoamine)s or a combination thereof.
324. The mitochondrion according to embodiments 317, wherein the pegylated (PEG) cationic polymer is pegylated-polyethyleneimine, RGD-modified pegylated polyethyleneimine, pegylated polylysine, RGD-modified pegylated polylysine, histidylated polylysine, pegylated polyornithine, RGD-modified pegylated polyornithine, pegylated polyarginine, RGD-modified pegylated polyarginine, pegylated polypropyleneimine, RGD-modified pegylated polypropyleneimine, pegylated polyallylamine, RGD-modified pegylated polyallylamine, pegylated chitosan, RGD-modified pegylated chitosan, pegylated poly(2-(dimethylamino)ethyl methacrylate), RGD-modified pegylated poly(2-(dimethylamino)ethyl methacrylate), pegylated poly(amidoamine) s RGD-modified pegylated poly(amidoamine)s or a combination thereof.
325. The mitochondrion according to embodiment 318, wherein the lipid formulation comprises DC-cholesterol (3β-[N—(N′,N′-Dimethylaminoethane)-carbamoyl]cholesterol hydrochloride), DLinDMA (1,2-dilinoleyloxy-3-dimethylaminopropane), DLinMC3DMA (dilinoleylmethyl-4-dimethylaminobutyrate), DODMA (1,2-dioleyloxy-3-dimethylaminopropane), DOGS (dioctadecylamidoglycylspermine), DOSPA (2,3-dioleyloxy-N-[2 (sperminecarboxamido)ethyl]-N,N-dimethyl-1-propanaminium), DOTAP (1,2-dioleoyl-3-trimethylammonium-propane chloride), DOTMA (1,2-di-O-octadecenyl-3-trimethylammonium propane chloride), UGG (unsaturated guanidinium glycoside), DOPE (1,2-Dioleoyl-sn-glycerophosphoethanolamine), lipofectamine or a combination thereof.
326. The mitochondrion according to embodiment 325, wherein the lipid formulation further comprises another lipid, preferably wherein said lipid is cholesterol, a substituted or unsubstituted cholesterol, a cholesterol derivative, such as a hydroxylated cholesterol derivative (e.g., a hydroxycholesterol), a PEG-lipid, DMPC (1,2-Dimyristoyl-sn-glycero-3-phosphocholine), DSPC (1,2-Distearoyl-sn-glycero-3-phosphocholine), DODAP (1,2-dioleoyl-3-dimethylammonium propane), DDA (dimethyldioctadecylammonium), 1,2-dioleoyl-sn-glycero-3-phosphate, 1,2-dimyristoyl-sn-glycero-3-phosphate, bis(monooleoylglycerol)phosphate or a combination thereof.
327. The mitochondrion according to embodiment 313, wherein the mitochondrion is linked to and/or enveloped in a zwitterionic protective polymer, optionally wherein the zwitterionic protective polymer is electrostatically linked to the one or more nucleic acid molecule(s).
328. The mitochondrion according to embodiment 327, wherein the zwitterionic protective polymer is selected from: poly(2-methacryloyloxyethyl phosphorylcholine) (PMPC), polyethyleneimine-g-poly(2-methacryloyloxyethyl phosphorylcholine) (PEI-g-PMPC), co-assembly of cationic (carboxyl-functionalized) and anionic (amino-functionalized) copolyesters based on poly(ε-caprolactone)-block-poly(butylene fumarate)-block-poly(ε-caprolactone) (PCL-b-PBF-b-PCL), poly(lactic-co-glycolic acid) (PLGA)-PCB block copolymers (PLGA-b-PCB).
329. A composition comprising a plurality of mitochondria according to any one of embodiments 281 to 328.
330. A pharmaceutical composition comprising a plurality of mitochondria according to any one of embodiments 281 to 328 and a pharmaceutically acceptable carrier.
331. The pharmaceutical composition according to embodiment 330, wherein the pharmaceutical composition is formulated as a solution.
332. The pharmaceutical composition according to embodiment 330, wherein the pharmaceutical composition is formulated as an aerosol.
333. The mitochondrion according to any one of embodiments 281 to 328, the composition according to embodiment 329 or the pharmaceutical composition according to any one of embodiments 330 to 332 for use as a medicament.
334. The mitochondrion according to any one of embodiments 281 to 328, the composition according to embodiment 329 or the pharmaceutical composition according to any one of embodiments 330 to 332 for use in gene therapy.
335. The mitochondrion according to any one of embodiments 281 to 328, the composition according to embodiment 329 or the pharmaceutical composition according to any one of embodiments 330 to 332 for use in the treatment of cardiovascular diseases, in particular for use in the treatment of ischemic heart disease, ischemia-reperfusion injury or atherosclerosis.
336. The mitochondrion according to any one of embodiments 281 to 328, the composition according to embodiment 329 or the pharmaceutical composition according to any one of embodiments 330 to 332 for use in the treatment of aging related diseases, in particular for use in the treatment of sarcopenia, Parkinson's disease or Hutchinson-Gilford progeria syndrome.
337. The mitochondrion according to any one of embodiments 281 to 328, the composition according to embodiment 329 or the pharmaceutical composition according to any one of embodiments 330 to 332 for use in the treatment of cancer.
338. The mitochondrion according to any one of embodiments 281 to 328, the composition according to embodiment 329 or the pharmaceutical composition according to any one of embodiments 330 to 332 for use in in vitro, ex vivo, or in vivo genome editing.
339. The mitochondrion according to any one of embodiments 281 to 328, the composition according to embodiment 329 or the pharmaceutical composition according to any one of embodiments 330 to 332 for use in radiation therapy.
340. A method for attaching two or more of (i) to (iii):
   i) one or more nucleic acid molecule(s);
   ii) one or more polypeptide(s);
   iii) and/or one or more drug(s);
to the outer membrane of a mitochondrion, the method comprising the steps of:
   a) providing a preparation of mitochondria;
   b) contacting the mitochondria provided in step (a) with one or more nucleic acid molecule(s), one or more polypeptide(s) and/or one or more drug(s), optionally in the presence of a positively-charged species; and
   c) attaching the one or more nucleic acid molecule(s), one or more polypeptide(s) and/or one or more drug(s) to the mitochondria, optionally via the positively-charged species.
341. The method according to embodiment 340, wherein
   a) the one or more nucleic acid molecule(s), one or more polypeptide(s) and/or one or more drug(s) are contacted with the positively-charged species to form a positively-charged complex before the positively-charged complex is contacted with the mitochondria; or
   b) the mitochondrion is contacted with the positively-species and subsequently contacted with the one or more nucleic acid molecule(s), one or more polypeptide(s) and/or one or more drug(s).
342. The method according to embodiment 340 or 341, wherein the mitochondria are contacted with one or more nucleic acid molecule(s), one or more polypeptide(s), and/or one or more drug(s) and the positively-charged species in a suitable buffer.
343. The method according to embodiment 342, wherein the buffer comprises or consists of HEPES, EGTA, Trehalose CHES and sodium phosphate dibasic dihydrate, preferably wherein buffer comprises a mixture of a Solution X comprising or consisting of HEPES, EGTA and Trehalose and of a Solution Y comprising or consisting of CHES and sodium phosphate dibasic dihydrate, more preferably, wherein the buffer comprises a 4:1 mixture of Solution X comprising or consisting of 20 mM HEPES, 1 mM EGTA and 300 mM Trehalose (pH 7.2) and Solution Y comprising or consisting of 0.1 M CHES (pH 10) and 0.2 M sodium phosphate dibasic dihydrate.
344. The method according to any one of embodiments 340 to 343, wherein the mitochondria are contacted with the one or more nucleic acid molecule(s), one or more polypeptide(s) and/or one or more drug(s) and the positively-charged species at room temperature for at least 5 minutes, such as at least 10 minutes, 20 or 30 minutes.
345. The method according to any one of embodiments 340 to 344, wherein the mitochondria are contacted with the one or more nucleic acid molecule(s), one or more polypeptide(s) and/or one or more drug(s) and the positively-charged species in the dark.
346. The method according to any one of embodiments 340 to 344, wherein the positively-charged species is a polycationic species, wherein the polycationic species is a linear or branched polycationic polymer, optionally wherein the linear or branched polycationic polymer is electrostatically linked to the one or more nucleic acid molecule(s), one or more polypeptide(s) and/or one or more drug(s).
347. The method according to embodiment 346, wherein the linear or branched polycationic polymer is polylysine, histidylated polylysine, polyornithine, polyarginine, high-mobility group protein (HMG) 1 and 17, modified chitosan, cationized human serum albumin, polyethyleneimine (PEI), polypropyleneimine (PPI), a cationic dendrimer, poly(2-(dimethylamino)ethyl methacrylate) (PDMAEMA), a polyallylamine derivative, diethylaminoethyl (DEAE)-dextran, poly(N-alkyl-4-vinylpyridinium), a poly(amidoamine), cationic gelatin, cationic cellulose or a combination thereof.
348. The method according to any one of embodiments 340 to 345, wherein the positively-charged species is a positively-charged nanoparticle.
349. The method according to embodiment 348, wherein the method comprises a further step of
   a) attaching the one or more nucleic acid molecule(s), one or more polypeptide(s) and/or one or more drug(s) to the surface of the positively-charged nanoparticle; or
   b) encapsulating the one or more nucleic acid molecule(s), one or more polypeptide(s) and/or one or more drug(s) within the positively-charged nanoparticle.
350. The method according to embodiment 348 or 349, wherein the positively-charged nanoparticle is a lipid nanoparticle, a dendrimer nanoparticle, a micelle nanoparticle, a protein nanoparticle, a liposome, a non-porous silica nanoparticle, a mesoporous silica nanoparticle, a silicon nanoparticle, a gold nanoparticle, a gold nanowire, a silver nanoparticle, a platinum nanoparticle, a palladium nanoparticle, a titanium dioxide nanoparticle, a carbon nanotube, a carbon dot nanoparticle, a polymer nanoparticle, a zeolite nanoparticle, an aluminium oxide nanoparticle, a hydroxyapatite nanoparticle, a quantum dot nanoparticle, a zinc oxide nanoparticle, a zirconium oxide nanoparticle, graphene or a graphene oxide nanoparticle.
351. A method for covalently attaching two or more of (i) to (iii):
   i) one or more nucleic acid molecule(s);
   ii) one or more polypeptide(s);
   iii) and/or one or more drug(s);
to the outer membrane of a mitochondrion, the method comprising the steps of:
   a) providing a preparation of mitochondria;
   b) providing one or more nucleic acid molecule(s), one or more polypeptide(s) and/or one or more drug(s) that have been modified to comprise an activated ester; and
   c) attaching the one or more nucleic acid molecule(s), one or more polypeptide(s) and/or one or more drug(s) provided in step (b) to an amine comprised in a polypeptide in the outer membrane of the mitochondria.
352. The method according to embodiment 351, wherein the activated ester is an N-hydroxysuccinimide (NHS) ester.
353. A method for covalently attaching two or more of (i) to (iii):
   i) one or more nucleic acid molecule(s);
   ii) one or more polypeptide(s);
   iii) and/or one or more drug(s);
to the outer membrane of a mitochondrion, the method comprising the steps of:
   a) providing a preparation of mitochondria;
   b) encapsulating one or more nucleic acid molecule(s), one or more polypeptide(s) and/or one or more drug(s) in a nanoparticle, wherein the surface of the nanoparticle comprises an activated ester; and
   c) attaching the nanoparticle provided in step (b) to an amine comprised in a polypeptide in the outer membrane of the mitochondria.
354. The method according to embodiment 353, wherein the activated ester is an NHS ester.
355. The mitochondrion according to any one of embodiments 211 to 252, the composition according to item 253 or the pharmaceutical composition according to any one of items 253 to 255 for use in the treatment of kidney diseases, in particular for use in the treatment of autosomal dominant polycystic kidney disease, Alport syndrome, Nephronophthisis, or Fabry disease.

What is claimed is:

1.-79. (canceled)

80. A mitochondrion comprising one or more payload(s) attached to the outer membrane of the mitochondrion, wherein the payload(s) is indirectly electrostatically attached to the outer membrane of the mitochondrion.

81. The mitochondrion of claim 80, wherein the payload is one or more of:
i) a nucleic acid molecule;
ii) a polypeptide;
iii) a drug; or
iv) a combination of one or more of (i) to (iii).

82. The mitochondrion of claim 80 or 81, wherein the payload is charged.

83. The mitochondrion of any one of claims 80 to 82, wherein the payload has the same net charge as the net charge of the mitochondrion.

84. The mitochondrion of claim 83, wherein the payload and mitochondrion both have a net negative charge and wherein the payload is attached to the mitochondrion via a positively-charged species.

85. The mitochondrion of claim 84, wherein the positively-charged species is a polycationic species.

86. The mitochondrion of claim 85, wherein the polycationic species is a linear or branched polycationic polymer.

87. The mitochondrion according to claim 86, wherein the linear or branched polycationic polymer is polylysine, histidylated polylysine, polyornithine, polyarginine, high-mobility group protein (HMG) 1 and 17, modified chitosan, cationized human serum albumin, polyethyleneimine (PEI), polypropyleneimine (PPI), a cationic dendrimer, poly(2-(dimethylamino)ethyl methacrylate) (PDMAEMA), a polyallylamine derivative, diethylaminoethyl (DEAE)-dextran, poly(N-alkyl-4-vinylpyridinium), a poly(amidoamine), cationic gelatin, cationic cellulose or a combination thereof.

88. The mitochondrion of claim 84, wherein the positively-charged species is a positively-charged nanoparticle or a positively-charged particle.

89. The mitochondrion of claim 88, wherein the one or more nucleic acid molecule(s) is attached to the surface of the positively-charged nanoparticle or to the positively-charged particle; or encapsulated in the positively-charged nanoparticle or in the positively-charged particle.

90. The mitochondrion of any one of claims 88 to 89, wherein the positively-charged nanoparticle and/or particle is a lipid nanoparticle/particle, a dendrimer nanoparticle/particle, a micelle nanoparticle/particle, a protein nanoparticle/particle, a liposome, a non-porous silica nanoparticle/particle, a mesoporous silica nanoparticle/particle, a silicon nanoparticle/particle, a gold nanoparticle/particle, a gold nanowire, a silver nanoparticle/particle, a platinum nanoparticle/particle, a palladium nanoparticle/particle, a titanium dioxide nanoparticle/particle, a carbon nanotube, a carbon dot nanoparticle/particle, a polymer nanoparticle/particle, a zeolite nanoparticle/particle, an aluminium oxide nanoparticle/particle, a hydroxyapatite nanoparticle/particle, a quantum dot nanoparticle/particle, a zinc oxide nanoparticle/particle, a zirconium oxide nanoparticle/particle, graphene or a graphene oxide nanoparticle/particle.

91. The mitochondrion of any one of claims 80 to 82, wherein the payload has a different net charge as the net charge of the mitochondrion.

92. The mitochondrion of claim 91, wherein the payload and the mitochondrion are attached via a zwitterionic species.

93. The mitochondrion of claim 91, wherein the payload is uncharged and wherein the payload is attached to a positively-charged species.

94. The mitochondrion of claim 93, wherein the positively-charged species is as defined in any one of claims 85 to 90.

95. The mitochondrion of claim 81 wherein the one or more nucleic acid molecule(s) is
(i) electrostatically linked to an antibody, optionally wherein the antibody is a modified antibody, optionally wherein the modified antibody possesses one or more positive charges; or
(ii) encapsulated in a nanoparticle, wherein the nanoparticle is electrostatically linked to an antibody, optionally wherein the antibody is a modified antibody, optionally wherein the modified antibody possesses one or more positive charges.

96. The mitochondrion according to claim 95 (i) or (ii), wherein the antibody specifically binds to an antigen comprised in the outer membrane of the mitochondrion, wherein the antigen is OPA1, TOM70, TOMM20, Mitofusin 1, Mitofusin 2 or VDAC1.

97. The mitochondrion of any one of claims 80 to 96, wherein the mitochondrion is linked to and/or enveloped in a protective layer.

98. The mitochondrion of claim 97, wherein the protective layer is a protective polymer.

99. The mitochondrion of claim 98, wherein the protective polymer is
(i) a linear or branched cationic polymer, optionally wherein the linear or branched cationic polymer is electrostatically linked to the one or more payload(s);
(ii) a linear or branched cationic block copolymer, optionally wherein the linear or branched cationic block copolymer is electrostatically linked to the one or more payload(s);
(iii) a cationic graft (g) copolymer, optionally wherein the cationic graft (g) copolymer is electrostatically linked to the one or more payload(s); or
(iv) a linear or branched pegylated (PEG) cationic polymer, optionally wherein the linear or branched pegylated (PEG) cationic polymer is electrostatically linked to the one or more payload(s).

100. The mitochondrion of claim 97, wherein the protective layer is a lipid formulation, optionally wherein the lipid formulation is a cationic lipid formulation, further optionally wherein the cationic lipid formulation is electrostatically linked to the one or more payload(s).

101. The mitochondrion of any one of claims 97 to 100, wherein the protective layer is linked to a targeting moiety.

102. The mitochondrion of any one of claims 97 to 101, wherein the protective layer is linked to an antibody, optionally wherein the protective layer linked to an antibody is electrostatically linked to the one or more payload(s); or a carbohydrate, optionally wherein the protective layer linked to a carbohydrate is electrostatically linked to the one or more payload(s).

103. The mitochondrion of claim 99, wherein
(i) the linear or branched cationic polymer is polyethyleneimine, RGD-modified polyethyleneimine, polylysine, RGD-modified polylysine, polyornithine, RGD-modified polyornithine, polyarginine, RGD modified polyarginine, polypropyleneimine, RGD-modified polypropyleneimine, polyallylamine, RGD-modified polyallylamine, chitosan, RGD-modified chitosan, poly(2-(dimethylamino)ethyl methacrylate), RGD-modified poly(2-(dimethylamino)ethyl methacrylate), poly(amidoamine)s, RGD-modified poly(amidoamine)s or a combination thereof;
(ii) the cationic block copolymer is poly(ethylene glycol)-block-polyethyleneimine, RGD-modified poly(ethylene glycol)-block-polyethyleneimine, poly(ethylene glycol)-block-polylysine, RGD-modified poly(ethylene glycol)-block-polylysine, poly(ethylene glycol)-block-polyornithine, RGD-modified poly(ethylene glycol)-block-polyornithine, poly(ethylene glycol)-block-polyarginine, RGD-modified poly(ethylene glycol)-block-polyarginine, poly(ethylene glycol)-block-polypropyleneimine, RGD-modified poly(ethylene glycol)-block-polypropyleneimine, poly(ethylene glycol)-block-polyallylamine, RGD-modified poly(ethylene glycol)-block-polyallylamine, poly(ethylene glycol)-block-poly(2-(dimethylamino)ethyl methacrylate), RGD-modified poly(ethylene glycol)-block-poly(2-(dimethylamino)ethyl methacrylate), poly(ethylene glycol)-block-poly(amidoamine)s, RGD-modified poly(ethylene glycol)-block-poly(amidoamine)s or a combination thereof;
(iii) the cationic graft (g) copolymer is poly(ethylene glycol)-g-polyethyleneimine, RGD-modified poly(ethylene glycol)-g-polyethyleneimine, poly(ethylene glycol)-g-polylysine, RGD-modified poly(ethylene glycol)-g-polylysine, poly(ethylene glycol)-g-polyornithine, RGD-modified poly(ethylene glycol)-g-polyornithine, poly(ethylene glycol)-g-polyarginine, RGD-modified poly(ethylene glycol)-g-polyarginine, poly(ethylene glycol)-g-polypropyleneimine, RGD-modified poly(ethylene glycol)-g-polypropyleneimine, poly(ethylene glycol)-g-polyallylamine, RGD-modified poly(ethylene glycol)-g-polyallylamine, poly(ethylene glycol)-g-poly(2-(dimethylamino)ethyl methacrylate), RGD-modified poly(ethylene glycol)-g-poly(2-(dimethylamino)ethyl methacrylate), poly(ethylene glycol)-g-poly(amidoamine)s, RGD-modified poly(ethylene glycol)-g-poly(amidoamine)s or a combination thereof; or
(iv) the pegylated (PEG) cationic polymer is pegylated-polyethyleneimine, RGD-modified pegylated polyethyleneimine, pegylated polylysine, RGD-modified pegylated polylysine, histidylated polylysine, pegylated polyornithine, RGD-modified pegylated polyornithine, pegylated polyarginine, RGD-modified pegylated polyarginine, pegylated polypropyleneimine, RGD-modified pegylated polypropyleneimine, pegylated polyallylamine, RGD-modified pegylated polyallylamine, pegylated chitosan, RGD-modified pegylated chitosan, pegylated poly(2-(dimethylamino)ethyl methacrylate), RGD-modified pegylated poly(2-(dimethylamino)ethyl methacrylate), pegylated poly(amidoamine)s RGD-modified pegylated poly(amidoamine)s or a combination thereof.

104. The mitochondrion of claim 100, wherein the lipid formulation comprises DC-cholesterol (3β-[N—(N′,N′-Dimethylaminoethane)-carbamoyl]cholesterol hydrochloride), DLinDMA (1,2-dilinoleyloxy-3-dimethylaminopropane), DLinMC3DMA (dilinoleylmethyl-4-dimethylaminobutyrate), DODMA (1,2-dioleyloxy-3-dimethylaminopropane), DOGS (dioctadecylamidoglycylspermine), DOSPA (2,3-dioleyloxy-N-[2 (sperminecarboxamido)ethyl]-N,N-dimethyl-1-propanaminium), DOTAP (1,2-dioleoyl-3-trimethylammonium-propane chloride), DOTMA (1,2-di-Octadecenyl-3-trimethylammonium propane chloride), UGG (unsaturated guanidinium glycoside), DOPE (1,2-Dioleoyl-sn-glycerophosphoethanolamine), lipofectamine or a combination thereof.

105. The mitochondrion of claim 104, wherein the lipid formulation further comprises another lipid, preferably wherein said lipid is cholesterol, a substituted or unsubstituted cholesterol, a cholesterol derivative, such as a hydroxylated cholesterol derivative (e.g., a hydroxycholesterol), a PEG-lipid, DMPC (1,2-Dimyristoyl-sn-glycero-3-phosphocholine), DSPC (1,2-Distearoyl-sn-glycero-3-phosphocholine), DODAP (1,2-Dioleoyl-3-trimethylammonium propane), DDA (dimethyldioctadecylammonium), 1,2-dioleoyl-sn-glycero-3-phosphate, 1,2-dimyristoyl-sn-glycero-3-phosphate, bis(monooleoylglycerol)phosphate or a combination thereof.

106. The mitochondrion of claim 98, wherein the mitochondrion is linked to and/or enveloped in a zwitterionic protective polymer, optionally wherein the zwitterionic protective polymer is electrostatically linked to the one or more payload(s).

107. The mitochondrion of claim 106, wherein the zwitterionic protective polymer is selected from: poly(2-methacryloyloxyethyl phosphorylcholine) (PMPC), polyethyleneimine-g-poly(2-methacryloyloxyethyl phosphorylcholine) (PEI-g-PMPC), co-assembly of cationic (carboxyl-functionalized) and anionic (amino-functionalized) copolyesters based on poly(ε-caprolactone)-block-poly(butylene fumarate)-block-poly(ε-caprolactone) (PCL-b-PBF-b-PCL), poly(lactic-co-glycolic acid) (PLGA)-PCB block copolymers (PLGA-b-PCB).

108. A composition comprising a plurality of mitochondria according to any one of claims 80 to 107.

109. A pharmaceutical composition comprising a plurality of mitochondria according to any one of claims 80 to 107 and a pharmaceutically acceptable carrier.

110. The pharmaceutical composition of claim 109, wherein the pharmaceutical composition is formulated as a solution or as an aerosol.

111. The mitochondrion of any one of claims 80 to 107, the composition according to claim 108 or the pharmaceutical composition according to any one of claims 109 to 110 for use as a medicament.

112. The mitochondrion of any one of claims 80 to 107, the composition according to claim 108 or the pharmaceutical composition according to any one of claims 109 to 110 for use in gene therapy.

113. The mitochondrion according to any one of claims 80 to 107, the composition according to claim 108 or the pharmaceutical composition according to any one of claims 109 to 110 for use in the treatment of:
(i) cardiovascular diseases, in particular for use in the treatment of ischemic heart disease, ischemia-reperfusion injury, or atherosclerosis;
(ii) aging related diseases, in particular for use in the treatment of, sarcopenia, Parkinson's disease or Hutchinson-Gilford progeria syndrome;
(iii) kidney diseases, in particular for use in the treatment of autosomal dominant polycystic kidney disease, Alport syndrome, Nephronophthisis, or Fabry disease; or
(iv) cancer.

114. The mitochondrion according to any one of claims 80 to 107, the composition according to claim 108 or the pharmaceutical composition according to any one of claims 109 to 110 for use in in vitro, ex vivo, or in vivo genome editing.

115. The mitochondrion according to any one of claims 80 to 107, the composition according to claim 108 or the pharmaceutical composition according to any one of claims 109 to 111 for use in radiation therapy.

116. A method for delivering a payload to a target organ, the method comprising a step of administering the pharmaceutical composition according to any one of claims 109 to 110 into the bloodstream of a subject in need, wherein the pharmaceutical composition is administered into the bloodstream upstream of the target organ.

117. A method for delivering a payload to the lung, the method comprising a step of administering the pharmaceutical composition according to claim 110 to a subject in need, wherein the pharmaceutical composition is administered by inhalation.

118. A method for attaching a payload to the outer membrane of a mitochondrion, the method comprising the steps of:
a) providing a preparation of mitochondria;
b) contacting the mitochondria provided in step (a) with at least one payload in the presence of a positively-charged species; and
c) indirectly electrostatically attaching the at least one payload to the mitochondria via the positively-charged species.

119. The method of claim 118, wherein
a) the at least one payload is simultaneously contacted with the positively-charged species and the mitochondria;
b) the at least one payload is contacted with the positively-charged species to form a positively-charged complex before the positively-charged complex is contacted with the mitochondria; or
c) the mitochondrion is contacted with the positively-charged species and subsequently contacted with the at least one payload.

120. The method of claim 117 or 119, wherein the mitochondria are contacted with the at least one payload and the positively-charged species in a suitable buffer.

121. The method of claim 120, wherein the buffer comprises or consists of HEPES, EGTA, Trehalose, CHES and sodium phosphate dibasic dihydrate, preferably wherein buffer comprises a mixture of a Solution X comprising or consisting of HEPES, EGTA and Trehalose and of a Solution Y comprising or consisting of CHES and sodium phosphate dibasic dihydrate, more preferably, wherein the buffer comprises a 4:1 mixture of Solution X comprising or consisting of 20 mM HEPES, 1 mM EGTA and 300 mM Trehalose (pH 7.2) and Solution Y comprising or consisting of 0.1 M CHES (pH 10) and 0.2 M sodium phosphate dibasic dihydrate.

122. The method of any one of claims 118 to 121, wherein the mitochondria are contacted with the at least one payload, and the positively-charged species at room temperature for at least 5 minutes, such as at least 10 minutes, 20, 30, 40, 50, 60 or 120 minutes.

123. The method of any one of claims 118 to 122, wherein the mitochondria are contacted with the at least one payload and the positively-charged species in the dark.

124. The method of any one of claims 118 to 123, wherein the payload is a nucleic acid molecule which is DNA or RNA.

125. The method of any one of claims 118 to 124, wherein the positively-charged species is a polycationic species, wherein the polycationic species is a linear or branched polycationic polymer, optionally wherein the linear or branched polycationic polymer is electrostatically linked to the at least one payload(s).

126. The method of claim 125, wherein the linear or branched polycationic polymer is polylysine, histidylated polylysine, polyornithine, polyarginine, high-mobility group protein (HMG) 1 and 17, a modified chitosan, cationized human serum albumin, polyethyleneimine (PEI), polypropyleneimine (PPI), a cationic dendrimer, poly(2-(dimethylamino)ethyl methacrylate) (PDMAEMA), a polyallylamine derivative, diethylaminoethyl (DEAE)-dextran poly(N-alkyl-4-vinylpyridinium), a poly(amidoamine), cationic gelatin, cationic cellulose or a combination thereof.

127. The method of any one of claims 118 to 124, wherein the positively-charged species is a positively-charged nanoparticle.

128. The method of claim 127, wherein the method comprises a further step of
a) attaching the at least one payload to the surface of the positively-charged nanoparticle; or
b) encapsulating the at least one payload within the positively-charged nanoparticle.

129. The method of claim 127 or 128, wherein the positively-charged nanoparticle is a lipid nanoparticle, a dendrimer nanoparticle, a micelle nanoparticle, a protein nanoparticle, a liposome, a non-porous silica nanoparticle, a mesoporous silica nanoparticle, a silicon nanoparticle, a gold nanoparticle, a gold nanowire, a silver nanoparticle, a platinum nanoparticle, a palladium nanoparticle, a titanium dioxide nanoparticle, a carbon nanotube, a carbon dot nanoparticle, a polymer nanoparticle, a zeolite nanoparticle, an aluminium oxide nanoparticle, a hydroxyapatite nanoparticle, a quantum dot nanoparticle, a zinc oxide nanoparticle, a zirconium oxide nanoparticle, graphene or a graphene oxide nanoparticle.

130. A method for preparing a mitochondrion comprising a payload, wherein the method comprises the steps of:
a) providing a preparation of mitochondria;
b) contacting the mitochondria with
i) a positively-charged species if the payload and mitochondrion both have a net negative charge;
ii) the payload if the payload has a different net charge as the net charge of the mitochondrion, optionally further a zwitterionic species; or
iii) a payload attached to a positively-charged species if the payload is uncharged;
c) obtaining mitochondria according to any one of claims 80 to 96.

131. The method of claim 130 further comprising subsequent to step c) a step of contacting the mitochondria with components to form a protective layer, and a step of obtaining mitochondria according to any one of claims 97 to 107.

132. The method of any one of claims 118 to 131, wherein an amount of 50 μg to 200 μg of mitochondria are contacted with 0.1 to 50 μmol of the payload and 0.02 to 10 μg, preferably 0.02 to 5 μg, of the positively-charged species.

133. The method according to any one of claims 118 to 131, wherein the mitochondrion comprises a positively-charged species, wherein the positively-charged species is a polycationic polymer according to any of the previous claims, and wherein the ratio of the polycationic polymer to the protective layer is about 1:2.

134. The method according to any of the previous method claims, wherein 50 μg to 200 μg of mitochondria are contacted with 0.1 to 50 μmol of payload and 0.2 to 10 μg of the protective layer.

135. A method for delivering a payload
to the kidney, the method comprising a step of administering the pharmaceutical composition according to claims 109 to 115 into the renal artery of a subject in need;
to the heart, the method comprising a step of administering the pharmaceutical composition according to claims 109 to 115 into the intracoronary of a subject in need;
to the liver, the method comprising a step of administering the pharmaceutical composition according to claims 109 to 115 into the hepatic artery or portal vein of a subject in need;
to the pancreas, the method comprising a step of administering the pharmaceutical composition according to claims 109 to 115 into the hepatic artery of a subject in need;
to the duodenum, the method comprising a step of administering the pharmaceutical composition according to claims 109 to 115 into the hepatic artery of a subject in need;
to the spleen, the method comprising a step of administering the pharmaceutical composition according to claims 109 to 115 into the splenic artery of a subject in need;
to the lung, the method comprising a step of administering the pharmaceutical composition according to claims 109 to 115 into the pulmonary artery of a subject in need;
to the intestines, the method comprising a step of administering the pharmaceutical composition according to claims 109 to 115 into the superior mesenteric artery of a subject in need; or
to the bladder, the method comprising a step of administering the pharmaceutical composition according to claims 109 to 115 into the superior and inferior vesical arteries of a subject in need.

136. A method for delivering a payload to a target organ, the method comprising a step of administering the pharmaceutical composition according to claims 109 to 115 into a subject in need, wherein the pharmaceutical composition is administered into the kidney or bladder or intestines or pancreas or duodenum or liver or lung or spleen through direct injection.
